(19) United States
(12) Patent Application Publication
(10) Pub. No.: US 2018/0221365 A1
(43) Pub. Date: Aug. 9, 2018

(54) NOVEL SIGMA-2 RECEPTOR BINDERS AND THEIR METHOD OF USE

(71) Applicant: Temple University-Of The Commonwealth System of Higher Education, Philadelphia, PA (US)

(72) Inventors: Daniel J. Canney, Ambler, PA (US); Benjamin E. Blass, Eagleville, PA (US); Rong Gao, Roscoe, IL (US); Kevin Blattner, Folsom, PA (US)

(21) Appl. No.: 15/573,504

(22) PCT Filed: May 11, 2016
(86) PCT No.: PCT/US2016/031780

§ 371 Date: Nov. 13, 2017

Related U.S. Application Data
(60) Provisional application No. 62/160,355, filed on May 12, 2015.

(51) Int. Cl. A61K 31/496; A61K 31/4709; A61K 31/4525; A61K 31/4725; A61P 35/00

(52) U.S. Cl. CPC A61K 31/496; A61K 31/4709; A61P 35/00; A61K 31/4725; A61K 31/4525

(57) ABSTRACT

Pharmaceutical compositions of the invention comprise functionalized lactone derivatives having a disease-modifying action in the treatment of diseases associated with dysregulation of sigma-2 receptor activity.

### CROSS-REFERENCE TO RELATED APPLICATIONS

This application claims priority from U.S. Provisional Application Ser. No. 62/160,355, filed May 12, 2015, the contents of which are incorporated by reference herein in its entirety.

### STATEMENT REGARDING FEDERALLY SPONSORED RESEARCH OR DEVELOPMENT

This invention was made with U.S. government support under Grant No. HHSN-271-2008-00025-C awarded by the National Institute of Mental Health. The U.S. government has certain rights in the invention.

### FIELD OF INVENTION

Embodiments of the invention are directed to novel compounds useful as sigma-2 receptor binders and their method of use. Embodiments are further directed to a novel chemotype useful for the treatment diseases that are associated with dysregulation of sigma-2 receptor activity.

### BACKGROUND OF THE INVENTION

The sigma-1 and sigma-2 receptors were first identified in the mid-1970s based on their interaction with radioligands. In 1976, a study of the physiological properties of (+)-SKF-10,047 (N-allylnormetazocine) and its structurally related benzomorphan analogues, morphine and ketazocine, in the chronic spinal dog model identified three receptor sub-types, the μ-opioid recptor, the κ-opioid receptor, and the o-receptor (sigma receptor) (Martin, W. R.; Eades, C. G.; Thompson, J. A.; Huppler, R. E.; Gilbert, P. E. The effects of morphine- and nalorphine-like drugs in the nondependent and morphine-dependent chronic spinal dog. J. Pharmacol. Exp. Ther. 1976, 197, 517-532). It was subsequently determined that (−)-SKF-10,047 binds to the μ-opioid recptor and the κ-opioid receptor, while (+)-SKF-10,047 binds selectively to the σ-receptor (sigma receptor), although the true function of the σ-receptor remained unknown (Matsumoto, R. R. Sigma Receptors: Historical Perspective and Background. In Sigma Receptors: Chemistry, Cell Biology and Clinical Implications; Matsumoto, R. R., Bowen, W. D., Su, T.-P., Eds.; Springer Science: New York, N.Y., 2007, pp 1-23; Collier, T. L.; Waterhouse, R. N.; Kassiou, M. Imaging sigma receptors: applications in drug development. Curr. Pharm. Des. 2007, 13, 51-72.) The availability of the σ-receptor selective radioligand [³H]o-ditolylguanidine (DTG) facilitated more detailed binding studies of ligand for the σ-receptor, and eventually lead to the identification of two distinct subtypes, the σ₁-receptor and the σ₂-receptor (Hellewell, S. B.; Bowen, W. D. A sigma-like binding site in rat pheochromocytoma (PC12) cells: decreased affinity for (+)-benzomorphans and lower molecular weight suggest a different sigma receptor form from that of guinea pig brain. Brain Res. 1990, 527, 244-253) Although the exact structure of the σ₂-receptor is unknown, recent photoaffinity labeling studies have suggested that the σ₂-receptor is synonymous with the progesterone receptor membrane component-1 (PGRMC1) (Xu, J. et al. Identification of the PGRMC1 protein complex as the putative sigma-2 receptor binding site. Nat. Commun. 2, 380, 2011).

The therapeutic utility of compounds capable of binding to the σ₂-receptor or modulating activity of the σ₂-receptor has also been explored. It has recently been discovered, for example, that compounds capable of binding to the σ₂-receptor can prevent the binding of beta amyloid protein (Aβ) oligomers to neurons, thereby preventing downstream synaptotoxicity. This aspect of σ₂-receptor binders provides an opportunity for the application of σ₂-receptor binders as treatment for Alzheimer's disease, mild cognitive impairments, and memory disorders. It has further been demonstrated that compounds capable of binding to the σ₂-receptor can displace beta amyloid protein (Aβ) oligomers from neurons, thereby preventing downstream synaptotoxicity. This aspect of σ₂-receptor binders also provides an opportunity for the application of σ₂-receptor binders as treatment for Alzheimer's disease, mild cognitive impairments, and memory disorders (Izzo, N. J. et al. Alzheimer's therapeutics targeting amyloid Beta 1-42 oligomers I: abeta 42 oligomer binding to specific neuronal receptors is displaced by drug candidates that improve cognitive deficits. PLoS One 9, e11111898, 2014; Izzo, N. J. et al. Alzheimer's Therapeutics Targeting Amyloid Beta 1-42 Oligomers II: Sigma-2/PGRMC1 Receptors Mediate Abeta 42 Oligomer Binding and Synaptotoxicity. PLoS One 9, e111899, 2014).

Separately, it has been demonstrated that expression of the σ₂-receptor is elevated in tumor cells as compared with normal cells. Cancer cells in which overexpression of the σ₂-receptor occurs, but is not limited to, pancreatic cancer, lung cancer, breast cancer, melanoma, prostate cancer, and ovarian cancer. It has been further discovered that compounds capable of binding to the σ₂-receptor modulate its activity and induce cancer cell death. As such, the σ₂-receptor is a viable target for the identification of anti-cancer agents, and compounds capable of binding to the σ₂-receptor represent an opportunity to develop new anti-cacer agents.

The dysregulation of sigma-2 receptor activity has also been impacted in a number of neuropsychiatric disorders including but not limited to generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia (Guo, L.; Zhen, X. Simga-2 Receptor ligands: Neurobiological effects. Current Medicincal Chemistry, 2015, 22, 8, 989-1003; Skuza, G. Pharmacology of sigma (o) receptor ligands from a behavioral perspective. Current Pharmaceutical Design, 2012, 18, 7, 863-874). As such, the σ₂-receptor is a viable target for the treatment of neuropsychiatric disorders including but not limited to generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia. Compounds that bind to the σ₂-receptor that are capable of modulating σ₂-receptor represent an opportunity to identify new treatments for a number of neuropsychiatric disorders including but not limited to generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia.

### BRIEF SUMMARY OF THE INVENTION

The present invention is directed toward novel sigma-2 receptor binders, compounds of formula (I),

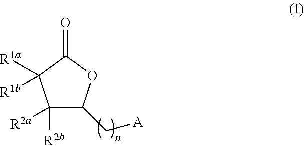

including hydrates, solvates, pharmaceutically acceptable salts, prodrugs and complexes thereof, wherein:

A is selected from the group consisting of

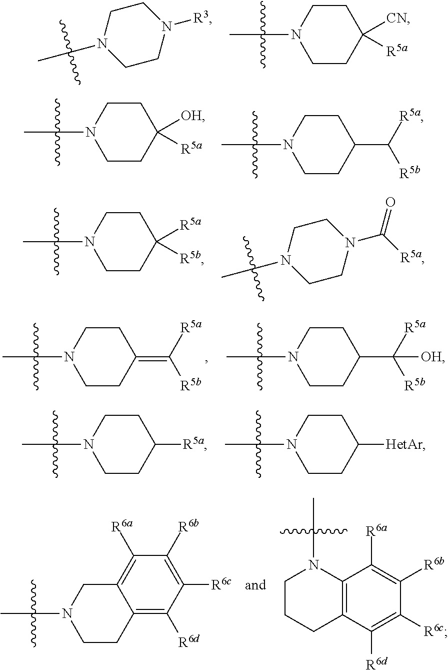

R^{1a} and R^{1b} are each independently selected from the group consisting of hydrogen, C₁₋₆ linear alkyl, C₁₋₆ branched alkyl, and optionally substituted aryl, or R^{1a} and R^{1b} may be taken together with the atom to which they are bound to form a ring having from 3 to 7 ring atoms;

R^{2a} and R^{2b} are each independently selected from the group consisting of hydrogen, C₁₋₆ linear alkyl, C₁₋₆ branched alkyl, and optionally substituted aryl, or R^{2a} and R^{2b} may be taken together with the atom to which they are bound to form a ring having from 3 to 7 ring atoms;

R³ is selected from the group consisting of C₁₋₆ linear alkyl, C₁₋₆ branched alkyl, C₃₋₇ cycloalkyl, optionally substituted aryl, optionally substituted heteroaryl,

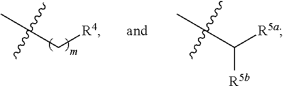

R⁴ is optionally substituted aryl;

R^{5a} and R^{5b} are each independently optionally substituted aryl;

R^{6a}, R^{6b}, R^{6c}, and R^{6d} are each independently selected from the group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁷, C(O)NHR⁷, C(O)N(R⁷)₂, SH, SC₁₋₆ alkyl, SO²NH₂, SO₂NHR⁷, SO₂R⁷, and NHSO₂R⁷;

Each occurrence of R⁷ is independently selected from the group consisting of C₁₋₆ linear alkyl, C₁₋₆ branched alkyl, and C₃₋₇ cycloalkyl;

HetAr is optionally substituted heteroaryl;

n is 1, 2, or 3; and

m is 1 or 2.

The present invention further relates to compositions comprising:

an effective amount of one or more compounds according to the present invention and an excipient.

The present invention also relates to a method for treating or preventing diseases that involve dysregulation of sigma-2 receptor activity. The method comprises administering to a subject an effective amount of a compound or composition according to the present invention.

The present invention further relates to a process for preparing the sigma-2 receptor binders modulators of the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

There is evidence that suggests a role for the sigma-2 receptor in a number of disease states including, but not limited to neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders. Sigma-2 receptor activity modulators are likely to have a beneficial effect on patients suffering from these diseases and disorders. The disorders in which sigma-2 receptor dysregulation plays a role and modulation of sigma-2 receptor receptor activity by a therapeutic agent may be a viable approach to therapeutic relief include, but are not limited to, neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders.

There is a long felt need for new sigma-2 receptor binders and sigma-2 receptor activity modulators that will provide therapeutic relief from patients suffering from diseases associated with dysregulation of the sigma-2 receptor. The invention addresses the need to identify novel sigma-2 receptor binders and sigma-2 receptor activity modulators capable of treating disease associated with dysregulation of sigma-2 receptor activity. The present invention addresses the need to develop new therapeutic agents for the treatment and prevention of neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders.

The sigma-2 receptor binders and sigma-2 receptor activity modulators of the present invention are capable of treating and preventing diseases associated with dysregulation of the sigma-2 receptor, for example neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders. Without wishing to be limited by theory, it is believed that the sigma-2 receptor binders and sigma-2 receptor activity modulators of the present invention can ameliorate, abate, or otherwise cause to be controlled, diseases and disorders associated with dysregulation of the sigma-2 receptor. The diseases and disorders include, but are not limited to neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders.

The sigma-2 receptor binders and sigma-2 receptor activity modulators of the present invention are also capable of treating and preventing diseases associated with overexpression of the sigma-2 receptor, for example neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders. Without wishing to be limited by theory, it is believed that the sigma-2 receptor binders and sigma-2 receptor activity modulators of the present invention can ameliorate, abate, otherwise cause to be controlled, diseases and disorders associated with overexpression of the sigma-2 receptor. The diseases and disorders include, but are not limited to neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, and schizophrenia, cancers such as pancreatic cancer, lung cancer, breast cancer, melanoma, prostate cancer, and ovarian cancer, as well as Alzheimer's disease, mild cognitive impairments, and memory disorders.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bonds. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “haloalkoxy” refers to the group —O-haloalkyl, wherein the haloalkyl group is as defined above. Examples of haloalkoxy groups include, but are not limited to, fluoromethoxy, difluoromethoxy, trifluoromethoxy, and pentafluoroethoxyl.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl, 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, 1H-benzo[d]imidazol-2(3H)-onyl, 1H-benzo[d]imidazolyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

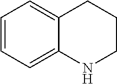

is, for the purposes of the present invention, considered a heterocyclic unit. 6,7-Dihydro-5H-cyclopentapyrimidine having the formula:

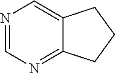

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

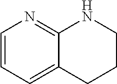

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine (I)), —CN, —NO₂, oxo (═O), —OR⁸, —SR⁸, —N(R⁸)₂, —NR⁸C(O)R⁸, —SO₂R⁸, —SO₂OR⁸, —SO₂N(R⁸)₂, —C(O)R⁸, —C(O)OR⁸, —C(O)N(R⁸)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, Oxo, and R⁸; wherein R⁸, at each occurrence, independently is hydrogen, —OR⁹, —SR⁹, —C(O)R⁹, —C(O)OR⁹, —C(O)N(R⁹)₂, —SO₂R⁹, —S(O)₂OR⁹, —N(R⁹)₂, —NR⁹C(O)R⁹, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁸ units taken together with the atom(s) to which they are bound form an optionally substituted carbocycle or heterocycle wherein said carbocycle or heterocycle has 3 to 7 ring atoms; wherein R⁹, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁹ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹⁰; for example, —OH, —OCH₃, —OCH₂CH₃, —OCH₂CH₂CH₃;
ii) —C(O)R¹⁰; for example, —COCH₃, —COCH₂CH₃, —COCH₂CH₂CH₃;
iii) —C(O)OR¹⁰; for example, —CO₂CH₃, —CO₂CH₂CH₃, —CO₂CH₂CH₂CH₃;
iv) —C(O)N(R¹⁰)₂; for example, —CONH₂, —CONHCH₃, —CON(CH₃)₂;
v) —N(R¹⁰)₂; for example, —NH₂, —NHCH₃, —N(CH₃)₂, —NH(CH₂CH₃);
vi) halogen: —F, —C₁, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹⁰; for example, —SO₂H; —SO₂CH₃; —SO₂C₆H₅;
ix) C₁-C₆ linear, branched, or cyclic alkyl;
x) Cyano
xi) Nitro;
xii) N(R¹⁰)C(O)R¹⁰;
xiii) Oxo (═O);
xiv) Heterocycle; and
xv) Heteroaryl.
   wherein each R¹⁰ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹⁰ units can be taken together to form a ring comprising 3-7 ring atoms. In certain aspects, each R¹⁰ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the sigma-2 receptor activity modulators and sigma-2 receptor binders described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R⁹)₂, each R⁹ may be the same or different than the other). Combinations of substituents and/or variables are permissible only if such combinations result in stable compounds

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

The sigma-2 receptor binders and sigma-2 receptor activity modulators

The sigma-2 receptor binders and sigma-2 receptor activity modulators of the present invention include all enantiomeric and diastereomeric forms thereof having the formula

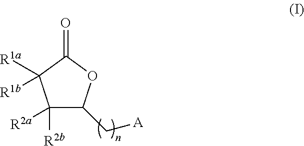

including hydrates, solvates, pharmaceutically acceptable salts, prodrugs and complexes thereof, wherein:

A is selected from the group consisting of

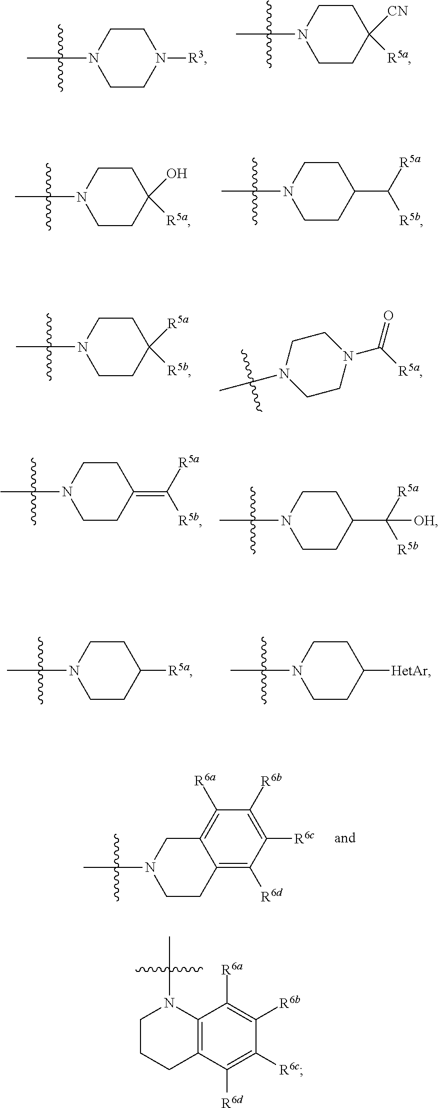

R^{1a} and R^{1b} are each independently selected from the group consisting of hydrogen, C₁₋₆ linear alkyl, C₁₋₆ branched alkyl, and optionally substituted aryl, or R^{1a} and R^{1b} may be taken together with the atom to which they are bound to form a ring having from 3 to 7 ring atoms;

R^{2a} and R^{2b} are each independently selected from the group consisting of hydrogen, C₁₋₆ linear alkyl, C₁₋₆ branched alkyl, and optionally substituted aryl, or R^{2a} and R^{2b} may be taken together with the atom to which they are bound to form a ring having from 3 to 7 ring atoms;

R³ is selected from the group consisting of C₁₋₆ linear alkyl, C₁₋₆ branched alkyl, C₃₋₇ cycloalkyl, optionally substituted aryl, optionally substituted heteroaryl,

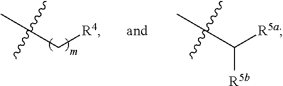

R⁴ is optionally substituted aryl;

R^{5a} and R^{5b} are each independently optionally substituted aryl;

R^{6a}, R^{6b}, R^{6c}, and R^{6d} are each independently selected from the group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁷, C(O)NHR⁷, C(O)N(R⁷)₂, SH, SC₁₋₆ alkyl, SO²NH₂, SO₂NHR⁷, SO₂R⁷, and NHSO₂R⁷;

each occurrence of R⁷ is independently selected from the group consisting of C₁₋₆ linear alkyl, C₁₋₆ branched alkyl, and C₃₋₇ cycloalkyl;

HetAr is optionally substituted heteroaryl;

n is 1, 2, or 3; and

m is 1 or 2.

The embodiments of the present invention include compounds having formula (II):

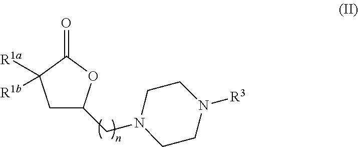

including hydrates, solvates, enantiomers, diastereomers, pharmaceutically acceptable salts, and complexes thereof.

The embodiments of the present invention include compounds having formula (III):

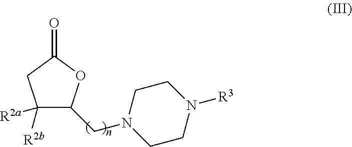

including hydrates, solvates, enantiomers, diastereomers, pharmaceutically acceptable salts, and complexes thereof.

The embodiments of the present invention include compounds having formula (IV):

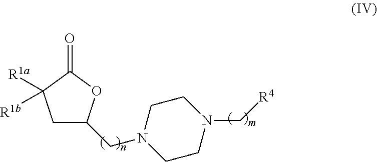

including hydrates, solvates, enantiomers, diastereomers, pharmaceutically acceptable salts, and complexes thereof.

The embodiments of the present invention include compounds having formula (V):

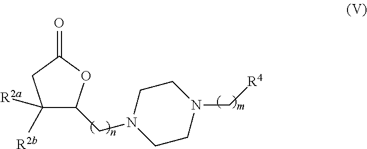

including hydrates, solvates, enantiomers, diastereomers, pharmaceutically acceptable salts, and complexes thereof.

The embodiments of the present invention include compounds having formula (VI):

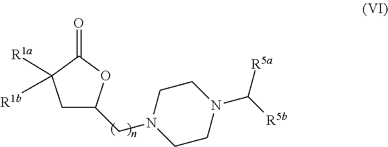

including hydrates, solvates, enantiomers, diastereomers, pharmaceutically acceptable salts, and complexes thereof.

The embodiments of the present invention include compounds having formula (VII):

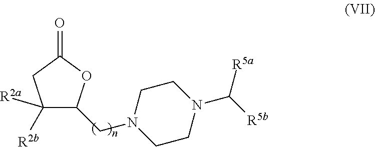

including hydrates, solvates, enantiomers, diastereomers, pharmaceutically acceptable salts, and complexes thereof.

The embodiments of the present invention include compounds having formula (VIII):

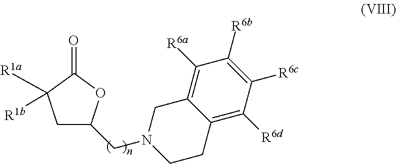

including hydrates, solvates, enantiomers, diastereomers, pharmaceutically acceptable salts, and complexes thereof.

The embodiments of the present invention include compounds having formula (IX):

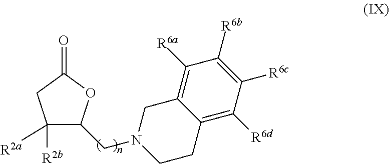

including hydrates, solvates, enantiomers, diastereomers, pharmaceutically acceptable salts, and complexes thereof.

The embodiments of the present invention include compounds having formula (X):

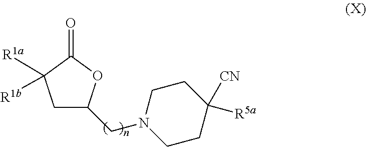

including hydrates, solvates, enantiomers, diastereomers, pharmaceutically acceptable salts, and complexes thereof.

The embodiments of the present invention include compounds having formula (XI):

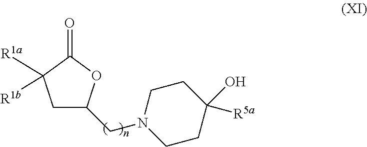

including hydrates, solvates, enantiomers, diastereomers, pharmaceutically acceptable salts, and complexes thereof.

The embodiments of the present invention include compounds having formula (XII):

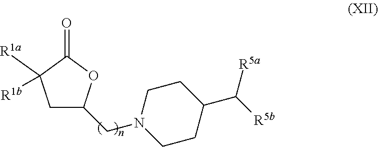

including hydrates, solvates, enantiomers, diastereomers, pharmaceutically acceptable salts, and complexes thereof.

The embodiments of the present invention include compounds having formula (XIII):

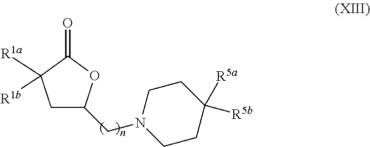

including hydrates, solvates, enantiomers, diastereomers, pharmaceutically acceptable salts, and complexes thereof.

The embodiments of the present invention include compounds having formula (XIV):

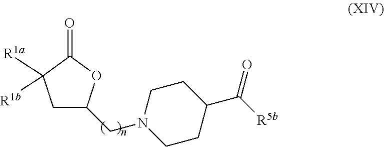

including hydrates, solvates, enantiomers, diastereomers, pharmaceutically acceptable salts, and complexes thereof.

The embodiments of the present invention include compounds having formula (XV):

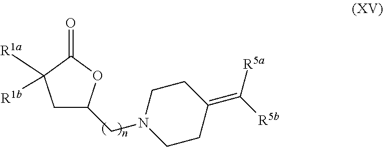

including hydrates, solvates, enantiomers, diastereomers, pharmaceutically acceptable salts, and complexes thereof.

The embodiments of the present invention include compounds having formula (XVI):

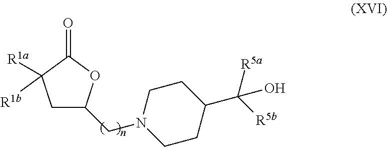

including hydrates, solvates, enantiomers, diastereomers, pharmaceutically acceptable salts, and complexes thereof.

The embodiments of the present invention include compounds having formula (XVII):

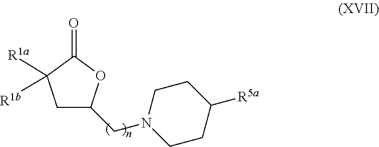

including hydrates, solvates, enantiomers, diastereomers, pharmaceutically acceptable salts, and complexes thereof.

The embodiments of the present invention include compounds having formula (XVIII):

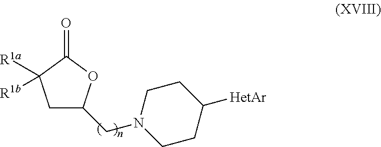

including hydrates, solvates, enantiomers, diastereomers, pharmaceutically acceptable salts, and complexes thereof.

The embodiments of the present invention include compounds having formula (XIX):

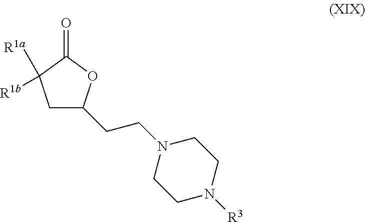

including hydrates, solvates, enantiomers, diastereomers, pharmaceutically acceptable salts, and complexes thereof.

In some embodiments A is

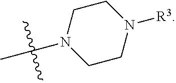

In some embodiments A is

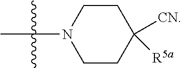

In some embodiments A is

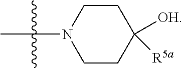

In some embodiments A is

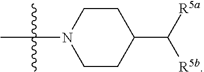

In some embodiments A is

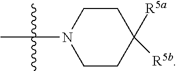

In some embodiments A is

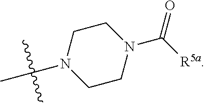

In some embodiments A is

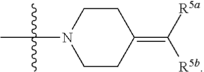

In some embodiments A is

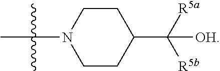

In some embodiments A is

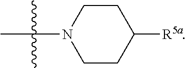

In some embodiments A is

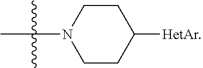

In some embodiments A is

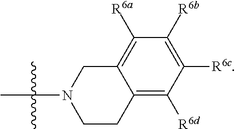

In some embodiments A is

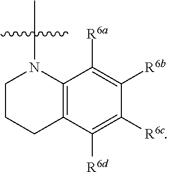

In some embodiments R^{1a} is hydrogen.

In some embodiments R^{1a} is C₁₋₆ linear alkyl.

In some embodiments R^{1a} is C₁₋₆ branched alkyl.

In some embodiments R^{1a} is optionally substituted aryl.

In some embodiments R^{1b} is hydrogen.

In some embodiments R^{1b} is C₁₋₆ linear alkyl.

In some embodiments R^{1b} is C₁₋₆ branched alkyl.

In some embodiments R^{1b} is optionally substituted aryl.

In some embodiments R^{1a} and R^{1b} are taken together with the atom to which they are bound to form a ring having 3 ring atoms.

In some embodiments R^{1a} and R^{1b} are taken together with the atom to which they are bound to form a ring having 4 ring atoms.

In some embodiments R^{1a} and R^{1b} are taken together with the atom to which they are bound to form a ring having 5 ring atoms.

In some embodiments R^{1a} and R^{1b} are taken together with the atom to which they are bound to form a ring having 6 ring atoms.

In some embodiments R^{1a} and R^{1b} are taken together with the atom to which they are bound to form a ring having 7 ring atoms.

In some embodiments R^{2a} is hydrogen.

In some embodiments R^{2a} is C₁₋₆ linear alkyl.

In some embodiments R^{2a} is C₁₋₆ branched alkyl.

In some embodiments R^{2a} is optionally substituted aryl.

In some embodiments R^{2b} is hydrogen.

In some embodiments R^{2b} is C₁₋₆ linear alkyl.

In some embodiments R^{2b} is C₁₋₆ branched alkyl.

In some embodiments R^{2b} is optionally substituted aryl.

In some embodiments R^{2a} and R^{2b} are taken together with the atom to which they are bound to form a ring having 3 ring atoms.

In some embodiments R^{2a} and R^{2b} are taken together with the atom to which they are bound to form a ring having 4 ring atoms.

In some embodiments R^{2a} and R^{2b} are taken together with the atom to which they are bound to form a ring having 5 ring atoms.

In some embodiments R^{2a} and R^{2b} are taken together with the atom to which they are bound to form a ring having 6 ring atoms.

In some embodiments R^{2a} and R^{2b} are taken together with the atom to which they are bound to form a ring having 7 ring atoms.

In some embodiments R³ is C₁₋₆ linear alkyl.

In some embodiments R³ is C₁₋₆ branched alkyl.

In some embodiments R³ is C₃₋₇ cycloalkyl.

In some embodiments R³ is optionally substituted aryl.

In some embodiments R³ is optionally substituted heteroaryl.

In some embodiments R³ is

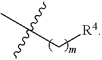

In some embodiments R³ is

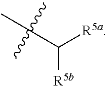

In some embodiments R⁴ is optionally substituted aryl.

In some embodiments R^{5a} is optionally substituted aryl.

In some embodiments R^{5b} is optionally substituted aryl.

In some embodiments R^{6a} is hydrogen.

In some embodiments R^{6a} is halogen.

In some embodiments R^{6a} is OH.

In some embodiments R^{6a} is C₁₋₆ linear alkyl.

In some embodiments R^{6a} is C₁₋₆ branched alkyl.

In some embodiments R^{6a} is C₁₋₆ alkoxy.

In some embodiments R^{6a} is C₁₋₆ haloalkyl.

In some embodiments R^{6a} is C₁₋₆ haloalkoxy.

In some embodiments R^{6a} is cyano.

In some embodiments R^{6a} is NH(C₁₋₆ alkyl).

In some embodiments R^{6a} is N(C₁₋₆ alkyl)₂.

In some embodiments R^{6a} is NHC(O)R⁷.

In some embodiments R^{6a} is C(O)NHR⁷.

In some embodiments R^{6a} is C(O)N(R⁷)₂.

In some embodiments R^{6a} is SH.

In some embodiments R^{6a} is SC₁₋₆ alkyl.

In some embodiments R^{6a} is SO²NH₂.

In some embodiments R^{6a} is SO₂NHR⁷.

In some embodiments R^{6a} is SO₂R⁷.

In some embodiments R^{6a} is NHSO₂R⁷.

In some embodiments R^{6b} is hydrogen.

In some embodiments R^{6b} is halogen.

In some embodiments R^{6b} is OH.

In some embodiments R^{6b} is C₁₋₆ linear alkyl.

In some embodiments R^{6b} is C₁₋₆ branched alkyl.

In some embodiments R^{6b} is C₁₋₆ alkoxy.

In some embodiments R^{6b} is C₁₋₆ haloalkyl.

In some embodiments R^{6b} is C₁₋₆ haloalkoxy.

In some embodiments R^{6b} is cyano.

In some embodiments R^{6b} is NH(C₁₋₆ alkyl).

In some embodiments R^{6b} is N(C₁₋₆ alkyl)₂.

In some embodiments R^{6b} is NHC(O)R⁷.

In some embodiments R^{6b} is C(O)NHR⁷.

In some embodiments R^{6b} is C(O)N(R⁷)₂.

In some embodiments R^{6b} is SH.

In some embodiments R^{6b} is SC₁₋₆ alkyl.

In some embodiments R^{6b} is SO²NH₂.

In some embodiments R^{6b} is SO₂NHR⁷.

In some embodiments R^{6b} is SO₂R⁷.

In some embodiments R^{6b} is NHSO₂R⁷.

In some embodiments R^{6c} is hydrogen.

In some embodiments R^{6c} is halogen.

In some embodiments R^{6c} is OH.

In some embodiments R^{6c} is C₁₋₆ linear alkyl.

In some embodiments R^{6c} is C₁₋₆ branched alkyl.

In some embodiments R^{6c} is C₁₋₆ alkoxy.

In some embodiments R^{6c} is C₁₋₆ haloalkyl.

In some embodiments R^{6c} is C₁₋₆ haloalkoxy.

In some embodiments R^{6c} is cyano.

In some embodiments R^{6c} is NH(C₁₋₆ alkyl)

In some embodiments R^{6c} is N(C₁₋₆ alkyl)₂.

In some embodiments R^{6c} is NHC(O)R⁷.

In some embodiments R^{6c} is C(O)NHR⁷.

In some embodiments R^{6c} is C(O)N(R⁷)₂.

In some embodiments R^{6c} is SH.

In some embodiments R^{6c} is SC₁₋₆ alkyl.

In some embodiments R^{6c} is SO²NH₂.

In some embodiments R^{6c} is SO₂NHR⁷.

In some embodiments R^{6c} is SO₂R⁷.

In some embodiments R^{6c} is NHSO₂R⁷.

In some embodiments R^{6d} is hydrogen.

In some embodiments R^{6d} is halogen.

In some embodiments R^{6d} is OH.

In some embodiments R^{6d} is C₁₋₆ linear alkyl.

In some embodiments R^{6d} is C₁₋₆ branched alkyl.

In some embodiments R^{6d} is C₁₋₆ alkoxy.

In some embodiments R^{6d} is C₁₋₆ haloalkyl.

In some embodiments R^{6d} is C₁₋₆ haloalkoxy.

In some embodiments R^{6d} is cyano.

In some embodiments R^{6d} is NH(C₁₋₆ alkyl).

In some embodiments R^{6d} is N(C₁₋₆ alkyl)₂.

In some embodiments R^{6d} is NHC(O)R⁷.

In some embodiments R^{6d} is C(O)NHR⁷.

In some embodiments R^{6d} is C(O)N(R⁷)₂.

In some embodiments R^{6d} is SH.

In some embodiments R^{6d} is SC₁₋₆ alkyl.

In some embodiments R^{6d} is SO²NH₂.

In some embodiments R^{6d} is SO₂NHR⁷.

In some embodiments R^{6d} is SO₂R⁷.

In some embodiments R^{6d} is NHSO₂R⁷.

In some embodiments R⁷ is C₁₋₆ linear alkyl.

In some embodiments R⁷ is C₁₋₆ branched alkyl.

In some embodiments R⁷ is C₃₋₇ cycloalkyl.

HetAr is optionally substituted heteroaryl.

In some embodiments n is 1.

In some embodiments n is 2.

In some embodiments n is 3.

In some embodiments m is 1.

In some embodiments m is 2.

Exemplary embodiments include compounds having the formula (II) or a pharmaceutically acceptable salt form thereof:

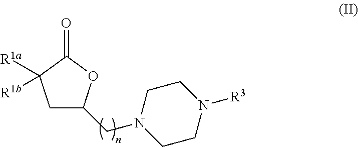

wherein non-limiting examples of R^{1a}, R^{1b}, R³ and “n” are defined herein below in Table 1.

TABLE 1

| Entry | n | R^{1a} | R^{1b} | R³ |
|---|---|---|---|---|
| 1 | 2 | CH₃ | CH₃ | 4-CH₃phenyl |
| 2 | 2 | CH₃ | CH₃ | 2-isopropylphenyl |
| 3 | 2 | CH₃ | CH₃ | 2-cyanophenyl |
| 4 | 1 | phenyl | phenyl | 4-CH₃phenyl |
| 5 | 2 | phenyl | phenyl | 4-CH₃phenyl |
| 6 | 3 | phenyl | phenyl | 4-CH₃phenyl |
| 7 | 2 | phenyl | phenyl | 2-isopropylphenyl |
| 8 | 1 | CH₂CH₃ | CH₂CH₃ | 4-CH₃phenyl |
| 9 | 2 | CH₂CH₃ | CH₂CH₃ | phenyl |
| 10 | 2 | CH₂CH₃ | CH₂CH₃ | 4-cyanophenyl |
| 11 | 2 | CH₂CH₃ | CH₂CH₃ | 2-methoxyphenyl |
| 12 | 2 | CH₂CH₃ | CH₂CH₃ | 4-nitrophenyl |
| 13 | 2 | CH₂CH₃ | CH₂CH₃ | 2-hydroxyphenyl |
| 14 | 2 | CH₂CH₃ | CH₂CH₃ | 4-CH₃phenyl |
| 15 | 2 | CH₂CH₃ | CH₂CH₃ | 4-hydroxyphenyl |
| 16 | 2 | CH₂CH₃ | CH₂CH₃ | 4-methoxyphenyl |
| 17 | 2 | CH₂CH₃ | CH₂CH₃ | 4-aminophenyl |
| 18 | 2 | CH₂CH₃ | CH₂CH₃ | 2,4-dimethylphenyl |
| 19 | 2 | CH₂CH₃ | CH₂CH₃ | 2-isopropylphenyl |
| 20 | 2 | CH₂CH₃ | CH₂CH₃ | 2-methylphenyl |
| 21 | 2 | CH₂CH₃ | CH₂CH₃ | 2,6-dimethylphenyl |
| 22 | 2 | CH₂CH₃ | CH₂CH₃ | 2-pyridinyl |
| 23 | 2 | CH₂CH₃ | CH₂CH₃ | cyclohexyl |
| 24 | 2 | CH₂CH₃ | CH₂CH₃ | 3-hydroxyphenyl |
| 25 | 2 | CH₂CH₃ | CH₂CH₃ | 3-methoxyphenyl |
| 26 | 2 | CH₂CH₃ | CH₂CH₃ | 2-cyanophenyl |

| | | | |
|---|---|---|---|
| 27 | 1 | —CH₂(CH₂)₃CH₂— | 4-CH₃phenyl |
| 28 | 2 | —CH₂(CH₂)₃CH₂— | 4-CH₃phenyl |
| 29 | 3 | —CH₂(CH₂)₃CH₂— | 4-CH₃phenyl |
| 30 | 2 | —CH₂(CH₂)₃CH₂— | 2-isopropylphenyl |
| 31 | 3 | —CH₂(CH₂)₃CH₂— | 2-isopropylphenyl |
| 32 | 1 | —CH₂(CH₂)₂CH₂— | 4-CH₃phenyl |
| 33 | 2 | —CH₂(CH₂)₂CH₂— | 4-CH₃phenyl |
| 34 | 3 | —CH₂(CH₂)₂CH₂— | 4-CH₃phenyl |
| 35 | 2 | —CH₂(CH₂)₂CH₂— | 2-isopropylphenyl |
| 36 | 3 | —CH₂(CH₂)₂CH₂— | 2-isopropylphenyl |
| 37 | 2 | —CH₂(CH₂)₂CH₂— | 2-cyano-4-nitrophenyl |
| 38 | 2 | —CH₂(CH₂)₂CH₂— | 4-benzo[c][1,2,5]thiadiazole |
| 39 | 2 | —CH₂(CH₂)₂CH₂— | 4-(anthracen-1-yl) |
| 40 | 2 | —CH₂(CH₂)₂CH₂— | 4-(naphthalen-1-yl) |
| 41 | 2 | —CH₂(CH₂)₂CH₂— | 2-acetamidophenyl |
| 42 | 2 | —CH₂(CH₂)₂CH₂— | 2-iodophenyl |
| 43 | 2 | —CH₂(CH₂)₂CH₂— | 2-pyrrol-1-yl-phenyl |
| 44 | 2 | —CH₂(CH₂)₂CH₂— | 2-phenylphenyl |
| 45 | 2 | —CH₂(CH₂)₂CH₂— | 2-morpholinophenyl |
| 46 | 2 | —CH₂(CH₂)₂CH₂— | 2,6-diisopropylphenyl |
| 47 | 2 | —CH₂(CH₂)₂CH₂— | 2-tert-butylphenyl |
| 48 | 2 | —CH₂(CH₂)₃CH₂— | 4-methoxyphenyl |
| 49 | 2 | —CH₂(CH₂)₃CH₂— | 3-hydroxyphenyl |
| 50 | 2 | —CH₂(CH₂)₃CH₂— | phenyl |
| 51 | 2 | —CH₂(CH₂)₃CH₂— | 3-methoxyphenyl |
| 52 | 2 | —CH₂(CH₂)₃CH₂— | 2-methoxyphenyl |
| 53 | 2 | —CH₂(CH₂)₃CH₂— | 2-hydroxyphenyl |
| 54 | 2 | —CH₂(CH₂)₃CH₂— | 2-pyridinyl |
| 55 | 2 | —CH₂(CH₂)₃CH₂— | 2-chlorophenyl |
| 56 | 2 | —CH₂(CH₂)₃CH₂— | 4-chlorophenyl |
| 57 | 2 | —CH₂(CH₂)₃CH₂— | 4-trifluoromethylphenyl |
| 58 | 2 | —CH₂(CH₂)₃CH₂— | 4-pyridinyl |
| 59 | 2 | —CH₂(CH₂)₃CH₂— | 4-hydroxyphenyl |
| 60 | 2 | —CH₂(CH₂)₃CH₂— | 3-pyridinyl |
| 61 | 2 | —CH₂(CH₂)₃CH₂— | 4-cyanophenyl |
| 62 | 2 | —CH₂(CH₂)₃CH₂— | 4-cyano-2-pyridinyl |
| 63 | 2 | —CH₂(CH₂)₃CH₂— | 4-trifluoromethyl-2-pyridinyl |
| 64 | 2 | —CH₂(CH₂)₃CH₂— | 4-chloro-2-pyridinyl |
| 65 | 2 | —CH₂(CH₂)₂CH₂— | 4-chloro-2-pyridinyl |
| 66 | 2 | —CH₂(CH₂)₂CH₂— | 4-trifluoromethyl-2-pyridinyl |
| 67 | 2 | —CH₂(CH₂)₂CH₂— | 4-methyl-2-pyridinyl |
| 68 | 2 | —CH₂(CH₂)₂CH₂— | 4-hydroxy-2-pyridinyl |
| 69 | 2 | —CH₂(CH₂)₂CH₂— | 4-cyano-2-pyridinyl |
| 70 | 2 | —CH₂(CH₂)₂CH₂— | 4-pyridinyl |
| 71 | 2 | —CH₂(CH₂)₂CH₂— | 4-fluoro-2-pyridinyl |
| 72 | 2 | —CH₂(CH₂)₃CH₂— | 1H-indol-5-yl |
| 73 | 2 | —CH₂(CH₂)₂CH₂— | 1H-indol-5-yl |
| 74 | 2 | —CH₂CH₂CH₂— | 1H-indol-5-yl |

Exemplary embodiments include compounds having the formula (III) or a pharmaceutically acceptable salt form thereof:

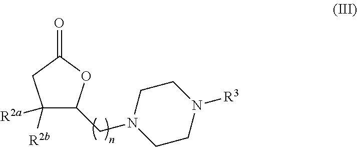

wherein non-limiting examples of R^{2a}, R^{2b}, R³ and “n” are defined herein below in Table 2.

TABLE 2

| Entry | n | R^{2a} | R^{2b} | R³ |
|---|---|---|---|---|
| 1 | 2 | CH₃ | CH₃ | 4-CH₃phenyl |
| 2 | 2 | CH₃ | CH₃ | 2-isopropylphenyl |
| 3 | 2 | CH₃ | CH₃ | 2-cyanophenyl |
| 4 | 1 | phenyl | phenyl | 4-CH₃phenyl |
| 5 | 2 | phenyl | phenyl | 4-CH₃phenyl |
| 6 | 3 | phenyl | phenyl | 4-CH₃phenyl |
| 7 | 2 | phenyl | phenyl | 2-isopropylphenyl |
| 8 | 1 | CH₂CH₃ | CH₂CH₃ | 4-CH₃phenyl |
| 9 | 2 | CH₂CH₃ | CH₂CH₃ | phenyl |
| 10 | 2 | CH₂CH₃ | CH₂CH₃ | 4-cyanophenyl |
| 11 | 2 | CH₂CH₃ | CH₂CH₃ | 2-methoxyphenyl |
| 12 | 2 | CH₂CH₃ | CH₂CH₃ | 4-nitrophenyl |
| 13 | 2 | CH₂CH₃ | CH₂CH₃ | 2-hydroxyphenyl |
| 14 | 2 | CH₂CH₃ | CH₂CH₃ | 4-CH₃phenyl |
| 15 | 2 | CH₂CH₃ | CH₂CH₃ | 4-hydroxyphenyl |
| 16 | 2 | CH₂CH₃ | CH₂CH₃ | 4-methoxyphenyl |
| 17 | 2 | CH₂CH₃ | CH₂CH₃ | 4-aminophenyl |
| 18 | 2 | CH₂CH₃ | CH₂CH₃ | 2,4-dimethylphenyl |
| 19 | 2 | CH₂CH₃ | CH₂CH₃ | 2-isopropylphenyl |
| 20 | 2 | CH₂CH₃ | CH₂CH₃ | 2-methylphenyl |
| 21 | 2 | CH₂CH₃ | CH₂CH₃ | 2,6-dimethylphenyl |
| 22 | 2 | CH₂CH₃ | CH₂CH₃ | 2-pyridinyl |
| 23 | 2 | CH₂CH₃ | CH₂CH₃ | cyclohexyl |
| 24 | 2 | CH₂CH₃ | CH₂CH₃ | 3-hydroxyphenyl |
| 25 | 2 | CH₂CH₃ | CH₂CH₃ | 3-methoxyphenyl |
| 26 | 2 | CH₂CH₃ | CH₂CH₃ | 2-cyanophenyl |

| | | | |
|---|---|---|---|
| 27 | 1 | —CH₂(CH₂)₃CH₂— | 4-CH₃phenyl |
| 28 | 2 | —CH₂(CH₂)₃CH₂— | 4-CH₃phenyl |
| 29 | 3 | —CH₂(CH₂)₃CH₂— | 4-CH₃phenyl |
| 30 | 2 | —CH₂(CH₂)₃CH₂— | 2-isopropylphenyl |
| 31 | 3 | —CH₂(CH₂)₃CH₂— | 2-isopropylphenyl |
| 32 | 1 | —CH₂(CH₂)₂CH₂— | 4-CH₃phenyl |
| 33 | 2 | —CH₂(CH₂)₂CH₂— | 4-CH₃phenyl |
| 34 | 3 | —CH₂(CH₂)₂CH₂— | 4-CH₃phenyl |
| 35 | 2 | —CH₂(CH₂)₂CH₂— | 2-isopropylphenyl |
| 36 | 3 | —CH₂(CH₂)₂CH₂— | 2-isopropylphenyl |
| 37 | 2 | —CH₂(CH₂)₂CH₂— | 2-cyano-4-nitrophenyl |
| 38 | 2 | —CH₂(CH₂)₂CH₂— | 4-benzo[c][1,2,5]thiadiazole |
| 39 | 2 | —CH₂(CH₂)₂CH₂— | 4-(anthracen-1-yl) |
| 40 | 2 | —CH₂(CH₂)₂CH₂— | 4-(naphthalen-1-yl) |
| 41 | 2 | —CH₂(CH₂)₂CH₂— | 2-acetamidophenyl |
| 42 | 2 | —CH₂(CH₂)₂CH₂— | 2-iodophenyl |
| 43 | 2 | —CH₂(CH₂)₂CH₂— | 2-pyrrol-1-yl-phenyl |
| 44 | 2 | —CH₂(CH₂)₂CH₂— | 2-phenylphenyl |
| 45 | 2 | —CH₂(CH₂)₂CH₂— | 2-morpholinophenyl |
| 46 | 2 | —CH₂(CH₂)₂CH₂— | 2,6-diisopropylphenyl |
| 47 | 2 | —CH₂(CH₂)₂CH₂— | 2-tert-butylphenyl |

Exemplary embodiments include compounds having the formula (IV) or a pharmaceutically acceptable salt form thereof:

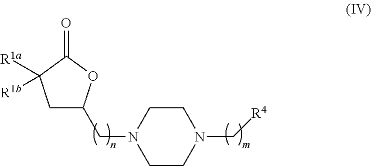

wherein non-limiting examples of R^{1a}, R^{1b}, R⁴, “m” and “n” are defined herein below in Table 3.

TABLE 3

| Entry | n | m | R^{1a} | R^{1b} | R⁴ |
|---|---|---|---|---|---|
| 1 | 1 | 1 | CH₃ | CH₃ | 4-fluorophenyl |
| 2 | 2 | 1 | CH₃ | CH₃ | 4-fluorophenyl |
| 3 | 2 | 2 | CH₃ | CH₃ | 4-fluorophenyl |
| 4 | 1 | 1 | CH₃ | CH₃ | phenyl |
| 5 | 2 | 1 | CH₃ | CH₃ | phenyl |
| 6 | 2 | 2 | CH₃ | CH₃ | phenyl |
| 7 | 1 | 1 | CH₂CH₃ | CH₂CH₃ | 4-fluorophenyl |
| 8 | 2 | 1 | CH₂CH₃ | CH₂CH₃ | 4-fluorophenyl |
| 9 | 2 | 2 | CH₂CH₃ | CH₂CH₃ | 4-fluorophenyl |
| 10 | 1 | 1 | CH₂CH₃ | CH₂CH₃ | phenyl |
| 11 | 2 | 1 | CH₂CH₃ | CH₂CH₃ | phenyl |
| 12 | 2 | 2 | CH₂CH₃ | CH₂CH₃ | phenyl |

Exemplary embodiments include compounds having the formula (V) or a pharmaceutically acceptable salt form thereof:

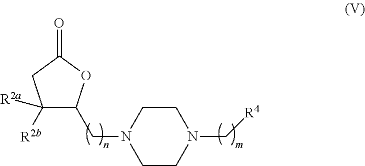

wherein non-limiting examples of R^{2a}, R^{2b}, R⁴, “m” and “n” are defined herein below in Table 4.

TABLE 4

| Entry | n | m | R^{2a} | R^{2b} | R⁴ |
|---|---|---|---|---|---|
| 1 | 1 | 1 | CH₃ | CH₃ | 4-fluorophenyl |
| 2 | 2 | 1 | CH₃ | CH₃ | 4-fluorophenyl |
| 3 | 2 | 2 | CH₃ | CH₃ | 4-fluorophenyl |
| 4 | 1 | 1 | CH₃ | CH₃ | phenyl |
| 5 | 2 | 1 | CH₃ | CH₃ | phenyl |
| 6 | 2 | 2 | CH₃ | CH₃ | phenyl |
| 7 | 1 | 1 | CH₂CH₃ | CH₂CH₃ | 4-fluorophenyl |
| 8 | 2 | 1 | CH₂CH₃ | CH₂CH₃ | 4-fluorophenyl |
| 9 | 2 | 2 | CH₂CH₃ | CH₂CH₃ | 4-fluorophenyl |
| 10 | 1 | 1 | CH₂CH₃ | CH₂CH₃ | phenyl |
| 11 | 2 | 1 | CH₂CH₃ | CH₂CH₃ | phenyl |
| 12 | 2 | 2 | CH₂CH₃ | CH₂CH₃ | phenyl |

Exemplary embodiments include compounds having the formula (VI) or a pharmaceutically acceptable salt form thereof:

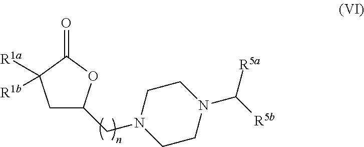

wherein non-limiting examples of R^{1a}, R^{1b}, R^{5a}, R^{5b}, and “n” are defined herein below in Table 5.

TABLE 5

| Entry | n | R^{1a} | R^{1b} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (VII) or a pharmaceutically acceptable salt form thereof:

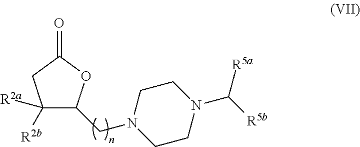

wherein non-limiting examples of R^{2a}, R^{2b}, R^{5a}, R^{5b}, and “n” are defined herein below in Table 6.

TABLE 6

| Entry | n | R^{2a} | R^{2a} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (VIII) or a pharmaceutically acceptable salt form thereof:

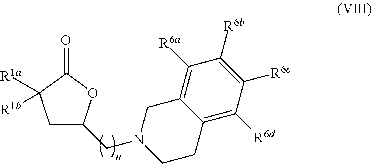

wherein non-limiting examples of R^{1a}, R^{1b}, R^{6a}, R^{6b}, R^{6c}, R^{6d}, and “n” are defined herein below in Table 7.

TABLE 7

| Entry | n | R^{1a} | R^{1b} | R^{6a} | R^{6b} | R^{6c} | R^{6d} |
|---|---|---|---|---|---|---|---|
| 1 | 1 | CH₃ | CH₃ | H | H | H | H |
| 2 | 2 | CH₃ | CH₃ | H | H | H | H |
| 3 | 1 | CH₂CH₃ | CH₂CH₃ | H | H | H | H |
| 4 | 2 | CH₂CH₃ | CH₂CH₃ | H | H | H | H |

| | | | | | | |
|---|---|---|---|---|---|---|
| 5 | 1 | —CH₂(CH₂)CH₂— | H | H | H | H |
| 6 | 1 | —CH₂(CH₂)₂CH₂— | H | H | H | H |
| 7 | 1 | —CH₂(CH₂)₃CH₂— | H | H | H | H |
| 8 | 2 | —CH₂(CH₂)CH₂— | H | H | H | H |
| 9 | 2 | —CH₂(CH₂)₂CH₂— | H | H | H | H |
| 10 | 2 | —CH₂(CH₂)₃CH₂— | H | H | H | H |

Exemplary embodiments include compounds having the formula (IX) or a pharmaceutically acceptable salt form thereof:

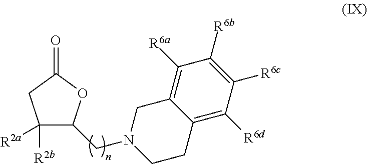

wherein non-limiting examples of R^{2a}, R^{2b}, R^{6a}, R^{6b}, R^{6c}, R^{6d}, and “n” are defined herein below in Table 8.

TABLE 8

| Entry | n | R^{2a} | R^{2b} | R^{6a} | R^{6b} | R^{6c} | R^{6d} |
|---|---|---|---|---|---|---|---|
| 1 | 1 | CH₃ | CH₃ | H | H | H | H |
| 2 | 2 | CH₃ | CH₃ | H | H | H | H |
| 3 | 1 | CH₂CH₃ | CH₂CH₃ | H | H | H | H |
| 4 | 2 | CH₂CH₃ | CH₂CH₃ | H | H | H | H |

| | | | | | | |
|---|---|---|---|---|---|---|
| 5 | 1 | —CH₂(CH₂)CH₂— | H | H | H | H |
| 6 | 1 | —CH₂(CH₂)₂CH₂— | H | H | H | H |
| 7 | 1 | —CH₂(CH₂)₃CH₂— | H | H | H | H |
| 8 | 2 | —CH₂(CH₂)CH₂— | H | H | H | H |
| 9 | 2 | —CH₂(CH₂)₂CH₂— | H | H | H | H |
| 10 | 2 | —CH₂(CH₂)₃CH₂— | H | H | H | H |

Exemplary embodiments include compounds having the formula (X) or a pharmaceutically acceptable salt form thereof:

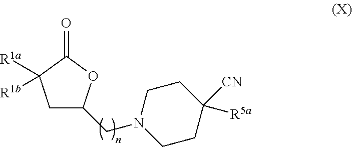

wherein non-limiting examples of R^{1a}, R^{1b}, R^{5a}, and “n” are defined herein below in Table 9.

TABLE 9

| Entry | n | R^{1a} | R^{1b} | R^{5a} |
|---|---|---|---|---|
| 1 | 1 | —CH₃ | —CH₃ | phenyl |
| 2 | 1 | —CH₂CH₃ | —CH₂CH₃ | phenyl |
| 3 | 2 | —CH₃ | —CH₃ | phenyl |
| 4 | 2 | —CH₂CH₃ | —CH₂CH₃ | phenyl |

| | | | |
|---|---|---|---|
| 5 | 1 | —CH₂(CH₂)CH₂— | phenyl |
| 6 | 1 | —CH₂(CH₂)₂CH₂— | phenyl |
| 7 | 1 | —CH₂(CH₂)₃CH₂— | phenyl |
| 8 | 2 | —CH₂(CH₂)CH₂— | phenyl |
| 9 | 2 | —CH₂(CH₂)₂CH₂— | phenyl |
| 10 | 2 | —CH₂(CH₂)₃CH₂— | phenyl |

Exemplary embodiments include compounds having the formula (XI) or a pharmaceutically acceptable salt form thereof:

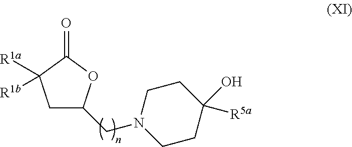

wherein non-limiting examples of R^{1a}, R^{1b}, R^{5a}, and “n” are defined herein below in Table 10.

TABLE 10

| Entry | n | R^{1a} | R^{1b} | R^{5a} |
|---|---|---|---|---|
| 1 | 1 | —CH₃ | —CH₃ | phenyl |
| 2 | 1 | —CH₂CH₃ | —CH₂CH₃ | phenyl |
| 3 | 2 | —CH₃ | —CH₃ | phenyl |
| 4 | 2 | —CH₂CH₃ | —CH₂CH₃ | phenyl |

| | | | |
|---|---|---|---|
| 5 | 1 | —CH₂(CH₂)CH₂— | phenyl |
| 6 | 1 | —CH₂(CH₂)₂CH₂— | phenyl |
| 7 | 1 | —CH₂(CH₂)₃CH₂— | phenyl |
| 8 | 2 | —CH₂(CH₂)CH₂— | phenyl |
| 9 | 2 | —CH₂(CH₂)₂CH₂— | phenyl |
| 10 | 2 | —CH₂(CH₂)₃CH₂— | phenyl |

Exemplary embodiments include compounds having the formula (XII) or a pharmaceutically acceptable salt form thereof:

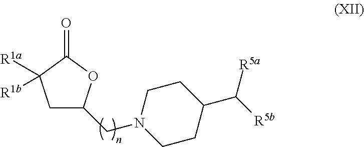

wherein non-limiting examples of R^{1a}, R^{1b}, R^{5a}, and “n” are defined herein below in Table 11.

TABLE 11

| Entry | n | R^{1a} | R^{1b} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (XIII) or a pharmaceutically acceptable salt form thereof:

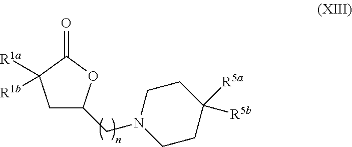

wherein non-limiting examples of R^{1a}, R^{1b}, R^{5a}, R^{5b}, and “n” are defined herein below in Table 12.

TABLE 12

| Entry | n | R^{1a} | R^{1b} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (XIV) or a pharmaceutically acceptable salt form thereof:

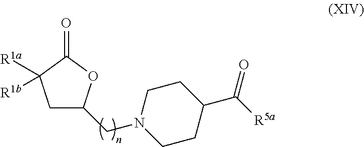

wherein non-limiting examples of R^{1a}, R^{1b}, R^{5a}, and “n” are defined herein below in Table 13.

TABLE 13

| Entry | n | R^{1a} | R^{1b} | R^{5a} |
|---|---|---|---|---|
| 1 | 1 | —CH₃ | —CH₃ | phenyl |
| 2 | 1 | —CH₂CH₃ | —CH₂CH₃ | phenyl |
| 3 | 2 | —CH₃ | —CH₃ | phenyl |
| 4 | 2 | —CH₂CH₃ | —CH₂CH₃ | phenyl |

| | | | |
|---|---|---|---|
| 5 | 1 | —CH₂(CH₂)CH₂— | phenyl |
| 6 | 1 | —CH₂(CH₂)₂CH₂— | phenyl |
| 7 | 1 | —CH₂(CH₂)₃CH₂— | phenyl |
| 8 | 2 | —CH₂(CH₂)CH₂— | phenyl |
| 9 | 2 | —CH₂(CH₂)₂CH₂— | phenyl |
| 10 | 2 | —CH₂(CH₂)₃CH₂— | phenyl |

Exemplary embodiments include compounds having the formula (XV) or a pharmaceutically acceptable salt form thereof:

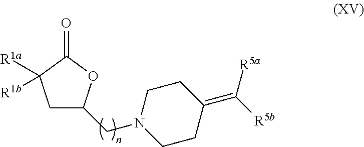

wherein non-limiting examples of R^{1a}, R^{1b}, R^{5a}, R^{5b}, and “n” are defined herein below in Table 14.

TABLE 14

| Entry | n | R^{1a} | R^{1b} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (XVI) or a pharmaceutically acceptable salt form thereof:

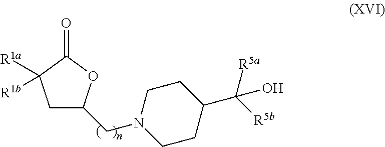

wherein non-limiting examples of R^{1a}, R^{1b}, R^{5a}, R^{5b}, and “n” are defined herein below in Table 15.

TABLE 15

| Entry | n | R^{1a} | R^{1b} | R^{5a} | R^{5b} |
|---|---|---|---|---|---|
| 1 | 1 | —CH₃ | —CH₃ | phenyl | phenyl |
| 2 | 1 | —CH₂CH₃ | —CH₂CH₃ | phenyl | phenyl |
| 3 | 2 | —CH₃ | —CH₃ | phenyl | phenyl |
| 4 | 2 | —CH₂CH₃ | —CH₂CH₃ | phenyl | phenyl |

| | | | | |
|---|---|---|---|---|
| 5 | 1 | —CH₂(CH₂)CH₂— | phenyl | phenyl |
| 6 | 1 | —CH₂(CH₂)₂CH₂— | phenyl | phenyl |
| 7 | 1 | —CH₂(CH₂)₃CH₂— | phenyl | phenyl |
| 8 | 2 | —CH₂(CH₂)CH₂— | phenyl | phenyl |
| 9 | 2 | —CH₂(CH₂)₂CH₂— | phenyl | phenyl |
| 10 | 2 | —CH₂(CH₂)₃CH₂— | phenyl | phenyl |

Exemplary embodiments include compounds having the formula (XVII) or a pharmaceutically acceptable salt form thereof:

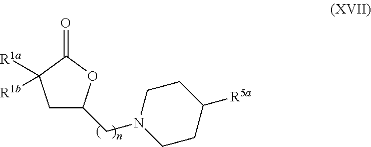

wherein non-limiting examples of R^{1a}, R^{1b}, R^{5a}, and “n” are defined herein below in Table 16.

TABLE 16

| Entry | n | R^{1a} | R^{1b} | R^{5a} |
|---|---|---|---|---|
| 1 | 1 | —CH₃ | —CH₃ | phenyl |
| 2 | 1 | —CH₂CH₃ | —CH₂CH₃ | phenyl |
| 3 | 2 | —CH₃ | —CH₃ | phenyl |
| 4 | 2 | —CH₂CH₃ | —CH₂CH₃ | phenyl |

| | | | |
|---|---|---|---|
| 5 | 1 | —CH₂(CH₂)CH₂— | phenyl |
| 6 | 1 | —CH₂(CH₂)₂CH₂— | phenyl |
| 7 | 1 | —CH₂(CH₂)₃CH₂— | phenyl |
| 8 | 2 | —CH₂(CH₂)CH₂— | phenyl |
| 9 | 2 | —CH₂(CH₂)₂CH₂— | phenyl |
| 10 | 2 | —CH₂(CH₂)₃CH₂— | phenyl |

Exemplary embodiments include compounds having the formula (XVIII) or a pharmaceutically acceptable salt form thereof:

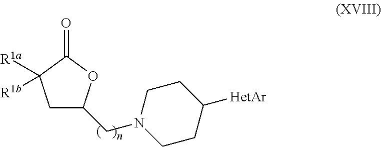

wherein non-limiting examples of R^{1a}, R^{1b}, HetAr, and “n” are defined herein below in Table 17.

TABLE 17

| Entry | n | R^{1a} | R^{1b} | HetAr |
|---|---|---|---|---|
| 1 | 2 | —CH₂CH₃ | —CH₂CH₃ | 2-1H-benzo[d]imidazolyl |
| 2 | 1 | —CH₃ | —CH₃ | 2-1H-benzo[d]imidazolyl |
| 3 | 1 | —CH₂CH₃ | —CH₂CH₃ | 2-1H-benzo[d]imidazolyl |
| 4 | 2 | —CH₃ | —CH₃ | 2-1H-benzo[d]imidazolyl |
| 5 | 2 | —CH₂CH₃ | —CH₂CH₃ | 2-1H-benzo[d]imidazolyl |

| | | | |
|---|---|---|---|
| 6 | 1 | —CH₂(CH₂)CH₂— | 2-1H-benzo[d]imidazolyl |
| 7 | 1 | —CH₂(CH₂)₂CH₂— | 2-1H-benzo[d]imidazolyl |
| 8 | 1 | —CH₂(CH₂)₃CH₂— | 2-1H-benzo[d]imidazolyl |
| 9 | 2 | —CH₂(CH₂)CH₂— | 2-1H-benzo[d]imidazolyl |
| 10 | 2 | —CH₂(CH₂)₂CH₂— | 2-1H-benzo[d]imidazolyl |
| 11 | 2 | —CH₂(CH₂)₃CH₂— | 2-1H-benzo[d]imidazolyl |

Exemplary embodiments include compounds having the formula (XIX) or a pharmaceutically acceptable salt form thereof:

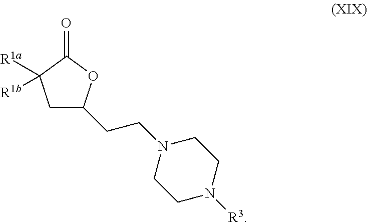

In one embodiment, R^{1a} and R^{1b} are each independently selected from the group consisting of methyl, ethyl, and a ring having four, five, or six carbon atoms formed by taking R^{1a} and R^{1b} together with the atom to which they are bound. In one embodiment, R³ is selected from the group consisting of optionally substituted aryl, optionally substituted heteroaryl.

In one embodiment, the compound of formula (XIX) is selected from the group consisting of:

| Entry | R^{1a} | R^{1b} | R³ |
|---|---|---|---|
| 1 | methyl | methyl | phenyl |
| 2 | methyl | methyl | 4-hydroxyphenyl |
| 3 | methyl | methyl | 4-cyanophenyl |
| 4 | methyl | methyl | 4-trifluorophenyl |
| 5 | methyl | methyl | 4-methylphenyl |
| 6 | methyl | methyl | 2-cyanophenyl |
| 7 | ethyl | ethyl | 4-hydroxyphenyl |
| 8 | ethyl | ethyl | 4-nitrophenyl |
| 9 | ethyl | ethyl | 2-methoxyphenyl |
| 10 | ethyl | ethyl | 4cyanophenyl |
| 11 | ethyl | ethyl | 2-hydroxyphenyl |
| 12 | ethyl | ethyl | phenyl |
| 13 | ethyl | ethyl | 4-aminophenyl |
| 14 | ethyl | ethyl | 4-methylphenyl |
| 15 | ethyl | ethyl | 3-methoxyphenyl |
| 16 | ethyl | ethyl | 3-hydroxyphenyl |
| 17 | ethyl | ethyl | 2-pyridyl |
| 18 | ethyl | ethyl | 2,6-dimethylphenyl |
| 19 | ethyl | ethyl | cyclohexyl |
| 20 | ethyl | ethyl | 2-methylphenyl |
| 21 | ethyl | ethyl | ethylphenyl |
| 22 | ethyl | ethyl | 2-isopropylphenyl |
| 23 | ethyl | ethyl | 2,4-dimethylphenyl |
| 24 | ethyl | ethyl | diphenylmethyl |
| 25 | phenyl | phenyl | 4-methylphenyl |

| | | |
|---|---|---|
| 26 | —CH₂CH₂CH₂CH₂CH₂— | 4-nitro-3-aminophenyl |
| 27 | —CH₂CH₂CH₂CH₂CH₂— | 5-(1H)-benzo[d]imidazol- |
| | | 2(3H)-oyl) |
| 28 | —CH₂CH₂CH₂CH₂CH₂— | 4-methoxyphenyl |
| 29 | —CH₂CH₂CH₂CH₂CH₂— | 3-hydroxyphenyl |
| 30 | —CH₂CH₂CH₂CH₂CH₂— | phenyl |
| 31 | —CH₂CH₂CH₂CH₂CH₂— | 3-methoxyphenyl |
| 32 | —CH₂CH₂CH₂CH₂CH₂— | 2-methyoxyphenyl |
| 33 | —CH₂CH₂CH₂CH₂CH₂— | 2-hydroxyphenyl |
| 34 | —CH₂CH₂CH₂CH₂CH₂— | 2-pyridyl |
| 35 | —CH₂CH₂CH₂CH₂CH₂— | 2-chlorophenyl |
| 36 | —CH₂CH₂CH₂CH₂CH₂— | 4-chlorophenyl |
| 37 | —CH₂CH₂CH₂CH₂CH₂— | 4-trifluoromethylphenyl |
| 38 | —CH₂CH₂CH₂CH₂CH₂— | 4-pyridyl |
| 39 | —CH₂CH₂CH₂CH₂CH₂— | 4-hydroxyphenyl |
| 40 | —CH₂CH₂CH₂CH₂CH₂— | 3-pyridyl |
| 41 | —CH₂CH₂CH₂CH₂CH₂— | 4-cyanophenyl |
| 42 | —CH₂CH₂CH₂CH₂CH₂— | 4-cyano-2-pyridyl |
| 43 | —CH₂CH₂CH₂CH₂CH₂— | 4-trifluoromethyl-2-pyridyl |
| 44 | —CH₂CH₂CH₂CH₂CH₂— | 4-chloro-2-pyridyl |
| 45 | —CH₂CH₂CH₂CH₂CH₂— | 5-indolyl |
| 46 | —CH₂CH₂CH₂CH₂CH₂— | 3-chloro-4-pyridyl |
| 47 | —CH₂CH₂CH₂CH₂CH₂— | 3-chloro-2-pyridyl |
| 48 | —CH₂CH₂CH₂CH₂CH₂— | 3-cyano-2-pyridyl |
| 49 | —CH₂CH₂CH₂CH₂CH₂— | 3-methoxy-2-pyridyl |
| 50 | —CH₂CH₂CH₂CH₂CH₂— | 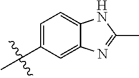 [SMILES: *c1ccc2[nH]c(C)nc2c1] |
| 51 | —CH₂CH₂CH₂CH₂— | 5-indolyl |
| 52 | —CH₂CH₂CH₂CH₂— | 2-isopropylphenyl |
| 53 | —CH₂CH₂CH₂CH₂— | 2-t-butylphenyl |
| 54 | —CH₂CH₂CH₂CH₂— | 2,6-diisopropylphenyl |
| 55 | —CH₂CH₂CH₂CH₂— | 2-morphilinophenyl |
| 56 | —CH₂CH₂CH₂CH₂— | 4-chloro-2-pyridyl |
| 57 | —CH₂CH₂CH₂CH₂— | 4-trifluoromethyl-2-pyridyl |
| 58 | —CH₂CH₂CH₂CH₂— | 4-methyl-2-pyridyl |
| 59 | —CH₂CH₂CH₂CH₂— | 4-hydroxy-2-pyridyl |
| 60 | —CH₂CH₂CH₂CH₂— | 4-cyano-2-pyridyl |
| 61 | —CH₂CH₂CH₂CH₂— | 4-pyridyl |
| 62 | —CH₂CH₂CH₂CH₂— | 4-fluoro-2-pyridyl |
| 63 | —CH₂CH₂CH₂CH₂— | 5-(1H)-benzo[d]imidazol- |
| | | 2(3H)-oyl) |
| 64 | —CH₂CH₂CH₂CH₂— | 4-(methylsulfonamide)phenyl |
| 65 | —CH₂CH₂CH₂CH₂— | 3-methoxy-2-pyridyl |
| 66 | —CH₂CH₂CH₂CH₂— | 3-chloro-2-pyridyl |
| 67 | —CH₂CH₂CH₂CH₂— | 3-chloro-4-pyridyl |
| 68 | —CH₂CH₂CH₂CH₂— | 3-cyano-2-pyridyl |
| 69 | —CH₂CH₂CH₂CH₂— | 2-methoxyphenyl |
| 70 | —CH₂CH₂CH₂CH₂— | 2-Hydroxyphenyl |
| 71 | —CH₂CH₂CH₂— | 5-indolyl |
| 72 | —CH₂CH₂CH₂— | 5-(1H)-benzo[d]imidazol- |
| | | 2(3H)-oyl) |
| 73 | —CH₂CH₂CH₂— | 2-methoxyphenyl |
| 74 | —CH₂CH₂CH₂— | 2-hydroxyphenyl |
| 75 | —CH₂CH₂CH₂— | 3-cyano-2-pyridyl |
| 76 | —CH₂CH₂CH₂— | 3-chloro-4-pyridyl |

Exemplary embodiments include compounds having the formula (I) or a pharmaceutically acceptable salt form thereof defined herein below in Table 18.

TABLE 18

| Entry | Structure |
|---|---|
| 1 | 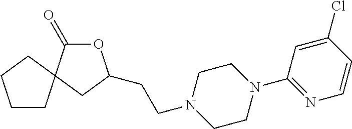 [SMILES: O=C1OC(CCN2CCN(c3cc(Cl)ccn3)CC2)CC12CCCC2] |
| 2 | 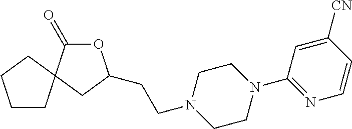 [SMILES: N#Cc1ccnc(N2CCN(CCC3CC4(CCCC4)C(=O)O3)CC2)c1] |
| 3 | 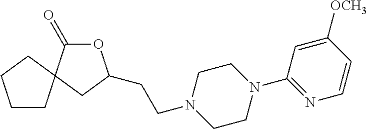 [SMILES: COc1ccnc(N2CCN(CCC3CC4(CCCC4)C(=O)O3)CC2)c1] |
| 4 | 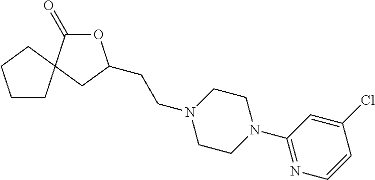 [SMILES: O=C1OC(CCN2CCN(c3cc(Cl)ccn3)CC2)CC12CCCC2] |
| 5 | 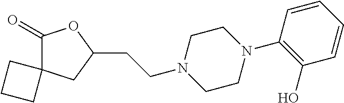 [SMILES: O=C1OC(CCN2CCN(c3ccccc3O)CC2)CC12CCC2] |
| 6 | 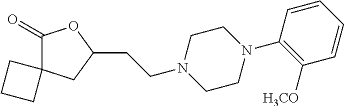 [SMILES: COc1ccccc1N1CCN(CCC2CC3(CCC3)C(=O)O2)CC1] |
| 7 | 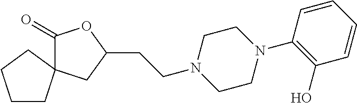 [SMILES: O=C1OC(CCN2CCN(c3ccccc3O)CC2)CC12CCCC2] |
| 8 | 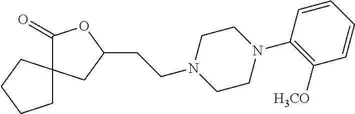 [SMILES: COc1ccccc1N1CCN(CCC2CC3(CCCC3)C(=O)O2)CC1] |
| 9 | 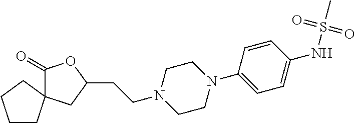 [SMILES: CS(=O)(=O)Nc1ccc(N2CCN(CCC3CC4(CCCC4)C(=O)O3)CC2)cc1] |
| 10 | 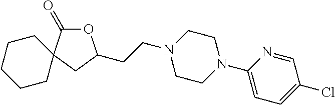 [SMILES: O=C1OC(CCN2CCN(c3ccc(Cl)cn3)CC2)CC12CCCCC2] |
| 11 | 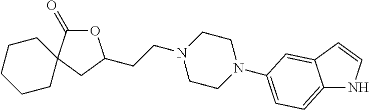 [SMILES: O=C1OC(CCN2CCN(c3ccc4[nH]ccc4c3)CC2)CC12CCCCC2] |
| 12 | 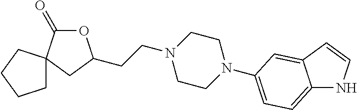 [SMILES: O=C1OC(CCN2CCN(c3ccc4[nH]ccc4c3)CC2)CC12CCCC2] |
| 13 | 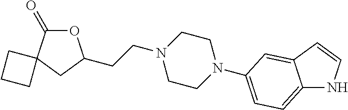 [SMILES: O=C1OC(CCN2CCN(c3ccc4[nH]ccc4c3)CC2)CC12CCC2] |
| 14 | 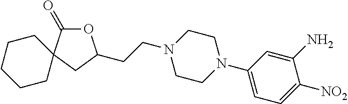 [SMILES: Nc1cc(N2CCN(CCC3CC4(CCCCC4)C(=O)O3)CC2)ccc1[N+](=O)[O-]] |
| 15 | 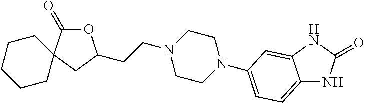 [SMILES: O=C1OC(CCN2CCN(c3ccc4[nH]c(=O)[nH]c4c3)CC2)CC12CCCCC2] |
| 16 | 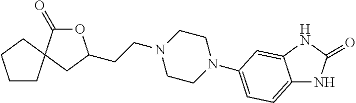 [SMILES: O=C1OC(CCN2CCN(c3ccc4[nH]c(=O)[nH]c4c3)CC2)CC12CCCC2] |
| 17 | 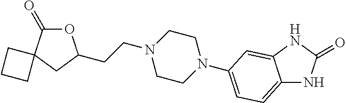 [SMILES: O=C1OC(CCN2CCN(c3ccc4[nH]c(=O)[nH]c4c3)CC2)CC12CCC2] |
| 18 | 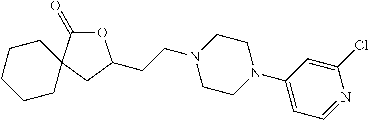 [SMILES: O=C1OC(CCN2CCN(c3ccnc(Cl)c3)CC2)CC12CCCCC2] |
| 19 | 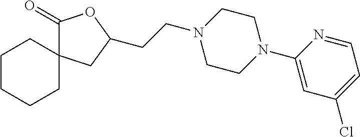 [SMILES: O=C1OC(CCN2CCN(c3cc(Cl)ccn3)CC2)CC12CCCCC2] |
| 20 | 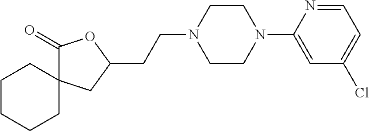 [SMILES: O=C1OC(CCN2CCN(c3cc(Cl)ccn3)CC2)CC12CCCCC2] |
| 21 | 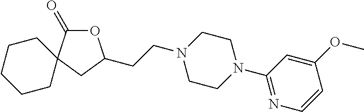 [SMILES: COc1ccnc(N2CCN(CCC3CC4(CCCCC4)C(=O)O3)CC2)c1] |
| 22 | 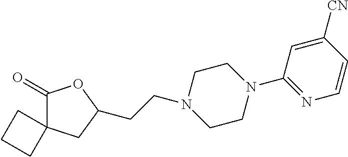 [SMILES: N#Cc1ccnc(N2CCN(CCC3CC4(CCC4)C(=O)O3)CC2)c1] |
| 23 | 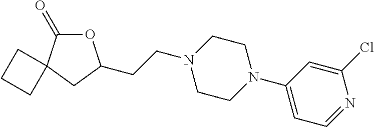 [SMILES: O=C1OC(CCN2CCN(c3ccnc(Cl)c3)CC2)CC12CCC2] |
| 24 | 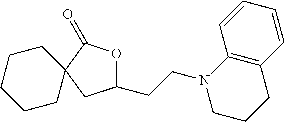 [SMILES: O=C1OC(CCN2CCCc3ccccc32)CC12CCCCC2] |
| 25 | 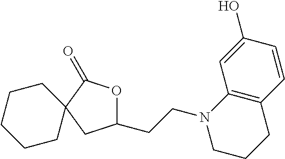 [SMILES: O=C1OC(CCN2CCCc3ccc(O)cc32)CC12CCCCC2] |
| 26 | 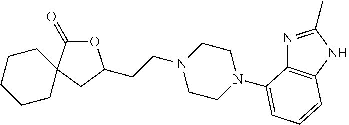 [SMILES: Cc1nc2c(N3CCN(CCC4CC5(CCCCC5)C(=O)O4)CC3)cccc2[nH]1] |
| 27 | 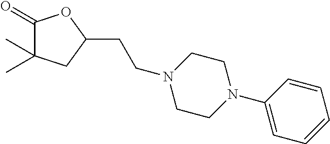 [SMILES: CC1(C)CC(CCN2CCN(c3ccccc3)CC2)OC1=O] |
| 28 | 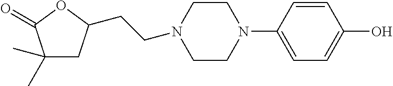 [SMILES: CC1(C)CC(CCN2CCN(c3ccc(O)cc3)CC2)OC1=O] |
| 29 | 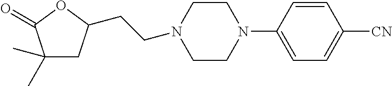 [SMILES: CC1(C)CC(CCN2CCN(c3ccc(C#N)cc3)CC2)OC1=O] |
| 30 | 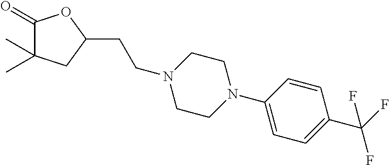 [SMILES: CC1(C)CC(CCN2CCN(c3ccc(C(F)(F)F)cc3)CC2)OC1=O] |
| 31 | 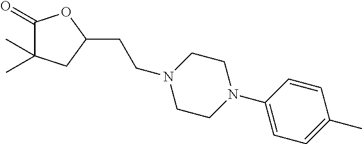 |

In one embodiment, the compound of the invention is selected from the group consisting of:
3-{2-[4-(5-chloro-pyridin-2-yl)-piperazin-1-yl]-ethyl}-2-oxa-spiro[4.5]decan-1-one;
3-{2-[4-(1H-indol-5-yl)-piperazin-1l-yl]-ethyl}-2-oxa-spiro[4.5]decan-1-one;
3-{2-[4-(1H-indol-5-yl)-piperazin-1-yl]-ethyl}-2-oxa-spiro[4.4]nonan-1-one;
7-{2-[4-(1H-indol-5-yl)-piperazin-1-yl]-ethyl}-6-oxa-spiro[3.4]octan-5-one;
3-(2-(4-(-3-amino-4-nitrophenyl)piperazin-1-yl)ethyl)-2-oxaspiro[4.5]decan-1-one;
5-(4-(2-(1-oxo-2-oxaspiro[4.5]decan-3-yl)ethyl)piperazin-1-yl)-1H-benzo[d]imidazol-2(3H)-one;
5-(4-(2-(1-oxo-2-oxaspiro[4.4]nonan-3-yl)piperazin-1-yl)-1H-benzo[d]imidazol-2(3H)-one;
5-(4-(2-(1-oxo-2-oxaspiro[3.4]octan-3-yl)piperazin-1-yl)-1H-benzo[d]imidazol-2(3H)-one;
3-(2-(4-(2-chloropyridin-4-yl)piperazin-1-yl)ethyl)-2-oxaspiro[4.5]decan-1-one;
3-{2-[4-(4-chloro-pyridin-2-yl)-piperazin-1-yl]-ethyl}-2-oxa-spiro[4.5]decan-1-one;
2-(4-(2-(1-oxo-2-oxaspiro[4.5]decan-3-yl)ethyl)piperazin-1-yl)isonicotinonitrile;
3-(2-(4-(4-methoxypyridin-2-yl)piperazin-1-yl)ethyl)-2-oxaspiro[4.5]decan-1-one;
2-(4-(2-(5-oxo-6-oxaspiro[3.4]octan-7-yl)ethyl)piperazin-1-yl)isonicotinonitrile;
7-(2-(4-(2-chloropyridin-4-yl)piperazin-1-yl)ethyl)-6-oxaspiro[3.4]octan-5-one;
3-(2-(3,4-dihydroquinolin-1 (2H)-yl)ethyl)-2-oxaspiro[4.5]decan-1-one;
3-(2-(7-hydroxy-3,4-dihydroquinolin-1 (2H)-yl)ethyl)-2-oxaspiro[4.5]decan-1-one;
3-(2-(4-(2-methyl-1H-benzo[d]imidazol-4-yl)piperazin-1-yl)ethyl)-2-oxaspiro[4.5]decan-1-one;
dihydro-3,3-dimethyl-5-(2-(4-phenylpiperazin-1-yl)ethyl)furan-2(3H)-one;
dihydro-5-(2-(4-(4-hydroxyphenyl)piperazin-1-yl)ethyl)-3,3-dimethylfuran-2(3H)-one;
4-(4-(2-(tetrahydro-4,4-dimethyl-5-oxofuran-2-yl)ethyl)piperazin-1-yl)benzonitrile;
5-(2-(4-(4-(trifluoromethyl)phenyl)piperazin-1-yl)ethyl)-dihydro-3,3-dimethylfuran-2(3H)-one;
dihydro-3,3-dimethyl-5-(2-(4-p-tolylpiperazin-1-yl)ethyl)furan-2(3H)-one;
3-(2-(4-(4-chloropyridin-2-yl)piperazin-1-yl)ethyl)-2-oxaspiro[4.4]nonan-1-one;
2-(4-(2-(1-oxo-2-oxaspiro[4.4]nonan-3-yl)ethyl)piperazin-1-yl)isonicotinonitrile;
3-(2-(4-(4-methoxypyridin-2-yl)piperazin-1-yl)ethyl)-2-oxaspiro[4.4]nonan-1-one;
3-(2-(4-(2-chloropyridin-4-yl)piperazin-1-yl)ethyl)-2-oxaspiro[4.4]nonan-1-one;
7-(2-(4-(2-hydroxyphenyl)piperazin-1-yl)ethyl)-6-oxaspiro[3.4]octan-5-one;
7-(2-(4-(2-methoxyphenyl)piperazin-1-yl)ethyl)-6-oxaspiro[3.4]octan-5-one;
3-(2-(4-(2-hydroxyphenyl)piperazin-1-yl)ethyl)-2-oxaspiro[4.4]nonan-1-one;
3-(2-(4-(2-methoxyphenyl)piperazin-1-yl)ethyl)-2-oxaspiro[4.4]nonan-1-one;
N-(4-(4-(2-(1-oxo-2-oxaspiro[4.4]nonan-3-yl)ethyl)piperazin-1-yl)phenyl)methanesulfonamide;
2-(4-(2-(4,4-diethyl-5-oxotetrahydrofuran-2-yl)ethyl)piperazin-1-yl)benzonitrile;
3,3-diethyl-5-(2-(4-(4-methoxyphenyl)piperazin-1-yl)ethyl)dihydrofuran-2(3H)-one;
3,3-diethyl-5-(2-(4-(4-hydroxyphenyl)piperazin-1-yl)ethyl)dihydrofuran-2(3H)-one;
3,3-diethyl-5-(2-(4-(4-nitrophenyl)piperazin-1-yl)ethyl)dihydrofuran-2(3H)-one;
3,3-diethyl-5-(2-(4-(2-methoxyphenyl)piperazin-1-yl)ethyl)dihydrofuran-2(3H)-one;
4-(4-(2-(4,4-diethyl-5-oxotetrahydrofuran-2-yl)ethyl)piperazin-1-yl)benzonitrile;
3,3-diethyl-5-(2-(4-(2-hydroxyphenyl)piperazin-1-yl)ethyl)dihydrofuran-2(3H)-one;
3,3-diethyl-5-(2-(4-phenylpiperazin-1-yl)ethyl)dihydrofuran-2(3H)-one;
5-(2-(4-(4-aminophenyl)piperazin-1-yl)ethyl)-3,3-diethyldihydrofuran-2(3H)-one;
3,3-diethyl-5-(2-(4-(p-tolyl)piperazin-1-yl)ethyl)dihydrofuran-2(3H)-one;
3,3-diethyl-5-(2-(4-(3-methoxyphenyl)piperazin-1-yl)ethyl)dihydrofuran-2(3H)-one;
3,3-diethyl-5-(2-(4-(3-hydroxyphenyl)piperazin-1-yl)ethyl)dihydrofuran-2(3H)-one;
3,3-diethyl-5-(2-(4-(pyridin-2-yl)piperazin-1-yl)ethyl)dihydrofuran-2(3H)-one;
5-(2-(4-(2,6-dimethylphenyl)piperazin-1-yl)ethyl)-3,3-diethyldihydrofuran-2(3H)-one;
5-(2-(4-cyclohexylpiperazin-1-yl)ethyl)-3,3-diethyldihydrofuran-2(3H)-one;
3,3-diethyl-5-(2-(4-(o-tolyl)piperazin-1-yl)ethyl)dihydrofuran-2(3H)-one;
3,3-diethyl-5-(2-(4-phenylpiperidin-1-yl)ethyl)dihydrofuran-2(3H)-one;
3,3-diethyl-5-(2-(4-phenethylpiperazin-1-yl)ethyl)dihydrofuran-2(3H)-one;
3,3-diethyl-5-(2-(4-(2-isopropylphenyl)piperazin-1-yl)ethyl)dihydrofuran-2(3H)-one;
5-(2-(4-(2,4-dimethylphenyl)piperazin-1-yl)ethyl)-3,3-diethyldihydrofuran-2(3H)-one;
3,3-diethyl-5-(2-(4-(4-fluorobenzyl)piperazin-1-yl)ethyl)dihydrofuran-2(3H)-one;
5-(2-(4-benzhydrylpiperazin-1-yl)ethyl)-3,3-diethyldihydrofuran-2(3H)-one;
5-(2-(3,4-dihydroisoquinolin-2(1H)-yl)ethyl)-3,3-diethyldihydrofuran-2(3H)-one;
2-(4-(2-(4,4-dimethyl-5-oxotetrahydrofuran-2-yl)ethyl)piperazin-1-yl)benzonitrile;
3,3-diethyl-5-(2-(4-(hydroxydiphenylmethyl)piperidin-1-yl)ethyl)dihydrofuran-2(3H)-one;
5-(2-(4-(diphenylmethylene)piperidin-1-yl)ethyl)-3,3-diethyldihydrofuran-2(3H)-one;
3-(2-(4-(2-isopropylphenyl)piperazin-1-yl)ethyl)-2-oxaspiro[4.4]nonan-1-one;
3-(2-(4-(2-isopropylphenyl)piperazin-1-yl)ethyl)-2-oxaspiro[4.5]decan-1-one;
5-(2-(4-(2-isopropylphenyl)piperazin-1-yl)ethyl)-3,3-diphenyldihydrofuran-2(3H)-one;
5-(2-(4-(1H-benzo[d]imidazol-2-yl)piperidin-1-yl)ethyl)-3,3-diethyldihydrofuran-2(3H)-one
5-(2-(4-benzoylpiperazin-1-yl)ethyl)-3,3-diethyldihydrofuran-2(3H)-one;
3-(2-(4-(2-(tert-butyl)phenyl)piperazin-1-yl)ethyl)-2-oxaspiro[4.4]nonan-1-one;
3-(2-(4-(2,6-diisopropylphenyl)piperazin-1-yl)ethyl)-2-oxaspiro[4.4]nonan-1-one;
3-(2-(4-(2-morpholinophenyl)piperazin-1-yl)ethyl)-2-oxaspiro[4.4]nonan-1-one;
3-(2-(4-([1,1′-biphenyl]-2-yl)piperazin-1-yl)ethyl)-2-oxaspiro[4.4]nonan-1-one;
3-(2-(4-(2-(1H-pyrrol-1-yl)phenyl)piperazin-1-yl)ethyl)-2-oxaspiro[4.4]nonan-1-one;
3-(2-(4-(2-iodophenyl)piperazin-1-yl)ethyl)-2-oxaspiro[4.4]nonan-1-one;
N-(2-(4-(2-(1-oxo-2-oxaspiro[4.4]nonan-3-yl)ethyl)piperazin-1-yl)phenyl)acetamide;
3-(2-(4-(naphthalen-1-yl)piperazin-1-yl)ethyl)-2-oxaspiro[4.4]nonan-1-one;
3-(2-(4-(anthracen-1-yl)piperazin-1-yl)ethyl)-2-oxaspiro[4.4]nonan-1-one;
3-(2-(4-(benzo[c][1,2,5]thiadiazol-4-yl)piperazin-1-yl)ethyl)-2-oxaspiro[4.4]nonan-1-one;
5-nitro-2-(4-(2-(1-oxo-2-oxaspiro[4.4]nonan-3-yl)ethyl)piperazin-1-yl)benzonitrile;
3-(2-(4,4-diphenylpiperidin-1-yl)ethyl)-2-oxaspiro[4.4]nonan-1-one;
3-(2-(4-benzhydrylpiperidin-1-yl)ethyl)-2-oxaspiro[4.4]nonan-1-one;
3-(2-(4-hydroxy-4-phenylpiperidin-1-yl)ethyl)-2-oxaspiro[4.4]nonan-1-one;
1-(2-(1-oxo-2-oxaspiro[4.4]nonan-3-yl)ethyl)-4-phenylpiperidine-4-carbonitrile;
1-(2-(4-(2-isopropylphenyl)piperazin-1-yl)ethyl)-2-oxaspiro[4.5]decan-3-one;
5-(2-(4-(2-isopropylphenyl)piperazin-1-yl)ethyl)-4,4-dimethyldihydrofuran-2(3H)-one;
3-(3-(4-(2-isopropylphenyl)piperazin-1-yl)propyl)-2-oxaspiro[4.4]nonan-1-one;
3-(3-(4-(2-isopropylphenyl)piperazin-1-yl)propyl)-2-oxaspiro[4.5]decan-1-one;
3,3-diphenyl-5-(2-(4-(p-tolyl)piperazin-1-yl)ethyl)dihydrofuran-2(3H)-one;
3,3-diphenyl-5-(3-(4-(p-tolyl)piperazin-1-yl)propyl)dihydrofuran-2(3H)-one;
3-(2-(4-(p-tolyl)piperazin-1-yl)ethyl)-2-oxaspiro[4.4]nonan-1-one;
3-(3-(4-(p-tolyl)piperazin-1-yl)propyl)-2-oxaspiro[4.4]nonan-1-one;
3-(2-(4-(p-tolyl)piperazin-1-yl)ethyl)-2-oxaspiro[4.5]decan-1-one;
3-(3-(4-(p-tolyl)piperazin-1-yl)propyl)-2-oxaspiro[4.5]decan-1-one;
3,3-diethyl-5-((4-(p-tolyl)piperazin-1-yl)methyl)dihydrofuran-2(3H)-one;
1-(2-(4-(p-tolyl)piperazin-1-yl)ethyl)-2-oxaspiro[4.5]decan-3-one;
4,4-dimethyl-5-(2-(4-(p-tolyl)piperazin-1-yl)ethyl)dihydrofuran-2(3H)-one;
3,3-diphenyl-5-((4-(p-tolyl)piperazin-1-yl)methyl)dihydrofuran-2(3H)-one;
3-((4-(p-tolyl)piperazin-1-yl)methyl)-2-oxaspiro[4.4]nonan-1-one;
3-((4-(p-tolyl)piperazin-1-yl)methyl)-2-oxaspiro[4.5]decan-1-one;
(S)-dihydro-5-(2-(4-(3-hydroxyphenyl)piperazin-1-yl)ethyl)-3,3-dimethylfuran-2(3H)-one;
(S)-dihydro-5-(2-(4-(3-methoxyphenyl)piperazin-1-yl)ethyl)-3,3-dimethylfuran-2(3H)-one;
(S)-dihydro-5-(2-(4-(4-methoxyphenyl)piperazin-1-yl)ethyl)-3,3-dimethylfuran-2(3H)-one;
(S)-dihydro-3,3-dimethyl-5-(2-(4-phenylpiperidin-1-yl)ethyl)furan-2(3H)-one;
(S)-dihydro-5-(2-(4-hydroxy-4-phenylpiperidin-1-yl)ethyl)-3,3-dimethylfuran-2(3H)-one;
(R)-dihydro-5-(2-(4-(3-hydroxyphenyl)piperazin-1-yl)ethyl)-3,3-dimethylfuran-2(3H)-one;
(R)-dihydro-5-(2-(4-(3-methoxyphenyl)piperazin-1-yl)ethyl)-3,3-dimethylfuran-2(3H)-one;
(R)-dihydro-5-(2-(4-(4-methoxyphenyl)piperazin-1-yl)ethyl)-3,3-dimethylfuran-2(3H)-one;
(R)-dihydro-3,3-dimethyl-5-(2-(4-phenylpiperidin-1-yl)ethyl)furan-2(3H)-one;
(R)-dihydro-5-(2-(4-hydroxy-4-phenylpiperidin-1-yl)ethyl)-3,3-dimethylfuran-2(3H)-one;
(S)-dihydro-5-(2-(4-(2-isopropylphenyl)piperazin-1-yl)ethyl)-3,3-dimethylfuran-2(3H)-one;
(R)-dihydro-5-(2-(4-(2-isopropylphenyl)piperazin-1-yl)ethyl)-3,3-dimethylfuran-2(3H)-one;
3,3-diethyl-dihydro-5-(3-(4-phenylpiperazin-1-yl)propyl)furan-2(3H)-one;
4-(4-(3-(4,4-diethyl-tetrahydro-5-oxofuran-2-yl)propyl)piperazin-1-yl)benzonitrile;
3,3-diethyl-dihydro-5-(3-(4-(4-hydroxyphenyl)piperazin-1-yl)propyl)furan-2(3H)-one;
3,3-diethyl-dihydro-5-(3-(4-(4-methoxyphenyl)piperazin-1-yl)propyl)furan-2(3H)-one;
3,3-diethyl-dihydro-5-(3-(4-p-tolylpiperazin-1-yl)propyl)furan-2(3H)-one;
3,3-diethyl-dihydro-5-(3-(4-(2-isopropylphenyl)piperazin-1-yl)propyl)furan-2(3H)-one;
3,3-diethyl-dihydro-5-(3-(4-(pyridin-2-yl)piperazin-1-yl)propyl)furan-2(3H)-one;
3,3-diethyl-5-(2-(4-(2-(trifluoromethyl)-1H-benzo[d]imidazol-6-yl)piperazin-1-yl)ethyl)-dihydrofuran-2(3H)-one;
3,3-diethyl-dihydro-5-(2-(4-(2-methyl-1H-benzo[d]imidazol-6-yl)piperazin-1-yl)ethyl)furan-2(3H)-one;
(S)-3,3-diethyl-dihydro-5-(2-(4-(pyridin-4-yl)piperazin-1-yl)ethyl)furan-2(3H)-one;
(R)-3,3-diethyl-dihydro-5-(2-(4-(pyridin-4-yl)piperazin-1-yl)ethyl)furan-2(3H)-one;
3,3-diethyl-dihydro-5-(2-(4-(pyrimidin-2-yl)piperazin-1-yl)ethyl)furan-2(3H)-one;
3,3-diethyl-5-(2-(4-(3-(trifluoromethyl)phenyl)piperazin-1-yl)ethyl)-dihydrofuran-2(3H)-one;
5-(2-(4-([1,1′-biphenyl]-2-yl)piperazin-1-yl)ethyl)-3,3-diethyldihydrofuran-2(3H)-one;
3,3-diethyl-dihydro-5-(2-(4-m-tolylpiperazin-1-yl)ethyl)furan-2(3H)-one;
5-(2-(4-(2,4-dichlorophenyl)piperazin-1-yl)ethyl)-3,3-diethyl-dihydrofuran-2(3H)-one;
5-(2-(4-(2-chlorophenyl)piperazin-1-yl)ethyl)-3,3-diethyldihydrofuran-2(3H)-one;
5-(2-(4-(3,5-dichlorophenyl)piperazin-1-yl)ethyl)-3,3-diethyldihydrofuran-2(3H)-one;
3,3-diethyl-5-(2-(4-(naphthalen-1-yl)piperazin-1-yl)ethyl)dihydrofuran-2(3H)-one;
3-(4-(2-(4,4-diethyl-5-oxotetrahydrofuran-2-yl)ethyl)piperazin-1-yl)benzonitrile;
5-(2-(4-(benzo[d]isoxazol-3-yl)piperazin-1-yl)ethyl)-3,3-diethyldihydrofuran-2(3H)-one;
3,3-diethyl-5-(2-(4-(2-(trifluoromethyl)phenyl)piperazin-1-yl)ethyl)dihydrofuran-2(3H)-one;
3,3-diethyl-5-(2-(4-(pyrazin-2-yl)piperazin-1-yl)ethyl)dihydrofuran-2(3H)-one;
3,3-diethyl-5-(2-(4-(pyrazin-2-yl)piperazin-1-yl)ethyl)dihydrofuran-2(3H)-one;
3,3-diethyl-5-(2-(4-(3-fluorophenyl)piperazin-1-yl)ethyl)dihydrofuran-2(3H)-one;
(R)-5-(2-(4-phenylpiperazin-1-yl)ethyl)dihydrofuran-2(3H)-one;
(5S)-3-methyl-5-(2-(4-phenylpiperazin-1-yl)ethyl)dihydrofuran-2(3H)-one;
(5R)-3-phenyl-5-(2-(4-phenylpiperazin-1-yl)ethyl)dihydrofuran-2(3H)-one;
(5R)-5-(2-(4-(4-hydroxyphenyl)piperazin-1-yl)ethyl)-3-phenyldihydrofuran-2(3H)-one;
(5R)-3-phenyl-5-(2-(4-(pyridin-4-yl)piperazin-1-yl)ethyl)dihydrofuran-2(3H)-one;
(5S)-3-phenyl-5-(2-(4-phenylpiperazin-1-yl)ethyl)dihydrofuran-2(3H)-one;
(5S)-5-(2-(4-(4-hydroxyphenyl)piperazin-1-yl)ethyl)-3-phenyldihydrofuran-2(3H)-one;
(5S)-3-phenyl-5-(2-(4-(pyridin-4-yl)piperazin-1-yl)ethyl)dihydrofuran-2(3H)-one;
3-(2-(4-(3-chloropyridin-4-yl)piperazin-1-yl)ethyl)-2-oxaspiro[4.5]decan-1-one;
3-(2-(4-(3-methylpyridin-4-yl)piperazin-1-yl)ethyl)-2-oxaspiro[4.5]decan-1-one;
3-(2-(4-(2-methylpyridin-4-yl)piperazin-1-yl)ethyl)-2-oxaspiro[4.5]decan-1-one;
4-(4-(2-(1-oxo-2-oxaspiro[4.5]decan-3-yl)ethyl)piperazin-1-yl)nicotinonitrile;
4-(4-(2-(1-oxo-2-oxaspiro[4.5]decan-3-yl)ethyl)piperazin-1-yl)picolinonitrile;
3-(2-(4-(2-methoxypyridin-4-yl)piperazin-1-yl)ethyl)-2-oxaspiro[4.5]decan-1-one;
4-(4-(2-(4,4-diethyl-5-oxotetrahydrofuran-2-yl)ethyl)piperazin-1-yl)phenyl acetate;
4-(4-(2-(4,4-diethyl-tetrahydro-5-oxofuran-2-yl)ethyl)piperazin-1-yl)phenyl 2-ethylbutanoate;
4-(4-(2-(4,4-diethyl-tetrahydro-5-oxofuran-2-yl)ethyl)piperazin-1-yl)phenyl isobutyrate;
4-(4-(2-(4,4-diethyl-5-oxotetrahydrofuran-2-yl)ethyl)piperazin-1-yl)phenyl ethyl carbonate;
4-(4-(2-(4,4-diethyl-5-oxotetrahydrofuran-2-yl)ethyl)piperazin-1-yl)phenyl methyl carbonate;
4-(4-(2-(4,4-diethyl-5-oxotetrahydrofuran-2-yl)ethyl)piperazin-1-yl)phenyl isopropyl carbonate;
4-(4-(2-(4,4-diethyl-5-oxotetrahydrofuran-2-yl)ethyl)piperazin-1-yl)phenyl dimethylcarbamate;
4-(4-(2-(4,4-diethyl-5-oxotetrahydrofuran-2-yl)ethyl)piperazin-1-yl)phenyl diethylcarbamate;
5-(2-(7-chloro-3,4-dihydroisoquinolin-2(1H)-yl)ethyl)-3,3-diethyl-dihydrofuran-2(3H)-one;
3,3-diethyl-5-(2-(7-fluoro-3,4-dihydroisoquinolin-2(1H)-yl)ethyl)-dihydrofuran-2(3H)-one;
and
5-(2-(7-bromo-3,4-dihydroisoquinolin-2(1H)-yl)ethyl)-3,3-diethyldihydrofuran-2(3H)-one;
or a pharmaceutically acceptable form thereof

In one embodiment, the compound is selected from the group consisting of:

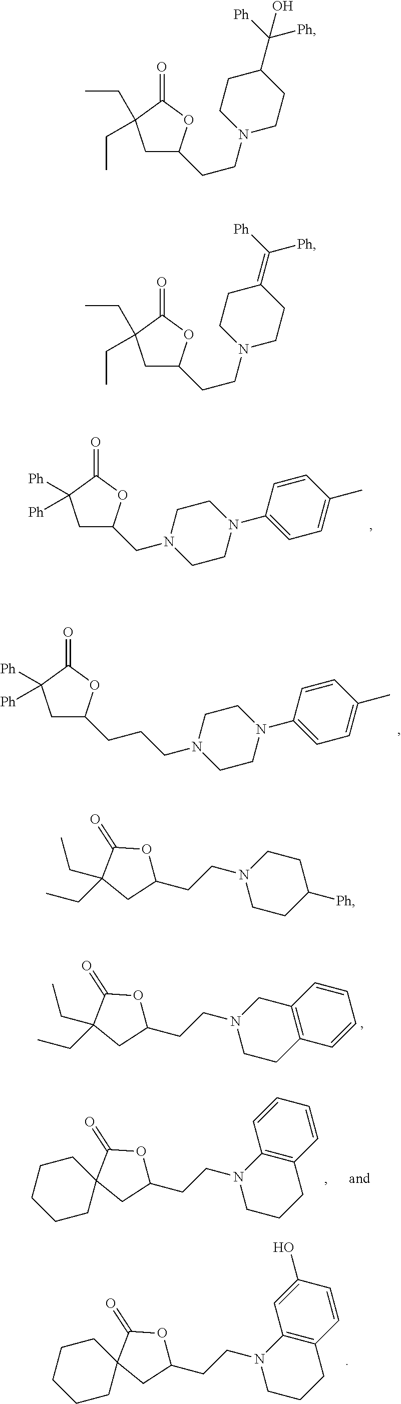

For the purposes of demonstrating the manner in which the compounds of the present invention are named and referred to herein, the compound having the formula:

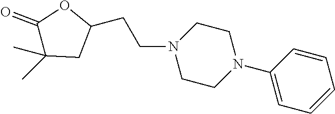 [SMILES: CC1(C)CC(CCN2CCN(c3ccccc3)CC2)OC1=O]

has the chemical name dihydro-3,3-dimethyl-5-(2-(4-phenylpiperazin-1-yl)ethyl)furan-2(3H)-one.

For the purposes of the present invention, a compound depicted by the racemic formula, for example:

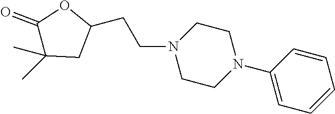 [SMILES: CC1(C)CC(CCN2CCN(c3ccccc3)CC2)OC1=O]

will stand equally well for either of the two enantiomers having the formula:

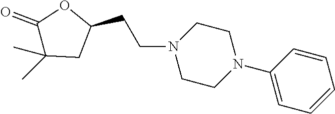 [SMILES: CC1(C)C[C@H](CCN2CCN(c3ccccc3)CC2)OC1=O]

or the formula:

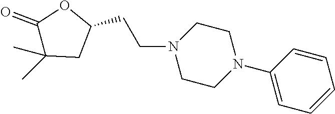 [SMILES: CC1(C)C[C@@H](CCN2CCN(c3ccccc3)CC2)OC1=O]

or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Methods

The present invention also relates to a method for treating or preventing diseases that involve dysregulation of sigma-2 receptor activity, for example neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, schizophrenia, Alzheimer's disease, mild cognitive impairment, and memory disorders, as well as cancer, for example pancreatic cancer, lung cancer, breast cancer, melanoma, prostate cancer, and ovarian cancer. The method comprises administering to a subject an effective amount of a compound or composition according to the present invention.

The present invention yet further relates to a method for treating or preventing diseases that involve dysregulation of sigma-2 receptor activity, for example neuropsychiatric disorders such as generalized anxiety disorder, social anxiety disorder, panic disorder, agoraphobia, obsessive-compulsive disorder post-traumatic stress disorder, depression, bipolar disorder, anorexia nervosa, bulimia nervosa, substance use disorders, schizophrenia, Alzheimer's disease, mild cognitive impairment, and memory disorders, as well as cancer, for example pancreatic cancer, lung cancer, breast cancer, melanoma, prostate cancer, and ovarian cancer. The method comprises administering to a subject a composition comprising an effective amount of one or more compounds according to the present invention and an excipient.

The present invention yet further relates to a method for treating or preventing diseases that involve overexpression of the sigma-2 receptor such as cancer, for example pancreatic cancer, lung cancer, breast cancer, melanoma, prostate cancer, and ovarian cancer. The method comprises administering to a subject an effective amount of a compound or composition according to the present invention.

The present invention yet further relates to a method for treating or preventing diseases that involve overexpression of the sigma-2 receptor such as cancer, for example pancreatic cancer, lung cancer, breast cancer, melanoma, prostate cancer, and ovarian cancer. The method comprises administering to a subject a composition comprising an effective amount of one or more compounds according to the present invention and an excipient.

The present invention also relates to a method for treating or preventing disease or conditions associated with dysregulation of sigma-2 receptor activity. The method comprises administering to a subject an effective amount of a compound or composition according to the present invention.

The present invention yet further relates to a method for treating or preventing disease or conditions associated with dysregulation of sigma-2 receptor activity. The method comprises administering to a subject a composition comprising an effective amount of one or more compounds according to the present invention and an excipient.

### Process

The present invention further relates to a process for preparing the sigma-2 receptor binders and sigma-2 receptor activity modulators of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

### General Synthetic Schemes for Preparation of Compounds

The reagents used in the preparation of the compounds of this invention can be either commercially obtained or can be prepared by standard procedures described in the literature. In accordance with this invention, compounds in the genus may be produced by one of the following reaction schemes.

Compounds of the disclosure may be prepared according to the process outlined in Scheme 1:

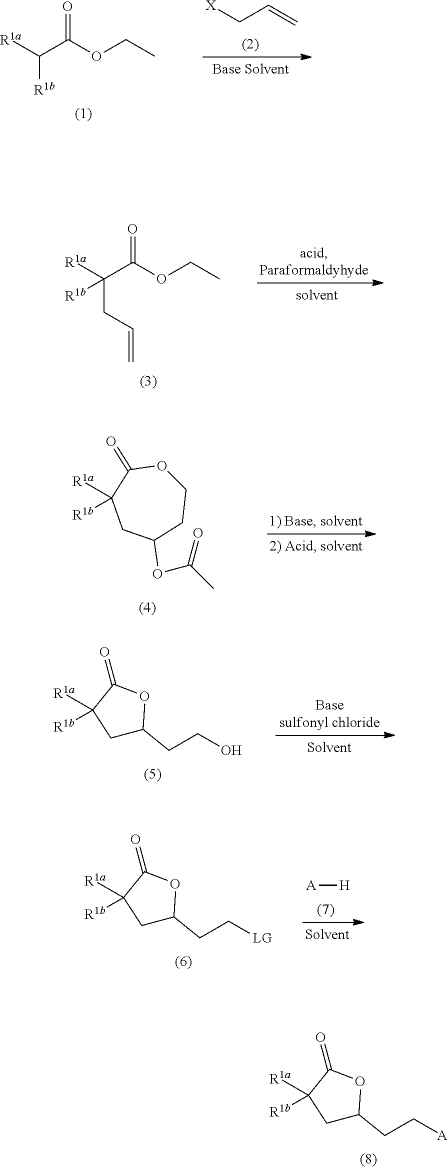

A suitably substituted compound of formula (1), a known compound or compound prepared by known methods, is reacted with a compound of the formula (2), wherein X is a leaving group such as chlorine, bromine, iodine, mesylate, tosylate, and the like, in the presence of a base such as lithium diisopropylamide, sodium diisopropylamide, potassium diisopropylamide, lithium bis(trimethylsilyl)amide, sodium bis(trimethylsilyl)amide, potassium bis(trimethylsilyl)amide, sodium hydride, optionally in the presence of hexamethylphosphoramide (HMPA), and the like in an organic solvent such as tetrahydrofuran, 1,4-dioxane, 1,2-dimethoxyethane, dimethylformamide, dimethylacetamide, and the like, to provide a compound of the formula (3). A compound of the formula (3) is then treated with paraformaldehyde in the presence of an acid such as sulfuric acid, hydrochloric acid, and the like, in an the presence of acetic acid, and optionally in an organic solvent such as methanol, ethanol, tetrahydrofuran, 1,4-dioxane, 1,2-dimethoxyethane, dimethylformamide, dimethylacetamide, and the like, optionally with heating, optionally with microwave irradiation to provide a compound of the formula (4). A compound of the formula (4) is then treated with a base such as sodium hydroxide, potassium hydroxide, lithium hydroxide, and the like, in an solvent such as water, methanol, ethanol, isopropanol, and the like, optionally with heating, and then treated with an acid such as sulfuric acid, hydrochloric acid, and the like, in a solvent such as water, methanol, ethanol, isopropanol, and the like, to provide a compound of the formula (5). A compound of the formula (5) is then converted to a compound of the formula (6), wherein LG is a mesylate, tosylate, nosylate, and the like, using methods that are known to one skilled in the art. Thus, a compound of the formula (5) is treated with a sulfonyl chloride such as methanesulfonyl chloride, toluenesulfonyl chloride p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (6). A compound of the formula (6) is reacted with a compound of the formula (7), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (8).

Alternatively, compounds of formula (8) may be prepared according to the process outlined in Scheme 2:

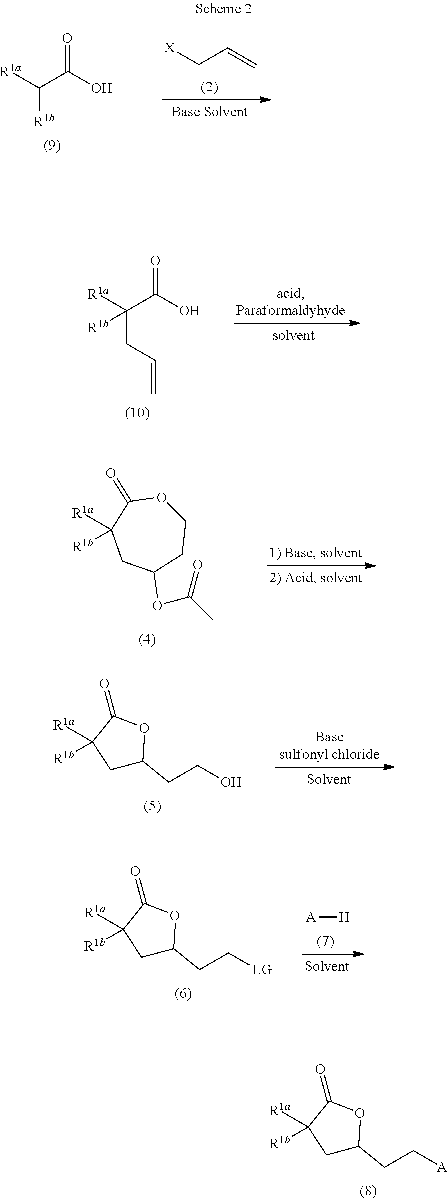

A suitably substituted compound of formula (9), a known compound or compound prepared by known methods, is reacted with a compound of the formula (2), wherein X is a leaving group such as chlorine, bromine, iodine, mesylate, tosylate, and the like, in the presence of a base such as lithium diisopropylamide, sodium diisopropylamide, potassium diisopropylamide, lithium bis(trimethylsilyl)amide, sodium bis(trimethylsilyl)amide, potassium bis(trimethylsilyl)amide, sodium hydride, n-butyl lithium, sec-butyl lithium, tert-butyl lithium, and the like, optionally in the presence of hexamethylphosphoramide (HMPA), in an organic solvent such as tetrahydrofuran, 1,4-dioxane, 1,2-dimethoxyethane, dimethylformamide, dimethylacetamide, and the like, to provide a compound of the formula (10). A compound of the formula (10) is then treated with paraformaldehyde in the presence of an acid such as sulfuric acid, hydrochloric acid, and the like, in the presence of acetic acid, and optionally in an organic solvent such as methanol, ethanol, tetrahydrofuran, 1,4-dioxane, 1,2-dimethoxyethane, dimethylformamide, dimethylacetamide, and the like, optionally with heating, optionally with microwave irradiation to provide a compound of the formula (4). A compound of the formula (4) is then treated with a base such as sodium hydroxide, potassium hydroxide, lithium hydroxide, and the like, in an solvent such as water, methanol, ethanol, isopropanol, and the like, optionally with heating, and then treated with an acid such as sulfuric acid, hydrochloric acid, and the like, in a solvent such as water, methanol, ethanol, isopropanol, and the like, optionally with heating, to provide a compound of the formula (5). A compound of the formula (5) is then converted to a compound of the formula (6), wherein LG is a mesylate, tosylate, nosylate, and the like, using methods that are known to one skilled in the art. Thus, a compound of the formula (5) is treated with a sulfonyl chloride such as methanesulfonyl chloride, toluenesulfonyl chloride p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (6). A compound of the formula (6) is reacted with a compound of the formula (7), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (8).

Compounds of formula (21) may be prepared according to the process outlined in Scheme 3.

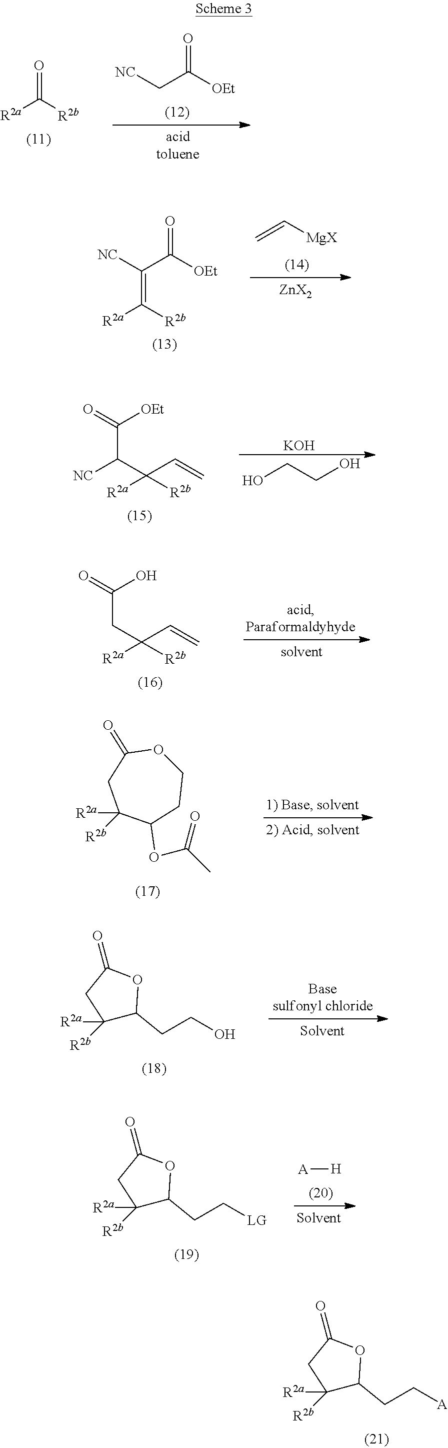

A suitably substituted compound of formula (11), a known compound or compound prepared by known methods, is reacted with a compound of the formula (12), in the presence of an ammonium salt such as ammonium acetate, ammonium formate, ammonium sulfate, ammonium chloride, and the like, in the presence of an acid such as formic acid, acetic acid, hydrochloric acid, sulfuric acid, and the like, in an organic solvent such as toluene, benzene, p-xylene, m-xylene, o-xylene, tetrahydrofuran, 1,4-dioxane, dimethylformamide, and the like, optionally with heating, optionally with microwave irradiation to provide a compound of the formula (13). A compound of the formula (13) is then reacted with a compound of the formula (14), wherein X is a halogen, in the presence of a compound of the formula ZnX₂, wherein X is a halogen, in an organic solvent such as tetrahydrofuran, 1,4-dioxane, 1,2-dimethoxyethane, dimethylformamide, dimethylacetamide, and the like to provide a compound of the formula (15). A compound of the formula (15) is then reacted with a base such as potassium hydroxide, sodium hydroxide, lithium hydroxide, potassium carbonate, sodium carbonate, lithium carbonate, and the like, in a solvent such as methanol, ethanol, isopropanol, ethylene glycol, propylene glycol, water, and the like, optionally with heating to provide a compound of the formula (16). A compound of the formula (16) is reacted with paraformaldehyde in the presence of an acid such as sulfuric acid, hydrochloric acid, and the like, in the presence of acetic acid, optionally in the presence of a solvent such as methanol, ethanol, tetrahydrofuran, 1,4-dioxane, dimethyl formamide, and the like, optionally with heating to provide a compound of the formula (17). A compound of the formula (17) is then treated with a base such as sodium hydroxide, potassium hydroxide, lithium hydroxide, and the like, in an solvent such as water, methanol, ethanol, isopropanol, and the like, optionally with heating, and then treated with an acid such as sulfuric acid, hydrochloric acid, and the like, in a solvent such as water, methanol, ethanol, isopropanol, and the like, optionally with heating, to provide a compound of the formula (18). A compound of the formula (18) is then converted to a compound of the formula (19), wherein LG is a mesylate, tosylate, nosylate, and the like, using methods that are known to one skilled in the art. Thus, a compound of the formula (18) is treated with a sulfonyl chloride such as methanesulfonyl chloride, toluenesulfonyl chloride p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (19). A compound of the formula (19) is reacted with a compound of the formula (20), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (21).

Compounds of formula (26) may be prepared according to the process outlined in Scheme 4.

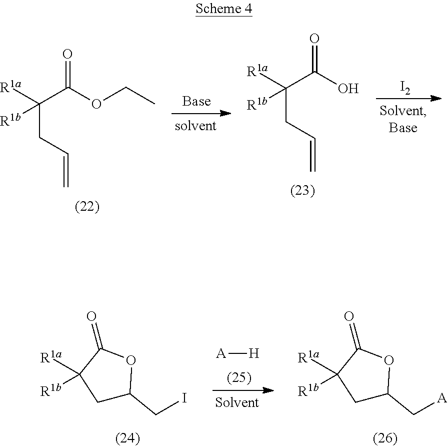

A compound of the formula (22) is reacted with a base such as sodium hydroxide, potassium hydroxide, lithium hydroxide, sodium carbonate, potassium carbonate, lithium carbonate and the like, in the presence of a solvent such as methanol, ethanol, isopropanol, water, and the like, optionally with heating, optionally with microwave irradiation to provide a compound of the formula (23). A compound of the formula (23) is then reacted with iodine in the presence of a base such as sodium bicarbonate, potassium bicarbonate, lithium bicarbonate, sodium carbonate, potassium carbonate, lithium bicarbonate, sodium hydroxide, potassium hydroxide, lithium hydroxide, and the like, in the presence of a solvent such as tetrahydrofuran, ethyl ether, 1,4-dioxane, and the like to provide a compound of the formula (24). A compound of the formula (24) is reacted with a compound of the formula (25), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (26).

Compounds of formula (35) may be prepared according to the process outlined in Scheme 5.

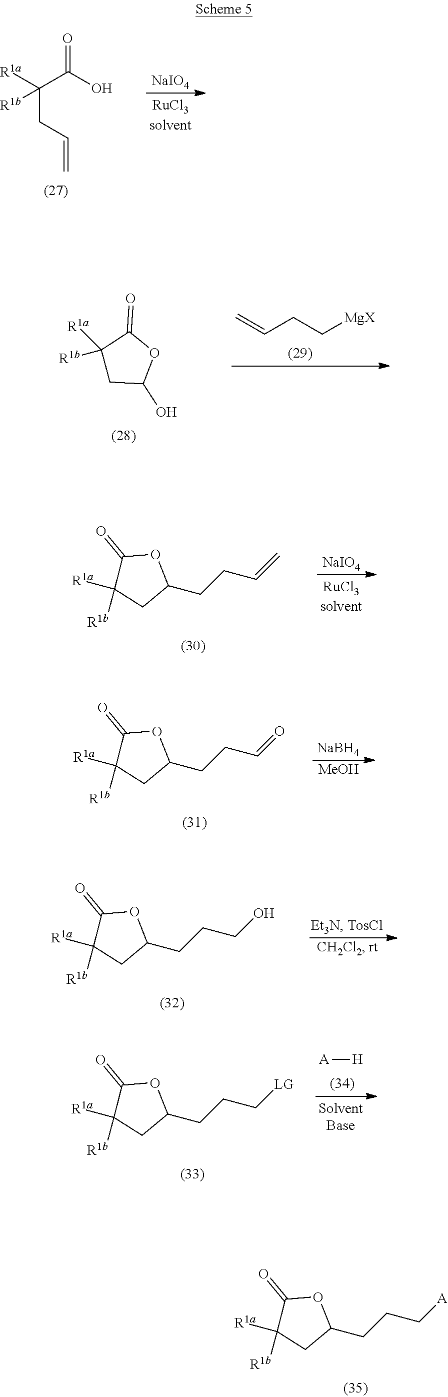

A compound of the formula (27) is reacted with ruthenium chloride in the presence of sodium periodate in a solvent such as acetonitrile, methanol, ethanol, isopropanol, and the like, to provide a compound of the formula (28). A compound of the formula (28) is reacted with a compound of the formula (29), a known compound or compound prepared by known methods, wherein x is a halogen, in the presence of a solvent such as ethyl ether, tetrahydrofuran, 1,4-dioxane and the like to provide a compound of the formula (30). A compound of the formula (30) is reacted with ruthenium chloride in the presence of sodium periodate in a solvent such as acetonitrile, methanol, ethanol, isopropanol, and the like, to provide a compound of the formula (31). A compound of the formula (31) is reacted with a reducing agent such as lithium borohydride, sodium borohydride, sodium cyanoborohydride and the like, in a solvent such as methanol, ethanol, isopropanol, acetonitrile, and the like to provide a compound of the formula (32). A compound of the formula (32) is then converted to a compound of the formula (33), wherein LG is a mesylate, tosylate, nosylate, and the like, using methods that are known to one skilled in the art. Thus, a compound of the formula (32) is treated with a sulfonyl chloride such as methanesulfonyl chloride, toluenesulfonyl chloride p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (33). A compound of the formula (33) is reacted with a compound of the formula (34), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (35).

Compounds of formula (44) may be prepared according to the process outlined in Scheme 6.

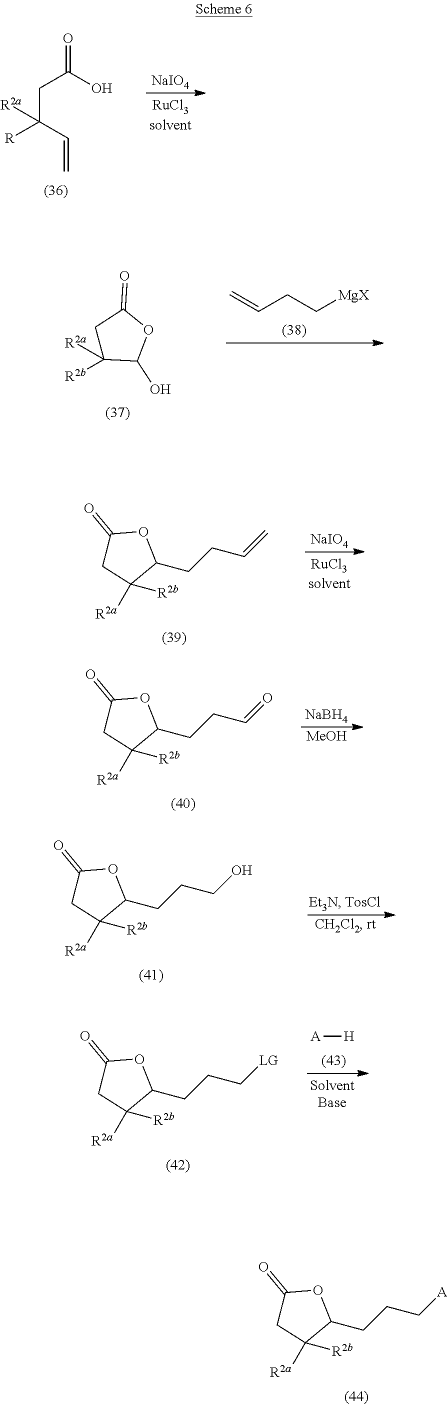

A compound of the formula (36) is reacted with ruthenium chloride in the presence of sodium periodate in a solvent such as acetonitrile, methanol, ethanol, isopropanol, and the like, to provide a compound of the formula (37). A compound of the formula (37) is reacted with a compound of the formula (38), a known compound or compound prepared by known methods, wherein x is a halogen, in the presence of a solvent such as ethyl ether, tetrahydrofuran, 1,4-dioxane and the like to provide a compound of the formula (39). A compound of the formula (39) is reacted with ruthenium chloride in the presence of sodium periodate in a solvent such as acetonitrile, methanol, ethanol, isopropanol, and the like, to provide a compound of the formula (40). A compound of the formula (40) is reacted with a reducing agent such as lithium borohydride, sodium borohydride, sodium cyanoborohydride and the like, in a solvent such as methanol, ethanol, isopropanol, acetonitrile, and the like to provide a compound of the formula (41). A compound of the formula (41) is then converted to a compound of the formula (42), wherein LG is a mesylate, tosylate, nosylate, and the like, using methods that are known to one skilled in the art. Thus, a compound of the formula (41) is treated with a sulfonyl chloride such as methanesulfonyl chloride, toluenesulfonyl chloride p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (42). A compound of the formula (42) is reacted with a compound of the formula (43), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (44).

Compounds of formula (48) may be prepared according to the process outlined in Scheme 7.

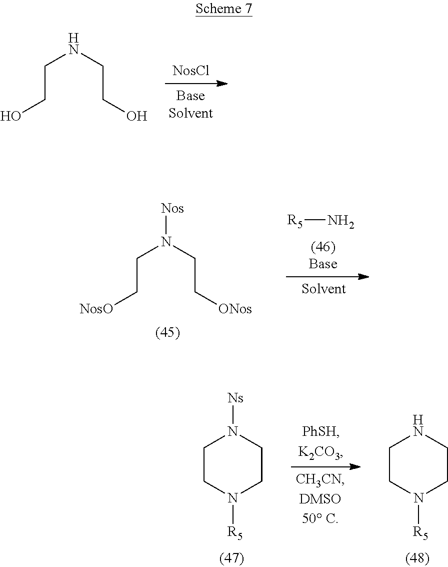

Diethanolamine is reacted with 4-nitrobenzenesulfonyl chloride (NosCl) in the presence of a base such as triethylamine, diisopropylethylamine, pyridine, 2,6-lutidine, and the like in a solvent such as tetrahydrofuran, 1,4-dioxane, methylene chloride and the like to provide a compound of the formula (45). A compound of the formula (45) is then reacted with a compound of the formula (46), a known compound or one prepared by known methods, in the presence of a base such as triethylamine, diisopropylethylamine, pyridine, 2,6-lutidine, and the like, in a solvent such as acetonitrile, methanol, ethanol, dimethyl formamide, optionally with heating, optionally with microwave irradiation to provide a compound of the formula (47). A compound of the formula (47) is reacted with a thiophenol in the presence of a base such as sodium bicarbonate, potassium bicarbonate, lithium bicarbonate, sodium carbonate, potassium carbonate, lithium bicarbonate, sodium hydroxide, potassium hydroxide, lithium hydroxide, and the like, in the presence of a solvent such as tetrahydrofuran, ethyl ether, 1,4-dioxane, acetonitrile and the like, optionally in the presence of dimethylsulfoxide, optionally with heating, optionally with microwave irradiation, to provide a compound of the formula (48).

Compounds of formula (58) may be prepared according to the process outlined in Scheme 8.

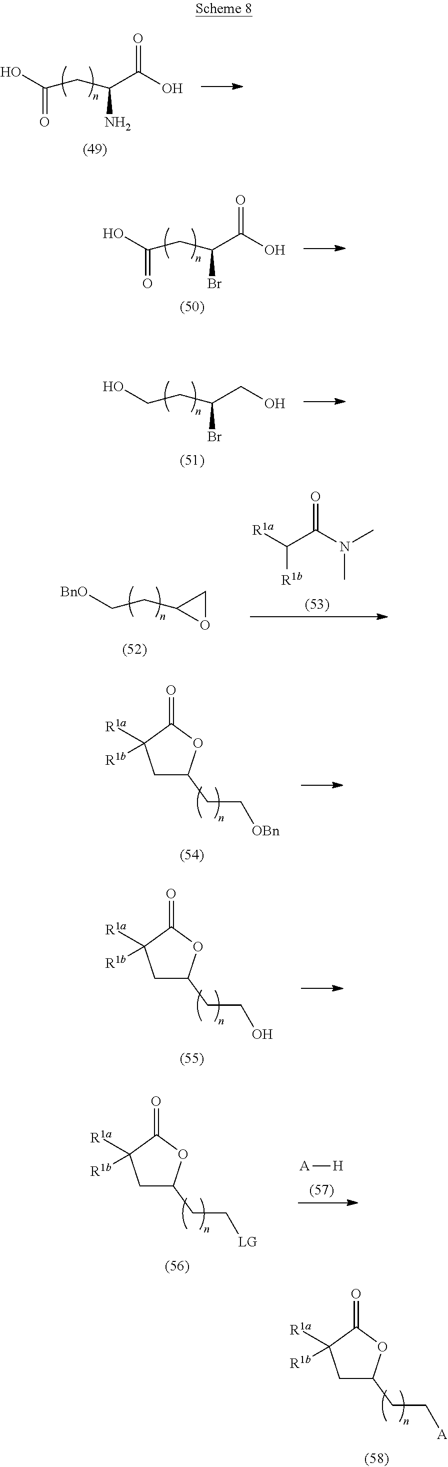

A compound of the formula (49), a known compound or a compound prepared by known methods, is reacted with a bromide salt such as sodium bromide, lithium bromide, tetrabutyl ammonium bromide, potassium bromide, and the like, in the presence of a nitrite salt such as sodium nitrite, potassium nitrite, lithium nitrite, tetrabutyl ammonium nitrite, and the like, in a solvent such as methanol, ethanol; tetrahydrofuran, 1,4-dioxane, N,N-dimethylformamide, methylene chloride, and the like, optionally with heating, optionally with microwave irradiation to provide a compound of the formula (50). A compound of the formula (50) is reacted with a reducing agent such as borane dimethyl sulfide, lithium aluminum hydride, and the like in the presence of a solvent such as tetrahydrofuran, 1,4-dioxane, methylene chloride, and the like to provide a compound of the formula (51). A compound of the formula (51) is reacted with benzyl bromide in the presence of a base such as lithium carbonate, sodium carbonate, potassium carbonate, triethyl amine, diisopropylethyl amine, puridine, 2,6-lutidinde, and the like, in the presence of a solvent such as tetrahydrofuran, 1,4-dioxane, methylene chloride, N,N-dimethylformamide, methylene chloride, and the like, optionally with heating, optionally with microwave irradiation to provide a compound of the formula (52). Alternatively, a compound of the formula (51) is reacted with benzyl chloride in the presence of a base such as lithium carbonate, sodium carbonate, potassium carbonate, triethyl amine, diisopropylethyl amine, puridine, 2,6-lutidinde, and the like, in the presence of a solvent such as tetrahydrofuran, 1,4-dioxane, methylene chloride, N,N-dimethylformamide, methylene chloride, and the like, optionally with heating, optionally with microwave irradiation to provide a compound of the formula (52). A compound of the formula (52) is reacted with a compound of the formula (53), a known compound or a compound prepared by known methods, in the presence of a base such as lithium diisopropylamide, sodium diisopropylamide, potassium diisopropylamide, lithium bis(trimethylsilyl)amide, sodium bis(trimethylsilyl)amide, potassium bis(trimethylsilyl)amide, sodium hydride, n-butyl lithium, sec-butyl lithium, tert-butyl lithium, and the like, optionally in the presence of hexamethylphosphoramide (HMPA), in the presence of a solvent such as tetrahydrofuran, 1,4-dioxane, 1,2-dimethoxyethane, methylene chloride, and the like, to provide a compound of the formula (54). A compound of the formula (54) is reacted with hydrogen in the presence of a palladium catalyst such as palladium on carbon, palladium acetate, palladium bis(triphenylphosphine) dichloride, palladium tetrakis(triphenylphospine), bis(acetonitrile), and the like, in an organic solvent such as methanol, ethanol, isopropanol, ethyl acetate, tetrahydrofuran, 1,4-dioxane, and the like to provide a compound of the formula (55). A compound of the formula (55) is then converted to a compound of the formula (56), wherein LG is a mesylate, tosylate, nosylate, and the like, using methods that are known to one skilled in the art. Thus, a compound of the formula (55) is treated with a sulfonyl chloride such as methanesulfonyl chloride, toluenesulfonyl chloride, p-nitrophenyl sulfonyl chloride, and the like, in the presence of a base such as triethylamine, diisopropyl amine, pyridine, 2,6-lutidine, and the like, in an organic solvent such as methylene chloride, dichloroethane, tetrahydrofuran, 1,4-dioxane, N, N-dimethylformamide, tetrahydrofuran, 1,4-dioxane and the like to provide a compound of the formula (56). Alternatively, a compound of the formula (55) is then converted to a compound of the formula (56), wherein LG is a bromine atom. Thus, a compound of the formula (55) is reacted with carbon tetrabromide, in the presence of triphenylphosphine, in a solvent such as tetrahydrofuran, 1,4-dioxane, methylene chloride, N,N-dimethylformamide, and the like, to provide a compound of the formula (56). A compound of the formula (56) is reacted with a compound of the formula (57), a known compound or compound prepared by known methods, in an organic solvent such as tetrahydrofuran, 1,4-dioxane, 1,2-dimethoxyethane, dimethylformamide, dimethylacetamide, and the like, optionally in the presence of a base such as triethylamine, diisopropylethylamine, pyridine, 2,6 lutidine, and the like, optionally with heating, optionally with microwave irradiation to provide a compound of the formula (58).

The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

### Formulations

The present invention also relates to compositions or formulations which comprise the sigma-2 receptor binders and sigma-2 receptor activity modulators according to the present invention. In general, the compositions of the present invention comprise an effective amount of one or more compounds of the disclosure and salts thereof according to the present invention which are effective for providing modulation of sigma-2 receptor activity; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known sigma-2 receptor activity modulators. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.]

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more compounds of the disclosure according to the present invention and one or more excipients; from about 0.01 mg to about 100 mg of one or more compounds of the disclosure according to the present invention and one or more excipients; and from about 0.1 mg to about 10 mg of one or more compounds of the disclosure according to the present invention; and one or more excipients.

### EXAMPLES

The practice of the invention is illustrated by the following non-limiting examples. The Examples provided below provide representative methods for preparing exemplary compounds of the present invention. The skilled practitioner will know how to substitute the appropriate reagents, starting materials and purification methods known to those skilled in the art, in order to prepare the compounds of the present invention.

### Synthesis of Exemplary Compounds of the Invention

In the examples that follow, ¹H-NMR spectra were obtained on a Varian Mercury 300-MHz NMR. Purity (%) and mass spectral data were determined with a Waters Alliance 2695 HPLC/MS (Waters Symmetry C18, 4.6×75 mm, 3.5 m) with a 2996 diode array detector from 210-400 nm.

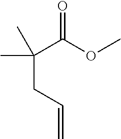

Preparation of methyl 2,2-dimethylpent-4-enoate: This reaction was performed in oven-dried glassware under a nitrogen atmosphere. To a well-stirred solution of freshly prepared lithium diisopropylammide (1M, 1.10 equiv) in dry 35 ml tetrahydrofuran, isobutyric acid methyl ester (3.32 g, 32.6 mmol, 1.0 equiv) was added dropwise during 0.5 hours at −78° C. The mixture was allowed to stir at this temperature for 30 min followed by the addition of allyl bromide (5.35 g, 44.0 mmol) and hexamethylphosphoramide (HMPA) (2.91 g, 16.3 mmol) dropwise over 0.5 h. The reaction mixture was stirred overnight at room temperature, quenched with 10% HCl (while cooling in ice bath) until acidic (pH=2). The organic layer was separated and the aqueous layer was extracted with hexanes (3×100 mL). The extract was washed with 10% NaHCO₃ (200 mL) and brine (200 mL). The solution was then dried over MgSO₄, concentrated in vacuo and distilled to give pure product. ¹H NMR (400 MHz, CDCl₃) δ 5.73 (dd, J=9.4, 17.7, 1H), 5.04 (dd, J=1.9, 13.5, 2H), 4.12 (q, J=7.1, 2H), 2.28 (d, J=7.4, 2H), 1.25 (t, J=7.1, 3H), 1.17 (s, 6H); ¹³C NMR (101 MHz, CDCl₃) δ 177.42, 134.42, 117.88, 77.68, 77.36, 77.04, 60.35, 44.91, 42.25, 24.92, 14.35

The following compounds can be prepared by the procedure of methyl 2,2-dimethylpent-4-enoate. The skilled practitioner will know how to substitute the appropriate reagents, starting materials and purification methods known to those skilled in the art, in order to prepare the compounds provided herein.

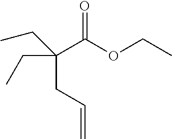

Preparation of ethyl 2,2-diethylpent-4-enoate: The title compound was prepared according to the procedure for methyl 2,2-dimethylpent-4-enoate, except 2-ethyl-butyric acid ethyl ester was substituted for isobutyric acid methyl ester. ¹H NMR (300 MHz, CDCl₃) δ 5.68 (dd, J=9.9, 17.2, 1H), 5.16-4.97 (m, 2H), 4.14 (q, J=7.1, 2H), 2.33 (d, J=7.4, 2H), 1.59 (dt, J=6.5, 7.5, 5H), 1.26 (t, J=7.1, 3H), 0.80 (t, J=7.5, 6H)

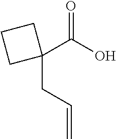

Preparation of 1-allylcyclobutanecarboxylic acid: This reaction was performed in oven-dried glassware under a nitrogen atmosphere. To a well-stirred solution of freshly prepared lithium diisopropylammide (1M, 10.76 mmol, 2.30 equiv) in dry 107 mL tetrahydrofuran, cyclobutanecarboxylic acid (4.68 g, 46.8 mmol, 1.0 equiv) was added dropwise during 0.5 hours at 0° C. The mixture was heated to 50° C. for 6 hours, then cooled to 0° C. followed by the addition of NaI (0.697 g, 4.68 mmol, 0.1 equiv) in one portion and a mixture of allyl bromide (7.58 g, 63.2 mmol, 1.35 equiv) and HMPA (4.18 g, 23.4 mmol, 0.5 equiv) dropwise over 0.5 hr. The reaction mixture was stirred overnight at room temperature, quenched with 10% HCl (while cooling in ice bath) until acidic (pH=2). The organic layer was separated and the aqueous layer was extracted with ether (3×250 mL). The organic phases were combined and washed with brine. The solution was then dried over MgSO₄ and concentrated in vacuo to afford a crude oil which was purified through flash chromatography (silica; ethyl acetate/hexanes, 1%-10%). ¹H NMR (400 MHz, CDCl₃) δ 5.77 (ddt, J=7.1, 10.2, 17.2, 1H), 5.17-4.99 (m, 2H), 2.59-2.38 (m, 4H), 2.07-1.84 (m, 4H). ¹³C NMR (101 MHz, CDCl₃) δ 184.04, 133.90, 118.19, 47.20, 41.74, 29.57, 15.65; Rf, 0.43 (hexane:ethyl acetate 10:1); HRMS (CI): [M+H], calcd for C₈H₁₃O₂, 141.0916; found 141.0911.

The following compounds can be prepared by the procedure of 1-allylcyclobutanecarboxylic acid. The skilled practitioner will know how to substitute the appropriate reagents, starting materials and purification methods known to those skilled in the art, in order to prepare the compounds provided herein.

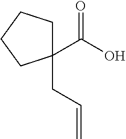

Preparation of 1-allylcyclopentanecarboxylic acid: The title compound was prepared according to the procedure for 1-allylcyclobutanecarboxylic acid, except cyclopentane carboxylic acid was substituted for cyclobutanecarboxylic acid. ¹H NMR (400 MHz, CDCl₃) δ 5.77 (ddt, J=7.2, 10.2, 17.4, 1H), 5.17-4.94 (m, 2H), 2.38 (d, J=7.2, 2H), 2.20-2.02 (m, 2H), 1.79-1.47 (m, 6H). ¹³C NMR (101 MHz, CDCl₃) δ 184.94, 134.96, 118.02, 53.75, 42.96, 35.89, 25.47. Rf, 0.50 (hexane:ethyl acetate 10:1); HRMS (CI): [M+H], calcd for C₉H₁₅O₂, 155.1072; found 155.1068.

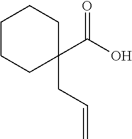

Preparation of 1-allylcyclohexanecarboxylic acid: The title compound was prepared according to the procedure for 1-allylcyclobutanecarboxylic acid, except cyclohexane carboxylic acid was substituted for cyclobutanecarboxylic acid. ¹H NMR (400 MHz, CDCl₃) δ 12.13 (broad, 1H), 5.83-5.63 (m, 1H), 5.12-5.00 (m, 2H), 2.27 (m, 2H), 2.04 (m, 2H), 1.66-1.50 (m, 3H), 1.49-1.33 (m, 2H), 1.33-1.17 (m, 3H).

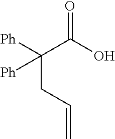 [SMILES: C=CCC(C(=O)O)(c1ccccc1)c1ccccc1]

Preparation of 2,2-diphenylpent-4-enoic acid: This reaction was performed in oven-dried glassware under a nitrogen atmosphere. To a well-stirred solution of 2.5M n-BuLi (8.27 ml, 20.68 mmol, 2.20 equiv) in hexanes, the starting acid (2.0 g, 9.4 mmol, 1.0 equiv) was dissolved in dry 100 ml tetrahydrofuran and added dropwise during 0.5 hours at −78° C. The mixture was allowed to stir for 1 hour, and then allyl bromide (1.52 g, 12.69 mmol, 1.35 equiv) was added dropwise over 0.5 h at the same temperature. The reaction mixture was stirred overnight at room temperature, quenched with 10% HCl (while cooling in ice bath) until acidic (pH=2). The organic layer was separated and the aqueous layer was extracted with ether (3×50 mL). The organic phases were combined and washed with brine. The solution was then dried over MgSO₄ and concentrated in vacuo to afford a crude oil which was purified through flash chromatography (silica; ethyl acetate/hexanes, 10%-20%) to provide 2,2-diphenylpent-4-enoic acid. ¹H NMR (400 MHz, CDCl₃) δ 7.37-7.17 (m, 10H), 5.58 (ddt, J=19.1, 9.6, 6.9 Hz, 1H), 4.98-4.85 (m, 2H), 3.16 (d, J=70 Hz, 2H).

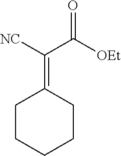 [SMILES: CCOC(=O)C(C#N)=C1CCCCC1]

Preparation of ethyl 2-cyano-2-cyclohexylideneacetate: A mixture of cyclohexanone (4.9 g, 50 mmol, 1 equiv), ethyl cyanoacetate (5.99 g, 53 mmol, 1.06 equiv), ammonium acetate (0.795 g, 15 mmol, 0.3 equiv), glacial acetic acid (3.0 g, 50 mmol, 1 equiv), and toluene (100 mL) was heated for 5 h at reflux under Dean-Stark conditions. The reaction mixture was cooled to room temperature and washed successively with water, NaHCO₃ solution, and brine. Drying, filtration, and evaporation of the organic phase provided crude oil that was purified via flash chromatography (silica; ethyl acetate/hexanes, 1%-10%). Yellowish oil (79% yield). ¹H NMR (400 MHz, CDCl₃) δ 4.26 (q, J=7.2 Hz, 2H), 3.03-2.89 (m, 2H), 2.74-2.52 (m, 2H), 1.92-1.61 (m, 6H), 1.34 (t, J=7.1 Hz, 3H); ¹³C NMR (101 MHz, CDCl₃) δ 180.15, 162.15, 115.75, 102.17, 61.84, 37.03, 31.71, 28.71, 28.39, 25.77, 14.21.

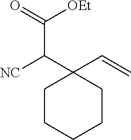 [SMILES: C=CC1(C(C#N)C(=O)OCC)CCCCC1]

Preparation of ethyl 2-cyano-2-(1-vinylcyclohexyl)acetate: This reaction was performed in oven-dried glassware under a nitrogen atmosphere. To well-stirred solution of zinc chloride (77.6 mL, 1M in THF, 2 equiv), vinylmagnesium bromide (227 ml, 0.7M in tetrahydrofuran, 4.1 equiv) was added dropwise at 0° C. The reaction mixture was then stirred for 15 min at room temperature. Then methyl magnesium bromide (24.5 ml, 3M in ether, 1.9 equiv) was added at 0° C. and the mixture was allowed to stir at room temperature for 15 min. Then ethyl 2-cyano-2-cyclohexylideneacetate (7.5 g, 38.81 mmol, 1 equiv) was dissolved in 10 mL of tetrahydrofuran and added to the mixture dropwise at 0° C. The mixture was allowed to stir at room temperature until the disappearance of the starting material. Reaction mixture was quenched with saturated NH₄Cl solution, extracted with ethyl acetate, dried over MgSO₄, concentrated in vacuo and purified by flash chromatography (silica; ethyl acetate/hexanes, 1%˜10%). Yellowish oil (73% yield). ¹H NMR (400 MHz, CDCl₃) δ 5.70 (dd, J=17.7, 10.9 Hz, 1H), 5.41-5.11 (m, 2H), 4.21 (m, 2H), 3.45 (s, 1H), 1.85 (dd, J=49.7, 13.3 Hz, 2H), 1.69-1.36 (m, 7H), 1.29 (t, J=7.1 Hz, 4H). ¹³C NMR (101 MHz, CDCl₃) δ 164.82, 139.76, 117.67, 115.64, 62.42, 48.75, 43.04, 33.82, 33.51, 25.71, 22.00, 21.94, 14.20.

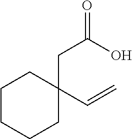

Preparation of 2-(1-vinylcyclohexyl)acetic acid: Ethyl 2-cyano-2-(1-vinylcyclohexyl)acetate (8.5 g, 38.41 mmol, 1.0 equiv) was gradually dissolved in a 15% (w/w) solution of KOH in ethylene glycol (5 mL) upon heating. The solution was further heated to reflux and was allowed to stir at that temperature the consumption of starting materials (6 h). The mixture was allowed to cool and was diluted with water (50 mL) and 1 N HCl (50 mL). Organics were extracted with ethyl acetate (3×200 mL). The combined organic layers were dried over MgSO₄, filtered, and concentrated in vacuo. The crude oil was purified via column chromatography column chromatography (Ethyl acetate/Hexanes, 5%-15%). Yellowish oil (82% yield), ¹H NMR (400 MHz, CDCl₃) δ 5.80 (dd, J=17.7, 10.9 Hz, 1H), 5.09 (ddd, J=18.7, 14.3, 1.0 Hz, 2H), 2.35 (s, 2H), 1.68 (dd, J=13.5, 9.2 Hz, 2H), 1.57-1.27 (m, 8H); ¹³C NMR (101 MHz, CDCl₃) δ 177.80, 144.72, 113.73, 45.77, 39.27, 35.70, 26.22, 22.20

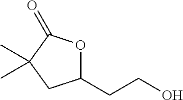

Preparation of 5-(2-hydroxy-ethyl)-3,3-dimethyl-dihydro-furan-2-one: A mixture of glacial acetic acid (28.6 g, 477 mmol, 53.6 equiv), paraformaldehyde (0.80 g, 26.7 mmol, 3.0 equiv) and H₂SO₄ (0.5 g, 4.45 mmol, 0.57 equiv) was stirred for 30 min at 70° C. before methyl 2,2-dimethylpent-4-enoate (1.26 g, 8.9 mmol, 1.0 equiv) was added dropwise during 10 min. The reaction mixture was then maintained at 70-80° C. and allowed to stir overnight. Acetic acid was removed under reduced pressure and the reaction was quenched with 10% NaHCO₃ solution. The mixture was then extracted with ethyl acetate (3×50 mL) and the combined organic phase was concentrated in vacuo to give a crude oil. The crude oil was used for next step without further purification.

A mixture of the crude oil (200 mg, 1.0 mmol, 1 equiv) and 30% NaOH (800 mg NaOH, 20 mmol, 20 equiv) aqueous solution was refluxed for 2 hours. The mixture was cooled in an ice bath and excess 30% H₂SO₄ was added until acidic (pH<2). The resulting mixture was extracted with ethyl acetate (3×25 mL), the combined organic phase was washed with 10% NaHCO₃, (50 mL), brine (50 mL), dried over MgSO₄ and concentrated in vacuo to give a crude product which was further purified by column chromatography (ethyl acetate/hexanes, 10%˜60%). ¹H NMR (400 MHz, CDCl₃) δ 4.70-4.60 (m, 1H), 3.90-3.78 (m, 2H), 2.22 (dd, J=5.9, 12.7, 1H), 1.98-1.87 (m, 2H), 1.80 (dd, J=5.9, 12.7, 1H), 1.28 (d, J=4.8, 6H). ¹³C NMR (101 MHz, CDCl₃) δ 182.26, 75.01, 59.58, 43.93, 40.62, 38.69, 25.31, 24.61; Rf, 0.34 (hexane:ethyl acetate 1:1); Anal. Calcd for C₈H₁₄O₃: C, 60.74; H, 8.92. Found: C, 60.47; H, 8.86.

The following compounds can be prepared by the procedure of 5-(2-Hydroxy-ethyl)-3,3-dimethyl-dihydro-furan-2-one. The skilled practitioner will know how to substitute the appropriate reagents, starting materials and purification methods known to those skilled in the art, in order to prepare the compounds provided herein.

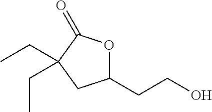

Preparation of 3,3-diethyl-5-(2-hydroxyethyl)dihydrofuran-2(3H)-one: The title compound was prepared according to the procedure for 5-(2-hydroxy-ethyl)-3,3-dimethyl-dihydro-furan-2-one, except ethyl 2,2-diethylpent-4-enoate was substituted for methyl 2,2-dimethylpent-4-enoate. ¹H NMR (400 MHz, CDCl₃) δ 4.62 (dtd, J=5.3, 7.3, 9.5, 1H), 3.78 (t, J=6.1, 2H), 3.20 (s, 1H), 2.19 (dd, J=6.8, 13.1, 1H), 1.97-1.81 (m, 3H), 1.70-1.56 (m, 4H), 0.93 (dt, J=7.5, 20.7, 6H); ¹³C NMR (101 MHz, CDCl₃) δ 181.46, 75.10, 58.91, 48.77, 39.13, 37.76, 29.21, 28.30, 8.83, 8.73; Rf, 0.36 (hexane:ethyl acetate 5:2); Anal. Calcd for C₁₀H₁₈O₃: C, 64.49; H, 9.74. Found: C, 64.20; H, 9.57.

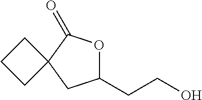 [SMILES: O=C1OC(CCO)CC12CCC2]

Preparation of 7-(2-hydroxyethyl)-6-oxaspiro[3.4]octan-5-one: The title compound was prepared according to the procedure for 5-(2-hydroxy-ethyl)-3,3-dimethyl-dihydro-furan-2-one, except 1-allylcyclobutanecarboxylic acid was substituted for methyl 2,2-dimethylpent-4-enoate: ¹H NMR (400 MHz, CDCl₃) δ 4.60-4.50 (m, 1H), 3.82 (t, J=5.9, 2H), 2.61-2.40 (m, 3H), 2.19-1.96 (m, 5H). 1.92-185 (m, 2H); ¹³C NMR (101 MHz, CDCl₃) δ 181.25, 75.46, 59.66, 44.62, 42.42, 38.47, 31.95, 29.64, 16.79; Rf, 0.40 (hexane:ethyl acetate 1:2); calcd for C₉H₁₅O₃, 171.1021; found 171.1016.

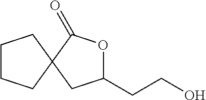 [SMILES: O=C1OC(CCO)CC12CCCC2]

Preparation of 3-(2-hydroxyethyl)-2-oxaspiro[4.4]nonan-1-one: The title compound was prepared according to the procedure for 5-(2-hydroxy-ethyl)-3,3-dimethyl-dihydro-furan-2-one, except 1-allylcyclopentanecarboxylic acid was substituted for methyl 2,2-dimethylpent-4-enoate: ¹H NMR (400 MHz, CDCl₃) δ 4.65-4.56 (m, 1H), 3.84-3.76 (m, 2H), 2.74 (s, 1H), 2.28 (dd, J=5.8, 12.6, 1H), 2.20-2.10 (m, 1H), 2.00-1.56 (m, 10H); ¹³C NMR (101 MHz, CDCl₃) δ 183.02, 75.77, 59.20, 50.35, 43.41, 38.41, 37.49, 36.93, 25.67, 25.58; Rf, 0.46 (hexane:ethyl acetate 1:2); HRMS (CI): [M+H], calcd for C₁₀H₁₇O₃, 185.1178; found 185.1171.

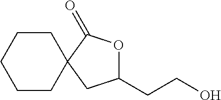 [SMILES: O=C1OC(CCO)CC12CCCCC2]

Preparation of 3-(2-hydroxyethyl)-2-oxaspiro[4.5]decan-1-one: The title compound was prepared according to the procedure for 5-(2-hydroxy-ethyl)-3,3-dimethyl-dihydro-furan-2-one, except 1-allylcyclohexanecarboxylic acid was substituted for methyl 2,2-dimethylpent-4-enoate: ¹H NMR (400 MHz, CDCl₃) δ 4.62 (m, 1H), 3.82 (t, J=5.9, 2H), 2.43 (dd, J=6.2, 12.9, 1H), 2.22 (s, 1H), 2.00-1.17 (m, 13H). ¹³C NMR (101 MHz, CDCl₃) δ 181.96, 75.37, 59.55, 45.13, 39.88, 38.91, 34.54, 31.71, 25.57, 22.42, 22.36; Rf, 0.46 (hexane:ethyl acetate 1:2); Anal. Calcd for C₁₁H₁₈O₃: C, 66.64; H, 9.15. Found: C, 66.48; H, 9.17.

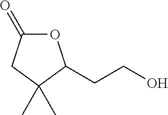

Preparation of 5-(2-hydroxyethyl)-4,4-dimethyldihydrofuran-2(3H)-one: The title compound was prepared according to the procedure for 5-(2-hydroxy-ethyl)-3,3-dimethyl-dihydro-furan-2-one, except 3,3-dimethyl-pent-4-enoic acid methyl ester was substituted for methyl 2,2-dimethylpent-4-enoate: ¹H NMR (400 MHz, CDCl₃) δ 4.27 (dd, J=9.4, 3.8 Hz, 1H), 3.88-3.66 (m, 2H), 2.81 (s, 1H), 2.35 (dd, J=43.6, 16.9 Hz, 2H), 1.82-1.66 (m, 2H), 1.14 (s, 3H), 1.01 (s, 3H); ¹³C NMR (101 MHz, CDCl₃) δ 176.44, 85.94, 59.72, 44.55, 39.17, 31.78, 25.04, 21.57.

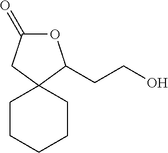 [SMILES: O=C1CC2(CCCCC2)C(CCO)O1]

Preparation of 1-(2-hydroxyethyl)-2-oxaspiro[4.5]decan-3-one: The title compound was prepared according to the procedure for 5-(2-hydroxy-ethyl)-3,3-dimethyl-dihydro-furan-2-one, except 2-(1-vinylcyclohexyl)acetic acid was substituted for methyl 2,2-dimethylpent-4-enoate: ¹H NMR (400 MHz, CDCl₃) δ 4.24 (dd, J=11.0, 2.4 Hz, 1H), 3.85-3.61 (m, 2H), 2.76 (s, 1H), 2.50 (d, J=17.2 Hz, 1H), 2.28 (d, J=17.3 Hz, 1H), 1.85-1.51 (m, 5H), 1.51-1.07 (m, 7H); ¹³C NMR (101 MHz, CDCl₃) δ 176.65, 86.03, 59.43, 42.78, 39.68, 34.94, 31.92, 29.72, 25.76, 23.09, 22.30.

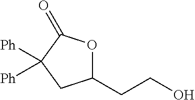 [SMILES: O=C1OC(CCO)CC1(c1ccccc1)c1ccccc1]

Preparation of 5-(2-hydroxyethyl)-3,3-diphenyldihydrofuran-2(3H)-one: A mixture of glacial acetic acid (28.6 g, 477 mmol, 53.6 equiv), paraformaldehyde (0.80 g, 26.7 mmol, 3.0 equiv) and H₂SO₄ (0.5 g, 4.45 mmol, 0.57 equiv) was stirred for 30 min at 70° C. before 2,2-diphenylpent-4-enoic acid (2.25 g, 8.9 mmol, 1.0 equiv) was added dropwise during 10 min. The reaction mixture was then maintained at 70-80° C. and allowed to stir overnight. Acetic acid was removed under reduced pressure and the reaction was quenched with 10% NaHCO₃ solution. The mixture was then extracted with ethyl acetate (3×50 mL) and the combined organic phase was concentrated in vacuo to give a crude oil. The crude oil was used for next step without further purification.

To a solution of crude oil (0.324 g, 1 mmol, 1 equiv) in THF (20 mL), 2% HCl (5.4 ml, 3 equiv) was added in one portion. The mixture was allowed to reflux for 48 hours followed by addition of brine (20 mL). The resulting mixture was extracted with ethyl acetate (3×20 mL). The combined organic phase was washed with 10% NaHCO₃, brine, dried over MgSO₄ and concentrated in vacuo to give crude oil which was then purified by column chromography (ethyl acetate/hexanes, 10%-50%). ¹H NMR (400 MHz, CDCl₃) δ 7.40-7.19 (m, 10H), 4.56 (m, 1H), 3.83 (t, J=6.0, 2H), 3.10 (dd, J=4.8, 13.0, 1H), 2.68 (dd, J=10.6, 13.0, 1H), 2.05-1.90 (m, 2H), 1.74 (s, 1H); ¹³C NMR (101 MHz, CDCl₃) δ 177.39, 142.28, 139.94, 129.29, 128.73, 128.08, 128.01, 127.68, 127.57, 75.28, 59.58, 58.36, 44.12, 38.01; Rf, 0.54 (Hexane:Ethyl Acetate 1:2); HRMS (CI): [M+H], calcd for C₁₀H₁₉O₃, 238.1334; found 283.1324.

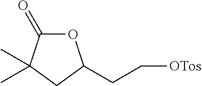

Preparation of 2-(4,4-dimethyl-5-oxotetrahydrofuran-2-yl)ethyl 4-methylbenzenesulfonate: To a stirred solution of 5-(2-hydroxy-ethyl)-3,3-dimethyl-dihydro-furan-2-one (0.316 g, 2 mmol, 1.0 equiv) and Et₃N (0.152 g, 1.5 mmol, 1.5 equiv) in dry dichloromethane, a solution of p-TosCl (0.475 g, 2.5 mmol, 1.25 equiv) in dichloromethane was added drop wise at 0° C. The resulting mixture was stirred at 0° C. for 1 hour and allowed to stir overnight at room temperature. Then, the reaction mixture was diluted with dichloromethane (50 mL), washed with 10% HCl, brine, dried over MgSO₄ and concentrated in vacuo to afford yellowish oil. This crude product was then purified by flash chromatography (silica gel; ethyl acetate/hexanes, 0%-40%) to afford the desired tosylate. ¹H NMR (300 MHz, CDCl₃) δ 7.72 (m, 2H), 7.29 (m, 2H), 4.39 (m, 1H), 4.10 (m, 2H), 2.38 (s, 3H), 2.09 (m, 1H), 1.93 (m, 2H), 1.65 (m, 1H), 1.16 (d, J=4.8, 6H); ¹³C NMR (101 MHz, CDCl₃)¹³C NMR (101 MHz, CDCl₃) δ 181.26, 145.16, 132.53, 130.03, 127.84, 77.68, 77.36, 77.04, 72.93, 66.83, 42.99, 40.23, 34.97, 24.82, 24.12, 21.57; HRMS (CI): [M+H] 313.1; Anal. Calcd for C₁₅H₂O₅S: C, 57.67; H, 6.45. Found: C, 57.85; H, 6.63.

The following compounds can be prepared by the procedure of 2-(4,4-dimethyl-5-oxotetrahydrofuran-2-yl)ethyl 4-methylbenzenesulfonate. The skilled practitioner will know how to substitute the appropriate reagents, starting materials and purification methods known to those skilled in the art, in order to prepare the compounds provided herein.

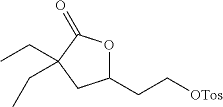

Preparation of 2-(4,4-diethyl-5-oxotetrahydrofuran-2-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 3,3-diethyl-5-(2-hydroxyethyl)dihydrofuran-2(3H)-one was substituted for 5-(2-hydroxy-ethyl)-3,3-dimethyl-dihydro-furan-2-one. ¹H NMR (300 MHz, CDCl₃) δ 7.79 (d, J=8.3 Hz, 2H), 7.36 (d, J=8.0 Hz, 2H), 4.55-4.33 (m, 1H), 4.14 (dd, J=6.5, 13.3 Hz, 3H), 2.46 (s, 3H), 2.21-1.84 (m, 3H), 1.83-1.68 (m, 1H), 1.58 (t, J=7.4 Hz, 4H), 0.89 (dt, J=7.5, 18.0 Hz, 6H); ¹³C NMR (101 MHz, CDCl₃) δ 180.33, 145.30, 132.72, 130.15, 128.03, 77.68, 77.36, 77.04, 73.18, 66.95, 48.67, 37.53, 35.82, 29.14, 28.23, 21.76, 8.81, 8.74. Anal. Calcd for C₁₇H₂₄O₅S: C, 59.98; H, 7.11. Found: C, 60.27; H, 7.25.

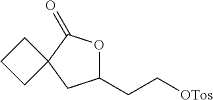

Preparation of 2-(5-oxo-6-oxaspiro[3.4]octan-7-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 7-(2-hydroxyethyl)-6-oxaspiro[3.4]octan-5-one was substituted for 5-(2-hydroxy-ethyl)-3,3-dimethyl-dihydro-furan-2-one. ¹H NMR (400 MHz, CDCl₃) δ 7.77 (d, J=8.3 Hz, 2H), 7.35 (d, J=8.0 Hz, 2H), 4.37 (tdd, J=8.8, 6.0, 4.3 Hz, 1H), 4.21-4.05 (m, 2H), 2.57-2.32 (m, 6H), 2.19-1.82 (m, 7H); ¹³C NMR (101 MHz, CDCl₃) δ 180.41, 145.24, 132.68, 130.10, 128.02, 73.38, 66.76, 44.33, 41.79, 35.10, 31.72, 29.28, 21.76, 16.51.

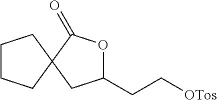

Preparation of 2-(1-oxo-2-oxaspiro[4.4]nonan-3-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 3-(2-hydroxyethyl)-2-oxaspiro[4.4]nonan-1-one was substituted for 5-(2-hydroxy-ethyl)-3,3-dimethyl-dihydro-furan-2-one. ¹H NMR (400 MHz, CDCl₃) δ 7.79 (d, J=8.3 Hz, 2H), 7.36 (d, J=8.0 Hz, 2H), 4.51-4.35 (m, 1H), 4.25-4.06 (m, 2H), 2.45 (s, 3H), 2.28-2.08 (m, 2H), 2.08-1.91 (m, 2H), 1.87-1.52 (m, 9H); ¹³C NMR (101 MHz, CDCl₃) δ 181.90, 145.26, 132.76, 130.12, 128.07, 73.71, 66.85, 50.19, 43.07, 37.44, 36.81, 35.19, 25.61, 25.50, 21.79.

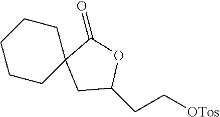

Preparation of 2-(1-oxo-2-oxaspiro[4.5]decan-3-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 3-(2-hydroxyethyl)-2-oxaspiro[4.5]decan-1-one was substituted for 5-(2-hydroxy-ethyl)-3,3-dimethyl-dihydro-furan-2-one. ¹H NMR (400 MHz, CDCl₃) δ 7.79 (d, J=8.3 Hz, 2H), 7.36 (d, J=8.0 Hz, 2H), 4.51-4.38 (m, 1H), 4.26-4.12 (m, 2H), 2.45 (s, 3H), 2.36 (dd, J=12.9, 6.2 Hz, 1H), 2.12-1.87 (m, 2H), 1.85-1.68 (m, 3H), 1.65-1.50 (m, 5H), 1.43-1.14 (m, 3H); ¹³C NMR (101 MHz, CDCl₃) δ 180.97, 145.27, 132.76, 130.12, 128.07, 73.28, 66.85, 44.96, 39.48, 35.58, 34.35, 31.52, 25.37, 22.23, 22.16, 21.80.

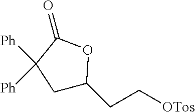

Preparation of 2-(5-oxo-4,4-diphenyltetrahydrofuran-2-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 5-(2-hydroxyethyl)-3,3-diphenyldihydrofuran-2(3H)-one was substituted for 5-(2-hydroxy-ethyl)-3,3-dimethyl-dihydro-furan-2-one. ¹H NMR (400 MHz, CDCl₃) δ 7.79-7.62 (m, 2H), 7.44-7.25 (m, 12H), 4.40 (dtd, J=13.2, 8.9, 4.6 Hz, 1H), 4.29-4.06 (m, 2H), 3.06 (dt, J=12.7, 6.4 Hz, 1H), 2.57 (dd, J=13.0, 10.5 Hz, 1H), 2.44 (s, 3H), 2.21-1.92 (m, 2H); ¹³C NMR (101 MHz, CDCl₃) δ 176.62, 145.30, 141.90, 139.38, 132.60, 130.12, 129.16, 128.59, 128.05, 127.73, 127.48, 127.41, 73.32, 66.57, 58.08, 43.51, 34.71, 21.81, 1.16.

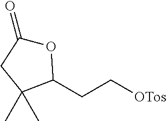

Preparation of 2-(3,3-dimethyl-5-oxotetrahydrofuran-2-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 5-(2-hydroxyethyl)-4,4-dimethyldihydrofuran-2(3H)-one was substituted for 5-(2-hydroxy-ethyl)-3,3-dimethyl-dihydro-furan-2-one. ¹H NMR (400 MHz, CDCl₃) δ 7.83-7.70 (m, 2H), 7.33 (m, 2H), 4.19 (ddd, J=10.4, 6.6, 4.0 Hz, 1H), 4.10 (m, 2H), 2.42 (s, 3H), 2.31 (dd, J=41.8, 16.9 Hz, 2H), 1.97-1.68 (m, 2H), 1.10 (s, 3H), 0.96 (s, 3H); ¹³C NMR (101 MHz, CDCl₃) δ 175.48, 145.19, 132.64, 130.03, 127.96, 83.78, 67.26, 44.35, 38.99, 29.01, 24.90, 21.68, 21.39. MS (LC/MS, M+H⁺): 313.1.

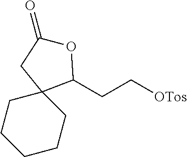

Preparation of 2-(3-oxo-2-oxaspiro[4.5]decan-1-yl)ethyl 4-methylbenzenesulfonate: The title compound was prepared according to the procedure for 2-(4,4-dimethyl-5-oxotetrahydrofuran-2-yl)ethyl 4-methylbenzenesulfonate, except 1-(2-hydroxyethyl)-2-oxaspiro[4.5]decan-3-one was substituted for 5-(2-hydroxy-ethyl)-3,3-dimethyl-dihydro-furan-2-one. ¹H NMR (400 MHz, CDCl₃) δ 7.75 (d, J=8.2 Hz, 2H), 7.32 (d, J=8.0 Hz, 2H), 4.19 (ddd, J=10.5, 6.7, 4.0 Hz, 1H), 4.15-4.01 (m, 2H), 2.48 (d, J=17.3 Hz, 1H), 2.41 (s, 3H), 2.26 (d, J=17.3 Hz, 1H), 1.94 (m, 1H), 1.84-1.68 (m, 1H), 1.59 (s, 3H), 1.48-1.10 (m, 7H); ¹³C NMR (101 MHz, CDCl₃) δ 175.63, 145.11, 132.66, 129.99, 127.91, 84.00, 67.29, 42.66, 39.48, 34.88, 29.63, 29.19, 25.67, 23.02, 22.24, 21.64. MS (LC/MS, M+H⁺): 353.1.

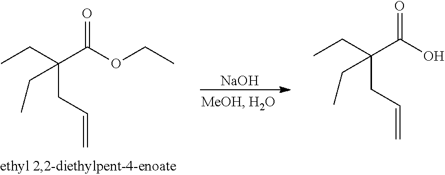

Preparation of 2,2-diethylpent-4-enoic acid: Ethyl 2,2-diethylpent-4-enoate (0.2 g, 0.28 mmol) is mixed with NaOH (0.4 g, 10 mmol), MeOH (2.5 mL) and H₂O (2.5 mL) in a microwave vial. The mixture was then heated in a microwave reactor at 160° C. for 2 hours. The mixture was then acidified with 10% HCl, washed with ether (3×30 ml). The combined organic phase was dried over MgSO₄ and concentrated in vacuo to give a crude product which was used in the next step without further purification.

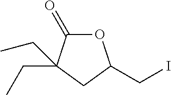

Preparation of 3,3-diethyl-5-(iodomethyl)dihydrofuran-2(3H)-one: 2,2-diethylpent-4-enoic acid (1.77 g, 11.67 mmol) was stirred with tetrahydrofuran (34 mL), ether (12 mL) and saturated NaHCO₃ solution (57 mL). The mixture was protected from sunlight. 12 was dissolved in 12 mL of tetrahydrofuran and added to the mixture in one portion at 0° C. The mixture was allowed to stir overnight at room temperature. Saturated sodium thiosulfate was added to the mixture to quench the reaction. The mixture was extracted with ethyl acetate (3×50 mL). The combined organic phase was dried over MgSO₄ and concentrated in vacuo to give a crude oil which was purified by flash chromatography (silica gel; ethyl acetate/hexanes, 0%-25%). ¹H NMR (400 MHz, CDCl₃) δ 4.42 (dtd, J=9.0, 7.3, 4.6 Hz, 1H), 3.41 (dd, J=10.2, 4.6 Hz, 1H), 3.23 (dd, J=10.2, 7.5 Hz, 1H), 2.25 (dd, J=13.3, 6.9 Hz, 1H), 1.86 (dd, J=13.3, 9.1 Hz, 1H), 1.63 (m, 4H), 0.94 (dt, J=10.4, 7.5 Hz, 6H). MS (LC/MS, M+H⁺): 283.0.

The following compounds can be prepared by the procedure of 3,3-diethyl-5-(iodomethyl)dihydrofuran-2(3H)-one. The skilled practitioner will know how to substitute the appropriate reagents, starting materials and purification methods known to those skilled in the art, in order to prepare the compounds provided herein.

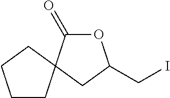 [SMILES: O=C1OC(CI)CC12CCCC2]

Preparation of 3-(iodomethyl)-2-oxaspiro[4.4]nonan-1-one: The title compound was prepared according to the procedure for 3,3-diethyl-5-(iodomethyl)dihydrofuran-2(3H)-one, except 1-allylcyclopentanecarboxylic acid was substituted for 2,2-diethylpent-4-enoic acid. ¹H NMR (400 MHz, CDCl₃) δ 4.48-4.34 (m, 1H), 3.39 (dd, J=10.2, 4.9 Hz, 1H), 3.23 (dd, J=10.2, 7.5 Hz, 1H), 2.35 (dd, J=12.9, 6.1 Hz, 1H), 2.20-2.04 (m, 1H), 1.93-1.54 (m, 8H); ¹³C NMR (101 MHz, CDCl₃) δ 181.57, 75.96, 50.71, 43.44, 37.84, 36.89, 25.45, 25.36, 7.02; MS (LC/MS, M+H⁺): 281.0.

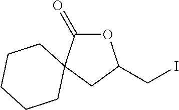 [SMILES: O=C1OC(CI)CC12CCCCC2]

Preparation of 3-(iodomethyl)-2-oxaspiro[4.5]decan-1-one: The title compound was prepared according to the procedure for 3,3-diethyl-5-(iodomethyl)dihydrofuran-2(3H)-one, except 1-allylcyclohexanecarboxylic acid was substituted for 2,2-diethylpent-4-enoic acid. ¹H NMR (400 MHz, CDCl₃) δ 4.42 (dtd, J=9.2, 6.9, 4.6 Hz, 1H), 3.41 (dd, J=10.3, 4.6 Hz, 1H), 3.26 (dd, J=10.2, 7.3 Hz, 1H), 2.50 (dd, J=13.1, 6.5 Hz, 1H), 1.85-1.49 (m, 8H), 1.44-1.20 (m, 3H); MS (LC/MS, M+H⁺): 295.0.

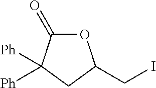 [SMILES: O=C1OC(CI)CC1(c1ccccc1)c1ccccc1]

Preparation of 5-(iodomethyl)-3,3-diphenyldihydrofuran-2(3H)-one: The title compound was prepared according to the procedure for 3,3-diethyl-5-(iodomethyl)dihydrofuran-2(3H)-one, except 2,2-diphenylpent-4-enoic acid was substituted for 2,2-diethylpent-4-enoic acid. ¹H NMR (400 MHz, CDCl₃) δ 7.27-7.16 (m, 10H), 4.29 (m, 1H), 3.37 (m, 1H), 3.24-3.13 (m, 2H), 2.61 (m, 1H); ¹³C NMR (101 MHz, CDCl₃) δ 176.41, 141.58, 139.40, 129.07, 128.53, 127.97, 127.67, 127.47, 127.29, 75.40, 58.70, 44.09, 5.94; HRMS (CI): [M+H] 379.1.

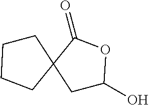 [SMILES: O=C1OC(O)CC12CCCC2]

Preparation of 3-hydroxy-2-oxaspiro[4.4]nonan-1-one: To a stirred mixture of 1-allylcyclopentanecarboxylic acid (10.93 g, 71 mmol, 1 equiv), RuCl₃ stock solution (0.514 g, 0.035M in water, 0.035 equiv) and CH₃CN (500 mL), NaIO₄ (30.8 g, 142 mmol, 2.04 equiv) was added in portions over a period of 30 min at room temperature. The suspension was allowed to stir at room temperature for another 30 min. The reaction was quenched with saturated aqueous solution of Na₂S₂O₃ and the two layers were separated. The aqueous layer was extracted with EtOAc (3×200 mL). The combined organic layer was washed with brine, dried over anhydrous MgSO₄, filtered, and concentrated. The residue was purified by flash column chromatography (silica gel; ethyl acetate/hexanes, 10%-50%) to give desired product. ¹H NMR (400 MHz, CDCl₃) δ 5.87 (s, 1H), 5.28 (s, 1H), 2.06 (dd, J=35.1, 28.9 Hz, 4H), 1.90-1.44 (m, 6H); ¹³C NMR (101 MHz, CDCl₃) δ 183.20, 49.58, 43.94, 38.28, 25.42.

The following compounds can be prepared by the procedure of 3-hydroxy-2-oxaspiro[4.4]nonan-1-one. The skilled practitioner will know how to substitute the appropriate reagents, starting materials and purification methods known to those skilled in the art, in order to prepare the compounds provided herein.

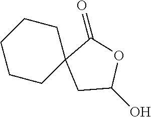 [SMILES: O=C1OC(O)CC12CCCCC2]

Preparation of 3-hydroxy-2-oxaspiro[4.5]decan-1-one: The title compound was prepared according to the procedure for 3-hydroxy-2-oxaspiro[4.4]nonan-1-one, except 1-allylcyclohexanecarboxylic acid was substituted for 1-allylcyclopentanecarboxylic acid. ¹H NMR (400 MHz, CDCl₃) δ 5.86 (t, J=4.5 Hz, 1H), 4.47 (broad, 1H), 2.18 (m, 2H), 1.83-1.43 (m, 7H), 1.32 (d, J=5.8 Hz, 3H); ¹³C NMR (101 MHz, CDCl₃) δ 181.91, 96.88, 44.52, 40.54, 34.06, 25.28, 22.23.

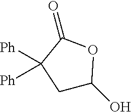 [SMILES: O=C1OC(O)CC1(c1ccccc1)c1ccccc1]

Preparation of 5-hydroxy-3,3-diphenyldihydrofuran-2(3H)-one: The title compound was prepared according to the procedure for 3-hydroxy-2-oxaspiro[4.4]nonan-1-one, except 2,2-diphenylpent-4-enoic acid was substituted for 1-allylcyclopentanecarboxylic acid. ¹H NMR (400 MHz, CDCl₃) δ 7.33-7.15 (m, 10H), 5.74 (t, J=5.2 Hz, 1H), 3.96 (broad, 1H), 3.15-2.81 (m, 2H).

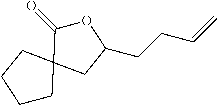

Preparation of 3-(but-3-en-1-yl)-2-oxaspiro[4.4]nonan-1-one: This reaction was performed in oven-dried glassware under a nitrogen atmosphere. To a well-stirred solution of freshly prepared but-1-ene magnesium bromide Grignard reagent (96 mmol, 1M, 3 equiv) in dry ether, 3-hydroxy-2-oxaspiro[4.4]nonan-1-one (5.0 g, 32.0 mmol, 1.0 equiv) was added dropwise during 0.5 hours at 0° C. The reaction mixture was stirred overnight at room temperature, quenched with 10% HCl (while cooling in ice bath) until acidic (pH=2). The organic layer was separated and the aqueous layer was extracted with ethyl acetate (3×200 mL). The extract was washed with 10% NaHCO₃ (100 mL) and brine (200 mL). The solution was then dried over MgSO₄, concentrated in vacuo and purified by flash column chromatography (silica gel; Ethyl acetate/Hexanes, 0%-25%) to give desired product. ¹H NMR (400 MHz, CDCl₃) δ 5.79 (ddt, J=16.9, 10.2, 6.7 Hz, 1H), 5.15-4.88 (m, 2H), 4.36 (ddt, J=9.7, 7.9, 5.5 Hz, 1H), 2.18 (m, 4H), 1.93-1.46 (m, 10H); ¹³C NMR (101 MHz, CDCl₃) δ 182.55, 137.26, 115.62, 77.19, 50.28, 43.24, 37.51, 36.91, 34.83, 29.70, 25.56, 25.47.

The following compounds can be prepared by the procedure of 3-(but-3-en-1-yl)-2-oxaspiro[4.4]nonan-1-one. The skilled practitioner will know how to substitute the appropriate reagents, starting materials and purification methods known to those skilled in the art, in order to prepare the compounds provided herein.

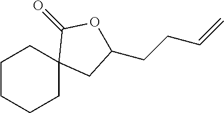

Preparation of 3-(but-3-en-1-yl)-2-oxaspiro[4.5]decan-1-one: The title compound was prepared according to the procedure for 3-(but-3-en-1-yl)-2-oxaspiro[4.4]nonan-1-one, except 3-hydroxy-2-oxaspiro[4.5]decan-1-one was substituted for 3-hydroxy-2-oxaspiro[4.4]nonan-1-one. ¹H NMR (400 MHz, CDCl₃) δ 5.80 (ddt, J=16.9, 10.2, 6.6 Hz, 1H), 5.17-4.89 (m, 2H), 4.48-4.31 (m, 1H), 2.36 (dd, J=12.9, 6.3 Hz, 1H), 2.30-2.08 (m, 2H), 1.87-1.17 (m, 13H); ¹³C NMR (101 MHz, CDCl₃) δ 181.68, 137.31, 115.67, 76.77, 45.04, 39.55, 35.31, 34.43, 31.70, 29.75, 25.42, 22.29, 22.22.

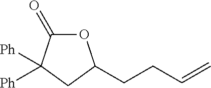 [SMILES: C=CCCC1CC(c2ccccc2)(c2ccccc2)C(=O)O1]

Preparation of 5-(but-3-en-1-yl)-3,3-diphenyldihydrofuran-2(3H)-one: The title compound was prepared according to the procedure for 3-(but-3-en-1-yl)-2-oxaspiro[4.4]nonan-1-one, except 5-hydroxy-3,3-diphenyldihydrofuran-2(3H)-one was substituted for 3-hydroxy-2-oxaspiro[4.4]nonan-1-one. ¹H NMR (400 MHz, CDCl₃) δ 7.33-7.06 (m, 10H), 5.70 (ddt, J=16.9, 10.2, 6.6 Hz, 1H), 5.05-4.85 (m, 2H), 4.27 (ddt, J=10.2, 7.8, 5.0 Hz, 1H), 2.96 (dd, J=12.9, 4.8 Hz, 1H), 2.50 (dd, J=13.0, 10.5 Hz, 1H), 2.27-1.98 (m, 2H), 1.86-1.59 (m, 2H); ¹³C NMR (101 MHz, CDCl₃) δ 177.10, 142.31, 139.92, 137.12, 128.98, 128.43, 127.77, 127.43, 127.23, 115.69, 76.69, 58.25, 43.70, 34.36, 29.58.

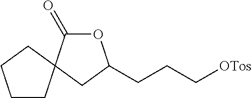

Preparation of 3-(1-oxo-2-oxaspiro[4.4]nonan-3-yl)propyl 4-methylbenzenesulfonate: To a stirred mixture of 3-(but-3-en-1-yl)-2-oxaspiro[4.4]nonan-1-one (0.194 g, 1 mmol, 1 equiv), RuCl₃ stock solution (7.2 mg, 0.035M in water, 0.035 equiv) and CH₃CN (6 mL), NaIO₄ (434 mg, 2.04 mmol, 2.04 equiv) was added in portions over a period of 5 min at room temperature. The suspension was allowed to stir at room temperature for another 30 min. The reaction was quenched with saturated aqueous solution of Na₂S₂O₃ and the two layers were separated. The aqueous layer was extracted with EtOAc (3×20 mL). The combined organic layer was washed with brine, dried over anhydrous MgSO₄, filtered, and concentrated. The crude aldehyde was used for the next step without further purification.

This reaction was performed in oven-dried glassware under a nitrogen atmosphere. To a well-stirred solution of the crude aldehyde (0.196 g, 1 mmol, 1 equiv) in dry methanol, NaBH₄ (74 mg, 2.0 mmol, 2 equiv) was added to the mixture in one portion at 0° C. The reaction mixture was stirred at room temperature for another 1 h, quenched with brine (while cooling in ice bath). The organic layer was separated and the aqueous layer was extracted with ethyl acetate (3×20 mL). The combined organic phase was then dried over MgSO₄, concentrated in vacuo. The crude alcohol was used for the next step without further purification.

To a stirred solution of the crude alcohol (0.396 g, 2 mmol, 1.0 equiv) and Et₃N (0.303 g, 3 mmol, 1.5 equiv) in dry dichloromethane, a solution of p-TosCl (0.475 g, 2.5 mmol, 1.25 equiv) in dichloromethane was added drop wise at 0° C. The resulting mixture was stirred at 0° C. for 1 hour and allowed to stir overnight at room temperature. Then, the reaction mixture was diluted with dichloromethane (50 mL), washed with 10% HCl, brine, dried over MgSO₄ and concentrated in vacuo to afford yellowish oil. This crude product was then purified by flash chromatography (silica gel; ethyl acetate/hexanes, 0%-40%) to afford desired tosylate. ¹H NMR (400 MHz, CDCl₃) δ 7.82-7.71 (m, 2H), 7.35 (m, 2H), 4.37-4.23 (m, 1H), 4.06 (qdd, J=10.0, 6.7, 5.2 Hz, 2H), 2.45 (s, 3H), 2.15 (m, 2H), 1.92-1.50 (m, 12H); ¹³C NMR (101 MHz, CDCl₃) δ 182.29, 145.03, 133.05, 130.04, 128.00, 76.90, 69.91, 50.24, 43.20, 37.53, 36.92, 31.74, 25.59, 25.49, 25.37, 21.76.

The following compounds can be prepared by the procedure of 3-(1-oxo-2-oxaspiro[4.4]nonan-3-yl)propyl 4-methylbenzenesulfonate. The skilled practitioner will know how to substitute the appropriate reagents, starting materials and purification methods known to those skilled in the art, in order to prepare the compounds provided herein.

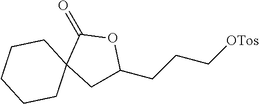

Preparation of 3-(1-oxo-2-oxaspiro[4.5]decan-3-yl)propyl 4-methylbenzenesulfonate: The title compound was prepared according to the procedure for 3-(1-oxo-2-oxaspiro[4.4]nonan-3-yl)propyl 4-methylbenzenesulfonate, except 3-(but-3-en-1-yl)-2-oxaspiro[4.5]decan-1-one was substituted for 3-(but-3-en-1-yl)-2-oxaspiro[4.4]nonan-1-one. ¹H NMR (400 MHz, CDCl₃) δ 7.78 (d, J=8.3 Hz, 2H), 7.35 (d, J=8.0 Hz, 2H), 4.39-4.26 (m, 1H), 4.16-3.97 (m, 2H), 2.44 (s, 3H), 2.32 (dt, J=15.8, 7.9 Hz, 1H), 1.98-1.13 (m, 16H); ¹³C NMR (101 MHz, CDCl₃) δ 181.36, 145.03, 133.05, 130.03, 127.99, 76.46, 69.91, 44.97, 39.54, 34.40, 32.15, 31.68, 25.37, 25.36, 22.25, 22.18, 21.76.

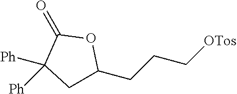

Preparation of 3-(5-oxo-4,4-diphenyltetrahydrofuran-2-yl)propyl 4-methylbenzenesulfonate: The title compound was prepared according to the procedure for 3-(1-oxo-2-oxaspiro[4.4]nonan-3-yl)propyl 4-methylbenzenesulfonate, except 5-(but-3-en-1-yl)-3,3-diphenyldihydrofuran-2(3H)-one was substituted for 3-(but-3-en-1-yl)-2-oxaspiro[4.4]nonan-1-one. ¹H NMR (400 MHz, CDCl₃) δ 7.81 (m, 2H), 7.38-7.26 (m, 12H), 4.31 (m, 1H), 4.08 (m, 2H), 3.05 (m, 1H), 2.60 (m, 1H), 2.45 (s, 3H), 1.80-1.65 (m, 2H); ¹³C NMR (101 MHz, CDCl₃) δ 176.90, 144.96, 141.91, 139.53, 132.88, 129.95, 128.99, 128.42, 127.87, 127.80, 127.65, 127.31, 127.28, 76.41, 69.80, 58.13, 43.50, 31.18, 25.25, 21.66; MS (LC/MS, M+H⁺): 451.1.

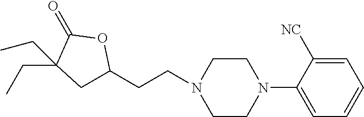 [SMILES: CCC1(CC)CC(CCN2CCN(c3ccccc3C#N)CC2)OC1=O]

Preparation of 2-(4-(2-(4,4-diethyl-5-oxotetrahydrofuran-2-yl)ethyl)piperazin-1-yl)benzonitrile: 2-(4,4-diethyl-5-oxotetrahydrofuran-2-yl)ethyl 4-methylbenzenesulfonate (0.102 g, 0.3 mmol, 1.0 equiv) was treated with 2-piperazin-1-yl-benzonitrile (168.3 mg, 0.9 mmol, 3.0 equiv) in dry tetrahydrofuran and refluxed for 72 hours. The tetrahydrofuran was evaporated under reduced pressure, the residue dissolved in dichloromethane, washed with H₂O, and brine, then dried over MgSO₄ and concentrated in vacuo to give a crude product which was purified by flash chromatography (silica gel; 2%-8% MeOH in dichloromethane) to afford pure product. The purified product was then dissolved in ether and treated with HCl solution (2.0 M in diethyl ether) to afford the hydrochloride salt which was recrystallized with isopropanol or a MeOH/Ether mixture. ¹H NMR (400 MHz, CDCl₃) δ 7.62-7.42 (m, 2H), 7.01 (dd, J=7.8, 5.0 Hz, 2H), 4.48 (dq, J=9.2, 6.7 Hz, 1H), 3.35-3.17 (m, 4H), 2.81-2.51 (m, 6H), 2.14 (dd, J=13.1, 6.8 Hz, 1H), 1.86 (m, 3H), 1.67-1.53 (m, 4H), 0.92 (dt, J=20.1, 7.5 Hz, 6H); ¹³C NMR (101 MHz, CDCl₃) δ 180.82, 155.57, 134.43, 133.95, 122.03, 118.81, 118.50, 106.13, 75.50, 54.44, 53.22, 51.34, 48.71, 37.75, 33.60, 29.35, 28.39, 8.89, 8.81; MS (LC/MS, M+H⁺): 356.2.

The following compounds can be prepared by the procedure of 2-(4-(2-(4,4-diethyl-5-oxotetrahydrofuran-2-yl)ethyl)piperazin-1-yl)benzonitrile. The skilled practitioner will know how to substitute the appropriate reagents, starting materials and purification methods known to those skilled in the art, in order to prepare the compounds provided herein.

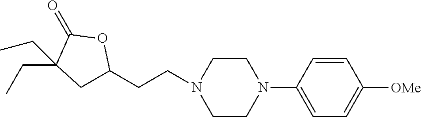 [SMILES: CCC1(CC)CC(CCN2CCN(c3ccc(OC)cc3)CC2)OC1=O]

Preparation of 3,3-diethyl-5-(2-(4-(4-methoxyphenyl)piperazin-1-yl)ethyl)dihydrofuran-2(3H)-one: The title compound was prepared according to the procedure for 2-(4-(2-(4,4-diethyl-5-oxotetrahydrofuran-2-yl)ethyl)piperazin-1-yl)benzonitrile, except 1-(4-Methoxy-phenyl)-piperazine was substituted for 2-piperazin-1-yl-benzonitrile. ¹H NMR (400 MHz, CDCl₃) δ 6.95-6.75 (m, 4H), 4.48 (ddd, J=19.8, 8.4, 6.4 Hz, 1H), 3.76 (s, 3H), 3.14-2.99 (m, 4H), 2.67-2.46 (m, 6H), 2.15-2.07 (m, 1H), 1.92-1.79 (m, 3H), 1.62 (qd, J=7.4, 4.7 Hz, 4H), 0.97-0.88 (m, 6H); ¹³C NMR (101 MHz, CDCl₃) δ 180.90, 153.93, 145.74, 118.29, 114.53, 75.71, 55.67, 54.59, 53.51, 50.69, 48.72, 37.81, 33.91, 29.35, 28.41, 8.90, 8.82. MS (LC/MS, M+H⁺): 361.2.

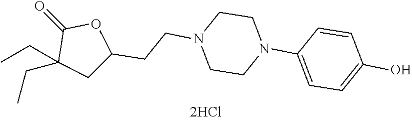 [SMILES: CCC1(CC)CC(CCN2CCN(c3ccc(O)cc3)CC2)OC1=O]

Preparation of 3,3-diethyl-5-(2-(4-(4-hydroxyphenyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 4-piperazin-1-yl-phenol was substituted for 2-piperazin-1-yl-benzonitrile. ¹H NMR (400 MHz, D₂O) δ 7.16 (d, J=9.0, 2H), 6.94 (d, J=9.0, 2H), 4.71 (d, J=10.6, 1H), 3.46 (ddd, J=15.7, 16.9, 22.4, 10H), 2.36 (dd, J=6.9, 13.5, 1H), 2.23 (dd, J=9.2, 19.4, 2H), 2.01 (dd, J=9.4, 13.5, 1H), 1.77-1.50 (m, 4H), 0.90 (dt, J=7.5, 12.5, 6H); ¹³C NMR (101 MHz, D₂O) δ 187.92, 155.62, 143.21, 123.52, 119.36, 79.53, 56.53, 54.17, 52.42, 52.07, 39.38, 32.83, 31.92, 30.68, 11.00, 10.87; MS (LC/MS, M+H⁺): 347.2. Anal. Calcd for C₂₀H₃₂C₁₂N₂O₃: C, 57.28; H, 7.69; N, 6.68; Found C, 57.53; H, 7.74; N, 6.62.

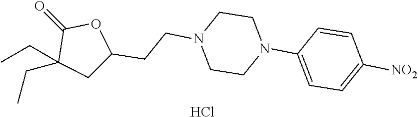 [SMILES: CCC1(CC)CC(CCN2CCN(c3ccc([N+](=O)[O-])cc3)CC2)OC1=O ; Cl]

Preparation of 3,3-diethyl-5-(2-(4-(4-nitrophenyl)piperazin-1-yl)ethyl)dihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-(4-nitro-phenyl)-piperazine was substituted for 2-piperazin-1-yl-benzonitrile. ¹H NMR (400 MHz, MeOH) δ 8.15 (d, J=9.3, 2H), 7.11 (d, J=9.4, 2H), 4.59 (dd, J=3.6, 6.5, 1H), 4.37-3.08 (m, 10H), 2.36-2.21 (m, 2H), 2.21-2.08 (m, 1H), 1.95 (dd, J=9.4, 13.2, 1H), 1.75-1.52 (m, 4H), 0.94 (dt, J=7.5, 13.2, 6H); ¹³C NMR (101 MHz, MeOH) δ 183.19, 156.05, 141.97, 127.53, 116.10, 77.01, 55.81, 53.54, 50.77, 50.50, 50.29, 50.07, 49.86, 49.65, 49.43, 49.22, 46.59, 39.19, 32.57, 30.89, 30.03, 9.85, 9.77; MS (LC/MS, M+H⁺): 376.2.

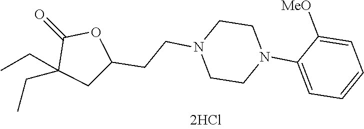 [SMILES: CCC1(CC)CC(CCN2CCN(c3ccccc3OC)CC2)OC1=O]

Preparation of 3,3-diethyl-5-(2-(4-(2-methoxyphenyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-(2-methoxy-phenyl)-piperazine was substituted for 2-piperazin-1-yl-benzonitrile. ¹H NMR (400 MHz, D₂O) δ 7.06 (ddd, J=7.8, 7.2, 1.5 Hz, 2H), 6.96 (dd, J=8.1, 1.3 Hz, 1H), 6.93-6.82 (m, 1H), 4.50 (dt, J=9.2, 7.5 Hz, 1H), 3.80 (s, 3H), 3.72-3.22 (m, 10H), 2.28-2.10 (m, 2H), 2.10-1.96 (m, 1H), 1.86 (dd, J=13.3, 9.4 Hz, 1H), 1.68-1.42 (m, 4H), 1.00-0.75 (m, 6H); ¹³C NMR (101 MHz, D₂O) δ 182.35, 153.90, 138.69, 126.90, 122.35, 120.49, 113.32, 76.15, 56.21, 54.97, 53.20, 49.93, 49.35, 38.35, 31.74, 30.05, 29.19, 9.00, 8.91; MS (LC/MS, M+H⁺): 361.2; Anal. Calcd for C₂₁H₃₄C₁₂N₂O₃: C, 58.20; H, 7.91; N, 6.46. Found: C, 58.05; H, 7.95; N, 6.39.

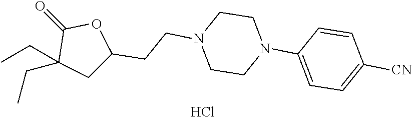 [SMILES: CCC1(CC)CC(CCN2CCN(c3ccc(C#N)cc3)CC2)OC1=O ; Cl]

Preparation of 4-(4-(2-(4,4-diethyl-5-oxotetrahydrofuran-2-yl)ethyl)piperazin-1-yl)benzonitrile hydrochloride: The title compound was prepared according to the procedure for 2-(4-(2-(4,4-diethyl-5-oxotetrahydrofuran-2-yl)ethyl)piperazin-1-yl)benzonitrile, except 4-piperazin-1-yl-benzonitrile was substituted for 2-piperazin-1-yl-benzonitrile. ¹H NMR (400 MHz, MeOD) δ 7.69-7.54 (m, 2H), 7.23-7.02 (m, 2H), 4.59 (ddd, J=15.8, 9.3, 3.7 Hz, 1H), 4.31-3.30 (m, 10H), 2.36-2.21 (m, 2H), 2.21-2.06 (m, 1H), 1.96 (dd, J=13.3, 9.4 Hz, 1H), 1.65 (ddd, J=17.4, 8.7, 6.2 Hz, 4H), 0.95 (dt, J=13.3, 7.5 Hz, 6H); ¹³C NMR (101 MHz, MeOD) δ 182.32, 153.74, 134.73, 120.40, 116.55, 102.99, 76.15, 54.93, 52.76, 49.91, 45.91, 38.33, 31.73, 30.04, 29.17, 9.00, 8.91; MS (LC/MS, M+H⁺): 356.2; Anal. Calcd for C₂₁H₃₀ClN₃O₂: C, 64.35; H, 7.72; N, 10.72. Found: C, 64.46; H, 7.65; N, 10.65.

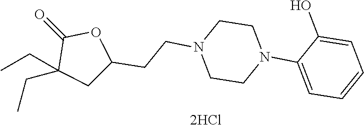 [SMILES: CCC1(CC)CC(CCN2CCN(c3ccccc3O)CC2)OC1=O]

Preparation of 3,3-diethyl-5-(2-(4-(2-hydroxyphenyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 2-piperazin-1-yl-phenol was substituted for 2-piperazin-1-yl-benzonitrile. ¹H NMR (400 MHz, D₂O) δ 7.35-7.16 (m, 2H), 7.00 (m, 2H), 4.74-4.65 (m, 1H), 3.91-3.34 (m, 10H), 2.40-2.10 (m, 3H), 1.99 (dd, J=13.5, 9.4 Hz, 1H), 1.72-1.48 (m, 4H), 0.87 (dt, J=13.0, 7.5 Hz, 6H); ¹³C NMR (101 MHz, D₂O) δ 185.26, 149.63, 133.87, 128.39, 121.34, 120.76, 116.89, 76.88, 53.96, 51.49, 49.77, 48.95, 36.75, 30.19, 29.27, 28.04, 8.37, 8.24; MS (LC/MS, M+H⁺): 347.2; Anal. Calcd for C₂₀H₃₂C₁₂N₂O₃: C, 57.28; H, 7.69; N, 6.68. Found: C, 57.37; H, 7.64; N, 6.59.

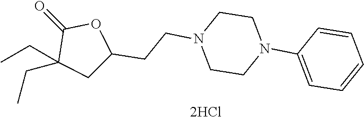 [SMILES: CCC1(CC)CC(CCN2CCN(c3ccccc3)CC2)OC1=O]

Preparation of 3,3-diethyl-5-(2-(4-phenylpiperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-phenyl-piperazine was substituted for 2-piperazin-1-yl-benzonitrile. ¹H NMR (400 MHz, D₂O) δ 7.43 (m, 2H), 7.27-7.13 (m, 3H), 4.69 (m, 1H), 4.11-3.09 (m, 10H), 2.39-2.07 (m, 3H), 1.98 (dd, J=13.4, 9.4 Hz, 1H), 1.61 (m, 4H), 0.87 (dt, J=12.1, 7.5 Hz, 6H); ¹³C NMR (101 MHz, D₂O) δ 187.92, 150.20, 132.89, 127.03, 121.14, 79.53, 56.52, 54.13, 52.41, 50.87, 39.37, 32.81, 31.91, 30.68, 11.00, 10.87; MS (LC/MS, M+H⁺): 331.2; Anal. Calcd for C₂₀H₃₂C₁₂N₂O₂: C, 59.55; H, 8.00; N, 6.94. Found: C, 59.62; H, 8.11; N, 6.90.

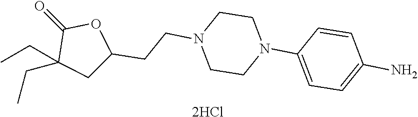 [SMILES: CCC1(CC)CC(CCN2CCN(c3ccc(N)cc3)CC2)OC1=O ; "2HCl"]

Preparation of 5-(2-(4-(4-aminophenyl)piperazin-1-yl)ethyl)-3,3-diethyldihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 4-piperazin-1-yl-phenylamine was substituted for 2-piperazin-1-yl-benzonitrile. ¹H NMR (400 MHz, MeOD) δ 7.31 (d, J=9.0 Hz, 2H), 7.16 (d, J=9.0 Hz, 2H), 4.58 (ddd, J=15.9, 9.3, 3.8 Hz, 1H), 4.06-3.30 (m, 10H), 2.38-2.06 (m, 3H), 1.95 (dd, J=13.3, 9.4 Hz, 1H), 1.78-1.50 (m, 4H), 0.94 (dt, J=13.4, 7.5 Hz, 6H); ¹³C NMR (101 MHz, MeOD) δ 182.43, 151.41, 124.89, 124.66, 118.73, 76.22, 54.91, 53.06, 49.94, 47.42, 38.34, 31.72, 30.04, 29.17, 9.00, 8.92. MS (LC/MS, M+H⁺): 346.2.

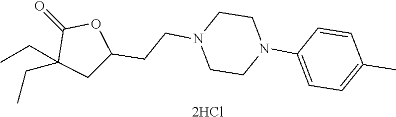 [SMILES: CCC1(CC)CC(CCN2CCN(c3ccc(C)cc3)CC2)OC1=O ; "2HCl"]

Preparation of 3,3-diethyl-5-(2-(4-(p-tolyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-p-tolyl-piperazine was substituted for 2-piperazin-1-yl-benzonitrile. ¹H NMR (400 MHz, MeOD) δ 7.25-7.13 (m, 4H), 4.62-4.45 (m, 1H), 4.05-3.28 (m, 10H), 2.30-2.01 (m, 6H), 1.88 (dd, J=13.3, 9.4 Hz, 1H), 1.58 (m, 4H), 0.87 (dt, J=13.7, 7.5 Hz, 6H); ¹³C NMR (101 MHz, MeOD) δ 182.36, 144.75, 136.86, 131.47, 119.95, 76.10, 54.89, 52.04, 50.53, 49.93, 38.31, 31.68, 30.03, 29.17, 20.76, 9.01, 8.92; MS (LC/MS, M+H⁺): 345.2; Anal. Calcd for C₂₁H₃₄C₁₂N₂O₂: C, 60.43; H, 8.21; N, 6.71. Found: C, 60.33; H, 8.20; N, 6.61.

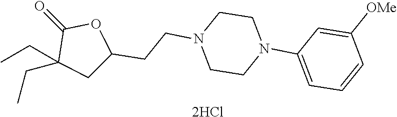 [SMILES: CCC1(CC)CC(CCN2CCN(c3cccc(OC)c3)CC2)OC1=O ; "2HCl"]

Preparation of 3,3-diethyl-5-(2-(4-(3-methoxyphenyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-(3-methoxy-phenyl)-piperazine was substituted for 2-piperazin-1-yl-benzonitrile. ¹H NMR (400 MHz, DMSO) δ 7.16 (t, J=8.2 Hz, 1H), 6.65-6.35 (m, 3H), 4.54 (s, 1H), 3.82 (d, J=8.9 Hz, 3H), 3.57 (s, 2H), 3.16 (dd, J=27.5, 16.8 Hz, 6H), 2.28-2.04 (m, 3H), 1.82 (dd, J=13.1, 9.4 Hz, 1H), 1.64-1.44 (m, 4H), 0.85 (dt, J=10.2, 7.5 Hz, 6H); ¹³C NMR (101 MHz, MeOD) δ 182.37, 162.23, 149.87, 131.32, 76.14, 55.89, 54.87, 52.48, 49.92, 38.31, 31.69, 30.03, 29.17, 9.01, 8.92. MS (LC/MS, M+H⁺): 361.2; Anal. Calcd for C₂₁H₃₄C₁₂N₂O₃: C, 58.20; H, 7.91; N, 6.46. Found: C, 58.24; H, 7.93; N, 6.46.

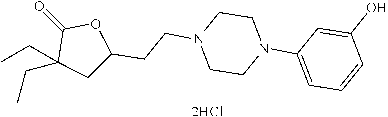 [SMILES: CCC1(CC)CC(CCN2CCN(c3cccc(O)c3)CC2)OC1=O ; "2HCl"]

Preparation of 3,3-diethyl-5-(2-(4-(3-hydroxyphenyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 3-piperazin-1-yl-phenol was substituted for 2-piperazin-1-yl-benzonitrile. ¹H NMR (400 MHz, DMSO) δ 6.93 (t, J=8.1, 1H), 6.33 (d, J=8.2, 1H), 6.27 (s, 1H), 6.21 (d, J=7.9, 1H), 4.43 (s, 10H), 3.64 (s, 2H), 3.47 (s, 2H), 3.12 (s, 2H), 3.00 (d, J=9.1, 4H), 2.16-1.92 (m, 3H), 1.78-1.67 (m, 1H), 1.44 (dd, J=7.4, 23.5, 4H), 0.75 (dt, J=7.4, 10.3, 6H); ¹³C NMR (101 MHz, MeOD) δ 179.73, 158.25, 150.60, 129.75, 107.64, 107.07, 103.23, 74.26, 51.93, 50.56, 50.39, 45.47, 36.40, 29.62, 28.26, 27.58, 8.50, 8.45; MS (LC/MS, M+H⁺): 347.2; Anal. Calcd for C₂₀H₃₂C₁₂N₂O₃: C, 57.28; H, 7.69; N, 6.68. Found: C, 57.33; H, 7.76; N, 6.62.

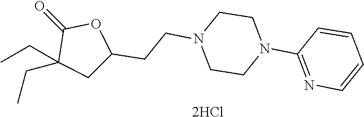 [SMILES: CCC1(CC)CC(CCN2CCN(c3ccccn3)CC2)OC1=O ; "2HCl"]

Preparation of 3,3-diethyl-5-(2-(4-(pyridin-2-yl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-pyridin-2-yl-piperazine was substituted for 2-piperazin-1-yl-benzonitrile. ¹H NMR (400 MHz, D₂O) δ 8.10 (ddd, J=9.1, 7.2, 1.8 Hz, 1H), 8.02 (dd, J=6.2, 1.7 Hz, 1H), 7.34 (d, J=9.2 Hz, 1H), 7.12 (t, J=6.7 Hz, 1H), 4.71 (ddd, J=16.0, 9.2, 3.6 Hz, 1H), 4.31-3.26 (m, 10H), 2.26 (m, 3H), 2.00 (dd, J=13.5, 9.4 Hz, 1H), 1.76-1.46 (m, 4H), 0.88 (dt, J=11.8, 7.5 Hz, 6H); ¹³C NMR (101 MHz, D₂O) δ 187.89, 155.57, 147.93, 140.42, 117.97, 115.85, 79.49, 56.71, 53.74, 52.39, 46.15, 39.38, 32.77, 31.88, 30.66, 26.67, 10.99, 10.86; MS (LC/MS, M+H⁺): 332.2.

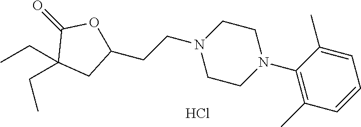 [SMILES: CCC1(CC)CC(CCN2CCN(c3c(C)cccc3C)CC2)OC1=O ; Cl]

Preparation of 5-(2-(4-(2,6-dimethylphenyl)piperazin-1-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2,6-dimethyl-phenyl)-piperazine was substituted for 2-piperazin-1-yl-benzonitrile. ¹H NMR (400 MHz, MeOH) δ 6.99 (s, 3H), 4.59 (ddd, J=16.1, 9.4, 3.8 Hz, 1H), 3.99-3.32 (m, 8H), 3.27-2.98 (m, 2H), 2.39-2.05 (m, 9H), 1.96 (dd, J=13.3, 9.4 Hz, 1H), 1.65 (m, 4H), 0.95 (dt, J=14.7, 7.5 Hz, 6H); ¹³C NMR (101 MHz, MeOH) δ 182.40, 147.75, 127.37, 76.24, 55.23, 54.81, 54.72, 49.92, 48.11, 38.34, 31.75, 30.05, 29.18, 9.01, 8.92; MS (LC/MS, M+H⁺): 359.3; Anal. Calcd for C₂₂H₃₅ClN₂O₂: C, 66.90; H, 8.93; N, 7.09. Found: C, 66.76, H, 8.89, N, 7.01.

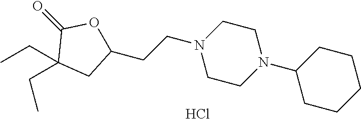 [SMILES: CCC1(CC)CC(CCN2CCN(C3CCCCC3)CC2)OC1=O ; Cl]

Preparation of 5-(2-(4-cyclohexylpiperazin-1-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-Cyclohexyl-piperazine was substituted for 2-piperazin-1-yl-benzonitrile. ¹H NMR (400 MHz, DMSO) δ 4.60-4.49 (m, 1H), 3.93-3.45 (m, 8H), 3.23 (s, 3H), 2.25-2.01 (m, 5H), 1.89-1.72 (m, 3H), 1.68-1.02 (m, 11H), 0.91-0.76 (m, 6H); ¹³C NMR (101 MHz, DMSO) δ 179.73, 74.15, 64.22, 52.26, 48.34, 47.85, 44.84, 36.45, 28.27, 27.60, 25.90, 24.57, 24.36, 8.54, 8.48; MS (LC/MS, M+H⁺): 337.3.

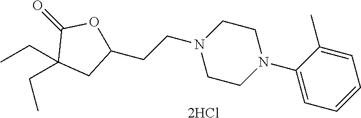 [SMILES: CCC1(CC)CC(CCN2CCN(c3ccccc3C)CC2)OC1=O ; "2HCl"]

Preparation of 3,3-diethyl-5-(2-(4-(o-tolyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-o-Tolyl-piperazine was substituted for 2-piperazin-1-yl-benzonitrile. ¹H NMR (400 MHz, MeOD) δ 7.21-6.90 (m, 4H), 4.62-4.45 (m, 1H), 3.65 (dd, J=9.6, 5.4 Hz, 2H), 3.43-3.26 (m, 4H), 3.24-3.07 (m, 4H), 2.34-2.02 (m, 6H), 1.90 (dd, J=13.3, 9.4 Hz, 1H), 1.60 (ddd, J=17.2, 8.6, 6.4 Hz, 4H), 0.89 (dt, J=14.0, 7.5 Hz, 6H); ¹³C NMR (101 MHz, MeOD) δ 182.40, 150.67, 134.07, 132.33, 127.96, 125.84, 120.39, 76.21, 54.96, 54.81, 53.99, 53.80, 50.26, 49.93, 38.33, 31.77, 30.05, 29.18, 17.84, 9.01, 8.92; MS (LC/MS, M+H⁺): 345.3.

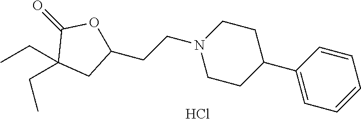 [SMILES: CCC1(CC)CC(CCN2CCC(c3ccccc3)CC2)OC1=O ; Cl]

Preparation of 3,3-diethyl-5-(2-(4-phenylpiperidin-1-yl)ethyl)dihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 4-phenyl-piperidine was substituted for 2-piperazin-1-yl-benzonitrile. ¹H NMR (400 MHz, D₂O) δ 7.39 (tt, J=7.3, 14.3, 5H), 4.71 (s, 1H), 3.72 (s, 2H), 3.36 (s, 2H), 3.17 (s, 2H), 2.98 (s, 1H), 2.37 (dd, J=6.9, 13.4, 1H), 2.31-2.10 (m, 4H), 2.02 (dd, J=9.4, 13.5, 3H), 1.78-1.53 (m, 4H), 0.92 (dt, J=7.5, 12.7, 6H); ¹³C NMR (101 MHz, D₂O) δ 187.89, 146.67, 131.85, 130.03, 129.64, 79.59, 52.33, 41.74, 39.25, 32.90, 31.85, 30.60, 10.89, 10.76; MS (LC/MS, M+H⁺): 330.2; Anal. Calcd for C₂₁H₃₂ClNO₂: C, 68.93; H, 8.81; N, 3.83. Found: C, 68.87; H, 8.93; N, 3.79.

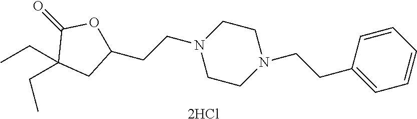 [SMILES: CCC1(CC)CC(CCN2CCN(CCc3ccccc3)CC2)OC1=O ; "2HCl"]

Preparation of 3,3-diethyl-5-(2-(4-phenethylpiperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-phenethyl-piperazine was substituted for 2-piperazin-1-yl-benzonitrile. ¹H NMR (400 MHz, D₂O) δ 7.39 (dd, J=7.3, 25.0, 5H), 4.71 (s, 1H), 3.71 (s, 7H), 3.60-3.52 (m, 2H), 3.44 (s, 2H), 3.18-3.09 (m, 2H), 2.35 (dd, J=6.9, 13.4, 1H), 2.16 (s, 2H), 2.05-1.92 (m, 1H), 1.75-1.49 (m, 4H), 0.98-0.79 (m, 6H); ¹³C NMR (101 MHz, D₂O) δ 187.86, 138.66, 132.14, 131.77, 130.51, 79.45, 60.57, 56.71, 52.37, 51.86, 51.79, 39.38, 32.87, 32.64, 31.85, 30.63, 10.98, 10.85; MS (LC/MS, M+H⁺): 359.3; Anal. Calcd for C₂₂H₃₆C₁₂N₂O₂: C, 61.25; H, 8.41; N, 6.49. Found: C, 60.95; H, 8.33; N, 6.42.

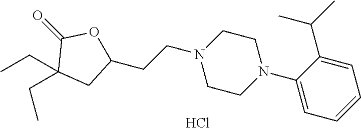 [SMILES: CCC1(CC)CC(CCN2CCN(c3ccccc3C(C)C)CC2)OC1=O ; Cl]

Preparation of 3,3-diethyl-5-(2-(4-(2-isopropylphenyl)piperazin-1-yl)ethyl)dihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2-Isopropyl-phenyl)-piperazine was substituted for 2-piperazin-1-yl-benzonitrile. ¹H NMR (400 MHz, DMSO) δ 7.30 (dd, J=7.4, 1.6 Hz, 1H), 7.23-7.08 (m, 3H), 4.66-4.43 (m, 1H), 3.54 (t, J=9.6 Hz, 2H), 3.41 (dd, J=13.7, 6.8 Hz, 1H), 3.33-3.12 (m, 6H), 3.02 (d, J=10.7 Hz, 2H), 2.31-2.03 (m, 3H), 1.83 (dd, J=13.2, 9.3 Hz, 1H), 1.69-1.34 (m, 4H), 1.16 (d, J=6.9 Hz, 6H), 0.85 (dt, J=10.6, 7.5 Hz, 6H); ¹³C NMR (101 MHz, DMSO) δ 179.77, 148.90, 143.85, 126.51, 125.20, 120.36, 74.31, 52.05, 51.57, 51.43, 49.55, 47.87, 36.43, 29.72, 28.36, 27.66, 26.24, 23.99, 8.55, 8.51; MS (LC/MS, M+H⁺): 373.3.

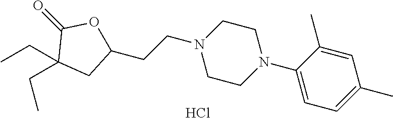 [SMILES: CCC1(CC)CC(CCN2CCN(c3ccc(C)cc3C)CC2)OC1=O ; Cl]

Preparation of 5-(2-(4-(2,4-dimethylphenyl)piperazin-1-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2,4-dimethyl-phenyl)-piperazine was substituted for 2-piperazin-1-yl-benzonitrile. ¹H NMR (400 MHz, DMSO) δ 7.11-6.75 (m, 3H), 4.55 (dt, J=11.8, 8.4 Hz, 1H), 3.53 (m, 2H), 3.33-3.02 (m, 8H), 2.31-2.07 (m, 9H), 1.83 (dd, J=13.2, 9.3 Hz, 1H), 1.67-1.39 (m, 4H), 0.85 (dt, J=10.6, 7.5 Hz, 6H); ¹³C NMR (101 MHz, DMSO) δ 179.75, 147.31, 132.64, 131.80, 131.62, 127.05, 118.82, 74.31, 52.11, 51.55, 51.38, 48.24, 47.85, 36.44, 29.71, 28.33, 27.64, 20.32, 17.27, 8.54, 8.49; MS (LC/MS, M+H⁺): 359.3.

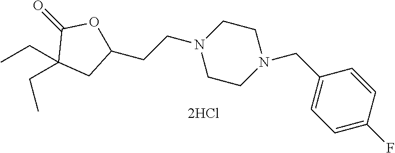 [SMILES: CCC1(CC)CC(CCN2CCN(Cc3ccc(F)cc3)CC2)OC1=O ; "2HCl"]

Preparation of 3,3-diethyl-5-(2-(4-(4-fluorobenzyl)piperazin-1-yl)ethyl)dihydrofuran-2(3H)-one dihydrochloride: The title compound was prepared according to the procedure for 2-(4-(2-(4,4-diethyl-5-oxotetrahydrofuran-2-yl)ethyl)piperazin-1-yl)benzonitrile, except 1-(4-Fluoro-benzyl)-piperazine was substituted for 2-piperazin-1-yl-benzonitrile. ¹H NMR (400 MHz, D₂O) δ 7.55 (dd, J=5.3, 8.6, 2H), 7.26 (t, J=8.7, 2H), 4.70 (m, 1H), 4.45 (m, 2H), 3.66 (broad, 8H), 3.55-3.37 (m, 2H), 2.34 (dd, J=6.9, 13.4, 1H), 2.30-2.08 (m, 2H), 1.98 (dd, J=9.5, 13.4, 1H), 1.63 (dddt, J=7.1, 14.0, 21.4, 28.4, 4H), 0.88 (dt, J=7.4, 14.7, 6H); ¹³C NMR (101 MHz, D₂O) δ 187.84, 167.92, 165.46, 136.47, 136.38, 126.74, 119.43, 119.21, 79.41, 62.68, 56.69, 52.36, 51.85, 51.11, 39.37, 32.84, 31.85, 30.63, 26.67, 10.97, 10.85; MS (LC/MS, M+H⁺): 363.2; Anal. Calcd for C₂₁H₃₃C₁₂FN₂O₂: C, 57.93; H, 7.64; N, 6.43. Found: C, 57.71; H, 7.69; N, 6.32.

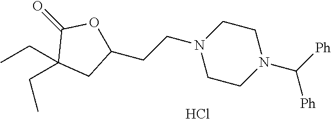 [SMILES: CCC1(CC)CC(CCN2CCN(C(c3ccccc3)c3ccccc3)CC2)OC1=O ; Cl]

Preparation of 5-(2-(4-benzhydrylpiperazin-1-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-benzhydryl-piperazine was substituted for 2-piperazin-1-yl-benzonitrile. ¹H NMR (400 MHz, D₂O) δ 7.67 (d, J=7.2, 4H), 7.50 (dq, J=7.1, 14.4, 6H), 4.67 (s, 1H), 3.82-3.35 (m, 10H), 2.33 (dd, J=6.9, 13.5, 1H), 2.18 (d, J=33.6, 2H), 1.98 (dd, J=9.5, 13.5, 1H), 1.62 (ddd, J=6.8, 14.3, 28.4, 4H), 0.98-0.79 (m, 6H); ¹³C NMR (101 MHz, D₂O) δ 187.81, 137.25, 132.85, 132.78, 130.96, 79.35, 78.44, 56.55, 52.34, 52.09, 51.50, 39.35, 32.76, 31.83, 30.61, 26.67, 10.97, 10.84; MS (LC/MS, M+H⁺): 421.3.

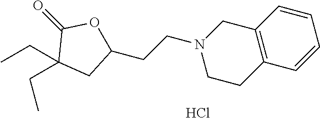 [SMILES: CCC1(CC)CC(CCN2CCc3ccccc3C2)OC1=O ; Cl]

Preparation of 5-(2-(3,4-dihydroisoquinolin-2(1H)-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1,2,3,4-tetrahydro-isoquinoline was substituted for 2-piperazin-1-yl-benzonitrile. ¹H NMR (400 MHz, MeOH) δ 7.39-7.17 (m, 4H), 4.63-4.54 (m, 1H), 4.49 (s, 2H), 3.75-3.63 (m, 2H), 3.54-3.37 (m, 2H), 3.22 (m, 2H), 2.36-2.24 (m, 2H), 2.23-2.08 (m, 1H), 1.95 (dd, J=9.4, 13.3, 1H), 1.75-1.53 (m, 4H), 0.94 (dt, J=7.5, 12.2, 6H); ¹³C NMR (101 MHz, MeOH) δ 183.24, 132.92, 130.75, 130.38, 129.74, 129.17, 128.70, 77.07, 55.67, 55.33, 55.28, 52.24, 39.25, 32.87, 30.89, 30.02, 27.35, 9.85, 9.77; MS (LC/MS, M+H⁺): 302.2; Anal. Calcd for C₁₉H₂₈ClNO₂: C, 67.54; H, 8.35; N, 4.15. Found: C, 67.60; H, 8.36; N, 4.14.

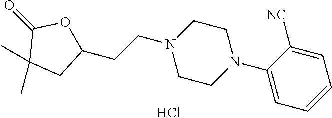 [SMILES: CC1(C)CC(CCN2CCN(c3ccccc3C#N)CC2)OC1=O ; Cl]

Preparation of 2-(4-(2-(4,4-dimethyl-5-oxotetrahydrofuran-2-yl)ethyl)piperazin-1-yl)benzonitrile hydrochloride: The title compound was prepared according to the procedure for 2-(4-(2-(4,4-diethyl-5-oxotetrahydrofuran-2-yl)ethyl)piperazin-1-yl)benzonitrile, except 2-Piperazin-1-yl-benzonitrile substituted for 2-piperazin-1-yl-benzonitrile, and 2-(4,4-dimethyl-5-oxotetrahydrofuran-2-yl)ethyl 4-methylbenzenesulfonate substituted for 2-(4,4-diethyl-5-oxotetrahydrofuran-2-yl)ethyl 4-methylbenzenesulfonate. ¹H NMR (400 MHz, MeOH) δ 7.74-7.57 (m, 2H), 7.33-7.16 (m, 2H), 4.71-4.55 (m, 1H), 4.00-3.30 (m, 10H), 2.40-2.09 (m, 3H), 1.90 (dd, J=10.0, 12.7, 1H), 1.27 (s, 6H); ¹³C NMR (101 MHz, MeOH) δ 184.38, 156.00, 136.47, 136.20, 125.87, 121.61, 119.60, 108.88, 76.79, 55.85, 55.67, 54.38, 50.88, 50.50, 50.29, 50.07, 49.86, 49.65, 49.43, 49.22, 44.60, 42.41, 31.98, 25.94, 25.38; MS (LC/MS, M+H⁺): 328.2; Anal. Calcd for C₁₉H₂₆ClN₃O₂: C, 62.71; H, 7.20; Cl, 9.74; N, 11.55. Found: C, 62.59; H, 7.18; N, 11.42.

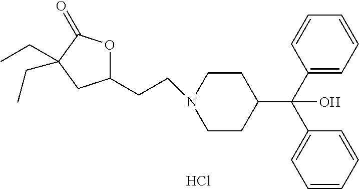 [SMILES: CCC1(CC)CC(CCN2CCC(C(O)(c3ccccc3)c3ccccc3)CC2)OC1=O ; Cl]

Preparation of 3,3-diethyl-5-(2-(4-(hydroxydiphenylmethyl)piperidin-1-yl)ethyl)dihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except diphenyl-piperidin-4-yl-methanol was substituted for 2-piperazin-1-yl-benzonitrile. ¹H NMR (400 MHz, MeOD) δ 7.57-7.46 (m, 4H), 7.30 (m, 4H), 7.18 (m, 2H), 4.56-4.45 (m, 1H), 3.57 (m, 2H), 3.33-3.16 (m, 6H), 3.05 (mz, 2H), 2.92-2.81 (m, 1H), 2.27 (dd, J=13.3, 6.7 Hz, 1H), 2.13 (m, 1H), 2.03 (d, J=2.0 Hz, 1H), 1.94-1.87 (m, 1H), 1.63 (ddd, J=16.2, 9.4, 6.1 Hz, 4H), 0.92 (dt, J=15.7, 7.5 Hz, 6H); ¹³C NMR (101 MHz, MeOD) δ 182.31, 147.09, 129.20, 127.69, 127.08, 79.80, 76.20, 49.89, 42.70, 38.36, 31.91, 30.01, 29.15, 8.97, 8.88; MS (LC/MS, M+H⁺): 436.3.

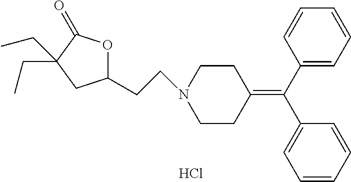 [SMILES: CCC1(CC)CC(CCN2CCC(=C(c3ccccc3)c3ccccc3)CC2)OC1=O ; Cl]

Preparation of 5-(2-(4-(diphenylmethylene)piperidin-1-yl)ethyl)-3,3-diethyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 4-Benzhydrylidene-piperidine was substituted for 2-piperazin-1-yl-benzonitrile. ¹H NMR (400 MHz, MeOD) δ 7.39-7.21 (m, 6H), 7.19-7.11 (m, 4H), 4.59-4.48 (m, 1H), 3.42-3.21 (m, 10H), 2.31-2.24 (m, 1H), 2.13 (m, 2H), 1.92 (dd, J=13.3, 9.4 Hz, 1H), 1.64 (m, 4H), 0.93 (dt, J=15.2, 7.5 Hz, 6H); ¹³C NMR (101 MHz, MeOD) δ 182.30, 142.47, 141.57, 130.38, 129.46, 128.33, 76.16, 54.88, 49.90, 38.36, 32.00, 30.04, 29.56, 29.17, 8.97, 8.89; MS (LC/MS, M+H⁺): 418.3.

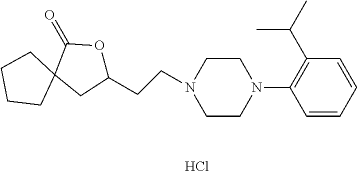 [SMILES: CC(C)c1ccccc1N1CCN(CCC2CC3(CCCC3)C(=O)O2)CC1 ; Cl]

Preparation of 3-(2-(4-(2-isopropylphenyl)piperazin-1-yl)ethyl)-2-oxaspiro[4.4]nonan-1-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2-isopropyl-phenyl)-piperazine substituted for 2-piperazin-1-yl-benzonitrile, and 2-(1-oxo-2-oxaspiro[4.4]nonan-3-yl)ethyl 4-methylbenzenesulfonate substituted for 2-(4,4-diethyl-5-oxotetrahydrofuran-2-yl)ethyl 4-methylbenzenesulfonate. ¹H NMR (400 MHz, D₂O) δ 7.51-7.39 (m, 1H), 7.25 (m, 3H), 4.68 (m, 1H), 3.78-3.08 (m, 11H), 2.42 (dd, J=12.8, 6.0 Hz, 1H), 2.22 (m, 2H), 2.09-1.93 (m, 2H), 1.90-1.60 (m, 7H), 1.20 (d, J=6.9 Hz, 6H); ¹³C NMR (101 MHz, D₂O) δ 189.43, 147.87, 130.06, 129.92, 129.00, 123.54, 79.88, 53.51, 52.89, 44.63, 40.17, 39.34, 32.14, 29.56, 28.12, 28.06, 26.11; MS (LC/MS, M+H⁺): 371.3.

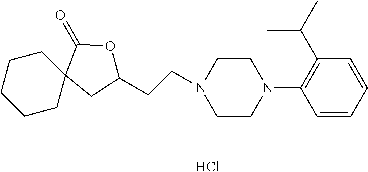 [SMILES: CC(C)c1ccccc1N1CCN(CCC2CC3(CCCCC3)C(=O)O2)CC1 ; Cl]

Preparation of 3-(2-(4-(2-isopropylphenyl)piperazin-1-yl)ethyl)-2-oxaspiro[4.5]decan-1-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2-isopropyl-phenyl)-piperazine substituted for 2-piperazin-1-yl-benzonitrile, and 2-(1-oxo-2-oxaspiro[4.5]decan-3-yl)ethyl 4-methylbenzenesulfonate substituted for 2-(4,4-diethyl-5-oxotetrahydrofuran-2-yl)ethyl 4-methylbenzenesulfonate. ¹H NMR (400 MHz, MeOD) δ 7.26-7.17 (m, 1H), 7.17-7.02 (m, 3H), 4.59-4.44 (m, 1H), 3.61 (m, 2H), 3.48-3.20 (m, 7H), 3.11 (m, 2H), 2.47 (dd, J=13.0, 6.2 Hz, 1H), 2.14 (ddd, J=18.5, 7.5, 3.2 Hz, 2H), 1.77-1.51 (m, 6H), 1.41 (m, 3H), 1.24 (s, 2H), 1.11 (d, J=6.9 Hz, 6H); ¹³C NMR (101 MHz, MeOD) δ 183.07, 149.17, 145.74, 127.93, 127.88, 127.39, 121.80, 76.35, 54.99, 53.88, 53.73, 51.71, 46.21, 40.01, 35.29, 32.65, 31.38, 28.11, 26.50, 24.44, 23.19, 23.11. MS (LC/MS, M+H⁺): 385.3.

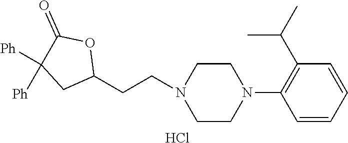 [SMILES: CC(C)c1ccccc1N1CCN(CCC2CC(c3ccccc3)(c3ccccc3)C(=O)O2)CC1 ; Cl]

Preparation of 5-(2-(4-(2-isopropylphenyl)piperazin-1-yl)ethyl)-3,3-diphenyldihydrofuran-2(3H)-one hydrochloride: The title compound was prepared according to the procedure for 2-(4-(2-(4,4-diethyl-5-oxotetrahydrofuran-2-yl)ethyl)piperazin-1-yl)benzonitrile, except 1-(2-isopropyl-phenyl)-piperazine substituted for 2-piperazin-1-yl-benzonitrile, and 2-(5-oxo-4,4-diphenyltetrahydrofuran-2-yl)ethyl 4-methylbenzenesulfonate substituted for 2-(4,4-diethyl-5-oxotetrahydrofuran-2-yl)ethyl 4-methylbenzenesulfonate. ¹H NMR (400 MHz, DMSO) δ 7.47-7.39 (m, 4H), 7.38-7.23 (m, 7H), 7.22-7.07 (m, 3H), 4.41 (dt, J=10.8, 5.3 Hz, 1H), 3.52 (m, 2H), 3.45-3.37 (m, 1H), 3.33 (m, 3H), 3.25-3.12 (m, 4H), 3.03 (m, 2H), 2.74-2.62 (m, 1H), 2.27 (m, 2H), 1.16 (d, J=6.9 Hz, 6H); ¹³C NMR (101 MHz, DMSO) δ 148.84, 128.94, 128.23, 127.53, 127.16, 126.48, 120.38, 74.84, 57.38, 49.55, 28.36, 26.23, 23.94; MS (LC/MS, M+H⁺): 469.3.

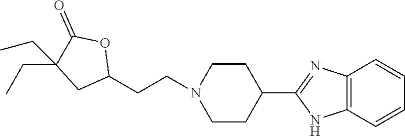 [SMILES: CCC1(CC)CC(CCN2CCC(c3nc4ccccc4[nH]3)CC2)OC1=O]

Preparation of 5-(2-(4-(1H-benzo[d]imidazol-2-yl)piperidin-1-yl)ethyl)-3,3-diethyldihydrofuran-2(3H)-one: 2-(4,4-diethyl-5-oxotetrahydrofuran-2-yl)ethyl 4-methylbenzenesulfonate (102 mg, 0.3 mmol, 1.0 equiv) was treated with 2-piperidin-4-yl-1H-benzoimidazole (181 mg, 0.9 mmol, 3.0 equiv) in dry CH₃CN (3 mL) and heated in microwave reactor at 120° C. for 1 h. The mixture was then filtered through a syringe filter and purified by HPLC [(CH₃CN/H₂O), 0%-100%]. The purified product was then partitioned with NaHCO₃ solution. The aqueous was extracted with dichloromethane (3×25 mL). The combined organic phase was then dried over MgSO₄, filtered, and concentrated to give the desired product. ¹H NMR (400 MHz, CDCl₃) δ 7.47 (m, 2H), 7.12 (m, 2H), 4.38 (m, 1H), 3.44 (s, 1H), 2.90 (m, 3H), 2.43 (m, 2H), 2.19-1.83 (m, 7H), 1.84-1.66 (m, 3H), 1.64-1.41 (m, 4H), 0.85 (dt, J=20.6, 7.4 Hz, 6H); ¹³C NMR (101 MHz, CDCl₃) δ 181.32, 157.79, 122.27, 75.98, 54.73, 53.78, 53.41, 48.79, 37.71, 36.74, 33.95, 30.91, 30.86, 29.33, 28.38, 8.89, 8.81; MS (LC/MS, M+H⁺): 370.2.

The following compounds can be prepared by the procedure of 5-(2-(4-(1H-benzo[d]imidazol-2-yl)piperidin-1-yl)ethyl)-3,3-diethyldihydrofuran-2(3H)-one. The skilled practitioner will know how to substitute the appropriate reagents, starting materials and purification methods known to those skilled in the art, in order to prepare the compounds provided herein.

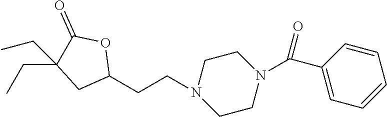 [SMILES: CCC1(CC)CC(CCN2CCN(C(=O)c3ccccc3)CC2)OC1=O]

Preparation of 5-(2-(4-benzoylpiperazin-1-yl)ethyl)-3,3-diethyldihydrofuran-2(3H)-one: The title compound was prepared according to the procedure for 5-(2-(4-(1H-benzo[d]imidazol-2-yl)piperidin-1-yl)ethyl)-3,3-diethyldihydrofuran-2(3H)-one, except phenyl-piperazin-1-yl-methanone was substituted for 2-piperazin-1-yl-benzonitrile. ¹H NMR (400 MHz, CDCl₃) δ 7.33 (s, 5H), 4.40 (tt, J=9.2, 7.1 Hz, 1H), 3.80-3.61 (broad, 2H), 3.37 (broad, J=3.3 Hz, 2H), 2.42 (ddd, J=43.0, 22.8, 14.8 Hz, 6H), 2.06 (dd, J=13.1, 6.8 Hz, 1H), 1.82-1.68 (m, 3H), 1.63-1.47 (m, 4H), 0.85 (dt, J=21.4, 7.5 Hz, 6H); ¹³C NMR (101 MHz, CDCl₃) δ 180.72, 170.27, 135.74, 129.70, 128.48, 127.02, 75.33, 54.33, 48.58, 37.63, 33.68, 29.20, 28.25, 8.78, 8.70; MS (LC/MS, M+H⁺): 359.2.

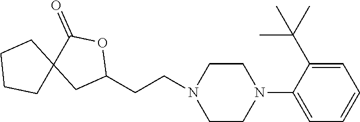 [SMILES: CC(C)(C)c1ccccc1N1CCN(CCC2CC3(CCCC3)C(=O)O2)CC1]

Preparation of 3-(2-(4-(2-(tert-butyl)phenyl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2-tert-Butyl-phenyl)-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.41-7.30 (m, 2H), 7.21 (m, 1H), 7.17-7.08 (m, 1H), 4.58-4.37 (m, 1H), 3.07-2.94 (m, 2H), 2.87 (m, 2H), 2.80 (m, 2H), 2.59 (m, 2H), 2.42-2.12 (m, 4H), 2.04-1.56 (m, 10H), 1.43 (s, 9H); ¹³C NMR (101 MHz, CDCl₃) δ 182.54, 153.27, 147.62, 127.00, 126.92, 126.09, 125.75, 76.36, 54.86, 54.15, 54.06, 53.66, 50.26, 43.41, 37.58, 37.04, 35.67, 33.18, 30.95, 25.63, 25.55; MS (LC/MS, M+H⁺): 385.1.

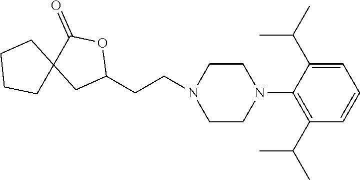 [SMILES: CC(C)c1cccc(C(C)C)c1N1CCN(CCC2CC3(CCCC3)C(=O)O2)CC1]

Preparation of 3-(2-(4-(2,6-diisopropylphenyl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2,6-diisopropyl-phenyl)-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.17 (m, 1H), 7.12-7.05 (m, 2H), 4.49 (tt, J=8.2, 5.6 Hz, 1H), 3.48 (dt, J=13.8, 6.9 Hz, 2H), 3.22-3.07 (broad, 4H), 2.58 (m, 6H), 2.32-2.11 (m, 2H), 2.01-1.58 (m, 10H), 1.19 (d, J=6.9 Hz, 12H); ¹³C NMR (101 MHz, CDCl₃) δ 149.23, 126.71, 124.09, 76.42, 55.23, 54.64, 51.03, 50.26, 43.44, 37.61, 37.09, 33.18, 28.28, 25.64, 25.55, 24.44; MS (LC/MS, M+H⁺): 413.2

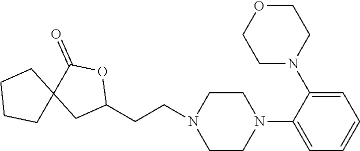 [SMILES: O=C1OC(CCN2CCN(c3ccccc3N3CCOCC3)CC2)CC12CCCC2]

Preparation of 3-(2-(4-(2-morpholinophenyl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4-(2-piperazin-1-yl-phenyl)-morpholine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate: ¹H NMR (400 MHz, CDCl₃) δ 7.12-6.82 (m, 4H), 4.58-4.43 (m, 1H), 3.96-3.74 (m, 4H), 3.18 (broad, 8H), 2.76-2.41 (m, 6H), 2.35-2.08 (m, 2H), 2.02-1.55 (m, 10H); ¹³C NMR (101 MHz, CDCl₃) δ 182.46, 144.70, 144.54, 123.15, 123.05, 118.70, 118.30, 76.27, 67.70, 54.75, 54.10, 50.20, 50.02, 49.41, 43.35, 37.52, 36.99, 33.15, 25.58, 25.49; MS (LC/MS, M+H⁺):

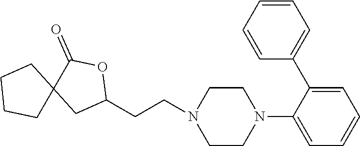 [SMILES: O=C1OC(CCN2CCN(c3ccccc3-c3ccccc3)CC2)CC12CCCC2]

Preparation of 3-(2-(4-([1,1′-biphenyl]-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-biphenyl-2-yl-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.55 (dd, J=5.1, 3.3 Hz, 2H), 7.31 (dd, J=10.4, 4.7 Hz, 2H), 7.20 (m, 3H), 6.98 (m, 2H), 4.44-4.23 (m, 1H), 2.77 (broad, 4H), 2.49-2.17 (m, 6H), 2.10 (d, J=5.8 Hz, 2H), 1.86-1.42 (m, 10H); ¹³C NMR (101 MHz, CDCl₃) δ 182.45, 150.24, 141.23, 135.03, 131.55, 128.93, 128.37, 128.26, 126.82, 122.72, 118.27, 76.26, 54.59, 53.43, 50.98, 50.18, 43.32, 37.51, 36.98, 33.08, 25.57, 25.49; MS (LC/MS, M+H⁺): 405.1.

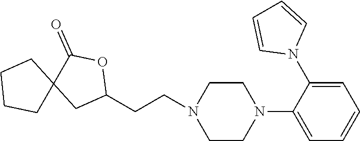 [SMILES: O=C1OC(CCN2CCN(c3ccccc3-n3cccc3)CC2)CC12CCCC2]

Preparation of 3-(2-(4-(2-(1H-pyrrol-1-yl)phenyl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2-pyrrol-1-yl-phenyl)-piperazine substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.23-7.06 (m, 2H), 7.05-6.89 (m, 4H), 6.20 (m, 2H), 4.45-4.26 (m, 1H), 2.64 (broad, 4H), 2.51-2.28 (m, 6H), 2.19-1.95 (m, 2H), 1.91-1.45 (m, 10H); ¹³C NMR (101 MHz, CDCl₃) δ 182.47, 146.37, 133.69, 127.59, 126.62, 122.56, 121.25, 118.90, 109.06, 76.23, 54.55, 53.55, 50.19, 49.97, 43.33, 37.51, 36.98, 33.04, 25.58, 25.49; MS (LC/MS, M+H⁺): 394.1.

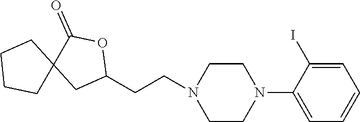 [SMILES: O=C1OC(CCN2CCN(c3ccccc3I)CC2)CC12CCCC2]

Preparation of 3-(2-(4-(2-iodophenyl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2-iodo-phenyl)-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.84 (dd, J=7.8, 1.4 Hz, 1H), 7.31 (td, J=7.9, 1.5 Hz, 1H), 7.04 (dd, J=8.0, 1.4 Hz, 1H), 6.79 (td, J=7.7, 1.5 Hz, 1H), 4.59-4.39 (m, 1H), 3.02 (broad, 4H), 2.79-2.47 (m, 6H), 2.31-2.09 (m, 2H), 2.00-1.54 (m, 10H); ¹³C NMR (101 MHz, CDCl₃) δ 182.51, 153.44, 140.20, 129.34, 125.59, 121.13, 98.29, 76.29, 54.63, 53.58, 52.34, 50.27, 43.43, 37.59, 37.05, 33.13, 25.64, 25.56; MS (LC/MS, M+H⁺): 455.0

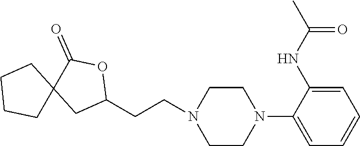 [SMILES: CC(=O)Nc1ccccc1N1CCN(CCC2CC3(CCCC3)C(=O)O2)CC1]

Preparation of N-(2-(4-(2-(1-oxo-2-oxaspiro[4.4]nonan-3-yl)ethyl)piperazin-1-yl)phenyl)acetamide: The title compound was prepared according to the procedure for 5-(2-(4-(1H-benzo[d]imidazol-2-yl)piperidin-1-yl)ethyl)-3,3-diethyldihydrofuran-2(3H)-one, except N-(2-piperazin-1-yl-phenyl)-acetamide was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.45 (s, 1H), 8.33 (d, J=8.0 Hz, 1H), 7.22-7.08 (m, 2H), 7.08-6.99 (m, 1H), 4.56-4.43 (m, 1H), 2.89 (broad, 4H), 2.61 (m, 6H), 2.31-2.09 (m, 5H), 1.98-1.54 (m, 10H); ¹³C NMR (101 MHz, CDCl₃) δ 182.47, 168.16, 140.92, 133.63, 125.67, 123.81, 120.55, 119.59, 76.13, 54.68, 54.21, 52.25, 50.26, 43.40, 37.59, 37.04, 33.21, 25.63, 25.54, 25.09; MS (LC/MS, M+H⁺): 386.1.

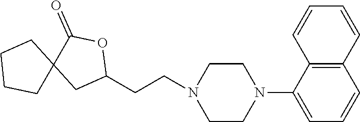 [SMILES: O=C1OC(CCN2CCN(c3cccc4ccccc34)CC2)CC12CCCC2]

Preparation of 3-(2-(4-(naphthalen-1-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-Naphthalen-1-yl-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.31-8.11 (m, 1H), 7.91-7.72 (m, 1H), 7.60-7.32 (m, 4H), 7.09 (dd, J=7.4, 0.8 Hz, 1H), 4.65-4.39 (m, 1H), 3.15 (broad, 4H), 2.65 (m, 6H), 2.36-2.08 (m, 2H), 2.04-1.55 (m, 10H); ¹³C NMR (101 MHz, CDCl₃) δ 182.54, 149.71, 134.88, 129.01, 128.52, 125.98, 125.93, 125.46, 123.68, 123.64, 114.75, 76.35, 54.76, 53.94, 53.06, 50.28, 43.45, 37.59, 37.06, 33.24, 25.65, 25.56; MS (LC/MS, M+H⁺): 479.1.

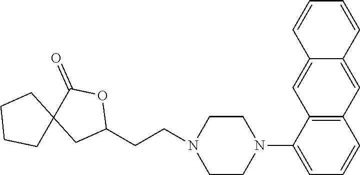 [SMILES: O=C1OC(CCN2CCN(c3cccc4cc5ccccc5cc34)CC2)CC12CCCC2]

Preparation of 3-(2-(4-(anthracen-1-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-anthracen-1-yl-piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.76 (s, 1H), 8.42 (s, 1H), 8.04 (ddd, J=9.6, 6.0, 3.2 Hz, 2H), 7.73 (d, J=8.5 Hz, 1H), 7.55-7.45 (m, 2H), 7.40 (dd, J=8.4, 7.3 Hz, 1H), 7.05 (d, J=6.9 Hz, 1H), 4.55 (tt, J=8.1, 5.5 Hz, 1H), 3.25 (broad, 4H), 2.96-2.60 (m, 6H), 2.34-2.17 (m, 2H), 1.76 (m, 10H); ¹³C NMR (101 MHz, CDCl₃) δ 182.53, 149.60, 133.09, 131.67, 131.35, 128.82, 127.96, 127.70, 126.75, 125.58, 125.40, 125.30, 123.85, 122.47, 113.38, 76.31, 54.74, 53.91, 53.03, 50.24, 43.38, 37.54, 37.01, 33.18, 25.61, 25.53; MS (LC/MS, M+H⁺): 429.1.

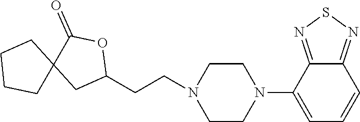 [SMILES: O=C1OC(CCN2CCN(c3cccc4nsnc34)CC2)CC12CCCC2]

Preparation of 3-(2-(4-(benzo[c][1,2,5]thiadiazol-4-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4-piperazin-1-yl-benzo[1,2,5]thiadiazole was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.53 (dd, J=8.7, 0.8 Hz, 1H), 7.45 (dd, J=8.7, 7.3 Hz, 1H), 6.72 (dd, J=7.3, 0.6 Hz, 1H), 4.59-4.40 (m, 1H), 3.56 (s, 4H), 2.74 (s, 4H), 2.61 (td, J=8.5, 6.3 Hz, 2H), 2.30-2.12 (m, 2H), 2.03-1.53 (m, 10H); ¹³C NMR (101 MHz, CDCl₃) δ 182.48, 156.74, 149.80, 144.28, 130.56, 113.70, 111.51, 76.22, 54.64, 53.30, 50.25, 50.17, 43.39, 37.55, 37.01, 33.10, 25.61, 25.52; MS (LC/MS, M+H⁺): 387.0.

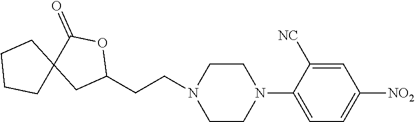 [SMILES: N#Cc1cc([N+](=O)[O-])ccc1N1CCN(CCC2CC3(CCCC3)C(=O)O2)CC1]

Preparation of 5-nitro-2-(4-(2-(1-oxo-2-oxaspiro[4.4]nonan-3-yl)ethyl)piperazin-1-yl)benzonitrile: The title compound was prepared according to the procedure for 5-(2-(4-(1H-benzo[d]imidazol-2-yl)piperidin-1-yl)ethyl)-3,3-diethyldihydrofuran-2(3H)-one, except 5-nitro-2-piperazin-1-yl-benzonitrile was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.41 (d, J=2.7 Hz, 1H), 8.25 (dd, J=9.3, 2.7 Hz, 1H), 6.97 (d, J=9.4 Hz, 1H), 4.48 (tt, J=8.1, 5.7 Hz, 1H), 3.63-3.41 (m, 4H), 2.78-2.50 (m, 6H), 2.34-2.03 (m, 2H), 1.95-1.54 (m, 10H); ¹³C NMR (101 MHz, CDCl₃) δ 182.31, 158.33, 139.60, 131.36, 129.00, 117.68, 117.01, 102.03, 75.83, 54.21, 52.75, 50.52, 50.14, 43.23, 37.46, 36.89, 32.90, 25.50, 25.41; MS (LC/MS, M+H⁺): 399.2.

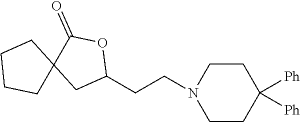 [SMILES: O=C1OC(CCN2CCC(c3ccccc3)(c3ccccc3)CC2)CC12CCCC2]

Preparation of 3-(2-(4,4-diphenylpiperidin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4,4-diphenyl-piperidine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.32-7.20 (m, 8H), 7.20-7.08 (m, 2H), 4.49-4.35 (m, 1H), 2.62-2.32 (m, 10H), 2.25-2.09 (m, 2H), 1.97-1.50 (m, 10H). 13C NMR (101 MHz, CDCl₃) δ 182.51, 128.48, 127.28, 125.83, 76.42, 54.71, 50.79, 50.23, 44.75, 43.37, 37.55, 37.02, 36.33, 33.28, 25.61, 25.53. MS (LC/MS, M+H⁺): 404.1.

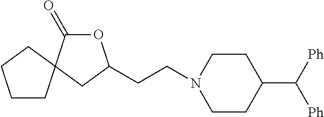 [SMILES: O=C1OC(CCN2CCC(C(c3ccccc3)c3ccccc3)CC2)CC12CCCC2]

Preparation of 3-(2-(4-benzhydrylpiperidin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4-benzhydryl-piperidine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.31-7.19 (m, 8H), 7.18-7.10 (m, 2H), 4.48-4.32 (m, 1H), 3.49 (d, J=10.9 Hz, 1H), 2.86 (d, J=11.6 Hz, 2H), 2.45 (ddd, J=11.9, 9.0, 5.9 Hz, 2H), 2.29-2.02 (m, 3H), 2.01-1.47 (m, 14H), 1.24 (d, J=12.1 Hz, 2H); ¹³C NMR (101 MHz, CDCl₃) δ 182.54, 143.89, 128.63, 128.16, 126.28, 76.53, 59.04, 54.93, 54.42, 53.95, 50.23, 43.39, 39.69, 37.55, 37.00, 33.34, 31.43, 25.62, 25.53; MS (LC/MS, M+H⁺): 418.1.

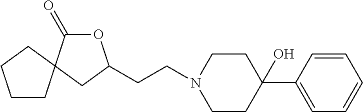 [SMILES: O=C1OC(CCN2CCC(O)(c3ccccc3)CC2)CC12CCCC2]

Preparation of 3-(2-(4-hydroxy-4-phenylpiperidin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4-phenyl-piperidin-4-ol was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.59-7.44 (m, 2H), 7.41-7.19 (m, 3H), 4.47 (ddt, J=10.6, 8.1, 5.5 Hz, 1H), 2.80 (m, 2H), 2.69-2.37 (m, 4H), 2.34-2.05 (m, 4H), 1.99-1.54 (m, 13H); ¹³C NMR (101 MHz, CDCl₃) δ 182.57, 148.43, 128.47, 127.14, 124.65, 76.47, 71.28, 54.78, 50.25, 49.89, 49.55, 43.39, 38.54, 37.55, 37.01, 33.34, 25.62, 25.53; MS (LC/MS, M+H⁺): 344.1.

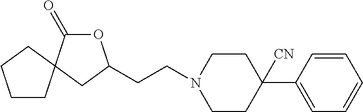 [SMILES: N#CC1(c2ccccc2)CCN(CCC2CC3(CCCC3)C(=O)O2)CC1]

Preparation of 1-(2-(1-oxo-2-oxaspiro[4.4]nonan-3-yl)ethyl)-4-phenylpiperidine-4-carbonitrile: The title compound was prepared according to the procedure for 5-(2-(4-(1H-benzo[d]imidazol-2-yl)piperidin-1-yl)ethyl)-3,3-diethyldihydrofuran-2(3H)-one, except 4-phenyl-piperidine-4-carbonitrile was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate: ¹H NMR (400 MHz, CDCl₃) δ 7.53-7.47 (m, 2H), 7.41 (m, 2H), 7.36-7.30 (m, 1H), 4.47 (tt, J=8.1, 5.5 Hz, 1H), 3.00 (m, 2H), 2.57 (m, 4H), 2.30-2.07 (m, 6H), 1.87 (m, 5H), 1.65 (m, 5H); ¹³C NMR (101 MHz, CDCl₃) δ 182.47, 140.21, 129.18, 128.28, 125.74, 122.11, 76.10, 54.44, 51.04, 50.79, 50.28, 43.39, 42.86, 37.61, 37.04, 36.66, 33.20, 25.64, 25.55; MS (LC/MS, M+H⁺): 353.1.

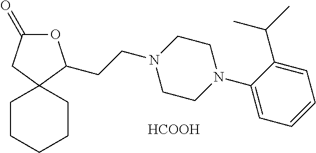 [SMILES: CC(C)c1ccccc1N1CCN(CCC2OC(=O)CC23CCCCC3)CC1 ; "HCOOH"]

Preparation of 1-(2-(4-(2-isopropylphenyl)piperazin-1-yl)ethyl)-2-oxaspiro[4.5]decan-3-one formate: 2-(3-oxo-2-oxaspiro[4.5]decan-1-yl)ethyl 4-methylbenzenesulfonate (105 mg, 0.3 mmol, 1.0 equiv) was treated with 1-(2-isopropyl-phenyl)-piperazine (183 mg, 0.9 mmol, 3.0 equiv) in dry CH₃CN (3 mL) and heated in microwave reactor at 120° C. for 1 h. The mixture was then filtered through a syringe filter and purified by HPLC (CH₃CN/H₂O, 0.1% formic acid), 0%-100%) to give desired product as its formic acid salt. ¹H NMR (400 MHz, CDCl₃) δ 8.40 (s, 1H), 8.17 (broad, 1H), 7.26 (t, J=3.7 Hz, 1H), 7.21-7.08 (m, 3H), 4.13 (dd, J=11.2, 1.7 Hz, 1H), 3.42 (dt, J=13.8, 6.9 Hz, 1H), 3.25-2.78 (m, 10H), 2.57 (d, J=17.3 Hz, 1H), 2.33 (d, J=17.3 Hz, 1H), 2.14-2.00 (m, 1H), 1.91 (m, 1H), 1.66 (m, 3H), 1.41 (m, 7H), 1.20 (d, J=6.9 Hz, 6H); ¹³C NMR (101 MHz, CDCl₃) δ 176.00, 166.89, 149.35, 144.59, 126.66, 126.65, 125.51, 120.93, 86.83, 55.45, 53.26, 51.36, 43.17, 39.66, 35.13, 29.91, 27.02, 25.79, 25.38, 24.12, 24.09, 23.21, 22.44; MS (LC/MS, M+H⁺): 385.3.

The following compounds can be prepared by the procedure of 1-(2-(4-(2-isopropylphenyl)piperazin-1-yl)ethyl)-2-oxaspiro[4.5]decan-3-one formate. The skilled practitioner will know how to substitute the appropriate reagents, starting materials and purification methods known to those skilled in the art, in order to prepare the compounds provided herein.

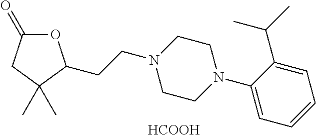 [SMILES: CC(C)c1ccccc1N1CCN(CCC2OC(=O)CC2(C)C)CC1 ; "HCOOH"]

Preparation of 5-(2-(4-(2-isopropylphenyl)piperazin-1-yl)ethyl)-4,4-dimethyldihydrofuran-2(3H)-one formate: The title compound was prepared according to the procedure for 1-(2-(4-(2-isopropylphenyl)piperazin-1-yl)ethyl)-2-oxaspiro[4.5]decan-3-one formate, except 2-(3,3-dimethyl-5-oxotetrahydrofuran-2-yl)ethyl 4-methylbenzenesulfonate was substituted for 2-(3-oxo-2-oxaspiro[4.5]decan-1-yl)ethyl 4-methylbenzenesulfonate. ¹H NMR (400 MHz, CDCl₃) δ 9.79 (broad, 2H), 8.35 (s, 1H), 7.30-7.21 (m, 1H), 7.21-7.09 (m, 3H), 4.12 (dd, J=11.0, 2.0 Hz, 1H), 3.51-2.85 (m, 11H), 2.51-2.27 (m, 2H), 2.22-2.06 (m, 1H), 2.03-1.88 (m, 1H), 1.29-1.12 (m, 9H), 1.03 (s, 3H); ¹³C NMR (101 MHz, CDCl₃) δ 175.64, 166.56, 148.82, 144.51, 126.74, 126.73, 125.83, 120.98, 86.27, 55.33, 50.72, 44.30, 39.45, 27.08, 25.09, 24.60, 24.11, 24.07, 21.63; MS (LC/MS, M+H⁺): 345.3.

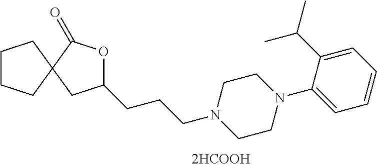 [SMILES: CC(C)c1ccccc1N1CCN(CCCC2CC3(CCCC3)C(=O)O2)CC1]

Preparation of 3-(3-(4-(2-isopropylphenyl)piperazin-1-yl)propyl)-2-oxaspiro[4.4]nonan-1-one formate: The title compound was prepared according to the procedure for 1-(2-(4-(2-isopropylphenyl)piperazin-1-yl)ethyl)-2-oxaspiro[4.5]decan-3-one formate, except 3-(1-oxo-2-oxaspiro[4.4]nonan-3-yl)propyl 4-methylbenzenesulfonate was substituted for 2-(3-oxo-2-oxaspiro[4.5]decan-1-yl)ethyl 4-methylbenzenesulfonate. ¹H NMR (400 MHz, CDCl₃) δ 8.38 (s, 1H), 7.33-7.23 (m, 1H), 7.20-7.10 (m, 3H), 6.10 (s, 3H), 4.49-4.32 (m, 1H), 3.47-2.97 (m, 11H), 2.29-2.08 (m, 2H), 2.08-1.51 (m, 12H), 1.20 (d, J=6.9 Hz, 6H); ¹³C NMR (101 MHz, CDCl₃) δ 182.39, 166.63, 148.82, 144.49, 126.78, 126.71, 125.87, 121.08, 77.22, 56.73, 52.62, 50.61, 50.30, 43.20, 37.56, 36.89, 32.86, 27.11, 25.64, 25.53, 24.11, 20.89; MS (LC/MS, M+H⁺): 385.3.

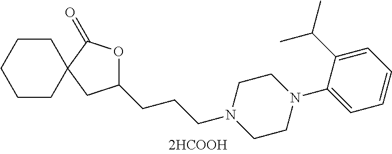 [SMILES: CC(C)c1ccccc1N1CCN(CCCC2CC3(CCCCC3)C(=O)O2)CC1]

Preparation of 3-(3-(4-(2-isopropylphenyl)piperazin-1-yl)propyl)-2-oxaspiro[4.5]decan-1-one formate: The title compound was prepared according to the procedure for 1-(2-(4-(2-isopropylphenyl)piperazin-1-yl)ethyl)-2-oxaspiro[4.5]decan-3-one formate, except 3-(1-oxo-2-oxaspiro[4.5]decan-3-yl)propyl 4-methylbenzenesulfonate was substituted for 2-(3-oxo-2-oxaspiro[4.5]decan-1-yl)ethyl 4-methylbenzenesulfonate. ¹H NMR (400 MHz, CDCl₃) δ 8.35 (s, 2H), 7.31-7.24 (m, 1H), 7.21-7.10 (m, 3H), 4.90 (broad, 4H), 4.50-4.31 (m, 1H), 3.46-2.96 (m, 11H), 2.47-2.34 (m, 1H), 2.07-1.29 (m, 15H), 1.20 (d, J=6.9 Hz, 6H); ¹³C NMR (101 MHz, CDCl₃) δ 181.47, 166.26, 148.70, 144.47, 126.81, 126.73, 125.94, 121.11, 56.72, 52.64, 50.50, 45.05, 39.56, 34.39, 33.20, 31.67, 27.13, 25.40, 24.11, 22.26, 22.20, 20.85; MS (LC/MS, M+H⁺): 399.3.

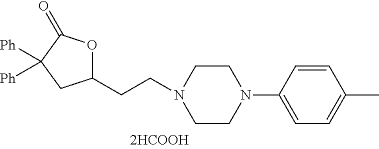 [SMILES: Cc1ccc(N2CCN(CCC3CC(c4ccccc4)(c4ccccc4)C(=O)O3)CC2)cc1]

Preparation of 3,3-diphenyl-5-(2-(4-(p-tolyl)piperazin-1-yl)ethyl)dihydrofuran-2(3H)-one formate: The title compound was prepared according to the procedure for 1-(2-(4-(2-isopropylphenyl)piperazin-1-yl)ethyl)-2-oxaspiro[4.5]decan-3-one formate, except 2-(5-oxo-4,4-diphenyltetrahydrofuran-2-yl)ethyl 4-methylbenzenesulfonate was substituted for 2-(3-oxo-2-oxaspiro[4.5]decan-1-yl)ethyl 4-methylbenzenesulfonate and 1-p-tolyl-piperazine was substituted for 1-(2-isopropyl-phenyl)-piperazine. ¹H NMR (400 MHz, CDCl₃) δ 8.24 (s, 2H), 7.98 (broad, J=8.4 Hz, 3H), 7.50-7.23 (m, 10H), 7.12 (m, 2H), 6.91-6.78 (m, 2H), 4.41 (ddd, J=10.1, 7.4, 4.0 Hz, 1H), 3.49-3.33 (m, 4H), 3.24 (m, 4H), 3.19-3.08 (m, 2H), 2.70 (dd, J=13.1, 10.4 Hz, 1H), 2.45-2.25 (m, 4H); ¹³C NMR (101 MHz, CDCl₃) δ 176.73, 165.78, 147.54, 141.56, 139.34, 131.52, 130.07, 129.20, 129.15, 128.62, 128.07, 127.74, 127.55, 127.36, 125.96, 117.57, 74.76, 58.06, 54.27, 52.27, 47.83, 43.33, 29.81, 20.62; MS (LC/MS, M+H⁺): 441.3.

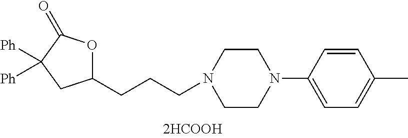 [SMILES: Cc1ccc(N2CCN(CCCC3CC(c4ccccc4)(c4ccccc4)C(=O)O3)CC2)cc1]

Preparation of 3,3-diphenyl-5-(3-(4-(p-tolyl)piperazin-1-yl)propyl)dihydrofuran-2(3H)-one formate: The title compound was prepared according to the procedure for 1-(2-(4-(2-isopropylphenyl)piperazin-1-yl)ethyl)-2-oxaspiro[4.5]decan-3-one formate, except 3-(5-oxo-4,4-diphenyltetrahydrofuran-2-yl)propyl 4-methylbenzenesulfonate was substituted for 2-(3-oxo-2-oxaspiro[4.5]decan-1-yl)ethyl 4-methylbenzenesulfonate and 1-p-tolyl-piperazine was substituted for 1-(2-isopropyl-phenyl)-piperazine. ¹H NMR (400 MHz, CDCl₃) δ 8.74 (broad, 3H), 8.28 (s, 2H), 7.50-7.23 (m, 10H), 7.12 (m, 2H), 6.85 (m, 2H), 4.52-4.22 (m, 1H), 3.52-3.21 (m, 8H), 3.17-3.02 (m, 3H), 2.62 (dd, J=13.0, 10.5 Hz, 1H), 2.31 (d, J=10.6 Hz, 3H), 2.10-1.69 (m, 4H); ¹³C NMR (101 MHz, CDCl₃) δ 177.06, 166.08, 147.50, 141.84, 139.54, 131.49, 130.07, 129.15, 128.56, 127.98, 127.77, 127.45, 127.41, 117.56, 76.63, 58.24, 56.59, 51.81, 47.68, 43.51, 32.21, 20.64, 20.61; MS (LC/MS, M+H⁺): 455.3.

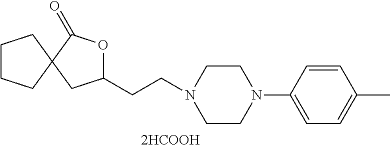 [SMILES: Cc1ccc(N2CCN(CCC3CC4(CCCC4)C(=O)O3)CC2)cc1]

Preparation of 3-(2-(4-(p-tolyl)piperazin-1-yl)ethyl)-2-oxaspiro[4.4]nonan-1-one formate: The title compound was prepared according to the procedure for 1-(2-(4-(2-isopropylphenyl)piperazin-1-yl)ethyl)-2-oxaspiro[4.5]decan-3-one formate, except 2-(1-oxo-2-oxaspiro[4.4]nonan-3-yl)ethyl 4-methylbenzenesulfonate was substituted for 2-(3-oxo-2-oxaspiro[4.5]decan-1-yl)ethyl 4-methylbenzenesulfonate and 1-p-tolyl-piperazine was substituted for 1-(2-isopropyl-phenyl)-piperazine. ¹H NMR (400 MHz, CDCl₃) δ 10.62 (broad, 3H), 8.28 (s, 2H), 7.08 (m, 2H), 6.87-6.74 (m, 2H), 4.43 (tdd, J=9.3, 5.8, 3.4 Hz, 1H), 3.42-3.29 (m, 4H), 3.29-3.13 (m, 5H), 3.11-3.01 (m, 1H), 2.30-2.01 (m, 7H), 1.89-1.52 (m, 8H); ¹³C NMR (101 MHz, CDCl₃) δ 182.00, 166.29, 147.64, 131.31, 130.02, 117.45, 75.24, 54.21, 52.15, 50.06, 47.84, 42.92, 37.49, 36.82, 30.40, 25.58, 25.47, 20.58; MS (LC/MS, M+H⁺): 343.2.

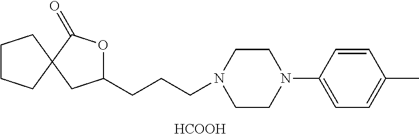 [SMILES: Cc1ccc(N2CCN(CCCC3CC4(CCCC4)C(=O)O3)CC2)cc1 ; "HCOOH"]

Preparation of 3-(3-(4-(p-tolyl)piperazin-1-yl)propyl)-2-oxaspiro[4.4]nonan-1-one formate: The title compound was prepared according to the procedure for 1-(2-(4-(2-isopropylphenyl)piperazin-1-yl)ethyl)-2-oxaspiro[4.5]decan-3-one formate, except 2-(1-oxo-2-oxaspiro[4.4]nonan-3-yl)propyl 4-methylbenzenesulfonate was substituted for 2-(3-oxo-2-oxaspiro[4.5]decan-1-yl)ethyl 4-methylbenzenesulfonate and 1-p-Tolyl-piperazine substituted was substituted for 1-(2-isopropyl-phenyl)-piperazine. ¹H NMR (400 MHz, CDCl₃) δ 8.33 (s, 1H), 7.38 (broad, 2H), 7.09 (d, J=8.3 Hz, 2H), 6.83 (d, J=8.5 Hz, 2H), 4.44-4.26 (m, 1H), 3.35 (m, 4H), 3.14 (broad, 4H), 2.92 (m, 2H), 2.30-2.09 (m, 5H), 1.99-1.52 (m, 12H); ¹³C NMR (101 MHz, CDCl₃) δ 182.40, 166.56, 147.93, 131.05, 130.01, 117.37, 77.22, 56.78, 51.92, 50.29, 48.02, 43.19, 37.56, 36.89, 32.95, 25.63, 25.52, 21.02, 20.60; MS (LC/MS, M+H⁺): 357.3.

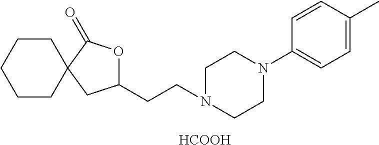 [SMILES: Cc1ccc(N2CCN(CCC3CC4(CCCCC4)C(=O)O3)CC2)cc1 ; "HCOOH"]

Preparation of 3-(2-(4-(p-tolyl)piperazin-1-yl)ethyl)-2-oxaspiro[4.5]decan-1-one formate: The title compound was prepared according to the procedure for 1-(2-(4-(2-isopropylphenyl)piperazin-1-yl)ethyl)-2-oxaspiro[4.5]decan-3-one formate, except 2-(1-oxo-2-oxaspiro[4.5]decan-3-yl)ethyl 4-methylbenzenesulfonate was substituted for 2-(3-oxo-2-oxaspiro[4.5]decan-1-yl)ethyl 4-methylbenzenesulfonate and 1-p-tolyl-piperazine was substituted for 1-(2-isopropyl-phenyl)-piperazine. ¹H NMR (400 MHz, CDCl₃) δ 8.27 (s, 2H), 8.01 (broad, 3H), 7.09 (d, J=8.2 Hz, 2H), 6.83 (t, J=5.6 Hz, 2H), 4.45 (tdd, J=9.5, 6.3, 3.3 Hz, 1H), 3.37 (t, J=4.9 Hz, 4H), 3.29-3.14 (m, 5H), 3.13-3.01 (m, 1H), 2.45 (dd, J=13.0, 6.2 Hz, 1H), 2.32-2.23 (m, 4H), 2.09-1.99 (m, 1H), 1.89-1.15 (m, 11H); ¹³C NMR (101 MHz, CDCl₃) δ 181.10, 166.08, 147.65, 131.40, 130.05, 117.52, 74.89, 54.33, 52.24, 47.88, 44.88, 39.43, 34.33, 31.64, 30.85, 25.33, 22.20, 22.13, 20.61; MS (LC/MS, M+H⁺): 357.2.

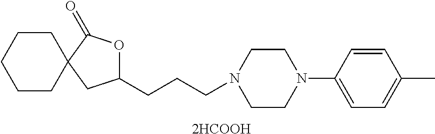 [SMILES: Cc1ccc(N2CCN(CCCC3CC4(CCCCC4)C(=O)O3)CC2)cc1 ; "2HCOOH"]

Preparation of 3-(3-(4-(p-tolyl)piperazin-1-yl)propyl)-2-oxaspiro[4.5]decan-1-one formate: The title compound was prepared according to the procedure for 1-(2-(4-(2-isopropylphenyl)piperazin-1-yl)ethyl)-2-oxaspiro[4.5]decan-3-one formate, except 2-(1-oxo-2-oxaspiro[4.5]decan-3-yl)ethyl 4-methylbenzenesulfonate was substituted for 3-(1-oxo-2-oxaspiro[4.5]decan-3-yl)propyl 4-methylbenzenesulfonate and 1-p-tolyl-piperazine was substituted for 1-(2-isopropyl-phenyl)-piperazine: ¹H NMR (400 MHz, CDCl₃) δ 8.65 (s, 3H), 8.28 (s, 2H), 7.09 (d, J=8.2 Hz, 2H), 6.82 (d, J=8.5 Hz, 2H), 4.40 (tdd, J=9.7, 6.2, 3.9 Hz, 1H), 3.39 (m, 3H), 3.28 (broad, 3H), 3.07 (m, 2H), 2.44-2.30 (m, 1H), 2.27 (s, 3H), 2.04-1.87 (m, 2H), 1.86-1.14 (m, 14H); ¹³C NMR (101 MHz, CDCl₃) δ 181.48, 166.12, 147.53, 131.46, 130.06, 117.55, 77.48, 77.16, 76.84, 76.71, 56.63, 51.81, 47.69, 45.02, 39.48, 34.35, 33.09, 31.63, 25.36, 22.22, 22.15, 20.70, 20.60; MS (LC/MS, M+H⁺): 371.3.

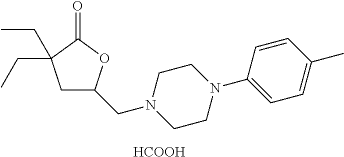 [SMILES: CCC1(CC)CC(CN2CCN(c3ccc(C)cc3)CC2)OC1=O ; "HCOOH"]

Preparation of 3,3-diethyl-5-((4-(p-tolyl)piperazin-1-yl)methyl)dihydrofuran-2(3H)-one formate: The title compound was prepared according to the procedure for 1-(2-(4-(2-isopropylphenyl)piperazin-1-yl)ethyl)-2-oxaspiro[4.5]decan-3-one formate, except 3,3-diethyl-5-(iodomethyl)dihydrofuran-2(3H)-one was substituted for 3-(1-oxo-2-oxaspiro[4.5]decan-3-yl)propyl 4-methylbenzenesulfonate and 1-p-tolyl-piperazine was substituted for 1-(2-isopropyl-phenyl)-piperazine: ¹H NMR (400 MHz, CDCl₃) δ 8.24 (s, 1H), 7.09 (d, J=8.2 Hz, 2H), 6.86-6.81 (m, 3H), 4.85-4.72 (m, 1H), 3.34-3.23 (m, 4H), 3.21-3.06 (m, 3H), 3.06-2.95 (m, 2H), 2.73 (dd, J=13.8, 8.0 Hz, 1H), 2.27 (s, 3H), 2.19 (dd, J=13.2, 6.8 Hz, 1H), 1.83 (dd, J=13.2, 9.9 Hz, 1H), 1.72-1.54 (m, 4H), 1.03-0.81 (m, 6H); ¹³C NMR (101 MHz, CDCl₃) δ 180.34, 165.78, 148.28, 130.67, 129.95, 117.16, 74.13, 62.06, 53.04, 48.67, 47.92, 36.20, 29.08, 28.14, 20.60, 8.83, 8.78; MS (LC/MS, M+H⁺): 331.2.

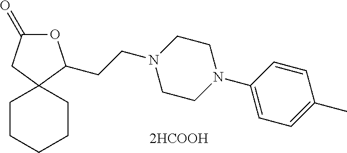 [SMILES: Cc1ccc(N2CCN(CCC3OC(=O)CC34CCCCC4)CC2)cc1 ; "2HCOOH"]

Preparation of 1-(2-(4-(p-tolyl)piperazin-1-yl)ethyl)-2-oxaspiro[4.5]decan-3-one formate: The title compound was prepared according to the procedure for 1-(2-(4-(2-isopropylphenyl)piperazin-1-yl)ethyl)-2-oxaspiro[4.5]decan-3-one formate, except 2-(3-oxo-2-oxaspiro[4.5]decan-1-yl)ethyl 4-methylbenzenesulfonate was substituted for 3-(1-oxo-2-oxaspiro[4.5]decan-3-yl)propyl 4-methylbenzenesulfonate and 1-p-tolyl-piperazine was substituted for 1-(2-isopropyl-phenyl)-piperazine. ¹H NMR (400 MHz, CDCl₃) δ 8.97 (broad, 3H), 8.27 (s, 2H), 7.10 (m, 2H), 6.84 (m, 2H), 4.10 (dd, J=11.2, 1.6 Hz, 1H), 3.45-3.14 (m, 9H), 3.06 (m, 1H), 2.57 (d, J=17.4 Hz, 1H), 2.42-2.23 (m, 4H), 2.22-2.06 (m, 1H), 2.06-1.88 (m, 1H), 1.66 (dd, J=18.5, 7.0 Hz, 3H), 1.55-1.14 (m, 7H); ¹³C NMR (101 MHz, CDCl₃) δ 175.86, 166.07, 147.63, 131.44, 130.06, 117.54, 86.41, 55.33, 52.26, 47.87, 43.16, 39.49, 35.06, 29.89, 25.70, 24.67, 23.14, 22.41, 20.61; MS (LC/MS, M+H⁺): 357.2.

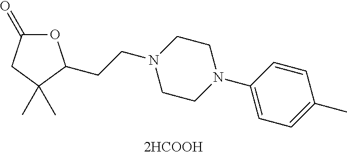 [SMILES: Cc1ccc(N2CCN(CCC3OC(=O)CC3(C)C)CC2)cc1]

Preparation of 4,4-dimethyl-5-(2-(4-(p-tolyl)piperazin-1-yl)ethyl)dihydrofuran-2(3H)-one formate: The title compound was prepared according to the procedure for 1-(2-(4-(2-isopropylphenyl)piperazin-1-yl)ethyl)-2-oxaspiro[4.5]decan-3-one formate, except 2-(3,3-dimethyl-5-oxotetrahydrofuran-2-yl)ethyl 4-methylbenzenesulfonate was substituted for 3-(1-oxo-2-oxaspiro[4.5]decan-3-yl)propyl 4-methylbenzenesulfonate and 1-p-tolyl-piperazine was substituted for 1-(2-isopropyl-phenyl)-piperazine. ¹H NMR (400 MHz, CDCl₃) δ 9.94 (s, 2H), 8.29 (s, 1H), 7.09 (m, 2H), 6.89-6.74 (m, 2H), 4.11 (dd, J=11.0, 2.0 Hz, 1H), 3.40-3.26 (m, 4H), 3.26-3.07 (m, 5H), 3.07-2.91 (m, 1H), 2.50-2.16 (m, 5H), 2.16-2.00 (m, 1H), 1.97-1.84 (m, 1H), 1.17 (s, 3H), 1.04 (s, 3H); ¹³C NMR (101 MHz, CDCl₃) δ 175.68, 166.33, 147.84, 131.14, 130.00, 117.39, 86.28, 55.26, 52.30, 48.06, 44.29, 39.43, 25.08, 24.66, 21.61, 20.59; MS (LC/MS, M+H⁺): 317.2.

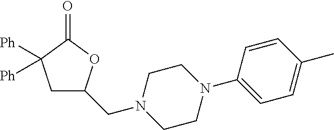 [SMILES: Cc1ccc(N2CCN(CC3CC(c4ccccc4)(c4ccccc4)C(=O)O3)CC2)cc1]

Preparation of 3,3-diphenyl-5-((4-(p-tolyl)piperazin-1-yl)methyl)dihydrofuran-2(3H)-one formate: 5-(iodomethyl)-3,3-diphenyldihydrofuran-2(3H)-one (113.4 mg, 0.3 mmol, 1.0 equiv) was treated with 1-p-tolyl-piperazine (158.4 mg. 0.9 mmol, 3.0 equiv) in dry CH₃CN (3 mL) and heated in microwave reactor at 170° C. for 1 h. The mixture was then filtered through a syringe filter and purified by HPLC (CH₃CN/H₂O), 0%-100%) to give desired product as its formic acid salt. ¹H NMR (400 MHz, CDCl₃) δ 7.47-7.22 (m, 10H), 7.10 (d, J=8.3 Hz, 2H), 6.87 (t, J=5.7 Hz, 2H), 4.60 (dq, J=9.0, 4.9 Hz, 1H), 3.25-3.12 (m, 4H), 3.06 (dd, J=13.0, 5.0 Hz, 1H), 2.85-2.74 (m, 5H), 2.74-2.64 (m, 2H), 2.29 (s, 3H); ¹³C NMR (101 MHz, CDCl₃) δ 177.14, 149.25, 142.10, 139.71, 129.78, 129.51, 129.10, 128.54, 127.92, 127.86, 127.51, 127.40, 116.59, 76.19, 62.06, 57.77, 54.06, 49.85, 41.96, 20.56; MS (LC/MS, M+H⁺): 427.2.

The following compounds can be prepared by the procedure of 3,3-diphenyl-5-((4-(p-tolyl)piperazin-1-yl)methyl)dihydrofuran-2(3H)-one formate. The skilled practitioner will know how to substitute the appropriate reagents, starting materials and purification methods known to those skilled in the art, in order to prepare the compounds provided herein.

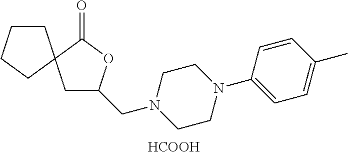 [SMILES: Cc1ccc(N2CCN(CC3CC4(CCCC4)C(=O)O3)CC2)cc1 ; "HCOOH"]

Preparation of 3-((4-(p-tolyl)piperazin-1-yl)methyl)-2-oxaspiro[4.4]nonan-1-one formate: The title compound was prepared according to the procedure for 3,3-diphenyl-5-((4-(p-tolyl)piperazin-1-yl)methyl)dihydrofuran-2(3H)-one formate, except 3-(iodomethyl)-2-oxaspiro[4.4]nonan-1-one was substituted for 5-(iodomethyl)-3,3-diphenyldihydrofuran-2(3H)-one. ¹H NMR (400 MHz, CDCl₃) δ 8.24 (s, 1H), 7.09 (d, J=8.3 Hz, 2H), 6.84 (dd, J=9.0, 2.3 Hz, 2H), 6.37 (broad, 2H), 4.75 (dddd, J=10.1, 8.0, 6.0, 2.1 Hz, 1H), 3.33-3.17 (m, 4H), 3.16-2.99 (m, 3H), 2.99-2.89 (m, 2H), 2.75 (dd, J=13.8, 8.0 Hz, 1H), 2.34-2.23 (m, 4H), 2.23-2.12 (m, 1H), 1.95-1.53 (m, 8H); ¹³C NMR (101 MHz, CDCl₃) δ 182.01, 165.87, 148.48, 130.45, 129.91, 117.06, 74.97, 61.76, 53.15, 49.53, 48.85, 41.63, 37.44, 36.72, 25.68, 25.56, 20.59; MS (LC/MS, M+H⁺): 329.2.

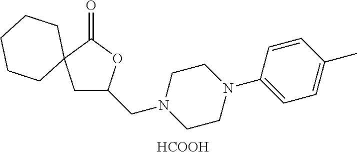 [SMILES: Cc1ccc(N2CCN(CC3CC4(CCCCC4)C(=O)O3)CC2)cc1 ; "HCOOH"]

Preparation of 3-((4-(p-tolyl)piperazin-1-yl)methyl)-2-oxaspiro[4.5]decan-1-one formate: The title compound was prepared according to the procedure for 3,3-diphenyl-5-((4-(p-tolyl)piperazin-1-yl)methyl)dihydrofuran-2(3H)-one formate, except 3-(iodomethyl)-2-oxaspiro[4.5]decan-1-one was substituted for 5-(iodomethyl)-3,3-diphenyldihydrofuran-2(3H)-one. ¹H NMR (400 MHz, CDCl₃) δ 8.23 (s, 1H), 7.09 (d, J=8.2 Hz, 2H), 6.91-6.75 (m, 2H), 6.32 (broad, 4H), 4.84 (dtd, J=10.0, 8.1, 1.5 Hz, 1H), 3.38-3.27 (m, 5H), 3.26-3.16 (m, 2H), 3.14-3.04 (m, 2H), 2.79 (dd, J=13.8, 8.3 Hz, 1H), 2.47 (dt, J=15.4, 7.7 Hz, 1H), 2.28 (s, 3H), 1.90-1.68 (m, 3H), 1.60 (m, J=19.3, 13.3, 7.1 Hz, 4H), 1.53-1.15 (m, 4H); ¹³C NMR (101 MHz, CDCl₃) δ 180.85, 165.73, 148.05, 130.96, 129.99, 117.31, 73.73, 61.62, 52.85, 48.44, 44.12, 38.06, 34.21, 31.38, 25.33, 22.22, 22.13, 20.61; MS (LC/MS, M+H⁺): 343.2.

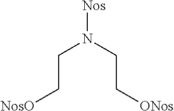

Preparation of (((4-nitrophenyl)sulfonyl)azanediyl)bis(ethane-2,1-diyl) bis(4-nitrobenzenesulfonate): To a stirred solution of diethanolamine (130.31 mmol, 1 equiv) and triethylamine (457.38 mmol, 3.5 equiv) in anhydrous THF (200 mL) at 0° C. under a nitrogen atmosphere, 4-nitrobenzenesulfonyl chloride (430.02 mmol, 3.3 equiv) was added portionwise. This mixture was stirred at 0° C. for 1 h then overnight at room temperature. At the conclusion of this period the reaction mixture was concentrated at reduced pressure. The residue was dissolved in dichloromethane (200 mL), washed with water (25 mL), dried (MgSO₄), filtered and evaporated to afford an orange solid. Recrystallisation from methanol-THF gave the title compound as yellowish crystal. ¹H NMR (400 MHz, acetone) δ 8.55 (d, J=9.0 Hz, 4H), 8.45 (d, J=8.9 Hz, 2H), 8.25 (d, J=9.0 Hz, 4H), 8.16 (d, J=8.9 Hz, 2H), 4.38 (t, J=5.6 Hz, 4H), 3.71 (t, J=5.6 Hz, 4H).

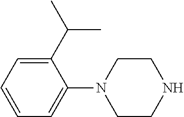

Preparation of 1-(2-isopropylphenyl)piperazine: The reactions were performed in a CEM microwave reaction system operated at 175° C. for 1 h. (((4-nitrophenyl)sulfonyl)azanediyl)bis(ethane-2,1-diyl) bis(4-nitrobenzenesulfonate) (660 mg, 1.0 mmol), 2-isopropyl-phenylamine (162 mg, 1.2 mmol), DIPEA (516 mg, 4.0 mmol) and CH₃CN (3 mL) were mixed in a microwave reaction vial (10 mL) fitted with a no-invasive vial cap. The reaction vials containing the mixture were reacted in the microwave for 1 h at 175° C. The typical reaction temperature-time profile is shown in the supporting material. After 1 h, the solvent was removed under reduced pressure. The residue was dissolved in dichloromethane and washed with HCl (10%, 3×30 mL) and saturated NaHCO₃ (40 mL). The organic phase was dried over MgSO₄ and concentrated in vacuo to afford the crude product. This crude product, 1-(2-isopropyl-phenyl)-4-(4-nitro-benzenesulfonyl)-piperazine, was filtered through a pad of silica (hexanes/dichloromethane 1:4, silica pad thickness: 10 cm, diameter: 4 cm) and used in subsequent reactions without further purification.

Potassium carbonate (3.52 g, 25.47 mmol) was added to a mixture of acetonitrile and dimethylsulfoxide (CH₃CN/DMSO 49:1) and heated to 50° C. Thiophenol (2.34 g, 21.23 mmol) was added dropwise via syringe to the mixture with stirring. After 30 min a solution of 1-(2-isopropyl-phenyl)-4-(4-nitro-benzenesulfonyl)-piperazine (825 mg, 2.12 mmol) in CH₃CN and DMSO (CH₃CN/DMSO 49:1) was added dropwise. The reaction mixture was stirred for 3 hours, quenched with excess NaOH solution (40%; also removed the unpleasant smell of PhSH) and concentrated under reduced pressure. The residue was extracted with dichloromethane (5×30 mL) and the organic phase was dried over MgSO₄, and concentrated in vacuo to give a crude oil. The oil was purified by reverse phase chromatography (CH₃CN in H₂O, gradient from 1%-100% with 0.1% formic acid) to afford the formic acid salt of the desired piperazine. The salt was dissolved in dichloromethane, washed with saturated NaHCO₃ solution, and the organic phase concentrated in vacuo to provide the product. IR (KBr, cm⁻¹): 3295, 2958, 2867, 2818, 1444, 1360, 1252, 1053, 932, 807, 762; ¹H NMR (400 MHz, CDCl₃) δ 7.23-7.14 (m, 1H), 7.13-6.97 (m, 3H), 3.43 (dt, J=13.8, 6.9 Hz, 1H), 3.25 (s, 1H), 3.07-2.94 (m, 4H), 2.81 (m, 4H), 1.13 (d, J=6.9 Hz, 6H); ¹³C NMR (101 MHz, CDCl₃) δ 151.1, 144.9, 126.7, 126.6, 124.9, 120.1, 54.3, 46.6, 27.0, 24.3; MS (LC/MS, M+H⁺): 205.1.

The following compounds can be prepared by the procedure of 1-(2-isopropylphenyl)piperazine. The skilled practitioner will know how to substitute the appropriate reagents, starting materials and purification methods known to those skilled in the art, in order to prepare the compounds provided herein.

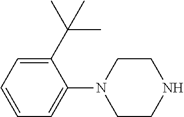 [SMILES: CC(C)(C)c1ccccc1N1CCNCC1]

Preparation of 1-(2-tert-butylphenyl)piperazine: The title compound was prepared according to the procedure for 1-(2-isopropylphenyl)piperazine, except 2-tert-butyl-phenylamine was substituted for 2-isopropyl-phenylamine. IR (KBr, cm⁻¹): 3330, 2952, 2824, 2715, 2474, 1452, 1216, 1136, 924, 764; ¹H NMR (400 MHz, CDCl₃) δ 7.46-7.35 (m, 2H), 7.33-7.16 (m, 2H), 3.62-3.41 (m, 4H), 3.32 (t, J=11.7 Hz, 2H), 3.01 (d, J=12.6 Hz, 2H), 1.87 (s, 1H), 1.50-1.35 (s, 9H); ¹³C NMR (101 MHz, CDCl₃) δ 151.5, 147.3, 127.7, 127.5, 127.3, 125.8, 51.0, 44.3, 35.7, 31.1. MS (LC/MS, M+H⁺): 219.1

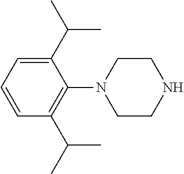 [SMILES: CC(C)c1cccc(C(C)C)c1N1CCNCC1]

Preparation of 1-(2,6-diisopropylphenyl)piperazine: The title compound was prepared according to the procedure for 1-(2-isopropylphenyl)piperazine, except 2,6-diisopropyl-phenylamine was substituted for 2-isopropyl-phenylamine: IR (KBr, cm⁻¹): 3295, 2958, 1653, 1444, 1252, 1053, 932, 807, 762; ¹H NMR (400 MHz, CDCl₃) δ 7.10 (dd, J=8.5, 6.7 Hz, 1H), 7.05-7.00 (m, 2H), 3.45 (dt, J=13.8, 6.9 Hz, 2H), 3.04 (m, 4H), 2.97-2.87 (m, 4H), 2.03 (s, 1H), 1.13 (d, J=6.9 Hz, 12H); ¹³C NMR (101 MHz, CDCl₃) δ 149.4, 146.4, 126.9, 124.3, 52.3, 47.4, 28.5, 24.7. HRMS (CI): [M+H], calcd for C₁₆H₂₆N₂, 247.2174; found 247.2175.

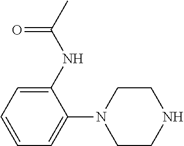 [SMILES: CC(=O)Nc1ccccc1N1CCNCC1]

Preparation of N-(2-(piperazin-1-yl)phenyl)acetamide: The title compound was prepared according to the procedure for 1-(2-isopropylphenyl)piperazine, except N-(2-amino-phenyl)-acetamide was substituted for 2-isopropyl-phenylamine: IR (KBr, cm⁻¹): 3318, 2945, 2827, 1673, 1589, 1517, 1449, 1370, 1233, 761; ¹H NMR (400 MHz, CDCl₃) δ 8.45 (s, 1H), 8.27 (d, J=8.0 Hz, 1H), 7.13-7.03 (m, 2H), 6.98 (td, J=7.6, 1.4 Hz, 1H), 3.00 (m, J=12.5, 8.0 Hz, 4H), 2.83-2.71 (m, 4H), 2.14 (s, 3H), 1.99 (s, J=10.8 Hz, 1H); ¹³C NMR (101 MHz, CDCl₃) δ 168.4, 141.6, 133.8, 125.8, 124.1, 120.9, 119.7, 53.7, 47.2, 25.3. MS (LC/MS, M+H⁺): 220.1.

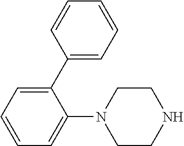 [SMILES: c1ccc(-c2ccccc2N2CCNCC2)cc1]

Preparation of 1-(biphenyl-2-yl)piperazine: The title compound was prepared according to the procedure for 1-(2-isopropylphenyl)piperazine, except biphenyl-2-ylamine was substituted for 2-isopropyl-phenylamine. IR (KBr, cm⁻¹): 3350, 3057, 2943, 2817, 1593, 1480, 1433, 1223, 770, 739, 699; ¹H NMR (400 MHz, CDCl₃) δ 7.55 (m, 2H), 7.38-7.25 (m, 2H), 7.25-7.14 (m, 3H), 7.05-6.90 (m, 2H), 2.73 (d, J=7.4 Hz, 8H), 2.38 (s, 1H); ¹³C NMR (101 MHz, CDCl₃) δ 150.9, 141.4, 135.4, 131.8, 129.2, 128.6, 128.5, 127.1, 123.1, 118.7, 52.5, 46.2. MS (LC/MS, M+H⁺): 239.1.

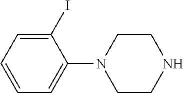 [SMILES: Ic1ccccc1N1CCNCC1]

Preparation of 1-(2-iodophenyl)piperazine: The title compound was prepared according to the procedure for 1-(2-isopropylphenyl)piperazine, except 2-iodo-phenylamine was substituted for 2-isopropyl-phenylamine. IR (KBr, cm⁻¹): 3292, 3053, 2943, 2819, 1578, 1468, 1225, 1012, 760; ¹H NMR (400 MHz, CDCl₃) δ 7.95-7.77 (m, 1H), 7.04 (dt, J=7.4, 3.7 Hz, 1H), 6.80 (td, J=7.8, 1.4 Hz, 1H), 3.20-2.88 (m, 8H), 1.25 (s, 1H); ¹³C NMR (101 MHz, CDCl₃) δ 153.9, 140.4, 129.6, 125.9, 121.5, 98.7, 53.5, 46.3. MS (LC/MS, M+H⁺): 288.9.

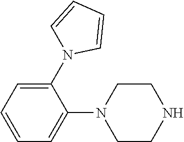 [SMILES: c1ccc(-n2cccc2)c(N2CCNCC2)c1]

Preparation of 1-(2-(1H-pyrrol-1-yl)phenyl)piperazine: The title compound was prepared according to the procedure for 1-(2-isopropylphenyl)piperazine, except 2-pyrrol-1-yl-phenylamine was substituted for 2-isopropyl-phenylamine: IR (KBr, cm⁻¹): 3309, 2943, 2822, 1598, 1503, 1449, 1319, 1235, 1069, 759, 727; ¹H NMR (400 MHz, CDCl₃) δ 7.24-7.07 (m, 2H), 7.03-6.88 (m, 4H), 6.20 (t, J=2.2 Hz, 2H), 2.85-2.68 (m, 4H), 2.56 (m, 4H), 1.61 (s, 1H); ¹³C NMR (101 MHz, CDCl₃) δ 147.0, 133.9, 127.7, 126.7, 122.7, 121.4, 119.2, 109.2, 51.7, 46.5; MS (LC/MS, M+H⁺): 228.1.

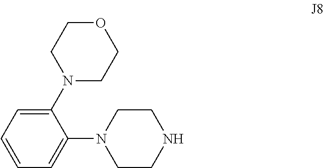 [SMILES: c1ccc(N2CCOCC2)c(N2CCNCC2)c1]

Preparation of 4-(2-(piperazin-1-yl)phenyl)morpholine: The title compound was prepared according to the procedure for 1-(2-isopropylphenyl)piperazine, except 2-morpholin-4-yl-phenylamine was substituted for 2-isopropyl-phenylamine. IR (KBr, cm⁻¹): 3313, 2950, 2817, 1591, 1493, 1446, 1227, 1117, 928, 764; ¹H NMR (400 MHz, CDCl₃) δ 6.91 (m, 2H), 6.88-6.78 (m, 2H), 3.83-3.68 (m, 4H), 3.07 (m, J=21.5 Hz, 8H), 2.92 (t, J=4.7 Hz, 4H), 1.80 (s, 1H); ¹³C NMR (101 MHz, CDCl₃) δ 145.4, 144.8, 123.3, 123.2, 119.0, 118.4, 67.8, 51.2, 50.2, 47.0; HRMS (CI): [M+H], calcd for C₁₄H₂₁N₃O, 248.1762; found, 248.1751.

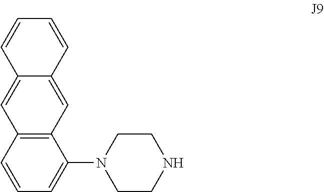 [SMILES: c1ccc2cc3c(N4CCNCC4)cccc3cc2c1]

Preparation of 1-(anthracen-1-yl)piperazine: The title compound was prepared according to the procedure for 1-(2-isopropylphenyl)piperazine, except anthracen-1-ylamine was substituted for 2-isopropyl-phenylamine. IR (KBr, cm⁻¹): 3320, 3049, 2942, 2819, 1670, 1618, 1454, 1246, 1133, 1007, 891, 733; ¹H NMR (400 MHz, CDCl₃) δ 8.66 (s, 1H), 8.29 (s, 1H), 7.91 (m, 2H), 7.60 (d, J=8.5 Hz, 1H), 7.41-7.32 (m, 2H), 7.32-7.22 (m, 1H), 6.91 (d, J=7.1 Hz, 1H), 3.24-2.81 (m, 8H), 1.86 (s, 1H); ¹³C NMR (101 MHz, CDCl₃) δ 150.4, 133.3, 131.9, 131.5, 129.0, 128.2, 128.0, 126.9, 125.8, 125.6, 125.5, 124.0, 122.7, 113.7, 54.8, 46.9; MS (LC/MS, M+H⁺): 263.0.

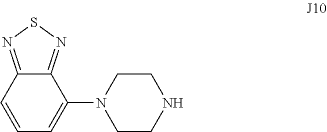 [SMILES: c1cc(N2CCNCC2)c2nsnc2c1]

Preparation of 4-(piperazin-1-yl)benzo[c][1,2,5]thiadiazole: The title compound was prepared according to the procedure for 1-(2-isopropylphenyl)piperazine, except benzo[1,2,5]thiadiazol-4-ylamine was substituted for 2-isopropyl-phenylamine. IR (KBr, cm⁻¹): 3300, 2945, 2828, 1663, 1538, 1487, 1250, 1102, 1023, 909, 803, 743; ¹H NMR (400 MHz, CDCl₃) δ 7.43 (m, 2H), 6.65 (d, J=6.8 Hz, 1H), 3.46-3.35 (m, 4H), 3.15-3.02 (m, 4H), 1.87 (s, 1H); ¹³C NMR (101 MHz, CDCl₃) δ 161.1, 156.9, 150.0, 145.0, 130.7, 113.8, 111.6, 51.7, 46.4; MS (LC/MS, M+H⁺): 221.0.

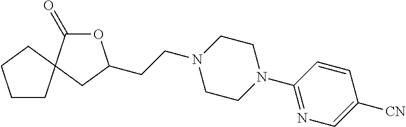 [SMILES: N#Cc1ccc(N2CCN(CCC3CC4(CCCC4)C(=O)O3)CC2)nc1]

Preparation of 6-(4-(2-(1-oxo-2-oxaspiro[4.4]nonan-3-yl)ethyl)piperazin-1-yl)nicotinonitrile: The title compound was prepared according to the procedure for 5-(2-(4-(1H-benzo[d]imidazol-2-yl)piperidin-1-yl)ethyl)-3,3-diethyldihydrofuran-2(3H)-one, except 6-(piperazin-1-yl)nicotinonitrile was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.45 (d, J=2 Hz, 1H), 7.73 (dd, J=9.1 Hz, 2 Hz, 1H), 6.68 (d, J=9.1 Hz, 1H), 4.43 (m, 1H), 3.22 (m, 4H), 2.30 (m, 2H), 2.20 (m, 3H), 1.85 (m, 6H), 1.72 (m, 5H), 1.60 (m, 2H). MS (LC/MS, M+H⁺): 356.20.

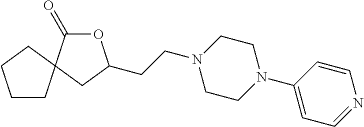 [SMILES: O=C1OC(CCN2CCN(c3ccncc3)CC2)CC12CCCC2]

Preparation of 3-(2-(4-(pyridin-4-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(pyridin-4-yl)piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.27 (d, J=6.4 Hz, 2H), 6.65 (d, J=6.4 Hz, 2H), 4.50 (m, 1H), 3.33 (t, J=5.2 Hz, 4H), 2.56 (m, 6H), 2.24 (m, 1H), 2.20 (m, 1H), 2.84 (m, 6H), 2.65 (m, 4H). MS (LC/MS, M+H⁺): 331.15.

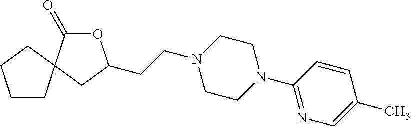 [SMILES: Cc1ccc(N2CCN(CCC3CC4(CCCC4)C(=O)O3)CC2)nc1]

Preparation of 3-(2-(4-(5-methylpyridin-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-methylpyridin-2-yl)piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.02 (b, 1H), 7.82 (dd, J=, 1H), 6.93 (d, J=, 1H), 4.43 (m, 1H), 4.03 (b, 4H), 3.43 (b, 4H), 3.29 (t, J=7.8 Hz, 2H), 2.34 (s, 3H), 2.30 (m, 1H), 2.16 (m, 3H), 1.85 (m, 4H), 1.70 (m, 3H), 1.68 (m, 1H). MS (LC/MS, M+H⁺): 345.2.

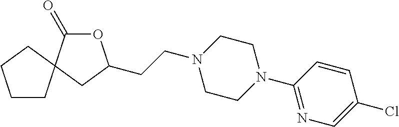 [SMILES: O=C1OC(CCN2CCN(c3ccc(Cl)cn3)CC2)CC12CCCC2]

Preparation of 3-(2-(4-(5-chloropyridin-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-chloropyridin-2-yl)piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.14 (d, J=2 Hz, 1H), 7.51 (dd, J=9.1 Hz, 2 Hz, 1H), 6.63 (d, J=9.1 Hz, 1H), 4.43 (m, 1H), 3.22 (m, 4H), 2.31 (m, 2H), 2.20 (m, 3H), 1.85 (m, 6H), 1.70 (m, 5H), 1.60 (m, 2H). MS (LC/MS, M+H⁺): 365.2.

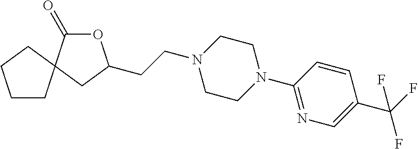 [SMILES: O=C1OC(CCN2CCN(c3ccc(C(F)(F)F)cn3)CC2)CC12CCCC2]

Preparation of 3-(2-(4-(5-(trifluoromethyl)pyridin-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-(trifluoromethyl)pyridin-2-yl)piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.39 (b, 1H), 7.62 (dd, J=9.1 Hz, 2 Hz, 1H), 6.63 (d, J=9.1 Hz, 1H), 4.50 (m, 1H), 3.64 (m, 5H), 2.55 (m, 6H), 2.22 (m, 3H), 1.85 (m, 5H), 1.70 (m, 3H). MS (LC/MS, M+H⁺): 399.2.

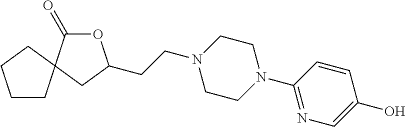 [SMILES: O=C1OC(CCN2CCN(c3ccc(O)cn3)CC2)CC12CCCC2]

Preparation of 3-(2-(4-(5-hydroxypyridin-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 6-(piperazin-1-yl)pyridin-3-ol was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.23 (m, 1H), 8.21 (m, 1H), 8.01 (d, J=2.6 Hz, 1H), 4.43 (m, 1H), 3.24 (m, 4H), 2.30 (m, 2H), 2.12 (m, 3H), 1.87 (m, 6H), 1.71 (m, 5H), 1.61 (m, 2H). MS (LC/MS, M+H⁺): 332.2.

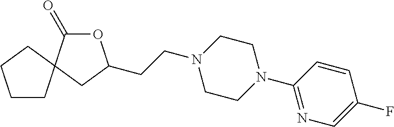 [SMILES: O=C1OC(CCN2CCN(c3ccc(F)cn3)CC2)CC12CCCC2]

Preparation of 3-(2-(4-(5-fluoropyridin-2-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-fluoropyridin-2-yl)piperazine was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.10 (d, J=2.9 Hz, 1H), 7.37 (m, 1H), 6.69 (d, J=2.9 Hz, 1H), 4.43 (m, 1H), 3.30 (m, 4H), 2.30 (m, 2H), 2.08 (m, 3H), 1.85 (m, 6H), 1.72 (m, 5H), 1.62 (m, 2H). MS (LC/MS, M+H⁺): 349.2.

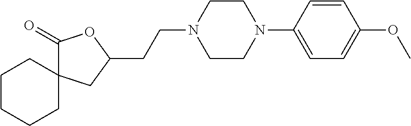 [SMILES: COc1ccc(N2CCN(CCC3CC4(CCCCC4)C(=O)O3)CC2)cc1]

Preparation of 3-(2-(4-(4-methoxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(4-methoxyphenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 6.83 (d, J=9.1 Hz, 2H), 6.76 (d, J=9.1 Hz, 2H), 4.42 (m, 1H), 3.69 (s, 3H), 3.02 (t, J=4.9 Hz, 4H), 2.55 (dd, J=7.9 Hz, J=4.1 Hz, 4H), 2.29 (m, 2H), 2.31 (m, 1H), 1.80 (m, 4H), 1.59 (m, 4H), 1.42 m, 1H), 1.25 (m, 4H). MS (LC/MS, M+H⁺): 373.2.

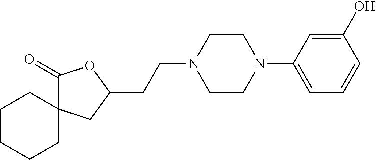 [SMILES: O=C1OC(CCN2CCN(c3cccc(O)c3)CC2)CC12CCCCC2]

Preparation of 3-(2-(4-(3-hydroxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 3-(piperazin-1-yl)phenol was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.10 (t, J=8.1 Hz, 1H), 6.50 (dd, J=8.2 Hz, J=2.0 Hz, 1H), 6.39 (t, J=2.3 Hz, 1H), 6.31 (dd, J=7.7 Hz, J=2.0 Hz, 1H) 4.49 (m, 1H), 3.18 (t, J=5 Hz, 4H) 2.60 (dd, J=8.9 Hz, J=3.9 Hz, 4H), 2.56 (m, 2H), 2.39 (m, 1H), 1.86 (m, 4H), 1.71 (m, 2H), 1.59 m, 4H), 1.37 (m, 3H). MS (LC/MS, M+H⁺): 359.2.

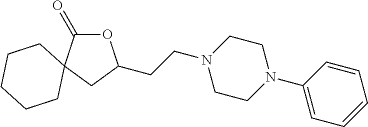 [SMILES: O=C1OC(CCN2CCN(c3ccccc3)CC2)CC12CCCCC2]

Preparation of 3-(2-(4-phenylpiperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-phenylpiperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.27 (t, J=7.3 Hz, 2H), 6.93 (d, J=8.2 Hz, J=2.0 Hz 1H), 6.85 (t, J=7.3 Hz, 1H), 4.50 (m, 1H), 3.21 (t, J=4.9 Hz, 4H) 2.61 (dd, J=8.8 Hz, J=3.9 Hz, 4H), 2.55 (m, 2H), 2.40 (m, 1H), 1.86 (m, 4H), 1.62 (m, 4H), 1.51 m, 2H), 1.30 (m, 3H). MS (LC/MS, M+H⁺): 343.2.

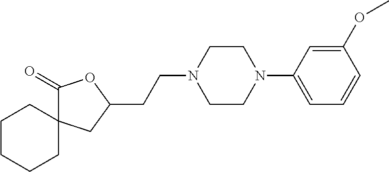 [SMILES: COc1cccc(N2CCN(CCC3CC4(CCCCC4)C(=O)O3)CC2)c1]

Preparation of 3-(2-(4-(3-methoxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(3-methoxyphenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.17 (t, J=8.2 Hz, 1H), 6.52 (dd, J=8.1 Hz, J=1.9 Hz 1H), 6.46 (t, J=2.3 Hz, 1H), 6.42 (dd, J=8.0 Hz, J=2.1 Hz, 1H) 4.49 (m, 1H), 3.79 (s, 3H), 3.19 (t, J=4.9 Hz, 4H) 2.59 (dd, J=8.8 Hz, J=3.9 Hz, 4H), 2.56 (m, 2H), 2.39 (m, 1H), 1.86 (m, 4H), 1.71 (m, 2H), 1.63 m, 4H), 1.37 (m, 3H). MS (LC/MS, M+H⁺): 373.2.

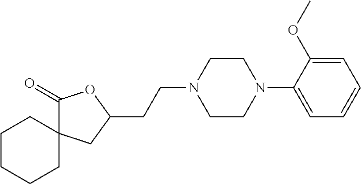 [SMILES: COc1ccccc1N1CCN(CCC2CC3(CCCCC3)C(=O)O2)CC1]

Preparation of 3-(2-(4-(2-methoxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2-methoxyphenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ δ 7.0 (m, 1H), 6.93 (m, 2H), 6.86 (dd, J=7.8 Hz, J=1.2 Hz, 1H), 4.49 (m, 1H), 3.86 (s, 3H), 3.09 (s, 4H) 2.60 (s, 4H), 2.58 (m, 2H), 2.40 (m, 1H), 1.90 (m, 4H), 1.71 (m, 2H), 1.61 m, 4H), 1.30 (m, 3H). MS (LC/MS, M+H⁺): 373.2.

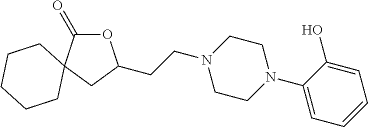 [SMILES: O=C1OC(CCN2CCN(c3ccccc3O)CC2)CC12CCCCC2]

Preparation of 3-(2-(4-(2-hydroxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 2-(piperazin-1-yl)phenol was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.1 (dd, J=7.8 Hz, J=1.4 Hz 1H), 7.0 (td, J=8 Hz, J=1.4 Hz 1H), 6.94 (dd, J=7.6 Hz, J=1.4 Hz, 1H), 6.85 (td, J=7.6 Hz, J=1.4 Hz 1H), 4.51 (m, 1H), 2.91 (t, J=4.8 Hz, 4H), 2.62 (m, 4H) 2.58 (m, 2H), 2.40 (m, 1H), 1.89 (m, 4H), 1.71 (m, 2H), 1.61 m, 4H), 1.30 (m, 3H). MS (LC/MS, M+H⁺): 359.2.

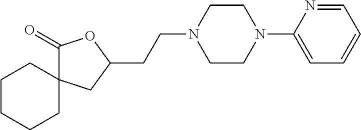 [SMILES: O=C1OC(CCN2CCN(c3ccccn3)CC2)CC12CCCCC2]

Preparation of 3-(2-(4-(pyridin-2-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(pyridin-2-yl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 6 8.19 (ddd, J=4.9 Hz, J=1.9 Hz, J=0.7 Hz 1H), 7.47 (ddd, J=8.8 Hz, J=7.1 Hz, J=1.9 Hz, 1H), 6.62 (m, 2H), 4.51 (m, 1H), 3.54 (t, J=4.3 Hz, 4H), 2.62 (m, 4H) 2.54 (m, 2H), 2.40 (m, 1H), 1.89 (m, 4H), 1.69 (m, 2H), 1.62 (m, 4H), 1.25 (m, 3H). MS (LC/MS, M+H⁺): 344.2.

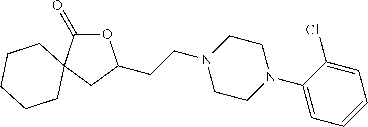 [SMILES: O=C1OC(CCN2CCN(c3ccccc3Cl)CC2)CC12CCCCC2]

Preparation of 3-(2-(4-(2-chlorophenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(2-chlorophenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.35 (dd, J=7.8 Hz, J=1.4 Hz 1H), 7.23 (td, J=8 Hz, J=1.4 Hz 1H), 7.0 (dd, J=8.9 Hz, J=1.4 Hz, 1H), 6.96 (td, J=7.6 Hz, J=1.4 Hz 1H), 4.51 (m, 1H), 3.08 (s, 4H), 2.62 (s, 4H) 2.58 (m, 2H), 2.40 (m, 1H), 1.89 (m, 4H), 1.71 (m, 2H), 1.61 m, 4H), 1.30 (m, 3H). MS (LC/MS, M+H⁺): 377.2.

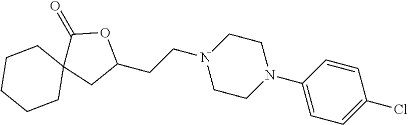 [SMILES: O=C1OC(CCN2CCN(c3ccc(Cl)cc3)CC2)CC12CCCCC2]

Preparation of 3-(2-(4-(4-chlorophenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(4-chlorophenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.20 (d, J=9.0 Hz, 2H), 6.83 (d, J=9.0 Hz, 2H), 4.50 (m, 1H), 3.06 (t, J=4.9 Hz, 4H), 2.60 (dd, J=8.8 Hz, J=4.0 Hz, 4H), 2.56 (m, 2H), 2.40 (m, 1H), 1.87 (m, 4H), 1.63 (m, 5H), 1.59 (m, 1H), 1.30 (m, 3H). MS (LC/MS, M+H⁺): 377.2.

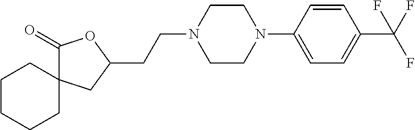 [SMILES: O=C1OC(CCN2CCN(c3ccc(C(F)(F)F)cc3)CC2)CC12CCCCC2]

Preparation of 3-(2-(4-(4-(trifluoromethyl)phenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(4-(trifluoromethyl)phenyl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.47 (d, J=8.6 Hz, 2H), 6.92 (d, J=8.7 Hz, 2H), 4.50 (m, 1H), 3.29 (t, J=5.0 Hz, 4H), 2.58 (dd, J=5.1 Hz, J=4.6 Hz, 4H), 2.52 (m, 2H), 2.40 (m, 1H), 1.87 (m, 4H), 1.63 (m, 6H), 1.59 (m, 1H), 1.26 (m, 3H). MS (LC/MS, M+H⁺): 411.2.

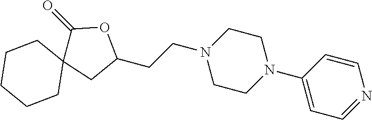 [SMILES: O=C1OC(CCN2CCN(c3ccncc3)CC2)CC12CCCCC2]

Preparation of 3-(2-(4-(pyridin-4-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(pyridin-4-yl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.27 (d, J=6.4 Hz, 2H), 6.65 (d, J=6.6 Hz, 2H), 4.50 (m, 1H), 3.34 (t, J=5.0 Hz, 4H), 2.56 (dd, J=5.1 Hz, J=4.4 Hz, 4H), 2.56 (m, 2H), 2.40 (m, 1H), 1.86 (m, 4H), 1.63 (m, 6H), 1.59 (m, 1H), 1.25 (m, 3H). MS (LC/MS, M+H⁺): 344.2.

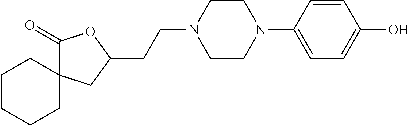 [SMILES: O=C1OC(CCN2CCN(c3ccc(O)cc3)CC2)CC12CCCCC2]

Preparation of 3-(2-(4-(4-hydroxyphenyl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 4-(piperazin-1-yl)phenol substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 6.84 (dd, J=6.5, J=2.3 Hz, 2H), 6.83 (dd, J=6.6, J=2.3 Hz, 2H), 4.49 (m, 1H), 3.08 (t, J=4.9 Hz, 4H), 2.60 (dd, J=8.4 Hz, J=3.9 Hz, 4H), 2.57 (m, 2H), 2.39 (m, 1H), 1.85 (m, 4H), 1.61 (m, 6H), 1.30 (m, 3H). MS (LC/MS, M+H⁺): 359.2.

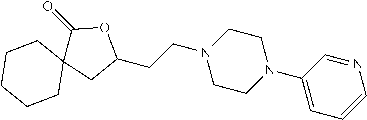 [SMILES: O=C1OC(CCN2CCN(c3cccnc3)CC2)CC12CCCCC2]

Preparation of 3-(2-(4-(pyridin-3-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(pyridin-3-yl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate: ¹H NMR (400 MHz, CDCl₃) δ 8.31 (d, J=1.6 Hz, 1H), 8.10 (dd, J=4.0 Hz, J=1.9 Hz, 1H), 7.16 (dd, J=4.0 Hz, J=1.1 Hz, 1H) 4.51 (m, 1H), 3.23 (t, J=5 Hz, 4H) 2.60 (dd, J=9.2 Hz, J=4.2 Hz, 4H), 2.58 (m, 2H), 2.41 (m, 1H), 1.86 (m, 4H), 1.66 (m, 6H), 1.31 (m, 3H). MS (LC/MS, M+H⁺): 344.2.

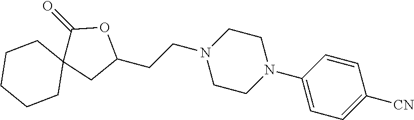 [SMILES: N#Cc1ccc(N2CCN(CCC3CC4(CCCCC4)C(=O)O3)CC2)cc1]

Preparation of 4-(4-(2-(1-oxo-2-oxaspiro[4.5]decan-3-yl)ethyl)piperazin-1-yl)benzonitrile: The title compound was prepared according to the procedure for 5-(2-(4-(1H-benzo[d]imidazol-2-yl)piperidin-1-yl)ethyl)-3,3-diethyldihydrofuran-2(3H)-one, except 4-(piperazin-1-yl)benzonitrile was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 7.49 (d, J=9.0, Hz, 2H), 6.85 (d, J=9.0 Hz, 2H), 4.50 (m, 1H), 3.32 (t, J=5.0 Hz, 4H), 2.60 (dd, J=13.9 Hz, J=5.2 Hz, 4H), 2.42 (m, 2H), 2.37 (m, 1H), 1.85 (m, 4H), 1.61 (m, 6H), 1.37 (m, 3H). MS (LC/MS, M+H⁺): 368.2.

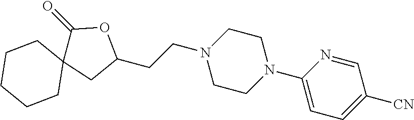 [SMILES: N#Cc1ccc(N2CCN(CCC3CC4(CCCCC4)C(=O)O3)CC2)nc1]

Preparation of 6-(4-(2-(1-oxo-2-oxaspiro[4.5]decan-3-yl)ethyl)piperazin-1-yl)nicotinonitrile: The title compound was prepared according to the procedure for 5-(2-(4-(1H-benzo[d]imidazol-2-yl)piperidin-1-yl)ethyl)-3,3-diethyldihydrofuran-2(3H)-one, except 6-(piperazin-1-yl)nicotinonitrile was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.18 (d, J=1.9 Hz, 1H), 7.38 (dd, J=9.0 Hz, J=2.3 Hz, 1H), 6.37 (d, J=9.0 Hz, 1H) 4.28 (m, 1H), 3.46 (t, J=3.7 Hz, 4H) 2.34 (dd, J=13.5 Hz, J=7.4 Hz, 4H), 2.29 (m, 2H), 2.17 (m, 1H), 1.65 (m, 4H), 1.45 (m, 6H), 1.16 (m, 3H). MS (LC/MS, M+H⁺): 369.2.

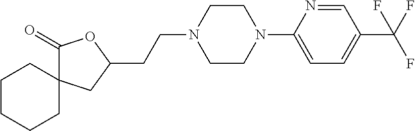 [SMILES: O=C1OC(CCN2CCN(c3ccc(C(F)(F)F)cn3)CC2)CC12CCCCC2]

Preparation of 3-(2-(4-(5-(trifluoromethyl)pyridin-2-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-(trifluoromethyl)pyridin-2-yl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) δ 8.41 (t, J=0.7 Hz, 1H), 7.64 (dd, J=10.4 Hz, J=2.4 Hz, 1H), 6.65 (d, J=9.0 Hz, 1H) 4.53 (m, 1H), 3.66 (t, J=3.9 Hz, 4H) 2.57 (dd, J=8.0 Hz, J=3.2 Hz, 4H), 2.54 (m, 2H), 2.40 (m, 1H), 1.86 (m, 4H), 1.69 (m, 6H), 1.35 (m, 3H). MS (LC/MS, M+H⁺): 412.2.

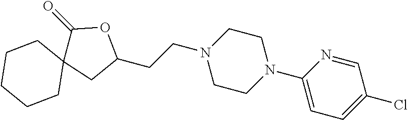 [SMILES: O=C1OC(CCN2CCN(c3ccc(Cl)cn3)CC2)CC12CCCCC2]

Preparation of 3-(2-(4-(5-chloropyridin-2-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 1-(5-chloropyridin-2-yl)piperazine was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) 8.12 (d, J=2.4 Hz, 1H), 7.43 (dd, J=9.0 Hz, J=2.6 Hz, 1H), 6.59 (d, J=9.0 Hz, 1H) 4.52 (m, 1H), 3.53 (pen, J=5.6 Hz, J=1.4 Hz, 4H) 2.57 (m, 4H), 2.54 (m, 2H), 2.43 (m, 1H), 1.86 (m, 4H), 1.69 (m, 6H), 1.35 (m 3H). MS (LC/MS, M+H⁺): 478.2.

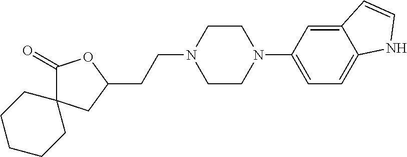 [SMILES: O=C1OC(CCN2CCN(c3ccc4[nH]ccc4c3)CC2)CC12CCCCC2]

Preparation of 3-(2-(4-(1H-indol-5-yl)piperazin-1-yl)ethyl)-2-oxaspiro[4.5]decan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 5-(piperazin-1-yl)-1H-indole was substituted for 2-piperazin-1-yl-benzonitrile, and 2-(1-oxo-2-oxaspiro[4.5]decan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) 8.34 (s, 1H), 8.11 (s, 1H), 7.31 (d, J=8.7 Hz, 1H), 7.19 (d, J=2.4 Hz, 2H), 6.94 (dd, J=9.0 Hz, J=2.2 Hz, 1H),6.47 (m, 1H), 4.47 (m, 1H), 3.28 (t, J=4.9 Hz, 4H) 2.96 (m, 4H), 2.85 (m, 2H), 2.43 (m, 1H), 2.07 (m, 2H), 1.67 (m, 8H), 1.35 (m, 3H). MS (LC/MS, M+H⁺): 382.2.

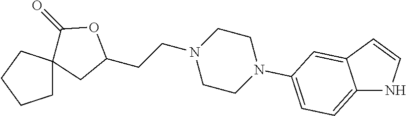 [SMILES: O=C1OC(CCN2CCN(c3ccc4[nH]ccc4c3)CC2)CC12CCCC2]

Preparation of 3-(2-(4-(1H-indol-5-yl)piperazin-1-yl)ethyl)-2-oxaspiro[4.4]nonan-1-one: The title compound was prepared according to the procedure for 5-(2-(4-(1H-benzo[d]imidazol-2-yl)piperidin-1-yl)ethyl)-3,3-diethyldihydrofuran-2(3H)-one, except 5-(piperazin-1-yl)-1H-indole was substituted for 2-piperazin-1-yl-benzonitrile, and 2-(1-oxo-2-oxaspiro[4.4]nonan-3-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) 8.31 (s, 1H), 8.10 (s, 1H), 7.31 (d, J=8.8 Hz, 1H), 7.18 (s, 2H), 6.94 (dd, J=8.7 Hz, J=2.2 Hz, 1H),6.47 (m, 1H), 4.47 (m, 1H), 3.28 (t, J=4.7 Hz, 4H) 2.96 (m, 4H), 2.86 (m, 2H), 2.27 (m, 1H), 2.07 (m, 2H), 1.85 (m, 9H). MS (LC/MS, M+H⁺): 369.2.

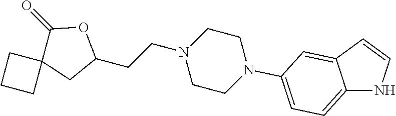 [SMILES: O=C1OC(CCN2CCN(c3ccc4[nH]ccc4c3)CC2)CC12CCC2]

Preparation of 7-(2-(4-(1H-indol-5-yl)piperazin-1-yl)ethyl)-6-oxaspiro[3.4]octan-5-one: The title compound was prepared according to the procedure for 5-(2-(4-(1H-benzo[d]imidazol-2-yl)piperidin-1-yl)ethyl)-3,3-diethyldihydrofuran-2(3H)-one, except 5-(piperazin-1-yl)-1H-indole was substituted for 2-piperazin-1-yl-benzonitrile, and 2-(5-oxo-6-oxaspiro[3.4]octan-7-yl)ethyl 4-methylbenzenesulfonate was substituted for 2-(4,4-diethyl-5-oxotetrahydrofuran-2-yl)ethyl 4-methylbenzenesulfonate. ¹H NMR (400 MHz, CDCl₃) 8.09 (s, 1H), 7.75 (s, 1H), 7.32 (d, J=9 Hz, 1H), 7.19 (s, 2H), 6.94 (dd, J=8.8 Hz, J=2.0 Hz, 1H),6.45 (m, 1H), 4.43 (m, 1H), 3.39 (m, 4H) 3.25 (m, 4H), 2.93 (m, 2H), 2.54 (m, 1H), 2.43 (m, 2H), 2.02 (m, 7H). MS (LC/MS, M+H⁺): 354.2.

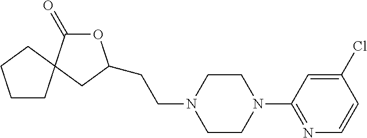 [SMILES: O=C1OC(CCN2CCN(c3cc(Cl)ccn3)CC2)CC12CCCC2]

Preparation of 3-(2-(4-(4-chloropyridin-2-yl)piperazin-1-yl)ethyl)-2-oxaspiro[4.4]nonan-1-one: To a small vial, 2-(1-oxo-2-oxaspiro[4.4]nonan-3-yl)ethyl 4-methylbenzenesulfonate (50 mg, 0.15 mmol, 1.0 equiv) was added and dissolved in 5 mL tetrahydrofuran. 1-(4-chloropyridin-2-yl)piperazine (58 mg, 0.30 mmol, 2.0 equiv) was then added. The reaction was stirred at 60° C. for 3 days. The solution was allowed to cool to room temperature and was then diluted with deionized water (5 mL) and ethyl acetate (5 mL). The layers were separated and the aqueous layer was washed 2×5 mL ethyl acetate. The organic layers were combined and dried over Na₂SO₄ and then concentrated under reduced pressure. The resulting crude oil was purified through flash chromatography (silica gel; methanol/dichloromethane, 0-10%). ¹H NMR (400 MHz, chloroform-d) δ 7.96 (d, J=6.0 Hz, 1H), 6.58 (d, J=2.3 Hz, 1H), 6.50 (dd, J=6.0, 2.3 Hz, 1H), 4.42 (m, 1H), 3.29 (br, 4H), 2.53 (br, 6H), 2.18 (m, 2H), 1.85-1.55 (m, 10H). LC/MS M+1=364.20.

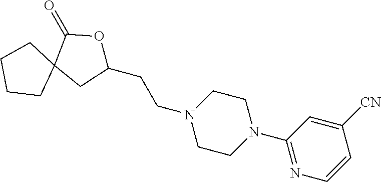 [SMILES: N#Cc1ccnc(N2CCN(CCC3CC4(CCCC4)C(=O)O3)CC2)c1]

Preparation of 2-(4-(2-(1-oxo-2-oxaspiro[4.4]nonan-3-yl)ethyl)piperazin-1-yl)isonicotinonitrile: The title compound was prepared according to the procedure for 3-(2-(4-(4-chloropyridin-2-yl)piperazin-1-yl)ethyl)-2-oxaspiro[4.4]nonan-1-one except 2-(piperazin-1-yl)isonicotinonitrile was substituted for 1-(4-chloropyridin-2-yl)piperazine. ¹H NMR (400 MHz, chloroform-d) δ 8.21 (d, 5.0 Hz, 1H), 6.73 (s, 1H), 6.69 (d, J=5.0 Hz, 1H), 4.42 (m, 1H), 3.52 (br, 4H), 4.50 (br H), 2.19 (m, 2H), 1.79-1.56 (m, 10H. LC/MS M+1=355.20.

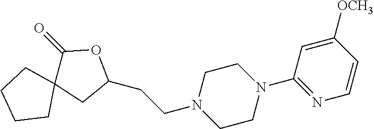 [SMILES: COc1ccnc(N2CCN(CCC3CC4(CCCC4)C(=O)O3)CC2)c1]

Preparation of 3-(2-(4-(4-methoxypyridin-2-yl)piperazin-1-yl)ethyl)-2-oxaspiro[4.4]nonan-1-one: The title compound was prepared according to the procedure for 3-(2-(4-(4-chloropyridin-2-yl)piperazin-1-yl)ethyl)-2-oxaspiro[4.4]nonan-1-one except 1-(4-methoxypyridin-2-yl)piperazine was substituted for 1-(4-chloropyridin-2-yl)piperazine. ¹H NMR (400 MHz, chloroform-d) δ 7.97 (d, J=5.8 Hz, 1H), 6.20 (dd, J=5.8, 2.0 Hz, 1H), 6.03 (d, J=2.0 Hz, 1H), 4.42 (m, 1H), 3.74 (s, 3H), 3.46 (br, 4H), 2.50 (br, 6H), 2.18 (m, 2H), 1.8-1.6 (m, 10H). LC/MS M+1=360.20.

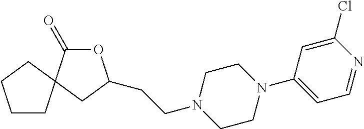 [SMILES: O=C1OC(CCN2CCN(c3ccnc(Cl)c3)CC2)CC12CCCC2]

Preparation of 3-(2-(4-(2-chloropyridin-4-yl)piperazin-1-yl)ethyl)-2-oxaspiro[4.4]nonan-1-one: The title compound was prepared according to the procedure for 3-(2-(4-(4-chloropyridin-2-yl)piperazin-1-yl)ethyl)-2-oxaspiro[4.4]nonan-1-one except 1-(2-chloropyridin-4-yl)piperazine was substituted for 1-(4-chloropyridin-2-yl)piperazine. ¹H NMR (400 MHz, chloroform-d) δ 8.19 (d, J=6.5 Hz, 1H), 6.71 (d, J=2.5 Hz, 1H), 6.68 (dd, J=6.5, 2.5 Hz, 1H), 4.36 (m, 1H), 3.76 (br, 4H), 3.30 (br, 6H), 2.26 (m, 2H), 1.80-1.52 (m, 10H). LC/MS M+1=364.20.

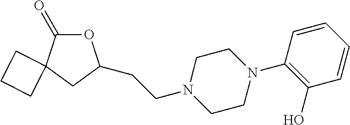 [SMILES: O=C1OC(CCN2CCN(c3ccccc3O)CC2)CC12CCC2]

Preparation of 7-(2-(4-(2-hydroxyphenyl)piperazin-1-yl)ethyl)-6-oxaspiro[3.4]octan-5-one: To a small vial, 2-(5-oxo-6-oxaspiro[3.4]octan-7-yl)ethyl 4-methylbenzenesulfonate (50 mg, 0.15 mmol, 1.0 equiv) was added and dissolved in 5 mL tetrahydrofuran. 2-(piperazin-1-yl)phenol (27.54 mg, 0.15 mmol, 1.0 equiv) was added along with 30 μL of triethylamine (0.21 mmol, 1.4 equiv). The reaction was stirred at 60° C. for 3 days. The solution was allowed to cool to room temperature and was then diluted with deionized water (5 mL) and ethyl acetate (5 mL). The layers were separated and the aqueous layer was washed 2×5 mL ethyl acetate. The organic layers were combined and dried over Na₂SO₄ and then concentrated under reduced pressure. The resulting crude oil was purified through flash chromatography (silica gel; methanol/dichloromethane, 0-10%). ¹H NMR (400 MHz, chloroform-d) δ 7.09 (m, 2H), 6.89 (dd, J=8.2, 1.2 Hz, 1H), 6.85 (td, J=7.5 1.2 Hz, 1H), 4.34 (m, 1H), 3.70 (b, 4H), 3.27-3.14 (m, 6H), 2.51 (m, 2H), 2.37 (m, 1H), 2.19 (m, 1H), 2.1-1.9 (m, 6H). LC/MS M+1=331.20.

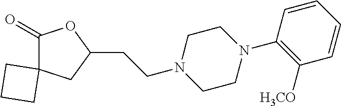 [SMILES: COc1ccccc1N1CCN(CCC2CC3(CCC3)C(=O)O2)CC1]

Preparation of 7-(2-(4-(2-methoxyphenyl)piperazin-1-yl)ethyl)-6-oxaspiro[3.4]octan-5-one: The title compound was prepared according to the procedure for 7-(2-(4-(2-hydroxyphenyl)piperazin-1-yl)ethyl)-6-oxaspiro[3.4]octan-5-one except 1-(2-methoxyphenyl)piperazine was substituted for 2-(piperazin-1-yl)phenol. ¹H NMR (400 MHz, chloroform-d) δ 7.19 (td, J=8.0, 1.5 Hz, 1H), 7.07 (dd, J=8.0, 1.5 Hz, 1H), 7.00 (td, J=8.0, 1.0 Hz, 1H), 6.95 (dd, J=8.0, 1.0 Hz, 1H), 4.41 (m, 1H), 3.90 (s, 3H), 3.8-3.2 (br, 10H), 2.58 (m, 2H), 2.45 (m, 1H), 2.24 (m, 1H), 2.20-2.00 (m, 6H). LC/MS M+1=345.20.

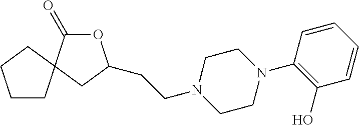 [SMILES: O=C1OC(CCN2CCN(c3ccccc3O)CC2)CC12CCCC2]

Preparation of 3-(2-(4-(2-hydroxyphenyl)piperazin-1-yl)ethyl)-2-oxaspiro[4.4]nonan-1-one: The title compound was prepared according to the procedure for 7-(2-(4-(2-hydroxyphenyl)piperazin-1-yl)ethyl)-6-oxaspiro[3.4]octan-5-one except 2-(1-oxo-2-oxaspiro[4.4]nonan-3-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 7.07 (m, 2H), 6.88 (dd, J=8.0, 0.8 Hz, 1H), 6.84 (td, J=7.7, 0.8 Hz, 1H), 4.38 (m, 1H), 3.69 (br, 4H), 3.35-3.05 (br, 6H), 2.22 (m, 2H), 2.08 (m, 2H), 1.80 (m, 4H), 1.63 (m, 3H), 1.56 (m, 1H). LC/MS M+1=345.20.

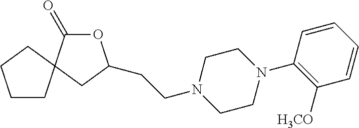 [SMILES: COc1ccccc1N1CCN(CCC2CC3(CCCC3)C(=O)O2)CC1]

Preparation of 3-(2-(4-(2-methoxyphenyl)piperazin-1-yl)ethyl)-2-oxaspiro[4.4]nonan-1-one: The title compound was prepared according to the procedure for 7-(2-(4-(2-hydroxyphenyl)piperazin-1-yl)ethyl)-6-oxaspiro[3.4]octan-5-one except 2-(1-oxo-2-oxaspiro[4.4]nonan-3-yl)ethyl 4-methylbenzenesulfonate was substituted for 2-(5-oxo-6-oxaspiro[3.4]octan-7-yl)ethyl 4-methylbenzenesulfonate and 1-(2-methoxyphenyl)piperazine was substituted for 2-(piperazin-1-yl)phenol. ¹H NMR (400 MHz, chloroform-d) δ 7.11 (td, J=8.0, 1.5 Hz, 1H), 6.99 (dd, J=8.0, 1.5 Hz, 1H), 6.90 (td, J=7.8, 1.2 Hz, 1H), 6.87 (dd, J=8.2, 1.2 Hz, 1H), 4.37 (m, 1H), 3.81 (s, 3H), 3.78-3.20 (br, 10H), 2.20 (m, 2H), 2.05 (m, 2H), 1.78 (m, 4H), 1.62 (m, 3H), 1.55 (m, 1H). LC/MS M+1=359.20.

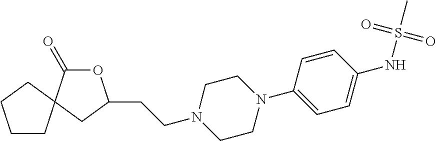 [SMILES: CS(=O)(=O)Nc1ccc(N2CCN(CCC3CC4(CCCC4)C(=O)O3)CC2)cc1]

Preparation of N-(4-(4-(2-(1-oxo-2-oxaspiro[4.4]nonan-3-yl)ethyl)piperazin-1-yl)phenyl)methanesulfonamide: The title compound was prepared according to the procedure for 7-(2-(4-(2-hydroxyphenyl)piperazin-1-yl)ethyl)-6-oxaspiro[3.4]octan-5-one except 2-(1-oxo-2-oxaspiro[4.4]nonan-3-yl)ethyl 4-methylbenzenesulfonate was substituted for 2-(5-oxo-6-oxaspiro[3.4]octan-7-yl)ethyl 4-methylbenzenesulfonate and N-(4-(piperazin-1-yl)phenyl)methanesulfonamide was substituted for 2-(piperazin-1-yl)phenol. ¹H NMR (400 MHz, chloroform-d) δ 7.07 (d, J=8.9 Hz, 2H), 6.81 (d, J=8.9 Hz, 2H), 4.40 (m, 1H), 3.11 (m, 4H), 2.87 (s, 3H), 2.49 (br, 6H), 2.17 (m, 2H), 1.90-1.60 (m, 10H). LC/MS M+1=422.2.

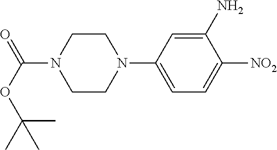 [SMILES: CC(C)(C)OC(=O)N1CCN(c2ccc([N+](=O)[O-])c(N)c2)CC1]

Preparation of tert-butyl 4-(3-amino-4-nitrophenyl) piperazine-1-carboxylate: To a solution of 5-chloro-2-nitrobenzenamine (1.5 g, 8.6 mmol) in 15 mL N, N-dimethyl acetamide, tert-butyl piperazine-1-carboxylate (2.4 g, 13.0 mmol) and potassium carbonate (3.6 g, 26 mmol) were added and stirred at 140° C. for 18 hours. The reaction was cooled to room temperature and filtered. The filtrate was concentrated to an oil under reduced pressure. The oil was diluted with water (200 mL) and filtered. The precipitate was washed with ether (50 mL) and dried under high vacuum to yield (1.57 g, 56%) of product as yellow solid. ¹H NMR (400 MHz, Chloroform-d) δ 8.03 (d, J=9.6 Hz, 1H), 6.25 (dd, J=9.6 Hz, J=2.6 Hz, 1H)), 6.17 (bs, 2H), 5.93 (d, J=2.6 Hz, 1H), 3.56 (t, J=5.5 Hz, 4H), 3.35 (t, J=5.5 Hz, 4H), 1.48 (s, 9H). LC/MS M+1=323.2.

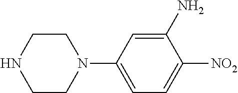 [SMILES: Nc1cc(N2CCNCC2)ccc1[N+](=O)[O-]]

Preparation of 2-nitro-5-(piperazin-1-yl) benzenamine: To a solution of tert-butyl 4-(3-amino-4-nitrophenyl) piperazine-1-carboxylate (0.1 g, 0.31 mmol) in 20 mL dichloromethane, 2 mL trifluroacetic acid was added and the reaction was stirred for 1 hour. The solvent was removed under reduced pressure. The oil obtained was dissolved in 20 mL methanol and amberlite IRA-400(OH) resin (1 g) was added and the reaction mixture was stirred for 30 min. The resin was filtered and the solvent was removed under vacuum to afford the desired product as a solid (0.030 g, 45%). LC/MS M+1=223.2.

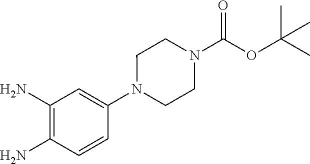 [SMILES: CC(C)(C)OC(=O)N1CCN(c2ccc(N)c(N)c2)CC1]

Preparation of tert-butyl 4-(3, 4-diaminophenyl) piperazine-1-carboxylate: To a solution of tert-butyl 4-(3-amino-4-nitrophenyl) piperazine-1-carboxylate (1 g, 3.1 mmol) in 50 mL methanol, 10% Pd/C (0.2 g) was added and stirred under a hydrogen gas at atmospheric pressure for 20 hours. The reaction mixture was filtered through a patch of celite and the solvent was removed under reduced pressure to afford the desired product as a solid (0.7 g, 77%). ¹H NMR (400 MHz, Chloroform-d) δ 6.57 (d, J=8.3 Hz, 1H), 6.28 (d, J=2.5 Hz, 1H), 6.24 (dd, J=8.3 Hz, J=2.5 Hz, 1H), 3.49 (t, J=4.9 Hz, 4H), 2.90 (t, J=4.8 Hz, 4H), 1.40 (s, 9H). LC/MS M+1=293.2.

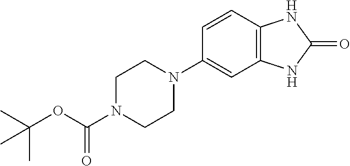 [SMILES: CC(C)(C)OC(=O)N1CCN(c2ccc3[nH]c(=O)[nH]c3c2)CC1]

Preparation of tert-butyl 4-(2, 3-dihydro-2-oxo-1H-benzo[d]imidazol-6-yl) piperazine-1-carboxylate: To a solution of tert-butyl 4-(3,4-diaminophenyl)piperazine-1-carboxylate (0.44 g, 1.50 mmol) in 25 mL dichlromethane, imidazole (0.27 g, 1.65 mmol) and triethylamine (0.45 g, 4.5 mmol) were added and their reaction mixture was stirred for 18 hours. The solvent was removed under reduced pressure and the oil obtained was purified by flash chromatography to afford the desired product as a solid (0.17 g, 36%). ¹H NMR (400 MHz, Chloroform-d) δ 8.0 (s, 1H), 7.8 (s, 1H), 6.85 (d, J=8.4 Hz, 1H), 6.62 (dd, J=8.4 Hz, J=2.52 Hz, 1H), 6.60 (s, 1H), 3.51 (t, J=4.9 Hz, 4H), 2.96 (t, J=5.2 Hz, 4H), 1.42 (s, 9H). LC/MS M+1=319.2.

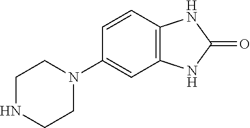 [SMILES: O=c1[nH]c2ccc(N3CCNCC3)cc2[nH]1]

Preparation of 5-(piperazin-1-yl)-1H-benzo[d]imidazol-2(3H)-one: To a solution of tert-butyl 4-(2,3-dihydro-2-oxo-1H-benzo[d]imidazol-6-yl)piperazine-1-carboxylate (0.1 g, 0.31 mmol) in 3 mL Dichloromethane, 1 mL trifluroacetic acid was added and the reaction mixture was stirred for 2 hours. The solvent was removed under reduced pressure. The oil obtained was dissolved in 30 mL of methanol and Amberlite IRA-400(OH) resin (1 g) was added and the reaction mixture was stirred for 15 min. The resin was filtered and the solvent was removed under vacuum to afford the desired product as a solid (0.06 g, 92%). ¹H NMR (400 MHz, DMSO-d₆) δ 10.38 (s, 1H), 10.27 (s, 1H), 6.76 (d, J=8.4 Hz, 1H), 6.52 (dd, J=8.4 Hz, J=2.3 Hz, 1H), 6.49 (d, J=2.1 Hz, 1H), 3.88 (m, 4H), 2.81 (m, 4H). LC/MS M+23=241.10.

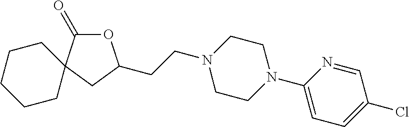 [SMILES: O=C1OC(CCN2CCN(c3ccc(Cl)cn3)CC2)CC12CCCCC2]

Preparation of 3-{2-[4-(5-chloro-pyridin-2-yl)-piperazin-1-yl]-ethyl}-2-oxa-spiro[4.5]decan-1-one: To a solution of 2-(1-oxo-2-oxaspiro[4.5]decan-3-yl)ethyl 4-methylbenzenesulfonate (0.050 g, 0.15 mmol) in 4 mL Tetrahydrofuran, 1-(5-chloropyridin-2-yl)piperazine (0.073 g, 0.37 mmol) was added and the reaction mixture was stirred at 60° C. for 48 hours. The precipitate was filtered. The filtrate was concentrated to an oil under reduced pressure. The crude oil was purified by reverse phase flash chromatography using acetonitrile/H₂O/0.1% formic acid as eluent (5% acetonitrile to 95% acetonitrile over a 15 minute gradient) to afforded 3-{2-[4-(5-chloro-pyridin-2-yl)-piperazin-1-yl]-ethyl}-2-oxa-spiro[4.5]decan-1-one (0.027 g, 51%) as an off white solid. ¹H NMR (400 MHz, chloroform-d) δ 8.12 (d, J=2.4 Hz, 1H), 7.43 (dd, J=9.0 Hz, J=2.6 Hz, 1H), 6.59 (d, J=9.0 Hz, 1H) 4.52 (m, 1H), 3.53 (pen, J=5.6 Hz, J=1.4 Hz, 4H) 2.57 (m, 4H), 2.54 (m, 2H), 2.43 (m, 1H), 1.86 (m, 4H), 1.69 (m, 6H), 1.35 (m, 3H). LC/MS M+1=378.2.

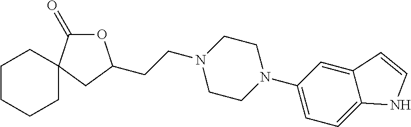 [SMILES: O=C1OC(CCN2CCN(c3ccc4[nH]ccc4c3)CC2)CC12CCCCC2]

Preparation of 3-{2-[4-(1H-Indol-5-yl)-piperazin-1-yl]-ethyl}-2-oxa-spiro[4.5]decan-1-one: The title compound was prepared according to the procedure for 3-{2-[4-(5-Chloro-pyridin-2-yl)-piperazin-1-yl]-ethyl}-2-oxa-spiro[4.5]decan-1-one except 5-(piperazin-1-yl)-1H-indole was substituted for 1-(5-chloropyridin-2-yl)piperazine. ¹H NMR (400 MHz, chloroform-d) δ 8.34 (s, 1H), 8.11 (s, 1H), 7.31 (d, J=8.7 Hz, 1H), 7.19 (d, J=2.4 Hz, 2H), 6.94 (dd, J=9.0 Hz, J=2.2 Hz, 1H),6.47 (m, 1H), 4.47 (m, 1H), 3.28 (t, J=4.9 Hz, 4H) 2.96 (m, 4H), 2.85 (m, 2H), 2.43 (m, 1H), 2.07 (m, 2H), 1.67 (m, 8H), 1.35 (m, 3H). LC/MS M+1=382.2.

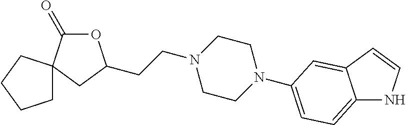 [SMILES: O=C1OC(CCN2CCN(c3ccc4[nH]ccc4c3)CC2)CC12CCCC2]

Preparation of 3-{2-[4-(1H-Indol-5-yl)-piperazin-1-yl]-ethyl}-2-oxa-spiro[4.4]nonan-1-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-indole was substituted for 1-(5-chloropyridin-2-yl)piperazine and 2-(1-oxo-2-oxaspiro[4.4]nonan-3-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 8.31 (s, 1H), 8.10 (s, 1H), 7.31 (d, J=8.8 Hz, 1H), 7.18 (s, 2H), 6.94 (dd, J=8.7 Hz, J=2.2 Hz, 1H),6.47 (m, 1H), 4.47 (m, 1H), 3.28 (t, J=4.7 Hz, 4H) 2.96 (m, 4H), 2.86 (m, 2H), 2.27 (m, 1H), 2.07 (m, 2H), 1.85 (m, 9H). LC/MS M+1=369.2.

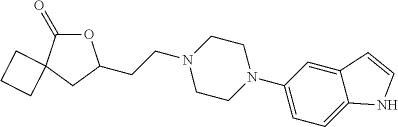 [SMILES: O=C1OC(CCN2CCN(c3ccc4[nH]ccc4c3)CC2)CC12CCC2]

Preparation of 7-{2-[4-(1H-Indol-5-yl)-piperazin-1-yl]-ethyl}-6-oxa-spiro[3.4]octan-5-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-indole was substituted for 1-(5-chloropyridin-2-yl)piperazine and 2-(5-oxo-6-oxaspiro[3.4]octan-7-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 8.09 (s, 1H), 7.75 (s, 1H), 7.32 (d, J=9 Hz, 1H), 7.19 (s, 2H), 6.94 (dd, J=8.8 Hz, J=2.0 Hz, 1H),6.45 (m, 1H), 4.43 (m, 1H), 3.39 (m, 4H) 3.25 (m, 4H), 2.93 (m, 2H), 2.54 (m, 1H), 2.43 (m, 2H), 2.02 (m, 7H). LC/MS M+1=354.2.

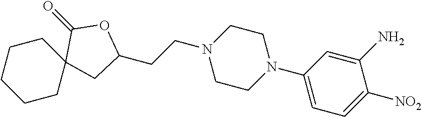 [SMILES: Nc1cc(N2CCN(CCC3CC4(CCCCC4)C(=O)O3)CC2)ccc1[N+](=O)[O-]]

Preparation of 3-(2-(4-(-3-amino-4-nitrophenyl)piperazin-1-yl)ethyl)-2-oxaspiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 2-nitro-5-(piperazin-1-yl)aniline was substituted for 1-(5-chloropyridin-2-yl)piperazine. ¹H NMR (400 MHz, chloroform-d) δ 7.95 (d, J=9.7 Hz, 1H), 6.19 (dd, J=9.7 Hz, J=2.5 Hz, 1H), 6.18 (bs, 2H), 5.89 (d, J=2.5 Hz, 1H), 4.42 (m, 1H), 3.34 (t, J=4.9 Hz, 4H) 2.96 (m, 4H), 2.32 (m, 1H), 1.85 (m, 3H), 1.74 (m, 3H), 1.58 (m, 3H), 1.42 (m, 1H), 1.30 (m, 1H), 1.18 (m, 1H). LC/MS M+1=403.2.

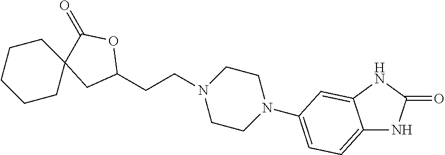 [SMILES: O=C1OC(CCN2CCN(c3ccc4[nH]c(=O)[nH]c4c3)CC2)CC12CCCCC2]

Preparation of 5-(4-(2-(1-oxo-2-oxaspiro[4.5]decan-3-yl)ethyl)piperazin-1-yl)-1H-benzo[d]imidazol-2(3H)-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-benzo[d]imidazol-2(3H)-one was substituted for 1-(5-chloropyridin-2-yl)piperazine. ¹H NMR (400 MHz, chloroform-d) δ 6.93 (d, J=9.1 Hz, 1H), 6.75 (d, J=2.2 Hz, 1H), 6.74 (d, J=2.2 Hz, 1H), 4.55 (m, 1H), 3.13 (t, J=4.4 Hz, 4H) 2.67 (m, 4H), 2.52 (m, 1H), 2.50 (m, 2H), 1.93 (m, 2H), 1.74 (m, 6H), 1.52 (m, 3H), 1.34 (m, 2H). LC/MS M+1=399.2.

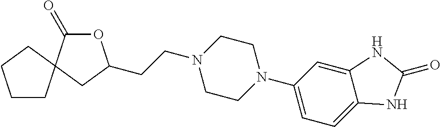 [SMILES: O=C1OC(CCN2CCN(c3ccc4[nH]c(=O)[nH]c4c3)CC2)CC12CCCC2]

Preparation of 5-(4-(2-(1-oxo-2-oxaspiro[4.4]nonan-3-yl)piperazin-1-yl)-1H-benzo[d]imidazol-2(3H)-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-benzo[d]imidazol-2(3H)-one was substituted for 1-(5-chloropyridin-2-yl)piperazine and 2-(1-oxo-2-oxaspiro[4.4]nonan-3-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 6.95 (d, J=9.2 Hz, 1H), 6.77 (d, J=2.2 Hz, 1H), 6.74 (d, J=2.2 Hz, 1H), 4.55 (m, 1H), 3.15 (t, J=4.4 Hz, 4H) 2.67 (t, J=4.9 Hz, 4H), 2.60 (m, 2H), 2.35 (m, 1H), 2.10 (m, 1H), 1.90 (m, 2H), 1.87 (m, 1H), 1.75 (m, 4H), 1.69 (m, 1H). LC/MS M+1=385.2.

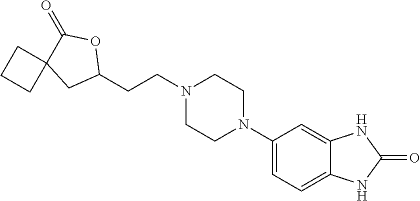 [SMILES: O=C1OC(CCN2CCN(c3ccc4[nH]c(=O)[nH]c4c3)CC2)CC12CCC2]

Preparation of 5-(4-(2-(1-oxo-2-oxaspiro[3.4]octan-3-yl)piperazin-1-yl)-1H-benzo[d]imidazol-2(3H)-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-benzo[d]imidazol-2(3H)-one was substituted for 1-(5-chloropyridin-2-yl)piperazine and 2-(5-oxo-6-oxaspiro[3.4]octan-7-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 6.95 (d, J=9.2 Hz, 1H), 6.77 (d, J=1.6 Hz, 1H), 6.75 (d, J=2.2 Hz, 1H), 4.55 (m, 1H), 3.15 (t, J=4.7 Hz, 4H) 2.67 (t, J=4.9 Hz, 4H), 2.50 (m, 4H), 2.07 (m, 4H), 1.92 (m, 4H). LC/MS M+1=371.2.

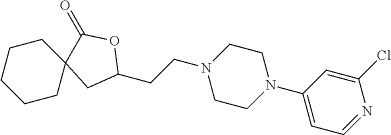 [SMILES: O=C1OC(CCN2CCN(c3ccnc(Cl)c3)CC2)CC12CCCCC2]

Preparation of 3-(2-(4-(2-chloropyridin-4-yl)piperazin-1-yl)ethyl)-2-oxaspiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 1-(2-chloropyridin-4-yl)piperazine was substituted for 1-(5-chloropyridin-2-yl)piperazine. ¹H NMR (400 MHz, MeOD) δ 7.76 (d, J=6.2 Hz, 1H), 6.69 (d, J=2.2 Hz, 1H), 6.55 (dd, J=6.2 Hz, J=2.3 Hz, 1H), 4.44 (m, 1H), 3.28 (t, J=5.0 Hz, 4H) 2.45 (m, 4H), 2.34 (m, 3H), 1.75 (m, 2H), 1.60 (m, 6H), 1.37 (m, 3H), 1.20 (m, 2H). LC/MS M+1=378.2.

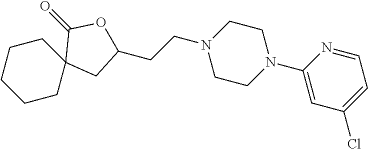 [SMILES: O=C1OC(CCN2CCN(c3cc(Cl)ccn3)CC2)CC12CCCCC2]

Preparation of 3-{2-[4-(4-chloro-pyridin-2-yl)-piperazin-1-yl]-ethyl}-2-oxa-spiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 1-(4-chloropyridin-2-yl)piperazine was substituted for 1-(5-chloropyridin-2-yl)piperazine. ¹H NMR (400 MHz, MeOD) δ 7.94 (d, J=6.2 Hz, 1H), 6.86 (d, J=2.3 Hz, 1H), 6.82 (dd, J=6.2 Hz, J=2.4 Hz, 1H), 4.58 (m, 1H), 3.45 (t, J=5.1 Hz, 4H) 2.62 (m, 4H), 2.53 (m, 3H), 1.92 (m, 2H), 1.75 (m, 6H), 1.53 (m, 3H), 1.36 (m, 2H). LC/MS M+1=378.2.

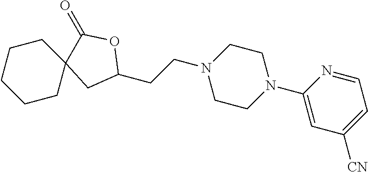 [SMILES: N#Cc1ccnc(N2CCN(CCC3CC4(CCCCC4)C(=O)O3)CC2)c1]

Preparation of 2-(4-(2-(1-oxo-2-oxaspiro[4.5]decan-3-yl)ethyl)piperazin-1-yl)isonicotinonitrile: The title compound was prepared according to the procedure for 3-{2-[4-(5-chloro-pyridin-2-yl)-piperazin-1-yl]-ethyl}-2-oxa-spiro[4.5]decan-1-one except 2-(piperazin-1-yl)isonicotinonitrile was substituted for 1-(5-chloropyridin-2-yl)piperazine. ¹H NMR (400 MHz, MeOD) δ 8.27 (d, J=5.0 Hz, 1H), 7.1 (s, 1H), 6.85 (dd, J=5.0 Hz, J=1.0 Hz, 1H), 4.59 (m, 1H), 3.64 (t, J=5.1 Hz, 4H) 2.62 (m, 4H), 2.52 (m, 3H), 1.94 (m, 2H), 1.75 (m, 6H), 1.54 (m, 3H), 1.36 (m, 2H). LC/MS M+1=369.2.

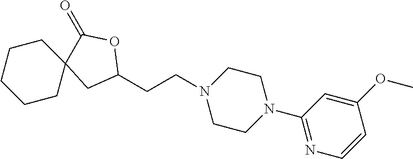 [SMILES: COc1ccnc(N2CCN(CCC3CC4(CCCCC4)C(=O)O3)CC2)c1]

Preparation of 3-(2-(4-(4-methoxypyridin-2-yl)piperazin-1-yl)ethyl)-2-oxaspiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 1-(4-methoxypyridin-2-yl)piperazine was substituted for 1-(5-chloropyridin-2-yl)piperazine. ¹H NMR (400 MHz, MeOD) δ 7.92 (d, J=5.9 Hz, 1H), 6.37 (dd, J=5.9 Hz, J=2.1 Hz, 1H), 6.29 (d, J=2.0 Hz, 1H), 4.57 (m, 1H), 3.84 (s, 1H), 3.52 (t, J=5.1 Hz, 4H) 2.62 (m, 4H), 2.55 (m, 3H), 1.94 (m, 2H), 1.75 (m, 6H), 1.53 (m, 3H), 1.36 (m, 2H). LC/MS M+1=374.2.

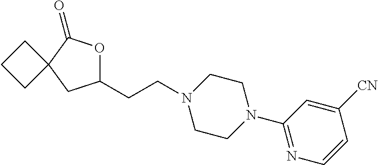 [SMILES: N#Cc1ccnc(N2CCN(CCC3CC4(CCC4)C(=O)O3)CC2)c1]

Preparation of 2-(4-(2-(5-oxo-6-oxaspiro[3.4]octan-7-yl)ethyl)piperazin-1-yl)isonicotinonitrile: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-benzo[d]imidazol-2(3H)-one was substituted for 2-(piperazin-1-yl)isonicotinonitrile and 2-(5-oxo-6-oxaspiro[3.4]octan-7-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, MeOD) δ 8.27 (dd, J=5.0 Hz, J=1.0 Hz, 1H), 7.1 (s, 1H), 6.85 (dd, J=5.0 Hz, J=1.1 Hz, 1H), 4.49 (m, 1H), 3.63 (t, J=5.1 Hz, 4H) 2.61 (m, 4H), 2.57 (m, 4H), 2.36 (m, 1H), 2.19 (m, 1H), 2.07 (m, 4H), 1.90 (m, 2H). LC/MS M+1=341.2.

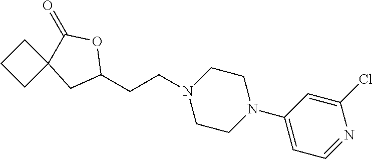 [SMILES: O=C1OC(CCN2CCN(c3ccnc(Cl)c3)CC2)CC12CCC2]

Preparation of 7-(2-(4-(2-chloropyridin-4-yl)piperazin-1-yl)ethyl)-6-oxaspiro[3.4]octan-5-one: The title compound was prepared according to the procedure for 3-{2-[4-(5-chloro-pyridin-2-yl)-piperazin-1-yl]-ethyl}-2-oxa-spiro[4.5]decan-1-one except 5-(piperazin-1-yl)-1H-benzo[d]imidazol-2(3H)-one was substituted for 1-(2-chloropyridin-4-yl)piperazine and 2-(5-oxo-6-oxaspiro[3.4]octan-7-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, MeOD) δ 7.93 (d, J=6.2 Hz, 1H), 6.83 (d, J=2.6 Hz, 1H), 6.82 (dd, J=6.2 Hz, J=2.4 Hz, 1H), 4.45 (m, 1H), 3.44 (t, J=5.1 Hz, 4H) 2.61 (m, 4H), 2.55 (m, 4H), 2.36 (m, 1H), 2.17 (m, 1H), 2.06 (m, 4H), 1.88 (m, 2H). LC/MS M+1=350.2.

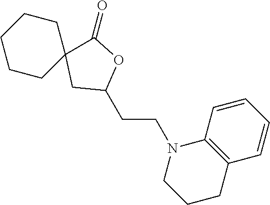 [SMILES: O=C1OC(CCN2CCCc3ccccc32)CC12CCCCC2]

Preparation of 3-(2-(3,4-dihydroquinolin-1 (2H)-yl)ethyl)-2-oxaspiro[4.5]decan-1-one: The title compound was prepared according to the procedure for 3-{2-[4-(5-chloro-pyridin-2-yl)-piperazin-1-yl]-ethyl)}-2-oxa-spiro[4.5]decan-1-one except 1,2,3,4-tetrahydroquinoline was substituted for 1-(5-chloropyridin-2-yl)piperazine. ¹H NMR (400 MHz, chloroform-d) δ 7.07 (t, J=8.9 Hz 1H), 6.98 (dd, J=1.6 Hz, J=0.8 Hz, 1H), 6.59 (m, 2H), 4.49 (m, 1H), 3.48 (m, 2H), 3.31 (t, J=5.6 Hz, 2H) 2.77 (t, J=6.3 Hz, 2H), 2.41 (m, 1H), 1.97 (m, 2H), 1.85 (m, 4H), 1.66 (m, 5H), 1.37 (m, 4H). LC/MS M+1=314.2.

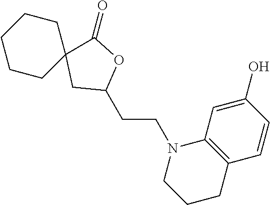 [SMILES: O=C1OC(CCN2CCCc3ccc(O)cc32)CC12CCCCC2]

Preparation of 3-(2-(7-hydroxy-3,4-dihydroquinolin-1 (2H)-yl)ethyl)-2-oxaspiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 1,2,3,4-tetrahydroquinolin-7-ol was substituted for 1-(5-chloropyridin-2-yl)piperazine. ¹H NMR (400 MHz, chloroform-d) δ 6.78 (d, J=8.0 Hz 1H),6.13 (d, J=2.4 Hz, 1H), 6.08 (dd, J=8.0 Hz, J=2.4 Hz, 1H), 5.36 (s, 1H), 4.46 (m, 1H), 3.40 (m, 2H), 3.25 (t, J=5.1 Hz, 2H) 2.66 (t, J=6.3 Hz, 2H), 2.38 (m, 1H), 1.92 (m, 2H), 1.80 (m, 4H), 1.64 (m, 5H), 1.37 (m, 4H). LC/MS M+1=329.2.

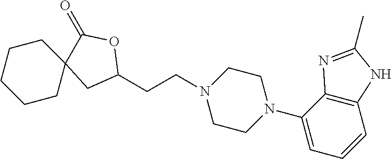 [SMILES: Cc1nc2c(N3CCN(CCC4CC5(CCCCC5)C(=O)O4)CC3)cccc2[nH]1]

Preparation of 3-(2-(4-(2-methyl-1H-benzo[d]imidazol-4-yl)piperazin-1-yl)ethyl)-2-oxaspiro[4.5]decan-1-one: The title compound was prepared according to the procedure for 3-{2-[4-(5-chloro-pyridin-2-yl)-piperazin-1-yl]-ethyl}-2-oxa-spiro[4.5]decan-1-one except 12-methyl-7-(piperazin-1-yl)-1H-benzo[d]imidazole was substituted for 1-(5-chloropyridin-2-yl)piperazine. ¹H NMR (400 MHz, MeOD) δ 7.17 (m, 2H), 6.76 (dd, J=6.7 Hz, J=2.4 Hz, 1H), 4.60 (m, 1H), 3.73 (m, 2H) 3.49 (m, 4H) 3.19 (m, 4H), 3.07 (m, 2H), 2.60 (s, 3H), 2.11 (m, 2H), 1.79 (m, 5H), 1.54 (m, 3H), 1.38 (m, 2H). LC/MS M+1=397.2.

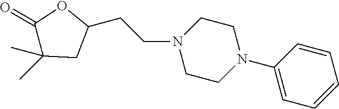 [SMILES: CC1(C)CC(CCN2CCN(c3ccccc3)CC2)OC1=O]

Preparation of dihydro-3,3-dimethyl-5-(2-(4-phenylpiperazin-1-yl)ethyl)furan-2(3H)-one: The title compound was prepared according to the procedure for 3-{2-[4-(5-Chloro-pyridin-2-yl)-piperazin-1-yl]-ethyl}-2-oxa-spiro[4.5]decan-1-one except 1-phenylpiperazine substituted for 1-(2-chloropyridin-4-yl)piperazine and 2-(4,4-dimethyl-5-oxotetrahydrofuran-2-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 7.32 (m, 2H), 6.99 (d, J=7.9 Hz, 1H), 6.91 (t, J=7.2 Hz, 1H), 4.58 (m, 1H), 3.26 (t, J=5.0 Hz, 4H) 2.66 (m, 4H), 2.61 (m, 2H), 2.26 (m, 1H), 1.90 (m, 3H), 1.34 (s, 3H), 1.33 (s, 3H). LC/MS M+1=303.2.

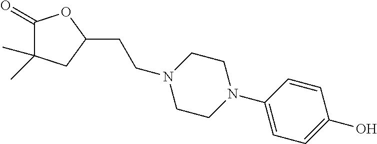 [SMILES: CC1(C)CC(CCN2CCN(c3ccc(O)cc3)CC2)OC1=O]

Preparation of dihydro-5-(2-(4-(4-hydroxyphenyl)piperazin-1-yl)ethyl)-3,3-dimethylfuran-2(3H)-one: The title compound was prepared according to the procedure for 3-{2-[4-(5-chloro-pyridin-2-yl)-piperazin-1-yl]-ethyl)}-2-oxa-spiro[4.5]decan-1-one except 4-(piperazin-1-yl)phenol substituted for 1-(2-chloropyridin-4-yl)piperazine and 2-(4,4-dimethyl-5-oxotetrahydrofuran-2-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 6.87 (m, 2H), 6.77 (m, 2H), 4.54 (m, 1H), 3.21 (t, J=4.9 Hz, 4H) 2.63 (m, 4H), 2.57 (m, 2H), 2.22 (m, 1H), 1.84 (m, 3H), 1.30 (s, 3H), 1.29 (s, 3H). LC/MS M+1=319.2.

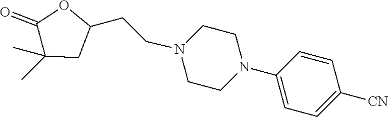 [SMILES: CC1(C)CC(CCN2CCN(c3ccc(C#N)cc3)CC2)OC1=O]

Preparation of 4-(4-(2-(tetrahydro-4,4-dimethyl-5-oxofuran-2-yl)ethyl)piperazin-1-yl)benzonitrile: The title compound was prepared according to the procedure for 3-{2-[4-(5-chloro-pyridin-2-yl)-piperazin-1-yl]-ethyl}-2-oxa-spiro[4.5]decan-1-one except 4-(piperazin-1-yl)benzonitrile substituted for 1-(2-chloropyridin-4-yl)piperazine and 2-(4,4-dimethyl-5-oxotetrahydrofuran-2-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 7.38 (d, J=9.0 Hz, 2H), 6.74 (d, J=9.0 Hz, 2H), 4.42 (m, 1H), 3.22 (t, J=5.0 Hz, 4H) 2.46 (m, 4H), 2.43 (m, 2H), 2.08 (m, 1H), 1.70 (m, 3H), 1.18 (s, 3H), 1.16 (s, 3H). LC/MS M+1=328.2.

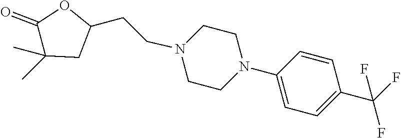 [SMILES: CC1(C)CC(CCN2CCN(c3ccc(C(F)(F)F)cc3)CC2)OC1=O]

Preparation of 5-(2-(4-(4-(trifluoromethyl)phenyl)piperazin-1-yl)ethyl)-dihydro-3,3-dimethylfuran-2(3H)-one: The title compound was prepared according to the procedure for 3-{2-[4-(5-chloro-pyridin-2-yl)-piperazin-1-yl]-ethyl}-2-oxa-spiro[4.5]decan-1-one except 1-(4-(trifluoromethyl)phenyl)piperazine substituted for 1-(2-chloropyridin-4-yl)piperazine and 2-(4,4-dimethyl-5-oxotetrahydrofuran-2-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 7.40 (d, J=8.7 Hz, 2H), 6.84 (d, J=8.7 Hz, 2H), 4.46 (m, 1H), 3.21 (t, J=5.0 Hz, 4H) 2.53 (m, 4H), 2.47 (m, 2H), 2.11 (m, 1H), 1.78 (m, 3H), 1.21 (s, 3H), 1.20 (s, 3H). LC/MS M+1=371.2.

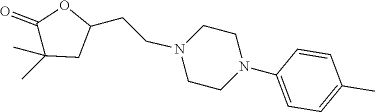 [SMILES: Cc1ccc(N2CCN(CCC3CC(C)(C)C(=O)O3)CC2)cc1]

Preparation of dihydro-3,3-dimethyl-5-(2-(4-p-tolylpiperazin-1-yl)ethyl)furan-2(3H)-one: The title compound was prepared according to the procedure for 3-{2-[4-(5-chloro-pyridin-2-yl)-piperazin-1-yl]-ethyl}-2-oxa-spiro[4.5]decan-1-one except 1-(p-tolyl)piperazine substituted for 1-(2-chloropyridin-4-yl)piperazine and 2-(4,4-dimethyl-5-oxotetrahydrofuran-2-yl)ethyl 4-methylbenzenesulfonate was substituted for 2-(5-oxo-6-oxaspiro[3.4]octan-7-yl)ethyl 4-methylbenzenesulfonate. ¹H NMR (400 MHz, chloroform-d) δ 7.09 (d, J=8.74 Hz, 2H), 6.86 (d, J=8.5 Hz, 2H), 4.54 (m, 1H), 3.17 (t, J=4.9 Hz, 4H) 2.64 (m, 4H), 2.58 (m, 2H), 2.24 (s, 3H), 1.94 (m, 1H), 1.86 (m, 3H), 1.30 (s, 3H), 1.28 (s, 3H). LC/MS M+1=317.2.

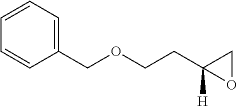 [SMILES: c1ccc(COCC[C@@H]2CO2)cc1]

Preparation of (R)-2-(2-(benzyloxy)ethyl)oxirane: To a solution of (R)-2-(oxiran-2-yl)ethan-1-ol (2.5 g, 28.4 mmol, 1.0 eq) in dry tetrahydrofuran (30 mL) was added 60% NaH (2.27 g, 56.8 mmol, 2 eq) at 0° C. Tetrabutylammonium iodide (13.5 mg, 0.05 mmol, 0.0017 eq) and benzyl bromide (4.06 mL, 34.1 mmol, 1.2 eq) were added at 0° C. The reaction was allowed to warm to room temperature and stir for 3 hours. Once the reaction was complete, the mixture was cooled to 0° C. and quenched with sat. NH₄Cl (aq.) solution until the pH was neutral (pH=7). The solution was diluted with 20 mL ethyl acetate and the layers were separated. The aqueous layer was further extracted with 2×10 mL ethyl acetate. The combined organic layers were then dried over Na₂SO₄ and concentrated in vacuo to afford a crude oil which was purified through flash chromatography (silica; ethyl acetate/hexanes, 0%-5%) to provide (R)-2-(2-(benzyloxy)ethyl)oxirane. ¹H NMR (400 MHz, CDCl₃) δ 7.42-7.25 (m, 5H), 4.56 (s, 2H), 3.65 (m, 2H), 3.10 (m, 1H), 2.80 (t, J=4.3 Hz, 1H), 2.55 (dd, J=2.6, 5.0 Hz, 1H), 1.94 (m, 1H), 1.81 (m, 1H). LC/MS M+1=m/z 178.8.

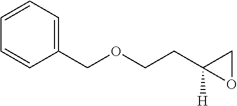 [SMILES: c1ccc(COCC[C@H]2CO2)cc1]

Preparation of (S)-2-(2-(benzyloxy)ethyl)oxirane: The title compound was prepared according to the procedure of (R)-2-(2-(benzyloxy)ethyl)oxirane using (S)-2-(oxiran-2-yl)ethan-1-ol. ¹H NMR (400 MHz, CDCl₃) δ 7.42-7.25 (m, 5H), 4.56 (s, 2H), 3.65 (m, 2H), 3.10 (m, 1H), 2.80 (t, J=4.3 Hz, 1H), 2.55 (dd, J=2.6, 5.0 Hz, 1H), 1.94 (m, 1H), 1.81 (m, 1H). LC/MS M+1=m/z 178.8.

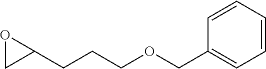 [SMILES: c1ccc(COCCCC2CO2)cc1]

Preparation of 2-(3-(benzyloxy)propyl)oxirane: To a cool (0° C.) solution of ((pent-4-en-1-yloxy)methyl)benzene (1.95 g, 11 mmol, 1 eq.) in dichloromethane (25 mL), 77% meta-Chloroperoxybenzoic acid (mCPBA) (4.2 g, 18.7 mmol, 1.7 eq.) was added in portions. The resulting mixture was allowed to stir at 0° C. for 30 minutes, then warmed to room temperature and stirred overnight. The mixture was then filtered through a plug of Celite, washed with dichloromethane and the solution was extracted with 1N NaOH (3×15 mL). The combined organic layers were dried over Na₂SO₄ and concentrated in vacuo to afford a crude oil of 2-(3-(benzyloxy)propyl)oxirane which was used in the next step without further purification. ¹H NMR (400 MHz, CDCl₃) δ 7.41-7.24 (m, 5H), 4.55 (s, 2H), 3.55 (m, 2H), 2.95 (m, 1H), 2.75 (t, J=4.4 Hz, 1H), 2.44 (dd, J=2.7, 5.1 Hz, 1H), 1.90-1.55 (m, 4H). LC/MS M+1=m/z 193.8.

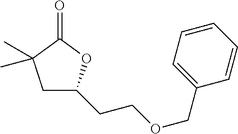 [SMILES: CC1(C)C[C@@H](CCOCc2ccccc2)OC1=O]

Preparation of (S)-5-(2-(benzyloxy)ethyl)-3,3-dimethyldihydrofuran-2(3H)-one: To solution of 1M lithium diisopropyl amide (LDA) (23.5 mL, 23.5 mmol, 2.3 eq.) at −78° C., N,N-dimethylisobutyramide (2.35 g, 20.5 mmol, 2 eq.) was added dropwise. The resulting mixture was allowed to stir at −78° C. for 30 minutes, then at 0° C. for fifteen minutes, then at room temperature for 5 minutes and finally cooled to 0° C. At 0° C., a solution of (R)-2-(2-(benzyloxy)ethyl)oxirane (1.82 g, 10.2 mmol, 1 eq.) in 5 mL dry tetrahydrofuran was added dropwise to the reaction mixture. After 20 minutes at 0° C., the reaction was warmed to room temperature and stirred until TLC showed complete consumption of epoxide. The reaction was then quenched with sat. NH₄Cl and the pH was adjusted to 7. The solution was diluted with 20 mL ethyl acetate and the layers were separated. The aqueous layer was further extracted with 2×10 mL ethyl acetate. The combined organic layers were then dried over Na₂SO₄ and concentrated in vacuo to afford a crude oil. This oil was then dissolved in 30 mL dichloromethane and 5 mL of trifluoroacetic acid was added dropwise. The solution stirred for 30 minutes before being diluted with 15 mL of dichloromethane and water. The layers were separated and the aqueous layer was extracted with 2×15 mL dichloromethane. The combined organic layers were then dried over Na₂SO₄ and concentrated in vacuo to afford a crude oil which was purified through flash chromatography (silica; ethyl acetate/hexanes, 0%-20%) to provide (S)-5-(2-(benzyloxy)ethyl)-3,3-dimethyldihydrofuran-2(3H)-one. ¹H NMR (400 MHz, CDCl₃) δ 7.41-7.26 (m, 5H), 4.66 (m, 1H), 4.54 (q, J=8.6 Hz, 2H), 3.71-3.58 (m, 2H), 2.18 (dd, J=5.9, 17.7 Hz, 1H), 2.06-1.89 (m, 2H), 1.79 (dd, J=10.0, 12.7 Hz, 1H), 1.28 (s, 3H), 1.27 (s, 3H). LC/MS M+1=m/z 248.8.

The following compounds were prepared according to the procedure of (S)-5-(2-(benzyloxy)ethyl)-3,3-dimethyldihydrofuran-2(3H)-one. One skilled in the art would know and understand which reagent to select in order to produce the following compounds.

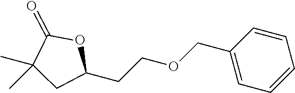 [SMILES: CC1(C)C[C@H](CCOCc2ccccc2)OC1=O]

(R)-5-(2-(benzyloxy)ethyl)-3,3-dimethyldihydrofuran-2(3H)-one: ¹H NMR (400 MHz, CDCl₃) δ 7.41-7.26 (m, 5H), 4.66 (m, 1H), 4.54 (q, J=8.6 Hz, 2H), 3.71-3.58 (m, 2H), 2.18 (dd, J=5.9, 17.7 Hz, 1H), 2.06-1.89 (m, 2H), 1.79 (dd, J=10.0, 12.7 Hz, 1H), 1.28 (s, 3H), 1.27 (s, 3H). LC/MS M+1=m/z 248.8.

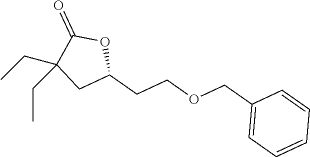 [SMILES: CCC1(CC)C[C@@H](CCOCc2ccccc2)OC1=O]

(S)-5-(2-(benzyloxy)ethyl)-3,3-diethyldihydrofuran-2(3H)-one: ¹H NMR (400 MHz, CDCl₃) δ 7.25-7.07 (m, 5H), 4.45 (m, 1H), 4.37 (q, J=5.7 Hz, 2H), 3.53-3.10 (m, 2H), 1.95 (dd, J=6.8, 13.2 Hz, 1H), 1.86-1.71 (m, 2H), 1.67 (dd, J=9.5, 13.3 Hz, 1H), 1.51-1.40 (m, 4H), 0.77 (dt, J=7.5, 20.4 Hz, 6H). LC/MS M+1=m/z 276.8.

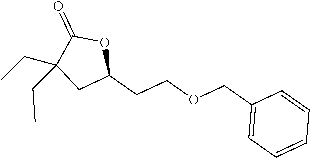 [SMILES: CCC1(CC)C[C@H](CCOCc2ccccc2)OC1=O]

(R)-5-(2-(benzyloxy)ethyl)-3,3-diethyldihydrofuran-2(3H)-one: ¹H NMR (400 MHz, CDCl₃) δ 7.25-7.07 (m, 5H), 4.45 (m, 1H), 4.37 (q, J=5.7 Hz, 2H), 3.53-3.10 (m, 2H), 1.95 (dd, J=6.8, 13.2 Hz, 1H), 1.86-1.71 (m, 2H), 1.67 (dd, J=9.5, 13.3 Hz, 1H), 1.51-1.40 (m, 4H), 0.77 (dt, J=7.5, 20.4 Hz, 6H). LC/MS M+1=m/z 276.8.

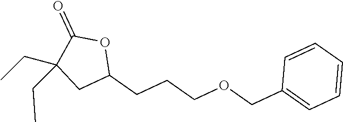 [SMILES: CCC1(CC)CC(CCCOCc2ccccc2)OC1=O]

5-(3-(benzyloxy)propyl)-3,3-diethyldihydrofuran-2(3H)-one: ¹H NMR (400 MHz, CDCl₃) δ 7.35-7.18 (m, 5H), 4.50 (s, 2H), 4.39 (m, 1H), 3.58-3.43 (m, 2H), 2.06 (dd, J=6.9, 13.1 Hz, 1H), 1.83-1.62 (m, 5H), 1.60-1.51 (m, 4H), 0.88 (dt, J=7.6, 20.2 Hz, 6H). LC/MS M+1=m/z 290.8.

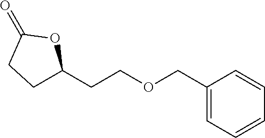 [SMILES: O=C1CC[C@H](CCOCc2ccccc2)O1]

(R)-5-(2-(benzyloxy)ethyl)dihydrofuran-2(3H)-one: ¹H NMR (400 MHz, CDCl₃) δ 7.40-7.20 (m, 5H), 4.69 (m, 1H), 4.55 (s, 2H), 3.70-3.62 (m, 2H), 2.54 (dd, J=6.9, 9.5 Hz, 2H), 2.35 (sex, J=6.2 Hz, 1H), 2.10-1.81 (m, 3H). LC/MS M+1=m/z 220.8.

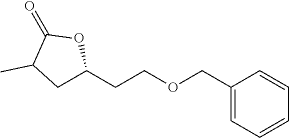

(R)-5-(2-(benzyloxy)ethyl)dihydrofuran-2(3H)-one: ¹H NMR (400 MHz, CDCl₃) δ 7.46-7.20 (m, 5H), 4.71 (m, 1H), 4.52 (m, 2H), 3.68-3.60 (m, 2H), 2.70 (sex, J=8.3 Hz, 1H), 2.35-1.98 (m, 4H), 0.87 (d, J=7.2 Hz, 3H). LC/MS M+1=m/z 234.8.

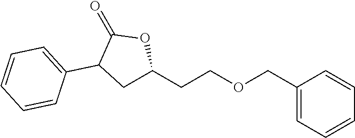

(5S)-5-(2-(benzyloxy)ethyl)-3-phenyldihydrofuran-2(3H)-one: ¹H NMR (400 MHz, CDCl₃) δ 7.47-7.17 (m, 10H), 4.88 (m, 0.5H), 4.74 (m, 0.5H), 4.55 (m, 2H), 3.96-3.86 (m, 1H), 3.76-3.63 (m, 2H), 3.00-2.85 (m, 0.5H), 2.84-2.73 (m, 0.5H) 2.60-2.41 (m, 1H), 2.20-1.93 (m, 2H). LC/MS M+1=m/z 296.8.

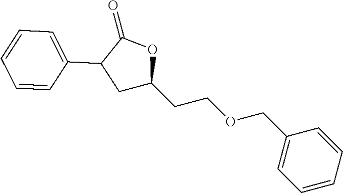

(5R)-5-(2-(benzyloxy)ethyl)-3-phenyldihydrofuran-2(3H)-one: ¹H NMR (400 MHz, CDCl₃) δ 7.47-7.17 (m, 10H), 4.88 (m, 0.5H), 4.74 (m, 0.5H), 4.55 (m, 2H), 3.96-3.86 (m, 1H), 3.76-3.63 (m, 2H), 3.00-2.85 (m, 0.5H), 2.84-2.73 (m, 0.5H) 2.60-2.41 (m, 1H), 2.20-1.93 (m, 2H). LC/MS M+1=m/z 296.8.

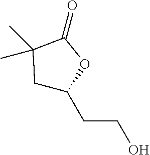

Preparation of (S)-5-(2-hydroxyethyl)-3,3-dimethyldihydrofuran-2(3H)-one: A solution of (S)-5-(2-(benzyloxy)ethyl)-3,3-dimethyldihydrofuran-2(3H)-one (1.75 g, 7.05 mmol, 1 eq.) in ethanol (65 mL) and 0.35 g of 10% palladium on carbon (20% wt) was stirred under atmosphere of hydrogen gas for 24 hours. The reaction was then filtered through a plug of celite, the celite was washed with excess ethanol, and the combined ethanol was concentrated in vacuo to afford a crude oil of (S)-5-(2-hydroxyethyl)-3,3-dimethyldihydrofuran-2(3H)-one which was used in the next step without further purification. ¹H NMR (400 MHz, CDCl₃) δ 4.70-4.60 (m, 1H), 3.90-3.78 (m, 2H), 2.22 (dd, J=5.9, 12.7, 1H), 1.98-1.87 (m, 2H), 1.80 (dd, J=5.9, 12.7, 1H), 1.28 (d, J=4.8, 6H).

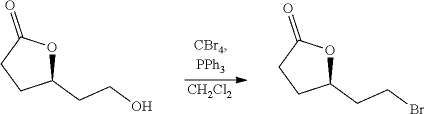

Preparation of (R)-5-(2-bromoethyl)dihydrofuran-2(3H)-one: To a solution of (R)-5-(2-hydroxyethyl)dihydrofuran-2(3H)-one (0.80 g, 6.15 mmol, 1 eq.) in methylene chloride (50 mL) was added triphenylphosphine (2.40 g, 9.22 mmol, 1.5 eq.). Carbon tetrabromide (5.07 g, 15.3 mmol, 2.5 eq.) was then added and the reaction was allowed to stir at room temperature overnight. The methylene chloride was removed under reduced pressure and the resulting residue was resuspended in ethyl acetate and filtered through a plug of Celite. The filtrate was dry loaded on Celite and purified through flash chromatography (silica; ethyl acetate/hexanes, 0%-35%) to provide (R)-5-(2-bromoethyl)dihydrofuran-2(3H)-one: ¹H NMR (400 MHz, CDCl₃) δ 4.69 (m, 1H), 3.52 (d, J=5.6 Hz, 1H), 3.50 (d, J=5.6 Hz, 1H), 2.58-2.50 (m, 2H), 2.39 (m, 1H), 2.30-2.18 (m, 1H), 2.17-2.05 (m, 1H), 1.88 (m, 1H). LC/MS M+1=m/z 192.8, 194.8.

The following compounds were prepared according to the procedures described herein. One skilled in the art would know and understand which reagent to select in order to produce the following compounds.

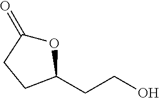

(R)-5-(2-hydroxyethyl)dihydrofuran-2(3H)-one: ¹H NMR (400 MHz, CDCl₃) δ 4.64 (m, 1H), 3.72 (t, J=5.8 Hz, 2H), 2.52-2.42 (m, 2H), 2.32 (sex, J=6.4 Hz, 1H), 1.93-1.76 (m, 3H). LC/MS M+1=m/z 130.8.

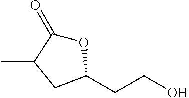

(5S)-5-(2-hydroxyethyl)-3-methyldihydrofuran-2(3H)-one: ¹H NMR (400 MHz, MeOD) δ 4.66 (m, 1H), 3.62 (m, 2H), 2.70 (m, 1H), 2.15 (m, 1H), 2.06-1.96 (m, 1H), 1.89-1.68 (m, 2H), 1.18 (d, J=7.5 Hz, 3H) LC/MS M+1=m/z 144.8.

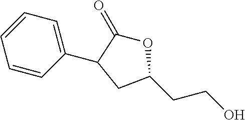

(5S)-5-(2-hydroxyethyl)-3-phenyldihydrofuran-2(3H)-one: ¹H NMR (400 MHz, MeOD) δ 7.34-7.02 (m, 5H), 4.75 (m, 0.5H), 4.61 (m, 0.5H), 3.92 (m, 0.5H), 3.69-3.57 (m, 2H), 2.69 (m, 0.5H), 2.49-2.31 (m, 1H), 2.04-1.71 (m, 3H). LC/MS M+1=m/z 206.8.

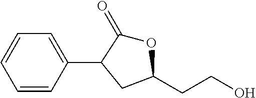

(5R)-5-(2-hydroxyethyl)-3-phenyldihydrofuran-2(3H)-one: ¹H NMR (400 MHz, MeOD) δ 7.34-7.02 (m, 5H), 4.75 (m, 0.5H), 4.61 (m, 0.5H), 3.92 (m, 0.5H), 3.69-3.57 (m, 2H), 2.69 (m, 0.5H), 2.49-2.31 (m, 1H), 2.04-1.71 (m, 3H). LC/MS M+1=m/z 206.8.

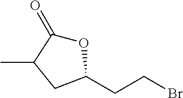

(5S)-5-(2-bromoethyl)-3-methyldihydrofuran-2(3H)-one: ¹H NMR (400 MHz, CDCl₃) δ 4.66 (m, 1H), 3.52-3.36 (m, 2H), 2.63 (m, 1H), 2.24-1.89 (m, 4H), 1.23 (d, J=7.3 Hz, 3H) LC/MS M+1=m/z 206.8, 208.8.

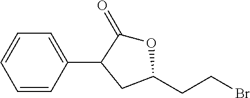

(5S)-5-(2-bromoethyl)-3-phenyldihydrofuran-2(3H)-one: ¹H NMR (400 MHz, CDCl₃) δ 7.45-7.21 (m, 5H), 4.88 (m, 0.5H), 4.75 (m, 0.5H), 3.94 (m, 1H), 3.58 (m, 2H), 2.85 (m, 0.5H), 2.61 (m, 0.5H), 2.49-2.29 (m, 1.5H), 2.29-2.15 (m, 1H), 2.14-2.03 (m, 0.5H). LC/MS M+1=m/z 268.8, 270.8.

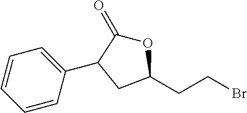

(5R)-5-(2-bromoethyl)-3-phenyldihydrofuran-2(3H)-one: ¹H NMR (400 MHz, CDCl₃) δ 7.45-7.21 (m, 5H), 4.88 (m, 0.5H), 4.75 (m, 0.5H), 3.94 (m, 1H), 3.58 (m, 2H), 2.85 (m, 0.5H), 2.61 (m, 0.5H), 2.49-2.29 (m, 1.5H), 2.29-2.15 (m, 1H), 2.14-2.03 (m, 0.5H). LC/MS M+1=m/z 268.8, 270.8.

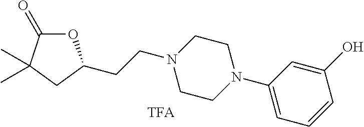 [SMILES: CC1(C)C[C@@H](CCN2CCN(c3cccc(O)c3)CC2)OC1=O]

(S)-dihydro-5-(2-(4-(3-hydroxyphenyl)piperazin-1-yl)ethyl)-3,3-dimethylfuran-2(3H)-one trifluoroacetate: ¹H NMR (400 MHz, CDCl₃) δ 7.16 (t, J=7.9, 1H), 6.50 (m, 3H), 4.50 (m, 1H), 4.01-2.86 (m, 10H), 2.26 (m, 2H), 2.06 (m, 1H), 1.80 (dd, J=10.0, 13.1, 1H), 1.30 (s, 3H), 1.28 (s, 3H) LC/MS M+1=m/z 319.1.

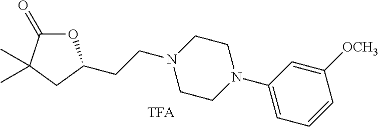 [SMILES: COc1cccc(N2CCN(CC[C@@H]3CC(C)(C)C(=O)O3)CC2)c1]

(S)-dihydro-5-(2-(4-(3-methoxyphenyl)piperazin-1-yl)ethyl)-3,3-dimethylfuran-2(3H)-one trifluoroacetate: ¹H NMR (400 MHz, CDCl₃) δ 7.22 (J=8.2, 1H), 6.54 (m, 2H), 6.47 (t, J=2.3, 1H), 4.49 (m, 1H), 3.94-2.80 (m, 13H), 2.26 (m, 2H), 2.07 (m, 1H), 1.79 (dd, J=10.0, 13.3, 1H), 1.28 (s, 3H), 1.26 (s, 3H) LC/MS [M+H]=m/z 333.1.

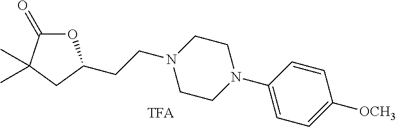 [SMILES: COc1ccc(N2CCN(CC[C@@H]3CC(C)(C)C(=O)O3)CC2)cc1]

(S)-dihydro-5-(2-(4-(4-methoxyphenyl)piperazin-1-yl)ethyl)-3,3-dimethylfuran-2(3H)-one trifluoroacetate: ¹H NMR (400 MHz, CDCl₃) δ 7.04 (d, J=8.8, 2H), 6.90 (d, J=8.8, 2H), 4.50 (m, 1H), 3.89-2.84 (m, 13H), 2.27 (m, 2H), 2.08 (m, 1H), 1.81 (dd, J=10.0, 13.0, 1H), 1.30 (s, 3H), 1.28 (s, 3H) LC/MS [M+H]=m/z 333.1.

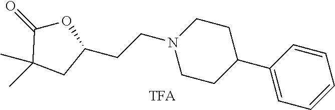 [SMILES: CC1(C)C[C@@H](CCN2CCC(c3ccccc3)CC2)OC1=O]

(S)-dihydro-3,3-dimethyl-5-(2-(4-phenylpiperidin-1-yl)ethyl)furan-2(3H)-one trifluoroacetate: ¹H NMR (400 MHz, MeOD) δ 7.32-7.13 (m, 5H), 4.58 (m, 1H), 3.11 (d, J=11.8, 2H), 2.59 (m, 3H), 2.28 (dd, J=6.2, 12.8, 1H), 2.18 (tt, J=3.5, 11.7, 2H), 1.99-1.72 (m, 7H), 1.27 (s, 3H), 1.1.26 (s, 3H), LC/MS [M+H]=m/z 302.1

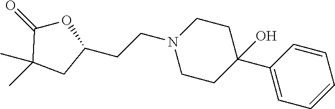 [SMILES: CC1(C)C[C@@H](CCN2CCC(O)(c3ccccc3)CC2)OC1=O]

(S)-dihydro-5-(2-(4-hydroxy-4-phenylpiperidin-1-yl)ethyl)-3,3-dimethylfuran-2(3H)-one: ¹H NMR (400 MHz, MeOD) δ 7.40 (dd, J=1.3, 8.5, 2H), 7.24 (t, J=7.4, 2H), 7.1 (tt, J=1.1, 7.4, 1H), 4.48 (m, 1H), 2.90 (d, J=11.3, 2H), 2.67 (m, 4H), 2.18 (dd, J=6.1, 12.6, 1H), 2.08 (td, J=4.5, 13.6, 2H), 1.89 (m, 2H), 1.72 (m, 3H), 1.16 (s, 3H), 1.15 (s, 3H), LC/MS [M+H]=m/z 302.1.

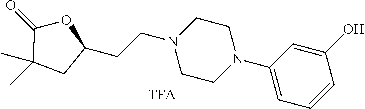 [SMILES: CC1(C)C[C@H](CCN2CCN(c3cccc(O)c3)CC2)OC1=O]

(R)-dihydro-5-(2-(4-(3-hydroxyphenyl)piperazin-1-yl)ethyl)-3,3-dimethylfuran-2(3H)-one trifluoroacetate: ¹H NMR (400 MHz, CDCl₃) δ 7.16 (t, J=7.9, 1H), 6.50 (m, 3H), 4.50 (m, 1H), 4.01-2.86 (m, 10H), 2.26 (m, 2H), 2.06 (m, 1H), 1.80 (dd, J=10.0, 13.1, 1H), 1.30 (s, 3H), 1.28 (s, 3H) LC/MS [M+H]=m/z 319.1.

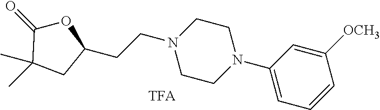 [SMILES: COc1cccc(N2CCN(CC[C@H]3CC(C)(C)C(=O)O3)CC2)c1]

(R)-dihydro-5-(2-(4-(3-methoxyphenyl)piperazin-1-yl)ethyl)-3,3-dimethylfuran-2(3H)-one trifluoroacetate: ¹H NMR (400 MHz, CDCl₃) δ 7.22 (J=8.2, 1H), 6.54 (m, 2H), 6.47 (t, J=2.3, 1H), 4.49 (m, 1H), 3.94-2.80 (m, 13H), 2.26 (m, 2H), 2.07 (m, 1H), 1.79 (dd, J=10.0, 13.3, 1H), 1.28 (s, 3H), 1.26 (s, 3H) LC/MS [M+H]=m/z 333.1.

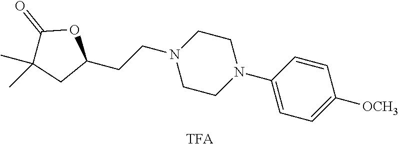 [SMILES: COc1ccc(N2CCN(CC[C@H]3CC(C)(C)C(=O)O3)CC2)cc1]

(R)-dihydro-5-(2-(4-(4-methoxyphenyl)piperazin-1-yl)ethyl)-3,3-dimethylfuran-2(3H)-one trifluoroacetate: ¹H NMR (400 MHz, CDCl₃) δ 7.04 (d, J=8.8, 2H), 6.90 (d, J=8.8, 2H), 4.50 (m, 1H), 3.89-2.84 (m, 13H), 2.27 (m, 2H), 2.08 (m, 1H), 1.81 (dd, J=10.0, 13.0, 1H), 1.30 (s, 3H), 1.28 (s, 3H) LC/MS [M+H]=m/z 333.1.

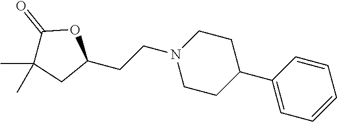 [SMILES: CC1(C)C[C@H](CCN2CCC(c3ccccc3)CC2)OC1=O]

(R)-dihydro-3,3-dimethyl-5-(2-(4-phenylpiperidin-1-yl)ethyl)furan-2(3H)-one: ¹H NMR (400 MHz, MeOD) δ 7.32-7.13 (m, 5H), 4.58 (m, 1H), 3.11 (d, J=11.8, 2H), 2.59 (m, 3H), 2.28 (dd, J=6.2, 12.8, 1H), 2.18 (tt, J=3.5, 11.7, 2H), 1.99-1.72 (m, 7H), 1.27 (s, 3H), 1.1.26 (s, 3H), LC/MS [M+H]=m/z 302.1.

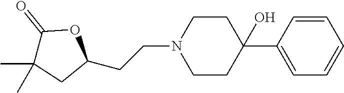 [SMILES: CC1(C)C[C@H](CCN2CCC(O)(c3ccccc3)CC2)OC1=O]

(R)-dihydro-5-(2-(4-hydroxy-4-phenylpiperidin-1-yl)ethyl)-3,3-dimethylfuran-2(3H)-one: ¹H NMR (400 MHz, MeOD) δ 7.40 (dd, J=1.3, 8.5, 2H), 7.24 (t, J=7.4, 2H), 7.1 (tt, J=1.1, 7.4, 1H), 4.48 (m, 1H), 2.90 (d, J=11.3, 2H), 2.67 (m, 4H), 2.18 (dd, J=6.1, 12.6, 1H), 2.08 (td, J=4.5, 13.6, 2H), 1.89 (m, 2H), 1.72 (m, 3H), 1.16 (s, 3H), 1.15 (s, 3H), LC/MS [M+H]=m/z 302.1.

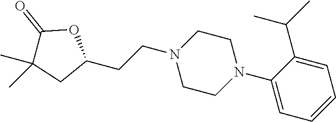 [SMILES: CC(C)c1ccccc1N1CCN(CC[C@@H]2CC(C)(C)C(=O)O2)CC1]

(S)-dihydro-5-(2-(4-(2-isopropylphenyl)piperazin-1-yl)ethyl)-3,3-dimethylfuran-2(3H)-one: ¹H NMR (400 MHz, CDCl₃) δ 7.30 (m, 1H), 7.20 (m, 2H), 7.14 (m, 1H), 4.52 (m, 1H), 3.75 (m, 2H), 3.35 (m, 6H), 3.08 (m, 4H), 2.32 (m, 2H), 2.08 (m, 1H), 1.81 (dd, J=10.3, 13.0, 1H), 1.30 (d, J=8.4, 6H), 1.22 (d, J=8.4, 6H) LC/MS [M+H]=m/z 345.2.

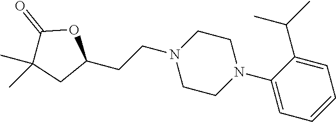 [SMILES: CC(C)c1ccccc1N1CCN(CC[C@H]2CC(C)(C)C(=O)O2)CC1]

(R)-dihydro-5-(2-(4-(2-isopropylphenyl)piperazin-1-yl)ethyl)-3,3-dimethylfuran-2(3H)-one: ¹H NMR (400 MHz, CDCl₃) δ 7.30 (m, 1H), 7.20 (m, 2H), 7.14 (m, 1H), 4.52 (m, 1H), 3.75 (m, 2H), 3.35 (m, 6H), 3.08 (m, 4H), 2.32 (m, 2H), 2.08 (m, 1H), 1.81 (dd, J=10.3, 13.0, 1H), 1.30 (d, J=8.4, 6H), 1.22 (d, J=8.4, 6H) LC/MS [M+H]=m/z 345.2.

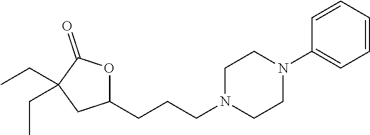 [SMILES: CCC1(CC)CC(CCCN2CCN(c3ccccc3)CC2)OC1=O]

3,3-diethyl-dihydro-5-(3-(4-phenylpiperazin-1-yl)propyl)furan-2(3H)-one: ¹H NMR (400 MHz, CDCl₃) δ 7.32 (td, J=1.1, 7.7, 2H), 7.00 (t, J=7.4, 1H), 6.95 (d, J=8.6, 1H), 4.40 (m, 1H), 3.70 (m, 4H), 3.35 (m, 2H), 3.16 (t, J=8.1, 2H), 3.01 (b, 2H), 2.16 (dd, J=6.7, 13.4, 1H), 2.01 (m, 2H), 1.81 (m, 2H), 1.64 (m, 5H), 0.94 (dt, J=7.5, 22.4, 6H) LC/MS [M+H]=m/z 345.2.

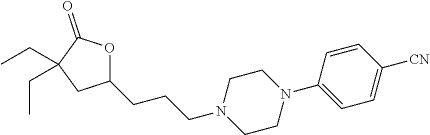 [SMILES: CCC1(CC)CC(CCCN2CCN(c3ccc(C#N)cc3)CC2)OC1=O]

4-(4-(3-(4,4-diethyl-tetrahydro-5-oxofuran-2-yl)propyl)piperazin-1-yl)benzonitrile: ¹H NMR (400 MHz, CDCl₃) δ 7.49 (d, J=8.9, 2H), 6.84 (d, J=8.9, 2H), 4.32 (m, 1H), 3.98-2.61 (m, 10H), 2.07 (dd, J=6.8, 13.5, 1H), 1.93 (m, 2H), 1.73 (m, 2H), 1.55 (m, 5H), 0.84 (dt, J=7.4, 22.4, 6H) LC/MS [M+H]=m/z 370.2.

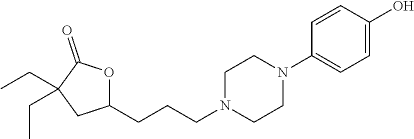 [SMILES: CCC1(CC)CC(CCCN2CCN(c3ccc(O)cc3)CC2)OC1=O]

3,3-diethyl-dihydro-5-(3-(4-(4-hydroxyphenyl)piperazin-1-yl)propyl)furan-2(3H)-one: ¹H NMR (400 MHz, CDCl₃) δ 6.73 (dd, J=8.5, 28.7, 4H), 4.31 (m, 1H), 3.78-2.76 (m, 10H), 2.07 (dd, J=6.7, 13.2, 1H), 1.87 (m, 2H), 1.70 (dd, J=9.5, 14.1, 2H), 1.53 (m, 5H), 0.83 (dt, J=7.4, 21.3, 6H) LC/MS [M+H]=m/z 361.2.

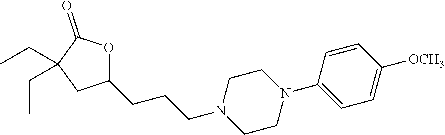 [SMILES: CCC1(CC)CC(CCCN2CCN(c3ccc(OC)cc3)CC2)OC1=O]

3,3-diethyl-dihydro-5-(3-(4-(4-methoxyphenyl)piperazin-1-yl)propyl)furan-2(3H)-one: ¹H NMR (400 MHz, CDCl₃) δ 7.03 (d, J=8.9, 2H), 6.89 (d, J=8.9, 2H), 4.40 (m, 1H), 3.90-2.96 (m, 13H), 2.15 (dd, J=6.8, 12.9, 1H), 1.99 (m, 2H), 1.80 (m, 2H), 1.64 (m, 5H), 0.92 (dt, J=7.6, 22.3, 6H) LC/MS [M+H]=m/z 375.2.

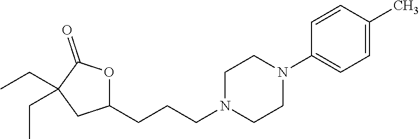 [SMILES: CCC1(CC)CC(CCCN2CCN(c3ccc(C)cc3)CC2)OC1=O]

3,3-diethyl-dihydro-5-(3-(4-p-tolylpiperazin-1-yl)propyl)furan-2(3H)-one: ¹H NMR (400 MHz, CDCl₃) δ 7.14 (d, J=8.3, 1H), 6.91 (d, J=8.4, 1H), 4.40 (m, 1H), 3.86-2.95 (m, 10H), 2.31 (s, 3H), 2.16 (dd, J=6.7, 13.0, 1H), 2.00 (m, 2H), 1.80 (m, 2H), 1.63 (m, 5H), 0.93 (dt, J=7.4, 22.7, 6H) LC/MS [M+H]=m/z 359.2.

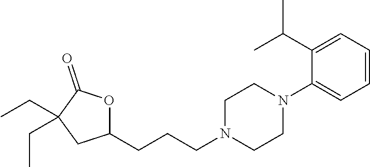 [SMILES: CCC1(CC)CC(CCCN2CCN(c3ccccc3C(C)C)CC2)OC1=O]

3,3-diethyl-dihydro-5-(3-(4-(2-isopropylphenyl)piperazin-1-yl)propyl)furan-2(3H)-one: ¹H NMR (400 MHz, CDCl₃) δ 7.30 (m, 1H), 7.27-7.11 (m, 3H), 4.41 (m, 1H), 3.77 (t, J=10.8, 2H), 3.64-3.14 (m, 9H), 2.11 (dd, J=6.8, 13.1, 1H), 1.93 (m, 2H), 1.77 (m, 2H), 1.57 (m, 5H), 1.16 (s, 3H), 1.14 (s, 3H), 0.84 (dt, J=7.4, 21.4, 6H) LC/MS [M+H]=m/z 387.2.

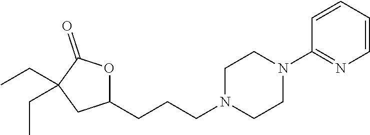 [SMILES: CCC1(CC)CC(CCCN2CCN(c3ccccn3)CC2)OC1=O]

3,3-diethyl-dihydro-5-(3-(4-(pyridin-2-yl)piperazin-1-yl)propyl)furan-2(3H)-one: ¹H NMR (400 MHz, CDCl₃) δ 8.15 (d, J=4.8, 1H), 7.97 (t, J=8.3, 1H), 7.11 (d, J=8.9, 1H), 7.03 (t, J=6.3, 1H), 4.40 (m, 1H), 4.10 (b, 4H), 3.48 (b, 4H), 3.19 (b, 2H), 2.14 (dd, J=6.8, 13.3, 1H), 1.98 (m, 2H), 1.78 (dd, J=9.3, 15.0, 2H), 1.62 (m, 5H), 0.90 (dt, J=7.5, 20.9, 6H) LC/MS [M+H]=m/z 346.2.

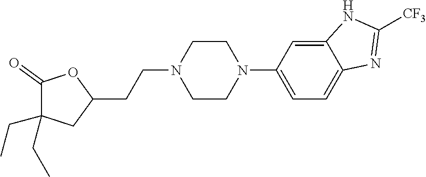 [SMILES: CCC1(CC)CC(CCN2CCN(c3ccc4nc(C(F)(F)F)[nH]c4c3)CC2)OC1=O]

3,3-diethyl-5-(2-(4-(2-(trifluoromethyl)-1H-benzo[d]imidazol-6-yl)piperazin-1-yl)ethyl)-dihydrofuran-2(3H)-one: ¹H NMR (400 MHz, CDCl₃) δ 7.59 (b, 1H), 7.02 (b, 1H), 6.85 (b, 1H), 4.43 (m, 1H), 3.11 (m, 4H), 2.61-2.39 (m, 6H), 2.08 (dd, J=6.9, 13.0, 1H), 1.79 (m, 3H), 1.56 (m, 4H), 0.85 (dt, J=7.6, 19.5, 6H) LC/MS [M+H]=m/z 439.2.

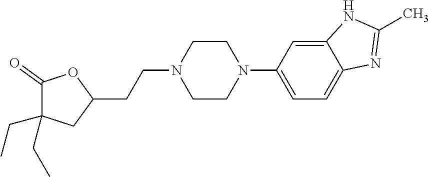 [SMILES: CCC1(CC)CC(CCN2CCN(c3ccc4nc(C)[nH]c4c3)CC2)OC1=O]

3,3-diethyl-dihydro-5-(2-(4-(2-methyl-1H-benzo[d]imidazol-6-yl)piperazin-1-yl)ethyl)furan-2(3H)-one: ¹H NMR (400 MHz, CDCl₃) δ 7.35 (d, J=8.8, 1H), 6.95 (s, 1H), 6.86 (dd, J=2.3, 8.7 1H), 4.42 (m, 1H), 3.10 (m, 4H), 2.61-2.42 (m, 9H), 2.07 (dd, J=6.6, 12.9, 1H), 1.80 (m, 3H), 1.56 (m, 4H), 0.86 (dt, J=7.3, 19.5, 6H) LC/MS [M+H]=m/z 385.1.

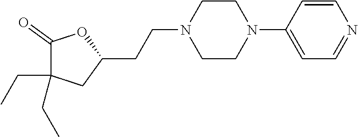 [SMILES: CCC1(CC)C[C@@H](CCN2CCN(c3ccncc3)CC2)OC1=O]

(S)-3,3-diethyl-dihydro-5-(2-(4-(pyridin-4-yl)piperazin-1-yl)ethyl)furan-2(3H)-one: ¹H NMR (400 MHz, CDCl₃) δ 8.25 (b, 2H), 6.67 (d, J=5.2 Hz, 2H), 4.49 (m, 1H), 3.35 (t, J=5.2 Hz, 4H), 2.55 (m, 6H), 2.15 (dd, J=6.7, 12.8 Hz, 1H), 1.86 (m, 3H), 1.63 (m, 4H), 0.93 (dt J=7.6, 19.2 Hz, 6H), LC/MS [M+H]=m/z 332.2.

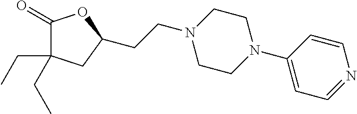 [SMILES: CCC1(CC)C[C@H](CCN2CCN(c3ccncc3)CC2)OC1=O]

(R)-3,3-diethyl-dihydro-5-(2-(4-(pyridin-4-yl)piperazin-1-yl)ethyl)furan-2(3H)-one: ¹H NMR (400 MHz, CDCl₃) δ 8.25 (b, 2H), 6.67 (d, J=5.2 Hz, 2H), 4.49 (m, 1H), 3.35 (t, J=5.2 Hz, 4H), 2.55 (m, 6H), 2.15 (dd, J=6.7, 12.8 Hz, 1H), 1.86 (m, 3H), 1.63 (m, 4H), 0.93 (dt J=7.6, 19.2 Hz, 6H), LC/MS [M+H]=m/z 332.2.

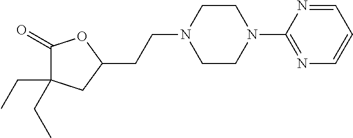 [SMILES: CCC1(CC)CC(CCN2CCN(c3ncccn3)CC2)OC1=O]

3,3-diethyl-dihydro-5-(2-(4-(pyrimidin-2-yl)piperazin-1-yl)ethyl)furan-2(3H)-one: ¹H NMR (400 MHz, CDCl₃) δ 8.28 (d, J=4.7 Hz, 2H), 6.46 (d, J=4.8 Hz, 1H), 4.47 (m, 1H), 3.81 (t, J=4.9 Hz, 4H), 2.52 (m, 6H), 2.13 (dd, J=6.8, 13 Hz, 1H), 1.86 (m, 3H), 1.61 (m, 4H), 0.91 (dt J=7.5, 20 Hz, 6H), LC/MS [M+H]=m/z 333.2.

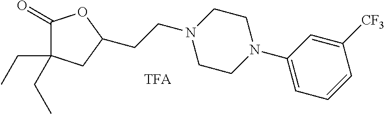 [SMILES: CCC1(CC)CC(CCN2CCN(c3cccc(C(F)(F)F)c3)CC2)OC1=O]

3,3-diethyl-5-(2-(4-(3-(trifluoromethyl)phenyl)piperazin-1-yl)ethyl)-dihydrofuran-2(3H)-one trifluoroacetate: ¹H NMR (400 MHz, CDCl₃) δ 7.42 (t, J=8.4 Hz, 1H), 7.23 (d, J=8.1 Hz, 1H), 7.10 (s, 1H), 7.09 (dd, J=2.2, 8.1 Hz, 1H), 4.46 (m, 1H), 3.76 (b, 4H), 3.33 (m, 4H), 3.06 (b, 2H), 2.28 (m, 1H), 2.22 (dd, J=6.7, 12.6 Hz, 1H), 2.05 (m, 1H), 1.86 (dd, J=9.4, 13.1 Hz, 1H), 1.63 (m, 4H) 0.92 (dt, J=7.4, 16.6 Hz, 6H) LC/MS [M+H]=m/z 399.2.

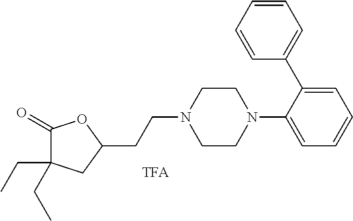 [SMILES: CCC1(CC)CC(CCN2CCN(c3ccccc3-c3ccccc3)CC2)OC1=O]

5-(2-(4-([1,1′-biphenyl]-2-yl)piperazin-1-yl)ethyl)-3,3-diethyldihydrofuran-2(3H)-one trifluoroacetate: ¹H NMR (400 MHz, CDCl₃) δ 7.54 (dt, 1.5, 7.0 Hz, 2H), 7.44 (tt, J=1.3, 7.7 Hz, 2H), 7.37 (dt, 1.3, 7.4 Hz, 1H), 7.33 (dd, 1.8, 7.5 Hz, 1H), 7.28 (m, 1H), 7.18 (td, J=0.9, 7.4 Hz, 1H), 7.05 (dd, J=0.9, 8.0 Hz, 1H), 4.41 (m, 1H), 3.52 (d, J=10.9 Hz, 2H), 3.26 (td, J=4.2, 11.9 Hz, 1H), 3.15 (m, 5H), 2.74 (td, J=3.8, 11.2 Hz, 1H), 2.65 (td, J=3.8, 11.2 Hz, 1H), 2.20 (m, 2H), 1.94 (m, 1H), 1.82 (dd, J=9.4, 13.1 Hz, 1H), 1.62 (m, 4H), 0.92 (dt, J=7.5, 19.8 Hz, 6H) LC/MS [M+H]=m/z 407.3.

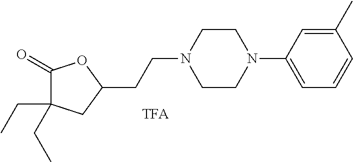 [SMILES: CCC1(CC)CC(CCN2CCN(c3cccc(C)c3)CC2)OC1=O]

3,3-diethyl-dihydro-5-(2-(4-m-tolylpiperazin-1-yl)ethyl)furan-2(3H)-one trifluoroacetate: ¹H NMR (400 MHz, CDCl₃) δ 7.20 (t, J=7.9 Hz, 1H), 6.82 (d, J=7.6 Hz, 1H), 6.76 (s, 1H), 6.75 (d, J=7.6 Hz, 1H), 4.45 (m, 1H), 4.15-2.70 (b, 10H), 2.33 (s, 3H), 2.28 (m, 1H), 2.21 (dd, J=6.7, 13.1 Hz, 1H), 2.04 (m, 1H), 1.85 (dd, J=9.3, 13.1 Hz, 1H), 1.63 (m, 4H) 0.92 (dt, J=7.4, 16.8 Hz, 6H) LC/MS [M+H]=m/z 345.2.

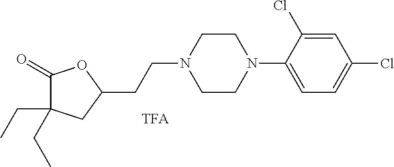 [SMILES: CCC1(CC)CC(CCN2CCN(c3ccc(Cl)cc3Cl)CC2)OC1=O]

5-(2-(4-(2,4-dichlorophenyl)piperazin-1-yl)ethyl)-3,3-diethyl-dihydrofuran-2(3H)-one trifluoroacetate: ¹H NMR (400 MHz, CDCl₃) δ 7.41 (d, J=2.4 Hz, 1H), 7.25 (dd, J=2.5, 8.5 Hz, 1H), 7.01 (d, J=8.5 Hz, 1H), 4.46 (m, 1H), 3.76 (t, J=11.5 Hz, 2H), 3.35 (m, 6H), 3.11 (m, 2H), 2.29 (m, 1H), 2.23 (dd, J=6.8, 13.0 Hz, 1H), 2.04 (m, 1H), 1.86 (dd, J=9.3, 13.1 Hz, 1H) 1.64 (q, J=7.3 Hz, 4H), 0.93 (dt, J=7.4, 18.2 Hz, 6H) LC/MS [M+H]=m/z 399.10.

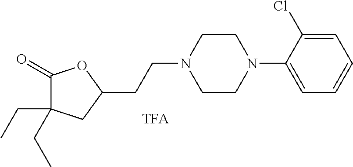 [SMILES: CCC1(CC)CC(CCN2CCN(c3ccccc3Cl)CC2)OC1=O]

5-(2-(4-(2-chlorophenyl)piperazin-1-yl)ethyl)-3,3-diethyldihydrofuran-2(3H)-one trifluoroacetate: ¹H NMR (400 MHz, CDCl₃) δ 7.38 (dd, J=1.6, 8.2 Hz, 1H), 7.25 (dt, J=1.4, 8.1 Hz, 1H), 7.06 (m, 2H), 4.46 (m, 1H), 3.74 (t, J=10.3 Hz, 2H), 3.45 (m, 2H), 3.39-3.20 (m, 4H), 3.12 (m, 2H), 2.28 (m, 1H), 2.21 (dd, J=6.9, 12.5 Hz, 1H), 2.05 (m, 1H) 1.85 (dd, J=9.2, 13.6 Hz, 1H), 1.62 (m, 4H), 0.92 (dt, J=7.5, 17.2 Hz, 6H) LC/MS [M+H]=m/z 365.20.

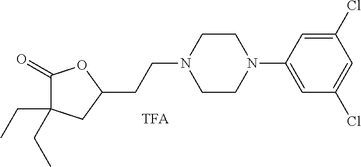 [SMILES: CCC1(CC)CC(CCN2CCN(c3cc(Cl)cc(Cl)c3)CC2)OC1=O]

5-(2-(4-(3,5-dichlorophenyl)piperazin-1-yl)ethyl)-3,3-diethyldihydrofuran-2(3H)-one trifluoroacetate: ¹H NMR (400 MHz, CDCl₃) δ 6.93 (t, J=1.6 Hz, 1H), 6.77 (d, J=1.7 Hz, 2H), 4.44 (m, 1H), 4.00-2.60 (b, 10H), 2.27 (m, 1H), 2.21 (dd, J=6.8, 13.1 Hz, 1H), 2.03 (m, 1H), 1.89 (dd, J=9.3, 13.4, 1H), 1.62 (m, 4H), 0.92 (dt, J=7.4, 16.6 Hz, 6H) LC/MS [M+H]=m/z 399.10.

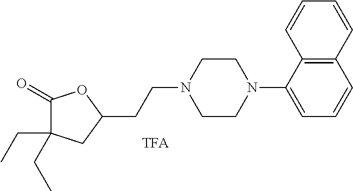 [SMILES: CCC1(CC)CC(CCN2CCN(c3cccc4ccccc34)CC2)OC1=O]

3,3-diethyl-5-(2-(4-(naphthalen-1-yl)piperazin-1-yl)ethyl)dihydrofuran-2(3H)-one trifluoro acetate: ¹H NMR (400 MHz, CDCl₃) δ 8.06 (m, 1H), 7.87 (m, 1H), 7.66 (d, J=8.3 Hz, 1H), 7.52 (m, 2H) 7.44 (t, J=8.1 Hz, 1H), 7.16 (dd, J=0.8, 7.5 Hz, 1H), 4.50 (m, 1H), 3.81 (t, J=10.1 Hz, 2H), 3.56-3.00 (m, 8H), 2.36 (m, 1H), 2.25 (dd, J=6.8, 13.4 Hz, 1H), 2.08 (m, 1H), 1.88 (dd, J=9.2, 13.3 Hz, 1H), 1.65 (q, J=7.0 Hz, 4H), 0.95 (dt, J=7.5, 17.8 Hz, 6H) LC/MS [M+H]=m/z 381.20.

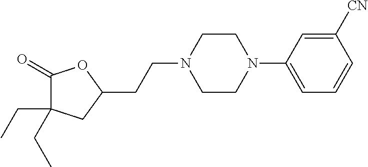 [SMILES: CCC1(CC)CC(CCN2CCN(c3cccc(C#N)c3)CC2)OC1=O]

3-(4-(2-(4,4-diethyl-5-oxotetrahydrofuran-2-yl)ethyl)piperazin-1-yl)benzonitrile: ¹H NMR (400 MHz, CDCl₃) δ 7.30 (m, 1H), 7.09 (m, 3H), 4.49 (m, 1H), 3.22 (t, J=5.0 Hz, 4H), 2.61 (t, J=5.2 Hz, 4H), 2.56 (m, 2H) 2.14 (dd, J=6.7, 13.1 Hz, 1H), 1.85 (m, 3H), 1.62 (m, 4H) 0.92 (dt, J=7.7, 19.0 Hz, 6H) LC/MS [M+H]=m/z 356.20.

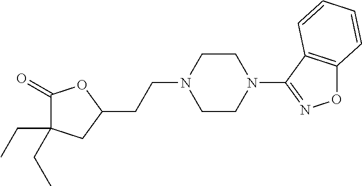 [SMILES: CCC1(CC)CC(CCN2CCN(c3noc4ccccc34)CC2)OC1=O]

5-(2-(4-(benzo[d]isoxazol-3-yl)piperazin-1-yl)ethyl)-3,3-diethyldihydrofuran-2(3H)-one: ¹H NMR (400 MHz, CDCl₃) δ 7.36 (dd, J=0.7, 7.7 Hz, 1H), 7.25 (d, J=7.8 Hz, 1H), 7.16 (td, J=1.0, 7.8 Hz, 1H), 7.02 (td, J=1.1, 7.7 Hz, 1H), 4.50 (m, 1H), 3.72 (t, J=5.3 Hz, 4H), 2.57 (m, 6H), 2.14 (dd, J=6.9, 13.1 Hz, 1H), 1.85 (m, 3H), 1.63 (m, 4H) 0.93 (dt, J=7.4, 20.0 Hz, 6H) LC/MS [M+H]=m/z 372.20

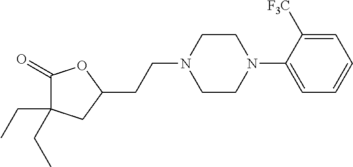 [SMILES: CCC1(CC)CC(CCN2CCN(c3ccccc3C(F)(F)F)CC2)OC1=O]

3,3-diethyl-5-(2-(4-(2-(trifluoromethyl)phenyl)piperazin-1-yl)ethyl)dihydrofuran-2(3H)-one: ¹H NMR (400 MHz, CDCl₃) δ 7.62 (d, J=8.1 Hz, 1H), 7.51 (t, J=7.7 Hz, 1H), 7.38 (d, J=8.0 Hz, 1H), 7.22 (t, J=7.7 Hz, 1H), 4.50 (m, 1H), 2.97 (t, J=4.6 Hz, 4H), 2.72-2.45 (m, 6H), 2.15 (dd, J=6.8, 13.1 Hz, 1H), 1.88 (m, 3H), 1.64 (m, 4H) 0.94 (dt, J=7.5, 21.5 Hz, 6H) LC/MS [M+H]=m/z 399.20.

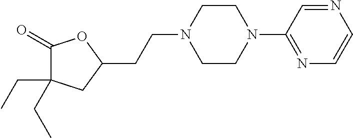 [SMILES: CCC1(CC)CC(CCN2CCN(c3cnccn3)CC2)OC1=O]

3,3-diethyl-5-(2-(4-(pyrazin-2-yl)piperazin-1-yl)ethyl)dihydrofuran-2(3H)-one: ¹H NMR (400 MHz, CDCl₃) δ 8.12 (d, J=1.4 Hz, 1H), 8.04 (dd, J=1.6, 2.6 Hz, 1H), 7.83 (d, J=2.6 Hz, 1H), 4.48 (m, 1H), 3.59 (t, J=5.1 Hz, 4H), 2.55 (m, 6H), 2.13 (dd, J=6.7, 12.3 Hz, 1H), 1.85 (m, 3H), 1.61 (m, 4H) 0.91 (dt, J=7.5, 19.4 Hz, 6H) LC/MS [M+H]=m/z 333.20.

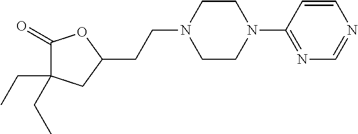 [SMILES: CCC1(CC)CC(CCN2CCN(c3ccncn3)CC2)OC1=O]

3,3-diethyl-5-(2-(4-(pyrazin-2-yl)piperazin-1-yl)ethyl)dihydrofuran-2(3H)-one: ¹H NMR (400 MHz, CDCl₃) δ 8.52 (s, 1H), 8.13 (d, J=6.2 Hz, 1H), 6.44 (dd, J=1.0, 6.3 Hz, 1H), 4.42 (m, 1H), 3.62 (b, 4H), 2.52 (m, 6H), 2.08 (dd, J=7.0, 13.1 Hz, 1H), 1.82 (q, J=6.8 Hz, 2H), 1.77 (dd, J=9.4, 13.1 Hz, 1H), 1.56 (m, 4H) 0.86 (dt, J=7.5, 20.0 Hz, 6H) LC/MS [M+H]=m/z 333.20.

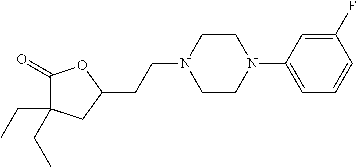 [SMILES: CCC1(CC)CC(CCN2CCN(c3cccc(F)c3)CC2)OC1=O]

3,3-diethyl-5-(2-(4-(3-fluorophenyl)piperazin-1-yl)ethyl)dihydrofuran-2(3H)-one: ¹H NMR (400 MHz, CDCl₃) δ 7.18 (q, J=7.0 Hz, 1H), 6.67 (dd, J=2.2, 8.4 Hz, 1H), 6.58 (dt, J=2.3, 12.4 Hz, 1H), 6.52 (td, J=2.4, 8.1 Hz, 1H), 4.49 (m, 1H), 3.20 (t, J=5.0 Hz, 4H), 2.60 (t, J=4.8 Hz, 4H), 2.55 (m, 2H), 2.14 (dd, J=6.7, 13.1 Hz, 1H), 1.86 (m, 3H), 1.63 (m, 4H) 0.93 (dt, J=7.5, 19.1 Hz, 6H) LC/MS [M+H]=m/z 349.20.

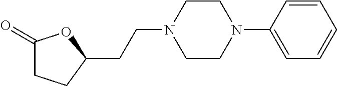 [SMILES: O=C1CC[C@H](CCN2CCN(c3ccccc3)CC2)O1]

(R)-5-(2-(4-phenylpiperazin-1-yl)ethyl)dihydrofuran-2(3H)-one: ¹H NMR (400 MHz, CDCl₃) δ 7.28 (m, 2H), 6.94 (dd, J=1.1, 8.9 Hz, 2H), 6.87 (tt, J=0.9, 7.2 Hz, 1H), 4.63 (m, 1H), 3.22 (t, J=5.1 Hz, 4H), 2.63 (t, J=4.7 Hz, 4H), 2.57 (m, 4H), 2.38 (m, 1H), 2.05-1.76 (m, 3H) LC/MS [M+H]=m/z 275.10.

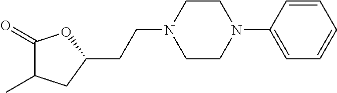

(5S)-3-methyl-5-(2-(4-phenylpiperazin-1-yl)ethyl)dihydrofuran-2(3H)-one: ¹H NMR (400 MHz, CDCl₃) δ 7.28 (t, J=7.9 Hz, 2H), 6.94 (d, J=8.1 Hz, 2H), 6.87 (t, J=7.2 Hz, 1H), 4.65 (m, 1H), 3.22 (t, J=5.1 Hz, 4H), 2.73 (sex, J=7.4 Hz, 1H), 2.63 (t, J=4.7 Hz, 4H), 2.57 (m, 2H), 2.18 (m, 1H), 2.06 (m, 1H), 1.88 (m, 2H), 1.31 (d, J=7.4 Hz, 3H) LC/MS [M+H]=m/z 289.10.

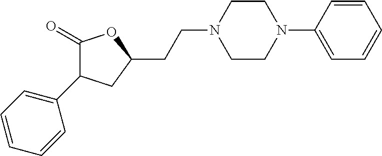

(5R)-3-phenyl-5-(2-(4-phenylpiperazin-1-yl)ethyl)dihydrofuran-2(3H)-one: ¹H NMR (400 MHz, CDCl₃) δ 7.28 (m, 2H), 7.24-7.12 (m, 5H), 6.85 (d, J=8.0 Hz, 2H), 6.78 (t, J=7.3 Hz, 1H), 4.68 (m, 0.4H), 4.54 (m, 0.6H), 3.88-3.74 (m, 1H), 3.12 (t, J=5.1 Hz, 4H), 2.70 (m, 0.6H), 2.61-2.41 (m, 6.2H), 2.40-2.30 (m, 0.4H), 2.11-1.68 (m, 2.8H) LC/MS [M+H]=m/z 351.2.

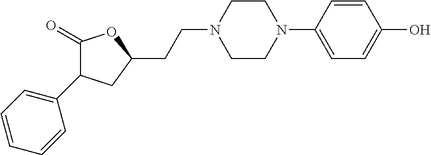

(5R)-5-(2-(4-(4-hydroxyphenyl)piperazin-1-yl)ethyl)-3-phenyldihydrofuran-2(3H)-one: ¹H NMR (400 MHz, CDCl₃) δ 7.27 (m, 2H), 7.19 (m, 3H), 6.73 (d, J=9.0 Hz, 2H), 6.62 (d, J=9.0 Hz, 2H), 4.62 (m, 0.4H), 4.48 (m, 0.6H), 3.78 (m, 1H), 2.99 (t, J=4.6 Hz, 4H), 2.67 (m, 0.6H), 2.61-2.37 (m, 6.2H), 2.31 (m, 0.4H), 2.08-1.65 (m, 2.8H) LC/MS [M+H]=m/z 367.1.

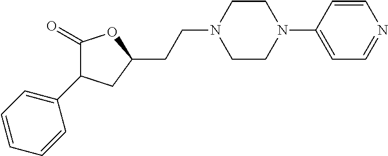

(5R)-3-phenyl-5-(2-(4-(pyridin-4-yl)piperazin-1-yl)ethyl)dihydrofuran-2(3H)-one: ¹H NMR (400 MHz, CDCl₃) δ 8.19 (d, J=6.4 Hz, 2H), 7.29 (m, 2H), 7.21 (m, 3H), 6.58 (d, J=6.6 Hz, 2H), 4.69 (m, 0.35H), 4.56 (m, 0.65H), 3.83 (m, 1H), 3.26 (t, J=5.1 Hz, 4H), 2.73 (m, 0.6H), 2.59-2.42 (m, 6.2H), 2.37 (m, 0.4H), 2.12-1.71 (m, 2.8H) LC/MS [M+H]=m/z 352.1.

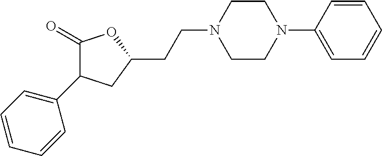

(5S)-3-phenyl-5-(2-(4-phenylpiperazin-1-yl)ethyl)dihydrofuran-2(3H)-one: ¹H NMR (400 MHz, CDCl₃) δ 7.28 (m, 2H), 7.24-7.12 (m, 5H), 6.85 (d, J=8.0 Hz, 2H), 6.78 (t, J=7.3 Hz, 1H), 4.68 (m, 0.4H), 4.54 (m, 0.6H), 3.88-3.74 (m, 1H), 3.12 (t, J=5.1 Hz, 4H), 2.70 (m, 0.6H), 2.61-2.41 (m, 6.2H), 2.40-2.30 (m, 0.4H), 2.11-1.68 (m, 2.8H) LC/MS [M+H]=m/z 351.2.

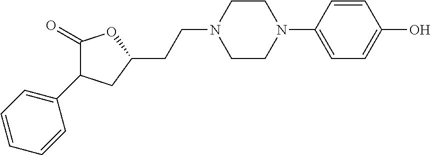

(5S)-5-(2-(4-(4-hydroxyphenyl)piperazin-1-yl)ethyl)-3-phenyldihydrofuran-2(3H)-one: ¹H NMR (400 MHz, CDCl₃) δ 7.27 (m, 2H), 7.19 (m, 3H), 6.73 (d, J=9.0 Hz, 2H), 6.62 (d, J=9.0 Hz, 2H), 4.62 (m, 0.4H), 4.48 (m, 0.6H), 3.78 (m, 1H), 2.99 (t, J=4.6 Hz, 4H), 2.67 (m, 0.6H), 2.61-2.37 (m, 6.2H), 2.31 (m, 0.4H), 2.08-1.65 (m, 2.8H) LC/MS [M+H]=m/z 367.1.

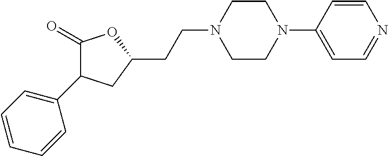

(5S)-3-phenyl-5-(2-(4-(pyridin-4-yl)piperazin-1-yl)ethyl)dihydrofuran-2(3H)-one: ¹H NMR (400 MHz, CDCl₃) δ 8.19 (d, J=6.4 Hz, 2H), 7.29 (m, 2H), 7.21 (m, 3H), 6.58 (d, J=6.6 Hz, 2H), 4.69 (m, 0.35H), 4.56 (m, 0.65H), 3.83 (m, 1H), 3.26 (t, J=5.1 Hz, 4H), 2.73 (m, 0.6H), 2.59-2.42 (m, 6.2H), 2.37 (m, 0.4H), 2.12-1.71 (m, 2.8H) LC/MS [M+H]=m/z 352.1.

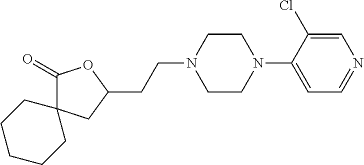 [SMILES: O=C1OC(CCN2CCN(c3ccncc3Cl)CC2)CC12CCCCC2]

3-(2-(4-(3-chloropyridin-4-yl)piperazin-1-yl)ethyl)-2-oxaspiro[4.5]decan-1-one: ¹H NMR (400 MHz, CDCl₃) 1H NMR (400 MHz, CDCl₃) δ 8.33 (s, 1H), 8.23 (d, J=5.4 Hz, 1H), 6.75 (d, J=5.6 Hz, 1H), 4.44 (m, 1H), 3.17 (t, J=4.7 Hz, 4H), 2.67-2.43 (m, 6H), 2.33 (dd, J=6.2, 12.7 Hz, 1H), 1.90-1.47 (m, 9H), 1.43 (m, 1H), 1.38-1.09 (m, 3H) LC/MS [M+H]=m/z 377.9.

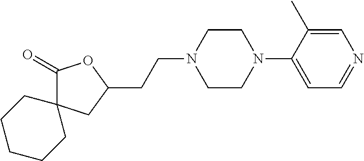 [SMILES: Cc1cnccc1N1CCN(CCC2CC3(CCCCC3)C(=O)O2)CC1]

3-(2-(4-(3-methylpyridin-4-yl)piperazin-1-yl)ethyl)-2-oxaspiro[4.5]decan-1-one: ¹H NMR (400 MHz, CDCl₃) 1H NMR (400 MHz, CDCl₃) δ 8.20 (m, 2H), 6.72 (d, J=5.3 Hz, 1H), 4.44 (m, 1H), 2.99 (t, J=4.8 Hz, 4H), 2.65-2.44 (m, 6H), 2.33 (dd, J=6.3, 12.6 Hz, 1H), 2.17 (s, 3H), 1.90-1.47 (m, 9H), 1.43 (m, 1H), 1.38-1.11 (m, 3H) LC/MS [M+H]=m/z 357.9.

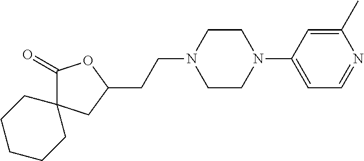 [SMILES: Cc1cc(N2CCN(CCC3CC4(CCCCC4)C(=O)O3)CC2)ccn1]

3-(2-(4-(2-methylpyridin-4-yl)piperazin-1-yl)ethyl)-2-oxaspiro[4.5]decan-1-one: ¹H NMR (400 MHz, CDCl₃) δ 8.00 (d, J=7.1 Hz, 1H), 6.47 (m, 2H), 4.43 (m, 1H), 3.29 (t, J=5.0 Hz, 4H), 2.57-2.43 (m, 6H), 2.41 (s, 3H), 2.33 (dd, J=6.4, 12.8 Hz, 1H), 1.88-1.47 (m, 9H), 1.42 (m, 1H), 1.38-1.09 (m, 3H) LC/MS [M+H]=m/z 358.0.

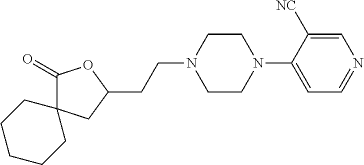 [SMILES: N#Cc1cnccc1N1CCN(CCC2CC3(CCCCC3)C(=O)O2)CC1]

4-(4-(2-(1-oxo-2-oxaspiro[4.5]decan-3-yl)ethyl)piperazin-1-yl)nicotinonitrile: ¹H NMR (400 MHz, CDCl₃) δ 8.56 (s, 1H), 8.39 (d, J=6.0 Hz, 1H), 6.73 (d, J=6.1 Hz, 1H), 4.52 (m, 1H), 3.57 (t, J=4.9 Hz, 4H), 2.75-2.51 (m, 6H), 2.41 (dd, J=6.2, 12.9 Hz, 1H), 2.01-1.55 (m, 9H), 1.50 (m, 1H), 1.47-1.13 (m, 3H) LC/MS [M+H]=m/z 368.9.

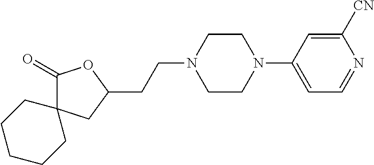 [SMILES: N#Cc1cc(N2CCN(CCC3CC4(CCCCC4)C(=O)O3)CC2)ccn1]

4-(4-(2-(1-oxo-2-oxaspiro[4.5]decan-3-yl)ethyl)piperazin-1-yl)picolinonitrile: ¹H NMR (400 MHz, CDCl₃) δ 8.22 (d, J=6.1 Hz, 1H), 6.96 (d, J=2.6 Hz, 1H), 6.69 (dd, J=2.7, 6.0 Hz, 1H), 4.44 (m, 1H), 3.32 (b, 4H), 2.68-2.40 (b, 6H), 2.33 (dd, J=6.3, 12.8 Hz, 1H), 1.91-1.47 (m, 9H), 1.42 (m, 1H), 1.38-1.07 (m, 3H) LC/MS [M+H]=m/z 368.9.

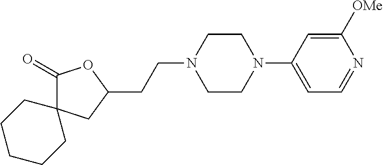 [SMILES: COc1cc(N2CCN(CCC3CC4(CCCCC4)C(=O)O3)CC2)ccn1]

3-(2-(4-(2-methoxypyridin-4-yl)piperazin-1-yl)ethyl)-2-oxaspiro[4.5]decan-1-one: ¹H NMR (400 MHz, CDCl₃) δ 7.90 (d, J=6.1 Hz, 1H), 6.40 (dd, J=2.3, 6.2 Hz, 1H), 6.06 (d, J=2.2 Hz, 1H), 4.51 (m, 1H), 3.90 (s, 3H), 3.31 (t, J=5.1 Hz, 4H), 2.67-2.49 (m, 6H), 2.41 (dd, J=6.2, 12.9 Hz, 1H), 1.98-1.55 (m, 9H), 1.50 (m, 1H), 1.46-1.16 (m, 3H) LC/MS [M+H]=m/z 373.9.

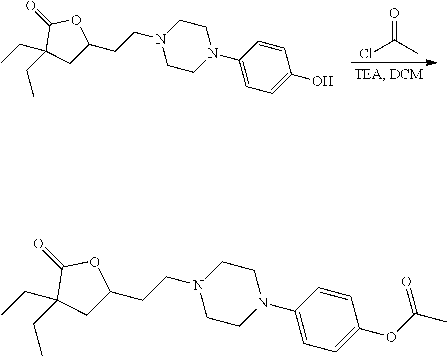

To a solution of 3,3-diethyl-5-(2-(4-(4-hydroxyphenyl)piperazin-1-yl)ethyl)dihydrofuran-2(3H)-one (50 mg, 0.144 mmol, 1 eq.) in dichloromethane (1.5 mL) was added trimethylamine (17.5 mg, 0.173 mmol, 1.2 eq.) and then the reaction was cooled to 0° C. Acetyl chloride (34 mg, 0.432 mmol, 3 eq.) was added dropwise and the resulting solution was allowed to stir at room temperature overnight. All volatiles were removed under reduced pressure and the remaining crude oil was purified through flash chromatography (silica; methanol/DCM, 0%-5%) to provide 4-(4-(2-(4,4-diethyl-5-oxotetrahydrofuran-2-yl)ethyl)piperazin-1-yl)phenyl acetate.

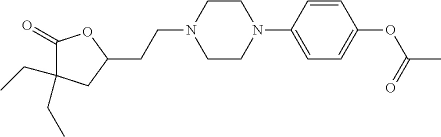 [SMILES: CCC1(CC)CC(CCN2CCN(c3ccc(OC(C)=O)cc3)CC2)OC1=O]

4-(4-(2-(4,4-diethyl-tetrahydro-5-oxofuran-2-yl)ethyl)piperazin-1-yl)phenyl acetate: ¹H NMR (400 MHz, CDCl₃) δ 6.98 (dt, J=3.3, 9.2 Hz, 2H), 6.91 (dt, J=3.3, 9.2 Hz, 2H), 4.50 (m, 1H), 3.18 (t, J=5.0, 4H), 2.62 (t, J=5 Hz, 4H), 2.57 (m, 2H), 2.28 (s, 3H), 2.15 (dd, J=6.9, 13.1 Hz, 1H), 1.88 (m, 3H), 1.64 (m, 4H), 0.94 (dt, J=7.4, 19.7, 6H) LC/MS [M+H]=m/z 388.51.

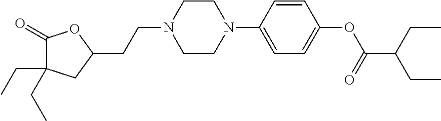 [SMILES: CCC(CC)C(=O)Oc1ccc(N2CCN(CCC3CC(CC)(CC)C(=O)O3)CC2)cc1]

4-(4-(2-(4,4-diethyl-tetrahydro-5-oxofuran-2-yl)ethyl)piperazin-1-yl)phenyl 2-ethylbutanoate: ¹H NMR (400 MHz, CDCl₃) δ 6.89 (dt, J=2.5, 9.2 Hz, 2H), 6.83 (dt, J=2.5, 9.2 Hz, 2H), 4.41 (m, 1H), 3.09 (t, J=4.6, 4H), 2.50 (m, 6H), 2.35 (m, 1H), 2.07 (dd, J=6.8, 13.1 Hz, 1H), 1.7 (m, 5H), 1.56 (m, 6H), 0.93 (t, J=7.5 Hz, 6H), 0.86 (dt, J=7.3, 19.8, 6H) LC/MS [M+H]=m/z 444.62.

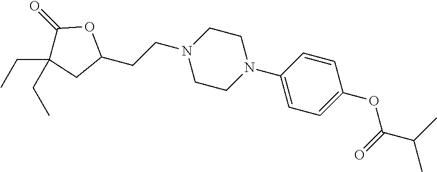 [SMILES: CCC1(CC)CC(CCN2CCN(c3ccc(OC(=O)C(C)C)cc3)CC2)OC1=O]

4-(4-(2-(4,4-diethyl-tetrahydro-5-oxofuran-2-yl)ethyl)piperazin-1-yl)phenyl isobutyrate: ¹H NMR (400 MHz, CDCl₃) δ 6.88 (dt, J=2.9, 9.2 Hz, 2H), 6.82 (dt, J=2.9, 9.2 Hz, 2H), 4.41 (m, 1H), 3.09 (t, J=4.8, 4H), 2.69 (sep, J=6.9 Hz, 1H), 2.53 (t, J=4.9 Hz, 4H), 2.49 (m, 2H), 2.07 (dd, J=6.8, 13.2 Hz, 1H), 1.80 (m, 3H), 1.53 (m, 4H), 1.22 (d, J=6.9 Hz, 6H), 0.85 (dt, J=7.4, 19.6 Hz, 6H) LC/MS [M+H]=m/z 416.56.

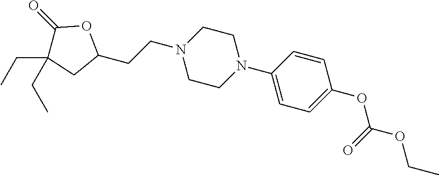 [SMILES: CCOC(=O)Oc1ccc(N2CCN(CCC3CC(CC)(CC)C(=O)O3)CC2)cc1]

4-(4-(2-(4,4-diethyl-5-oxotetrahydrofuran-2-yl)ethyl)piperazin-1-yl)phenyl ethyl carbonate: ¹H NMR (400 MHz, CDCl₃) δ 7.07 (d, J=9.2 Hz, 2H), 6.91 (d, J=9.0 Hz, 2H), 4.49 (m, 1H), 4.30 (q, J=7.2 Hz, 2H), 3.18 (t, J=4.9 Hz, 4H), 2.63 (t, J=4.9 Hz, 4H), 2.57 (m, 2H), 2.15 (dd, J=6.8, 13.2 Hz, 1H), 1.88 (m, 3H), 1.64 (m, 4H), 1.38 (t, J=7.1 Hz, 3H), 0.94 (dt, J=7.3, 19.5 Hz, 6H) LC/MS [M+H]=m/z 419.20.

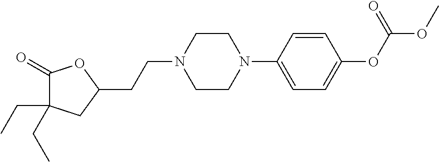 [SMILES: CCC1(CC)CC(CCN2CCN(c3ccc(OC(=O)OC)cc3)CC2)OC1=O]

4-(4-(2-(4,4-diethyl-5-oxotetrahydrofuran-2-yl)ethyl)piperazin-1-yl)phenyl methyl carbonate: ¹H NMR (400 MHz, CDCl₃) δ 7.06 (d, J=9.1 Hz, 2H), 6.90 (d, J=9.1 Hz, 2H), 4.49 (m, 1H), 3.89 (s, 3H), 3.18 (t, J=5.2 Hz, 4H), 2.63 (t, J=4.6 Hz, 4H), 2.58 (m, 2H), 2.15 (dd, J=6.4, 12.7 Hz, 1H), 1.88 (m, 3H), 1.64 (m, 4H) 0.94 (dt, J=7.3, 19.4 Hz, 6H) LC/MS [M+H]=m/z 405.20.

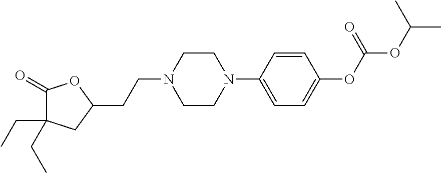 [SMILES: CCC1(CC)CC(CCN2CCN(c3ccc(OC(=O)OC(C)C)cc3)CC2)OC1=O]

4-(4-(2-(4,4-diethyl-5-oxotetrahydrofuran-2-yl)ethyl)piperazin-1-yl)phenyl isopropyl carbonate: ¹H NMR (400 MHz, CDCl₃) δ 7.07 (d, J=9.1 Hz, 2H), 6.90 (d, J=9.1 Hz, 2H), 4.96 (sep, J=6.3 Hz, 1H), 4.49 (m, 1H), 3.18 (t, J=5.2 Hz, 4H), 2.63 (t, J=4.9 Hz, 4H), 2.56 (m, 2H), 2.15 (dd, J=6.9, 13.1 Hz, 1H), 1.88 (m, 3H), 1.64 (m, 4H), 1.37 (d, J=6.3 Hz, 6H), 0.94 (dt, J=7.4, 19.4 Hz, 6H) LC/MS [M+H]=m/z 433.20.

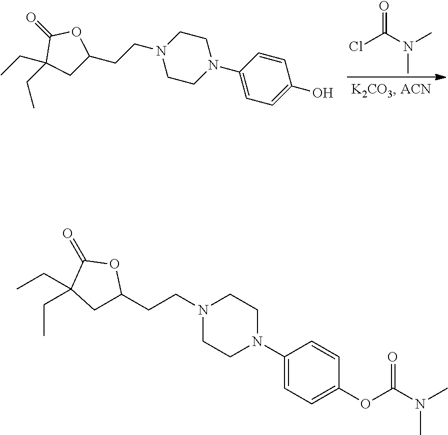

To a solution of 3,3-diethyl-5-(2-(4-(4-hydroxyphenyl)piperazin-1-yl)ethyl)dihydrofuran-2(3H)-one (30 mg, 0.086 mmol, 1 eq.) in acetonitrile (2.0 mL) was added K₂CO₃ (478 mg, 3.46 mmol, 40 eq.). Then, dimethylcarbamic chloride (27.8 mg, 0.259 mmol, 3 eq.) was added and the reaction mixture was allowed to stir at reflux overnight. The mixture was filtered through a glass pipet packed with glass wool and washed with acetonitrile. The filtrate was concentrated under reduced pressure to afford a crude oil which was purified through flash chromatography (silica; methanol/dichloromethane, 0%-10%) to provide 4-(4-(2-(4,4-diethyl-5-oxotetrahydrofuran-2-yl)ethyl)piperazin-1-yl)phenyl dimethylcarbamate.

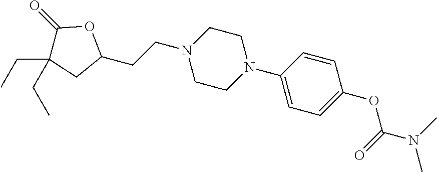 [SMILES: CCC1(CC)CC(CCN2CCN(c3ccc(OC(=O)N(C)C)cc3)CC2)OC1=O]

4-(4-(2-(4,4-diethyl-5-oxotetrahydrofuran-2-yl)ethyl)piperazin-1-yl)phenyl dimethylcarbamate: ¹H NMR (400 MHz, CDCl₃) δ 7.01 (d, J=8.9 Hz, 2H), 6.91 (d, J=9.0 Hz, 2H), 4.50 (m, 1H), 3.21 (t, J=4.8 Hz, 4H), 3.09 (s, 3H), 3.01 (s, 3H), 2.78-2.50 (m, 6H), 2.16 (dd, J=6.7, 13.1 Hz, 1H), 1.93 (m, 2H), 1.86 (dd, J=9.4, 13.1 Hz, 1H), 1.65 (m, 4H), 0.94 (dt, J=7.5, 19.7 Hz, 6H) LC/MS [M+H]=m/z 418.20.

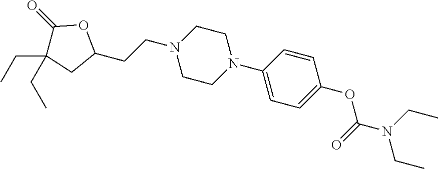 [SMILES: CCN(CC)C(=O)Oc1ccc(N2CCN(CCC3CC(CC)(CC)C(=O)O3)CC2)cc1]

4-(4-(2-(4,4-diethyl-5-oxotetrahydrofuran-2-yl)ethyl)piperazin-1-yl)phenyl diethylcarbamate: ¹H NMR (400 MHz, CDCl₃) δ 7.01 (d, J=9.0 Hz, 2H), 6.90 (d, J=9.0 Hz, 2H), 4.50 (m, 1H), 3.40 (b, 4H), 3.19 (t, J=4.8 Hz, 4H), 2.76-2.53 (m, 6H), 2.16 (dd, J=6.7, 13.1 Hz, 1H), 1.92 (q, J=7.1 Hz, 2H), 1.85 (dd, J=9.4, 13.0 Hz, 1H), 1.63 (m, 4H), 1.21 (b, 6H), 0.93 (dt, J=7.4, 19.4 Hz, 6H) LC/MS [M+H]=m/z 446.20.

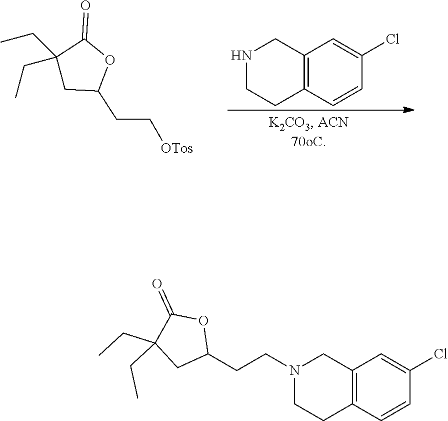

To a solution of 3,3-diethyl-5-(2-(4-(4-hydroxyphenyl)piperazin-1-yl)ethyl)dihydrofuran-2(3H)-one (50 mg, 0.147 mmol, 1 eq.) in acetonitrile (1.5 mL) was added K₂CO₃ (91 mg, 0.658 mmol, 4.5 eq.). Then, 7-chloro-1,2,3,4-tetrahydroisoquinoline (37 mg, 0.220 mmol, 1.5 eq.) was added and the reaction mixture was allowed to stir at reflux for 3 days. The mixture was filtered through a glass pipet packed with glass wool and washed with acetonitrile. The filtrate was concentrated under reduced pressure to afford a crude oil which was purified through flash chromatography (silica; methanol/dichloromethane, 0%-10%) to provide 5-(2-(7-chloro-3,4-dihydroisoquinolin-2(1H)-yl)ethyl)-3,3-diethyldihydrofuran-2(3H)-one.

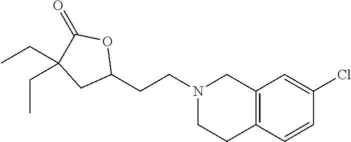 [SMILES: CCC1(CC)CC(CCN2CCc3ccc(Cl)cc3C2)OC1=O]

5-(2-(7-chloro-3,4-dihydroisoquinolin-2(1H)-yl)ethyl)-3,3-diethyl-dihydrofuran-2(3H)-one: 11H NMR (400 MHz, CDCl₃) δ 7.02 (dd, J=2.2, 8.2, 1H), 6.95 (m, 2H), 4.45 (m, 1H), 3.52 (s, 2H), 2.77 (m, 2H), 2.63 (m, 4H), 2.07 (dd, J=6.7, 13.0, 1H), 1.83 (m, 3H), 1.55 (qd, J=1.2, 7.3, 4H), 0.85 (dt, J=7.5, 15.3, 6H) LC/MS [M+H]=m/z 336.1.

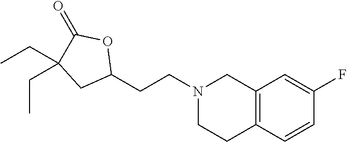 [SMILES: CCC1(CC)CC(CCN2CCc3ccc(F)cc3C2)OC1=O]

3,3-diethyl-5-(2-(7-fluoro-3,4-dihydroisoquinolin-2(1H)-yl)ethyl)-dihydrofuran-2(3H)-one: ¹H NMR (400 MHz, CDCl₃) δ 7.06 (dd, J=5.8, 8.3, 1H), 6.84 (td, J=2.7, 8.5 1H), 6.73 (dd, J=2.5, 9.5, 1H), 4.54 (m, 1H), 3.62 (s, 2H), 2.86 (m, 2H), 2.75 (m, 2H), 2.68 (m, 2H), 2.16 (dd, J=6.8, 13.0, 4H), 1.90 (m, 3H), 1.64 (qt, J=1.7, 7.6, 4H), 0.94 (dt, J=7.5, 15.8, 6H) LC/MS [M+H]=m/z 320.1.

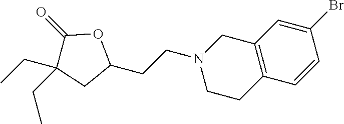 [SMILES: CCC1(CC)CC(CCN2CCc3ccc(Br)cc3C2)OC1=O]

5-(2-(7-bromo-3,4-dihydroisoquinolin-2(1H)-yl)ethyl)-3,3-diethyldihydrofuran-2(3H)-one: ¹H NMR (400 MHz, CDCl₃) δ 7.13 (dd, J=1.8, 8.0 Hz, 1H), 7.06 (d, J=1.4 Hz, 1H), 6.86 (d, J=8.6 Hz, 1H), 4.43 (m, 1H), 3.49 (s, 2H), 2.73 (t, J=5.4 Hz, 2H), 2.62 (m, 2H), 2.56 (m, 2H), 2.06 (dd, J=6.8, 13.0 Hz, 1H), 1.91-1.69 (m, 3H), 1.52 (q, J=7.6 Hz, 4H), 0.83 (dt, J=5.6, 12.8 Hz, 6H) LC/MS [M+H]=m/z 380.10.

### Procedures

### Example 2: Radiolabel Binding Studies for the Sigma-2 Receptor

The following procedure can be utilized in evaluating and selecting compounds as sigma-2 receptor binders and sigma-2 receptor activity modulators.

A solution of the compound to be tested is prepared as a 1 mg/ml stock in Assay Buffer or DMSO according to its solubility. A similar stock of the reference compound Haloperidol is also prepared as a positive control. Eleven dilutions (5× assay concentration) of the compound and Haloperidol are prepared in the Assay Buffer by serial dilution to yield final corresponding assay concentrations ranging from 10 pM to 10 μM.

A stock concentration of 5 nM ³H-1,3-di-(2-tolyl)guanidine (³H-DTG) is prepared in 50 mM Tris-HCl, 10 mM MgCl₂, 1 mM EDTA, pH 7.4 (Assay Buffer). Aliquots (50 μl) of radioligand are dispensed into the wells of a 96-well plate containing 100 μl of Assay Buffer. Duplicate 50-μl aliquots of the compound test and Haloperidol positive control reference compound serial dilutions are added.

Membrane fractions of cells expressing recombinant sigma-2 receptors (50 μL) are dispensed into each well. The membranes are prepared from stably transfected cell lines expressing sigma-2 receptors cultured on 10 cm plates by harvesting PBS-rinsed monolayers, resuspending and lysing in chilled, hypotonic 50 mM Tris-HCl, pH 7.4, centrifuging at 20,000×g, decanting the supernatant and storing at −80° C.; the membrane preparations are resuspended in 3 ml of chilled Assay Buffer and homogenized by several passages through a 26 gauge needle before using in the assay.

The 250 μl reactions are incubated at room temperature for 1.5 hours, then harvested by rapid filtration onto 0.3% polyethyleneimine-treated, 96-well filter mats using a 96-well Filtermate harvester. Four rapid 500 μl washes are performed with chilled Assay Buffer to reduce non-specific binding. The filter mats are dried, then scintillant is added to the filters and the radioactivity retained on the filters is counted in a Microbeta scintillation counter.

Raw data (dpm) representing total radioligand binding (i.e., specific+non-specific binding) are plotted as a function of the logarithm of the molar concentration of the competitor (i.e., test or reference compound). Non-linear regression of the normalized (i.e., percent radioligand binding compared to that observed in the absence of test or reference compound) raw data is performed in Prism 4.0 (GraphPad Software) using the built-in three parameter logistic model describing ligand competition binding to radioligand-labeled sites:

y=bottom+[(top-bottom)/(1+10×−log IC₅₀)]

where bottom equals the residual radioligand binding measured in the presence of 10 μM reference compound (i.e., non-specific binding) and top equals the total radioligand binding observed in the absence of competitor. The log IC₅₀ (i.e., the log of the ligand concentration that reduces radioligand binding by 50%) is thus estimated from the data and used to obtain the Ki by applying the Cheng-Prusoff approximation:

Ki=IC₅₀/(1+[ligand]/KD)

where [ligand] equals the assay radioligand concentration and KD equals the affinity constant of the radioligand for the target receptor.

Compounds are also screened at a single concentration of 10 μM using the same method described for the Radiolabel Binding Studies for sigma-2 receptors to determine the percent inhibition of ³H-DTG binding.

Results for representative compounds according to the present invention are listed in Table 19 and Table 20.

TABLE 19
Radiolabel Binding Studies for the sigma-2 receptors results
for exemplary compounds of the formula (XIX)
(XIX)

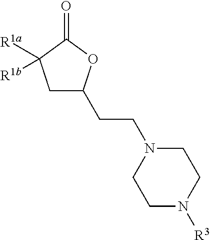

| En- | | | | Sigma-2 IC₅₀ |
|---|---|---|---|---|
| try | R^{1a} | R^{1b} | R³ | (nm) |
| 1 | methyl | methyl | phenyl | 753 |
| 2 | methyl | methyl | 4-hydroxyphenyl | 990 |
| 3 | methyl | methyl | 4-cyanophenyl | 532 |
| 4 | methyl | methyl | 4-trifluorophenyl | 96 |
| 5 | methyl | methyl | 4-methylphenyl | 232 |
| 6 | methyl | methyl | 2-cyanophenyl | 179 |
| 7 | ethyl | ethyl | 4-hydroxyphenyl | 13 |
| 8 | ethyl | ethyl | 4-nitrophenyl | 17 |
| 9 | ethyl | ethyl | 2-methoxyphenyl | 44 |
| 10 | ethyl | ethyl | 4cyanophenyl | 34 |
| 11 | ethyl | ethyl | 2-hydroxyphenyl | 77 |
| 12 | ethyl | ethyl | phenyl | 82 |
| 13 | ethyl | ethyl | 4-aminophenyl | 89 |
| 14 | ethyl | ethyl | 4-methylphenyl | 14 |
| 15 | ethyl | ethyl | 3-methoxyphenyl | 62 |
| 16 | ethyl | ethyl | 3-hydroxyphenyl | 146 |
| 17 | ethyl | ethyl | 2-pyridyl | 268 |
| 18 | ethyl | ethyl | 2,6-dimethylphenyl | 6.9 |
| 19 | ethyl | ethyl | cyclohexyl | 5.4 |
| 20 | ethyl | ethyl | 2-methylphenyl | 9.3 |
| 21 | ethyl | ethyl | ethylphenyl | 7.7 |
| 22 | ethyl | ethyl | 2-isopropylphenyl | 5.9 |
| 23 | ethyl | ethyl | 2,4-dimethylphenyl | 9.2 |
| 24 | ethyl | ethyl | diphenylmethyl | 3.4 |
| 25 | phenyl | phenyl | 4-methylphenyl | 95 |

| | | | |
|---|---|---|---|
| 26 | —CH₂CH₂CH₂CH₂CH₂— | 4-nitro-3-aminophenyl | 78 |
| 27 | —CH₂CH₂CH₂CH₂CH₂— | 5-(1H)-benzo[d] | 836 |
| | | imidazol-2(3H)-oyl) | |
| 28 | —CH₂CH₂CH₂CH₂CH₂— | 4-methoxyphenyl | 98.5 |
| 29 | —CH₂CH₂CH₂CH₂CH₂— | 3-hydroxyphenyl | 109 |
| 30 | —CH₂CH₂CH₂CH₂CH₂— | phenyl | 35 |
| 31 | —CH₂CH₂CH₂CH₂CH₂— | 3-methoxyphenyl | 36 |
| 32 | —CH₂CH₂CH₂CH₂CH₂— | 2-methyoxyphenyl | 33.5 |
| 33 | —CH₂CH₂CH₂CH₂CH₂— | 2-hydroxyphenyl | 53 |
| 34 | —CH₂CH₂CH₂CH₂CH₂— | 2-pyridyl | 107 |
| 35 | —CH₂CH₂CH₂CH₂CH₂— | 2-chlorophenyl | 5.7 |
| 36 | —CH₂CH₂CH₂CH₂CH₂— | 4-chlorophenyl | 5.2 |
| 37 | —CH₂CH₂CH₂CH₂CH₂— | 4-trifluoromethyl- | 6.1 |
| | | phenyl | |
| 38 | —CH₂CH₂CH₂CH₂CH₂— | 4-pyridyl | 91 |
| 39 | —CH₂CH₂CH₂CH₂CH₂— | 4-hydroxyphenyl | 14 |
| 40 | —CH₂CH₂CH₂CH₂CH₂— | 3-pyridyl | 512 |
| 41 | —CH₂CH₂CH₂CH₂CH₂— | 4-cyanophenyl | 33 |
| 42 | —CH₂CH₂CH₂CH₂CH₂— | 4-cyano-2-pyridyl | 66 |
| 43 | —CH₂CH₂CH₂CH₂CH₂— | 4-trifluoromethyl-2- | 6.2 |
| | | pyridyl | |
| 44 | —CH₂CH₂CH₂CH₂CH₂— | 4-chloro-2-pyridyl | 6.8 |
| 45 | —CH₂CH₂CH₂CH₂CH₂— | 5-indolyl | 143 |
| 46 | —CH₂CH₂CH₂CH₂CH₂— | 3-chloro-4-pyridyl | 57 |
| 47 | —CH₂CH₂CH₂CH₂CH₂— | 3-chloro-2-pyridyl | 1526 |
| 48 | —CH₂CH₂CH₂CH₂CH₂— | 3-cyano-2-pyridyl | 98 |
| 49 | —CH₂CH₂CH₂CH₂CH₂— | 3-methoxy-2-pyridyl | 151 |
| 50 | —CH₂CH₂CH₂CH₂CH₂— | 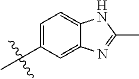 [SMILES: *c1ccc2[nH]c(C)nc2c1] | 287 |
| 51 | —CH₂CH₂CH₂CH₂— | 5-indolyl | 214 |
| 52 | —CH₂CH₂CH₂CH₂— | 2-isopropylphenyl | 8 |
| 53 | —CH₂CH₂CH₂CH₂— | 2-t-butylphenyl | 6.8 |
| 54 | —CH₂CH₂CH₂CH₂— | 2,6-diisopropylphenyl | 24 |
| 55 | —CH₂CH₂CH₂CH₂— | 2-morphilinophenyl | 23 |
| 56 | —CH₂CH₂CH₂CH₂— | 4-chloro-2-pyridyl | 30 |
| 57 | —CH₂CH₂CH₂CH₂— | 4-trifluoromethyl-2- | 12 |
| | | pyridyl | |
| 58 | —CH₂CH₂CH₂CH₂— | 4-methyl-2-pyridyl | 155 |
| 59 | —CH₂CH₂CH₂CH₂— | 4-hydroxy-2-pyridyl | 554 |
| 60 | —CH₂CH₂CH₂CH₂— | 4-cyano-2-pyridyl | 387 |
| 61 | —CH₂CH₂CH₂CH₂— | 4-pyridyl | 338 |
| 62 | —CH₂CH₂CH₂CH₂— | 4-fluoro-2-pyridyl | 95 |
| 63 | —CH₂CH₂CH₂CH₂— | 5-(1H)-benzo[d] | 10000 |
| | | imidazol-2(3H)-oyl) | |
| 64 | —CH₂CH₂CH₂CH₂— | 4-(methylsulfonamide) | 10000 |
| | | phenyl | |
| 65 | —CH₂CH₂CH₂CH₂— | 3-methoxy-2-pyridyl | 163 |
| 66 | —CH₂CH₂CH₂CH₂— | 3-chloro-2-pyridyl | 233 |
| 67 | —CH₂CH₂CH₂CH₂— | 3-chloro-4-pyridyl | 208 |
| 68 | —CH₂CH₂CH₂CH₂— | 3-cyano-2-pyridyl | 314 |
| 69 | —CH₂CH₂CH₂CH₂— | 2-methoxyphenyl | 29 |
| 70 | —CH₂CH₂CH₂CH₂— | 2-Hydroxyphenyl | 66 |
| 71 | —CH₂CH₂CH₂— | 5-indolyl | 156 |
| 72 | —CH₂CH₂CH₂— | 5-(1H)-benzo[d] | 10000 |
| | | imidazol-2(3H)-oyl) | |
| 73 | —CH₂CH₂CH₂— | 2-methoxyphenyl | 59 |
| 74 | —CH₂CH₂CH₂— | 2-hydroxyphenyl | 263 |
| 75 | —CH₂CH₂CH₂— | 3-cyano-2-pyridyl | 779.5 |
| 76 | —CH₂CH₂CH₂— | 3-chloro-4-pyridyl | 966 |

TABLE 20
Radiolabel Binding Studies for the sigma-2 receptors results for exemplary
compounds of the disclosure

| | | Sigma-2 |
|---|---|---|
| Entry | Structure | IC₅₀ (nm) |
| 1 | 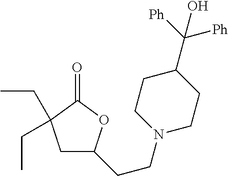 [SMILES: CCC1(CC)CC(CCN2CCC(C(O)(c3ccccc3)c3ccccc3)CC2)OC1=O] | 5.4 |
| 2 | 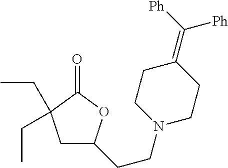 [SMILES: CCC1(CC)CC(CCN2CCC(=C(c3ccccc3)c3ccccc3)CC2)OC1=O] | 5.4 |
| 3 | 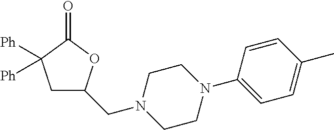 [SMILES: Cc1ccc(N2CCN(CC3CC(c4ccccc4)(c4ccccc4)C(=O)O3)CC2)cc1] | 235 |
| 4 | 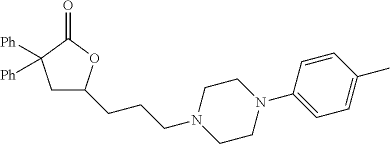 [SMILES: Cc1ccc(N2CCN(CCCC3CC(c4ccccc4)(c4ccccc4)C(=O)O3)CC2)cc1] | 14 |
| 5 | 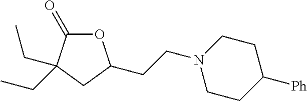 [SMILES: CCC1(CC)CC(CCN2CCC(c3ccccc3)CC2)OC1=O] | 6.4 |
| 6 | 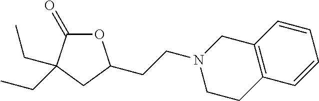 [SMILES: CCC1(CC)CC(CCN2CCc3ccccc3C2)OC1=O] | 6.1 |
| 7 | 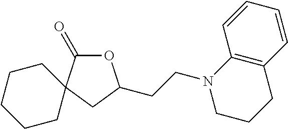 [SMILES: O=C1OC(CCN2CCCc3ccccc32)CC12CCCCC2] | 679 |
| 8 | 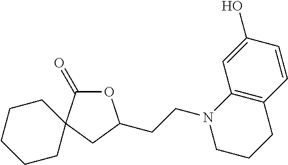 [SMILES: O=C1OC(CCN2CCCc3ccc(O)cc32)CC12CCCCC2] | 10000 |
| 9 | 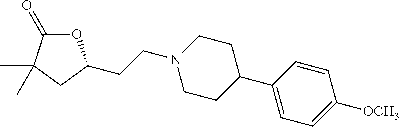 [SMILES: COc1ccc(C2CCN(CC[C@@H]3CC(C)(C)C(=O)O3)CC2)cc1] | 694 |
| 10 | 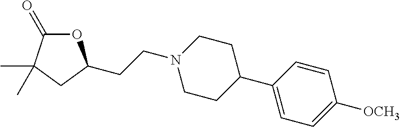 [SMILES: COc1ccc(C2CCN(CC[C@H]3CC(C)(C)C(=O)O3)CC2)cc1] | 391 |
| 11 | 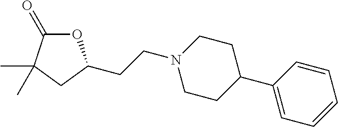 [SMILES: CC1(C)C[C@@H](CCN2CCC(c3ccccc3)CC2)OC1=O] | 19 |
| 12 | 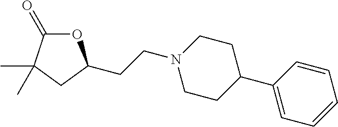 [SMILES: CC1(C)C[C@H](CCN2CCC(c3ccccc3)CC2)OC1=O] | 13 |
| 13 | 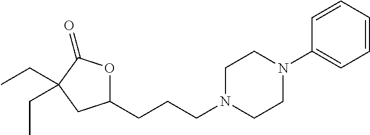 [SMILES: CCC1(CC)CC(CCCN2CCN(c3ccccc3)CC2)OC1=O] | 7.7 |
| 14 | 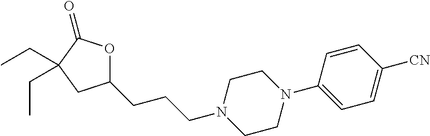 [SMILES: CCC1(CC)CC(CCCN2CCN(c3ccc(C#N)cc3)CC2)OC1=O] | 17 |
| 15 | 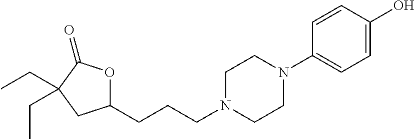 [SMILES: CCC1(CC)CC(CCCN2CCN(c3ccc(O)cc3)CC2)OC1=O] | 10 |
| 16 | 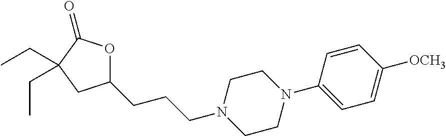 [SMILES: CCC1(CC)CC(CCCN2CCN(c3ccc(OC)cc3)CC2)OC1=O] | 33 |
| 17 | 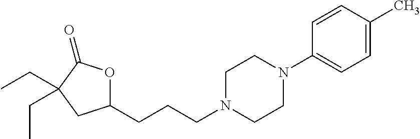 [SMILES: CCC1(CC)CC(CCCN2CCN(c3ccc(C)cc3)CC2)OC1=O] | 19 |
| 18 | 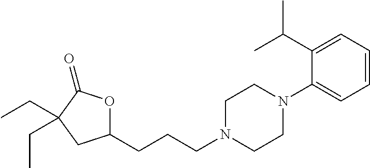 [SMILES: CCC1(CC)CC(CCCN2CCN(c3ccccc3C(C)C)CC2)OC1=O] | 7.5 |
| 19 | 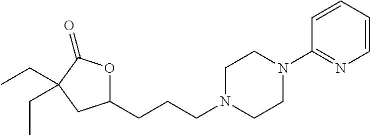 [SMILES: CCC1(CC)CC(CCCN2CCN(c3ccccn3)CC2)OC1=O] | 54 |
| 20 | 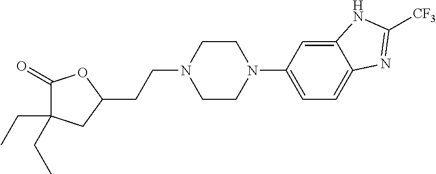 [SMILES: CCC1(CC)CC(CCN2CCN(c3ccc4nc(C(F)(F)F)[nH]c4c3)CC2)OC1=O] | 2031 |
| 21 | 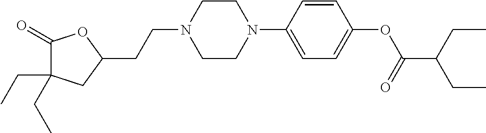 [SMILES: CCC(CC)C(=O)Oc1ccc(N2CCN(CCC3CC(CC)(CC)C(=O)O3)CC2)cc1] | 40 |
| 22 | 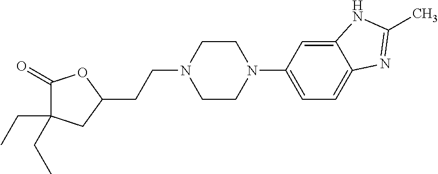 [SMILES: CCC1(CC)CC(CCN2CCN(c3ccc4nc(C)[nH]c4c3)CC2)OC1=O] | 3961 |
| 23 | 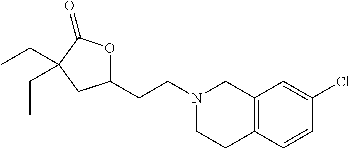 [SMILES: CCC1(CC)CC(CCN2CCc3ccc(Cl)cc3C2)OC1=O] | 2.8 |
| 24 | 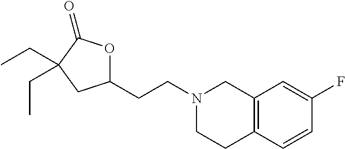 [SMILES: CCC1(CC)CC(CCN2CCc3ccc(F)cc3C2)OC1=O] | 7.4 |
| 25 | 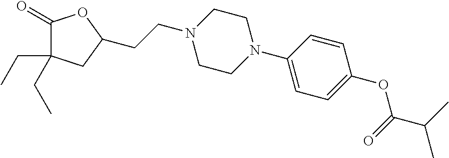 [SMILES: CCC1(CC)CC(CCN2CCN(c3ccc(OC(=O)C(C)C)cc3)CC2)OC1=O] | 105 |
| 26 | 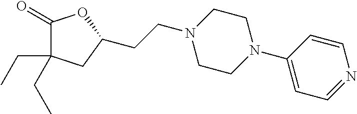 [SMILES: CCC1(CC)C[C@@H](CCN2CCN(c3ccncc3)CC2)OC1=O] | 273 |
| 27 | 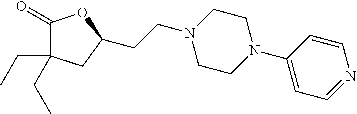 [SMILES: CCC1(CC)C[C@H](CCN2CCN(c3ccncc3)CC2)OC1=O] | 373 |
| 28 | 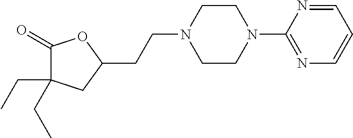 [SMILES: CCC1(CC)CC(CCN2CCN(c3ncccn3)CC2)OC1=O] | 1223 |
| 29 | 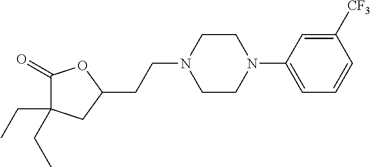 [SMILES: CCC1(CC)CC(CCN2CCN(c3cccc(C(F)(F)F)c3)CC2)OC1=O] | 12 |
| 30 | 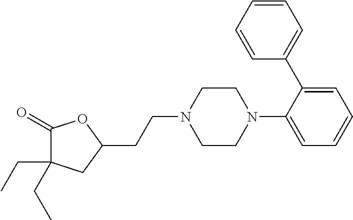 [SMILES: CCC1(CC)CC(CCN2CCN(c3ccccc3-c3ccccc3)CC2)OC1=O] | 5.6 |
| 31 | 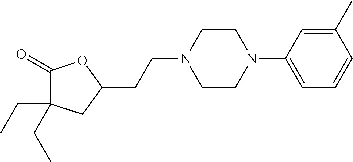 [SMILES: CCC1(CC)CC(CCN2CCN(c3cccc(C)c3)CC2)OC1=O] | 30 |
| 32 | 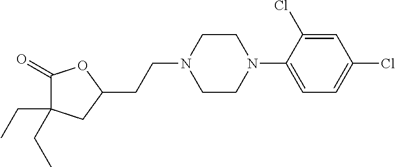 [SMILES: CCC1(CC)CC(CCN2CCN(c3ccc(Cl)cc3Cl)CC2)OC1=O] | 8.2 |

What is claimed is:

1. A method for treating a disease thats involve dysregulation of sigma-2 receptor activity said method comprising administering to a subject an effective amount of at least one compound of the formula (I): 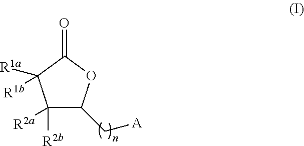
including hydrates, solvates, pharmaceutically acceptable salts, prodrugs and complexes thereof, wherein:
A is selected from the group consisting of 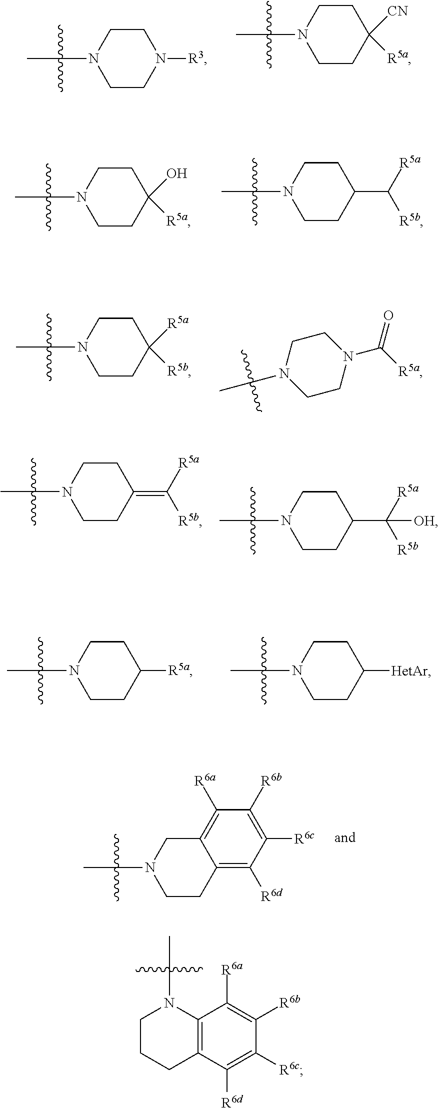
R^{1a} and R^{1b} are each independently selected from the group consisting of hydrogen, C₁₋₆ linear alkyl, C₁₋₆ branched alkyl, and optionally substituted aryl, or R^{1a} and R^{1b} may be taken together with the atom to which they are bound to form a ring having from 3 to 7 ring atoms;
R^{2a} and R^{2b} are each independently selected from the group consisting of hydrogen, C₁₋₆ linear alkyl, C₁₋₆ branched alkyl, and optionally substituted aryl, or R^{2a} and R^{2b} may be taken together with the atom to which they are bound to form a ring having from 3 to 7 ring atoms;
R³ is selected from the group consisting of C₁₋₆ linear alkyl, C₁₋₆ branched alkyl, C₃₋₇ cycloalkyl, optionally substituted aryl, optionally substituted heteroaryl, 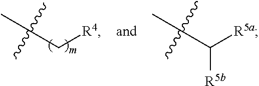
R⁴ is optionally substituted aryl;
R^{5a} and R^{5b} are each independently optionally substituted aryl;
R^{6a}, R^{6b}, R^{6c}, and R^{6d} are each independently selected from the group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁷, C(O)NHR⁷, C(O)N(R⁷)₂, SH, SC₁₋₆ alkyl, SO²NH₂, SO₂NHR⁷, SO₂R⁷, and NHSO₂R⁷;
each occurrence of R⁷ is independently selected from the group consisting of C₁₋₆ linear alkyl, C₁₋₆ branched alkyl, and C₃₋₇ cycloalkyl;
HetAr is optionally substituted heteroaryl;
n is 1, 2, or 3; and
m is 1 or 2.

2. The method of claim 1, wherein the compound of formula (I) is a compound of formula (II):

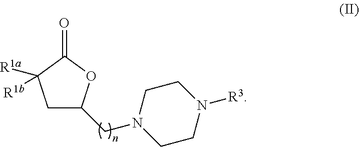

3. The method of claim 1, wherein the compound of formula (I) is a compound of formula (XIX):

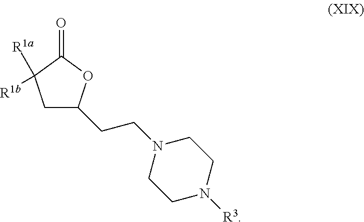

4. The method of claim 3, wherein R^{1a} and R^{1b} are each independently selected from the group consisting of methyl, ethyl, and a ring having four, five, or six carbon atoms formed by taking R^{1a} and R^{1b} together with the atom to which they are bound.

5. The method of claim 3, wherein R³ is selected from the group consisting of optionally substituted aryl, optionally substituted heteroaryl.

6. The method of claim 1, wherein the at least one compound is selected from the group consisting of
3-{2-[4-(5-chloro-pyridin-2-yl)-piperazin-1-yl]-ethyl}-2-oxa-spiro[4.5]decan-1-one;
3-{2-[4-(1H-Indol-5-yl)-piperazin-1-yl]-ethyl}-2-oxa-spiro[4.5]decan-1-one;
3-{2-[4-(1H-Indol-5-yl)-piperazin-1-yl]-ethyl}-2-oxa-spiro[4.4]nonan-1-one;
7-{2-[4-(1H-Indol-5-yl)-piperazin-1-yl]-ethyl}-6-oxa-spiro[3.4]octan-5-one;
3-(2-(4-(-3-amino-4-nitrophenyl)piperazin-1-yl)ethyl)-2-oxaspiro[4.5]decan-1-one;
5-(4-(2-(1-oxo-2-oxaspiro[4.5]decan-3-yl)ethyl)piperazin-1-yl)-1H-benzo[d]imidazol-2(3H)-one;
5-(4-(2-(1-oxo-2-oxaspiro[4.4]nonan-3-yl)piperazin-1-yl)-1H-benzo[d]imidazol-2(3H)-one;
5-(4-(2-(1-oxo-2-oxaspiro[3.4]octan-3-yl)piperazin-1-yl)-1H-benzo[d]imidazol-2(3H)-one;
3-(2-(4-(2-chloropyridin-4-yl)piperazin-1-yl)ethyl)-2-oxaspiro[4.5]decan-1-one;
3-{2-[4-(4-chloro-pyridin-2-yl)-piperazin-1-yl]-ethyl}-2-oxa-spiro[4.5]decan-1-one;
2-(4-(2-(1-oxo-2-oxaspiro[4.5]decan-3-yl)ethyl)piperazin-1-yl)isonicotinonitrile;
3-(2-(4-(4-methoxypyridin-2-yl)piperazin-1-yl)ethyl)-2-oxaspiro[4.5]decan-1-one;
2-(4-(2-(5-oxo-6-oxaspiro[3.4]octan-7-yl)ethyl)piperazin-1-yl)isonicotinonitrile;
7-(2-(4-(2-chloropyridin-4-yl)piperazin-1-yl)ethyl)-6-oxaspiro[3.4]octan-5-one;
3-(2-(3,4-dihydroquinolin-1 (2H)-yl)ethyl)-2-oxaspiro[4.5]decan-1-one;
3-(2-(7-hydroxy-3,4-dihydroquinolin-1 (2H)-yl)ethyl)-2-oxaspiro[4.5]decan-1-one;
3-(2-(4-(2-methyl-1H-benzo[d]imidazol-4-yl)piperazin-1-yl)ethyl)-2-oxaspiro[4.5]decan-1-one;
dihydro-3,3-dimethyl-5-(2-(4-phenylpiperazin-1-yl)ethyl)furan-2(3H)-one;
dihydro-5-(2-(4-(4-hydroxyphenyl)piperazin-1-yl)ethyl)-3,3-dimethylfuran-2(3H)-one;
4-(4-(2-(tetrahydro-4,4-dimethyl-5-oxofuran-2-yl)ethyl)piperazin-1-yl)benzonitrile;
5-(2-(4-(4-(trifluoromethyl)phenyl)piperazin-1-yl)ethyl)-dihydro-3,3-dimethylfuran-2(3H)-one;
dihydro-3,3-dimethyl-5-(2-(4-p-tolylpiperazin-1-yl)ethyl)furan-2(3H)-one;
3-(2-(4-(4-chloropyridin-2-yl)piperazin-1-yl)ethyl)-2-oxaspiro[4.4]nonan-1-one;
2-(4-(2-(1-oxo-2-oxaspiro[4.4]nonan-3-yl)ethyl)piperazin-1-yl)isonicotinonitrile;
3-(2-(4-(4-methoxypyridin-2-yl)piperazin-1-yl)ethyl)-2-oxaspiro[4.4]nonan-1-one;
3-(2-(4-(2-chloropyridin-4-yl)piperazin-1-yl)ethyl)-2-oxaspiro[4.4]nonan-1-one;
7-(2-(4-(2-hydroxyphenyl)piperazin-1-yl)ethyl)-6-oxaspiro[3.4]octan-5-one;
7-(2-(4-(2-methoxyphenyl)piperazin-1-yl)ethyl)-6-oxaspiro[3.4]octan-5-one;
3-(2-(4-(2-hydroxyphenyl)piperazin-1-yl)ethyl)-2-oxaspiro[4.4]nonan-1-one;
3-(2-(4-(2-methoxyphenyl)piperazin-1-yl)ethyl)-2-oxaspiro[4.4]nonan-1-one;
N-(4-(4-(2-(1-oxo-2-oxaspiro[4.4]nonan-3-yl)ethyl)piperazin-1-yl)phenyl)methanesulfonamide;
2-(4-(2-(4,4-diethyl-5-oxotetrahydrofuran-2-yl)ethyl)piperazin-1-yl)benzonitrile;
3,3-diethyl-5-(2-(4-(4-methoxyphenyl)piperazin-1-yl)ethyl)dihydrofuran-2(3H)-one;
3,3-diethyl-5-(2-(4-(4-hydroxyphenyl)piperazin-1-yl)ethyl)dihydrofuran-2(3H)-one;
3,3-diethyl-5-(2-(4-(4-nitrophenyl)piperazin-1-yl)ethyl)dihydrofuran-2(3H)-one;
3,3-diethyl-5-(2-(4-(2-methoxyphenyl)piperazin-1-yl)ethyl)dihydrofuran-2(3H)-one;
4-(4-(2-(4,4-diethyl-5-oxotetrahydrofuran-2-yl)ethyl)piperazin-1-yl)benzonitrile;
3,3-diethyl-5-(2-(4-(2-hydroxyphenyl)piperazin-1-yl)ethyl)dihydrofuran-2(3H)-one;
3,3-diethyl-5-(2-(4-phenylpiperazin-1-yl)ethyl)dihydrofuran-2(3H)-one;
5-(2-(4-(4-aminophenyl)piperazin-1-yl)ethyl)-3,3-diethyldihydrofuran-2(3H)-one;
3,3-diethyl-5-(2-(4-(p-tolyl)piperazin-1-yl)ethyl)dihydrofuran-2(3H)-one;
3,3-diethyl-5-(2-(4-(3-methoxyphenyl)piperazin-1-yl)ethyl)dihydrofuran-2(3H)-one;
3,3-diethyl-5-(2-(4-(3-hydroxyphenyl)piperazin-1-yl)ethyl)dihydrofuran-2(3H)-one;
3,3-diethyl-5-(2-(4-(pyridin-2-yl)piperazin-1-yl)ethyl)dihydrofuran-2(3H)-one;
5-(2-(4-(2,6-dimethylphenyl)piperazin-1-yl)ethyl)-3,3-diethyldihydrofuran-2(3H)-one;
5-(2-(4-cyclohexylpiperazin-1-yl)ethyl)-3,3-diethyldihydrofuran-2(3H)-one;
3,3-diethyl-5-(2-(4-(o-tolyl)piperazin-1-yl)ethyl)dihydrofuran-2(3H)-one;
3,3-diethyl-5-(2-(4-phenylpiperidin-1-yl)ethyl)dihydrofuran-2(3H)-one;
3,3-diethyl-5-(2-(4-phenethylpiperazin-1-yl)ethyl)dihydrofuran-2(3H)-one;
3,3-diethyl-5-(2-(4-(2-isopropylphenyl)piperazin-1-yl)ethyl)dihydrofuran-2(3H)-one;
5-(2-(4-(2,4-dimethylphenyl)piperazin-1-yl)ethyl)-3,3-diethyldihydrofuran-2(3H)-one;
3,3-diethyl-5-(2-(4-(4-fluorobenzyl)piperazin-1-yl)ethyl)dihydrofuran-2(3H)-one;
5-(2-(4-benzhydrylpiperazin-1-yl)ethyl)-3,3-diethyldihydrofuran-2(3H)-one;
5-(2-(3,4-dihydroisoquinolin-2(1H)-yl)ethyl)-3,3-diethyldihydrofuran-2(3H)-one;
2-(4-(2-(4,4-dimethyl-5-oxotetrahydrofuran-2-yl)ethyl)piperazin-1-yl)benzonitrile;
3,3-diethyl-5-(2-(4-(hydroxydiphenylmethyl)piperidin-1-yl)ethyl)dihydrofuran-2(3H)-one
5-(2-(4-(diphenylmethylene)piperidin-1-yl)ethyl)-3,3-diethyldihydrofuran-2(3H)-one;
3-(2-(4-(2-isopropylphenyl)piperazin-1-yl)ethyl)-2-oxaspiro[4.4]nonan-1-one;
3-(2-(4-(2-isopropylphenyl)piperazin-1-yl)ethyl)-2-oxaspiro[4.5]decan-1-one;
5-(2-(4-(2-isopropylphenyl)piperazin-1-yl)ethyl)-3,3-diphenyldihydrofuran-2(3H)-one;
5-(2-(4-(1H-benzo[d]imidazol-2-yl)piperidin-1-yl)ethyl)-3,3-diethyldihydrofuran-2(3H)-one;
5-(2-(4-benzoylpiperazin-1-yl)ethyl)-3,3-diethyldihydrofuran-2(3H)-one;
3-(2-(4-(2-(tert-butyl)phenyl)piperazin-1-yl)ethyl)-2-oxaspiro[4.4]nonan-1-one;
3-(2-(4-(2,6-diisopropylphenyl)piperazin-1-yl)ethyl)-2-oxaspiro[4.4]nonan-1-one;
3-(2-(4-(2-morpholinophenyl)piperazin-1-yl)ethyl)-2-oxaspiro[4.4]nonan-1-one;
3-(2-(4-([1,1′-biphenyl]-2-yl)piperazin-1-yl)ethyl)-2-oxaspiro[4.4]nonan-1-one;
3-(2-(4-(2-(1H-pyrrol-1-yl)phenyl)piperazin-1-yl)ethyl)-2-oxaspiro[4.4]nonan-1-one;
3-(2-(4-(2-iodophenyl)piperazin-1-yl)ethyl)-2-oxaspiro[4.4]nonan-1-one;
N-(2-(4-(2-(1-oxo-2-oxaspiro[4.4]nonan-3-yl)ethyl)piperazin-1-yl)phenyl)acetamide;
3-(2-(4-(naphthalen-1-yl)piperazin-1-yl)ethyl)-2-oxaspiro[4.4]nonan-1-one;
3-(2-(4-(anthracen-1-yl)piperazin-1-yl)ethyl)-2-oxaspiro[4.4]nonan-1-one;
3-(2-(4-(benzo[c][1,2,5]thiadiazol-4-yl)piperazin-1-yl)ethyl)-2-oxaspiro[4.4]nonan-1-one;
5-nitro-2-(4-(2-(1-oxo-2-oxaspiro[4.4]nonan-3-yl)ethyl)piperazin-1-yl)benzonitrile;
3-(2-(4,4-diphenylpiperidin-1-yl)ethyl)-2-oxaspiro[4.4]nonan-1-one;
3-(2-(4-benzhydrylpiperidin-1-yl)ethyl)-2-oxaspiro[4.4]nonan-1-one;
3-(2-(4-hydroxy-4-phenylpiperidin-1-yl)ethyl)-2-oxaspiro[4.4]nonan-1-one;
1-(2-(1-oxo-2-oxaspiro[4.4]nonan-3-yl)ethyl)-4-phenylpiperidine-4-carbonitrile;
1-(2-(4-(2-isopropylphenyl)piperazin-1-yl)ethyl)-2-oxaspiro[4.5]decan-3-one;
5-(2-(4-(2-isopropylphenyl)piperazin-1-yl)ethyl)-4,4-dimethyldihydrofuran-2(3H)-one;
3-(3-(4-(2-isopropylphenyl)piperazin-1-yl)propyl)-2-oxaspiro[4.4]nonan-1-one;
3-(3-(4-(2-isopropylphenyl)piperazin-1-yl)propyl)-2-oxaspiro[4.5]decan-1-one;
3,3-diphenyl-5-(2-(4-(p-tolyl)piperazin-1-yl)ethyl)dihydrofuran-2(3H)-one;
3,3-diphenyl-5-(3-(4-(p-tolyl)piperazin-1-yl)propyl)dihydrofuran-2(3H)-one;
3-(2-(4-(p-tolyl)piperazin-1-yl)ethyl)-2-oxaspiro[4.4]nonan-1-one;
3-(3-(4-(p-tolyl)piperazin-1-yl)propyl)-2-oxaspiro[4.4]nonan-1-one;
3-(2-(4-(p-tolyl)piperazin-1-yl)ethyl)-2-oxaspiro[4.5]decan-1-one;
3-(3-(4-(p-tolyl)piperazin-1-yl)propyl)-2-oxaspiro[4.5]decan-1-one;
3,3-diethyl-5-((4-(p-tolyl)piperazin-1-yl)methyl)dihydrofuran-2(3H)-one;
1-(2-(4-(p-tolyl)piperazin-1-yl)ethyl)-2-oxaspiro[4.5]decan-3-one;
4,4-dimethyl-5-(2-(4-(p-tolyl)piperazin-1-yl)ethyl)dihydrofuran-2(3H)-one;
3,3-diphenyl-5-((4-(p-tolyl)piperazin-1-yl)methyl)dihydrofuran-2(3H)-one;
3-((4-(p-tolyl)piperazin-1-yl)methyl)-2-oxaspiro[4.4]nonan-1-one;
3-((4-(p-tolyl)piperazin-1-yl)methyl)-2-oxaspiro[4.5]decan-1-one;
(S)-dihydro-5-(2-(4-(3-hydroxyphenyl)piperazin-1-yl)ethyl)-3,3-dimethylfuran-2(3H)-one;
(S)-dihydro-5-(2-(4-(3-methoxyphenyl)piperazin-1-yl)ethyl)-3,3-dimethylfuran-2(3H)-one;
(S)-dihydro-5-(2-(4-(4-methoxyphenyl)piperazin-1-yl)ethyl)-3,3-dimethylfuran-2(3H)-one;
(S)-dihydro-3,3-dimethyl-5-(2-(4-phenylpiperidin-1-yl)ethyl)furan-2(3H)-one;
(S)-dihydro-5-(2-(4-hydroxy-4-phenylpiperidin-1-yl)ethyl)-3,3-dimethylfuran-2(3H)-one;
(R)-dihydro-5-(2-(4-(3-hydroxyphenyl)piperazin-1-yl)ethyl)-3,3-dimethylfuran-2(3H)-one;
(R)-dihydro-5-(2-(4-(3-methoxyphenyl)piperazin-1-yl)ethyl)-3,3-dimethylfuran-2(3H)-one;
(R)-dihydro-5-(2-(4-(4-methoxyphenyl)piperazin-1-yl)ethyl)-3,3-dimethylfuran-2(3H)-one;
(R)-dihydro-3,3-dimethyl-5-(2-(4-phenylpiperidin-1-yl)ethyl)furan-2(3H)-one;
(R)-dihydro-5-(2-(4-hydroxy-4-phenylpiperidin-1-yl)ethyl)-3,3-dimethylfuran-2(3H)-one;
(S)-dihydro-5-(2-(4-(2-isopropylphenyl)piperazin-1-yl)ethyl)-3,3-dimethylfuran-2(3H)-one;
(R)-dihydro-5-(2-(4-(2-isopropylphenyl)piperazin-1-yl)ethyl)-3,3-dimethylfuran-2(3H)-one;
3,3-diethyl-dihydro-5-(3-(4-phenylpiperazin-1-yl)propyl)furan-2(3H)-one;
4-(4-(3-(4,4-diethyl-tetrahydro-5-oxofuran-2-yl)propyl)piperazin-1-yl)benzonitrile;
3,3-diethyl-dihydro-5-(3-(4-(4-hydroxyphenyl)piperazin-1-yl)propyl)furan-2(3H)-one;
3,3-diethyl-dihydro-5-(3-(4-(4-methoxyphenyl)piperazin-1-yl)propyl)furan-2(3H)-one;
3,3-diethyl-dihydro-5-(3-(4-p-tolylpiperazin-1-yl)propyl)furan-2(3H)-one;
3,3-diethyl-dihydro-5-(3-(4-(2-isopropylphenyl)piperazin-1-yl)propyl)furan-2(3H)-one;
3,3-diethyl-dihydro-5-(3-(4-(pyridin-2-yl)piperazin-1-yl)propyl)furan-2(3H)-one;
3,3-diethyl-5-(2-(4-(2-(trifluoromethyl)-1H-benzo[d]imidazol-6-yl)piperazin-1-yl)ethyl)-dihydrofuran-2(3H)-one;
3,3-diethyl-dihydro-5-(2-(4-(2-methyl-1H-benzo[d]imidazol-6-yl)piperazin-1-yl)ethyl)furan-2(3H)-one;
(S)-3,3-diethyl-dihydro-5-(2-(4-(pyridin-4-yl)piperazin-1-yl)ethyl)furan-2(3H)-one;
(R)-3,3-diethyl-dihydro-5-(2-(4-(pyridin-4-yl)piperazin-1-yl)ethyl)furan-2(3H)-one;
3,3-diethyl-dihydro-5-(2-(4-(pyrimidin-2-yl)piperazin-1-yl)ethyl)furan-2(3H)-one;
3,3-diethyl-5-(2-(4-(3-(trifluoromethyl)phenyl)piperazin-1-yl)ethyl)-dihydrofuran-2(3H)-one;
5-(2-(4-([1,1′-biphenyl]-2-yl)piperazin-1-yl)ethyl)-3,3-diethyldihydrofuran-2(3H)-one;
3,3-diethyl-dihydro-5-(2-(4-m-tolylpiperazin-1-yl)ethyl)furan-2(3H)-one;
5-(2-(4-(2,4-dichlorophenyl)piperazin-1-yl)ethyl)-3,3-diethyl-dihydrofuran-2(3H)-one;
5-(2-(4-(2-chlorophenyl)piperazin-1-yl)ethyl)-3,3-diethyldihydrofuran-2(3H)-one;
5-(2-(4-(3,5-dichlorophenyl)piperazin-1-yl)ethyl)-3,3-diethyldihydrofuran-2(3H)-one;
3,3-diethyl-5-(2-(4-(naphthalen-1-yl)piperazin-1-yl)ethyl)dihydrofuran-2(3H)-one;
3-(4-(2-(4,4-diethyl-5-oxotetrahydrofuran-2-yl)ethyl)piperazin-1-yl)benzonitrile;
5-(2-(4-(benzo[d]isoxazol-3-yl)piperazin-1-yl)ethyl)-3,3-diethyldihydrofuran-2(3H)-one;
3,3-diethyl-5-(2-(4-(2-(trifluoromethyl)phenyl)piperazin-1-yl)ethyl)dihydrofuran-2(3H)-one;
3,3-diethyl-5-(2-(4-(pyrazin-2-yl)piperazin-1-yl)ethyl)dihydrofuran-2(3H)-one;
3,3-diethyl-5-(2-(4-(pyrazin-2-yl)piperazin-1-yl)ethyl)dihydrofuran-2(3H)-one;
3,3-diethyl-5-(2-(4-(3-fluorophenyl)piperazin-1-yl)ethyl)dihydrofuran-2(3H)-one;
(R)-5-(2-(4-phenylpiperazin-1-yl)ethyl)dihydrofuran-2(3H)-one;
(5S)-3-methyl-5-(2-(4-phenylpiperazin-1-yl)ethyl)dihydrofuran-2(3H)-one;
(5R)-3-phenyl-5-(2-(4-phenylpiperazin-1-yl)ethyl)dihydrofuran-2(3H)-one;
(5R)-5-(2-(4-(4-hydroxyphenyl)piperazin-1-yl)ethyl)-3-phenyldihydrofuran-2(3H)-one;
(5R)-3-phenyl-5-(2-(4-(pyridin-4-yl)piperazin-1-yl)ethyl)dihydrofuran-2(3H)-one;
(5S)-3-phenyl-5-(2-(4-phenylpiperazin-1-yl)ethyl)dihydrofuran-2(3H)-one;
(5S)-5-(2-(4-(4-hydroxyphenyl)piperazin-1-yl)ethyl)-3-phenyldihydrofuran-2(3H)-one;
(5S)-3-phenyl-5-(2-(4-(pyridin-4-yl)piperazin-1-yl)ethyl)dihydrofuran-2(3H)-one;
3-(2-(4-(3-chloropyridin-4-yl)piperazin-1-yl)ethyl)-2-oxaspiro[4.5]decan-1-one;
3-(2-(4-(3-methylpyridin-4-yl)piperazin-1-yl)ethyl)-2-oxaspiro[4.5]decan-1-one;
3-(2-(4-(2-methylpyridin-4-yl)piperazin-1-yl)ethyl)-2-oxaspiro[4.5]decan-1-one;
4-(4-(2-(1-oxo-2-oxaspiro[4.5]decan-3-yl)ethyl)piperazin-1-yl)nicotinonitrile;
4-(4-(2-(1-oxo-2-oxaspiro[4.5]decan-3-yl)ethyl)piperazin-1-yl)picolinonitrile;
3-(2-(4-(2-methoxypyridin-4-yl)piperazin-1-yl)ethyl)-2-oxaspiro[4.5]decan-1-one;
4-(4-(2-(4,4-diethyl-5-oxotetrahydrofuran-2-yl)ethyl)piperazin-1-yl)phenyl acetate;
4-(4-(2-(4,4-diethyl-tetrahydro-5-oxofuran-2-yl)ethyl)piperazin-1-yl)phenyl 2-ethylbutanoate;
4-(4-(2-(4,4-diethyl-tetrahydro-5-oxofuran-2-yl)ethyl)piperazin-1-yl)phenyl isobutyrate;
4-(4-(2-(4,4-diethyl-5-oxotetrahydrofuran-2-yl)ethyl)piperazin-1-yl)phenyl ethyl carbonate;
4-(4-(2-(4,4-diethyl-5-oxotetrahydrofuran-2-yl)ethyl)piperazin-1-yl)phenyl methyl carbonate;
4-(4-(2-(4,4-diethyl-5-oxotetrahydrofuran-2-yl)ethyl)piperazin-1-yl)phenyl isopropyl carbonate;
4-(4-(2-(4,4-diethyl-5-oxotetrahydrofuran-2-yl)ethyl)piperazin-1-yl)phenyl dimethylcarbamate;
4-(4-(2-(4,4-diethyl-5-oxotetrahydrofuran-2-yl)ethyl)piperazin-1-yl)phenyl diethylcarbamate;
5-(2-(7-chloro-3,4-dihydroisoquinolin-2(1H)-yl)ethyl)-3,3-diethyl-dihydrofuran-2(3H)-one;
3,3-diethyl-5-(2-(7-fluoro-3,4-dihydroisoquinolin-2(1H)-yl)ethyl)-dihydrofuran-2(3H)-one; and
5-(2-(7-bromo-3,4-dihydroisoquinolin-2(1H)-yl)ethyl)-3,3-diethyldihydrofuran-2(3H)-one;
or a pharmaceutically acceptable form thereof.

7. The method of claim 1, wherein the compound is selected from the group consisting of:

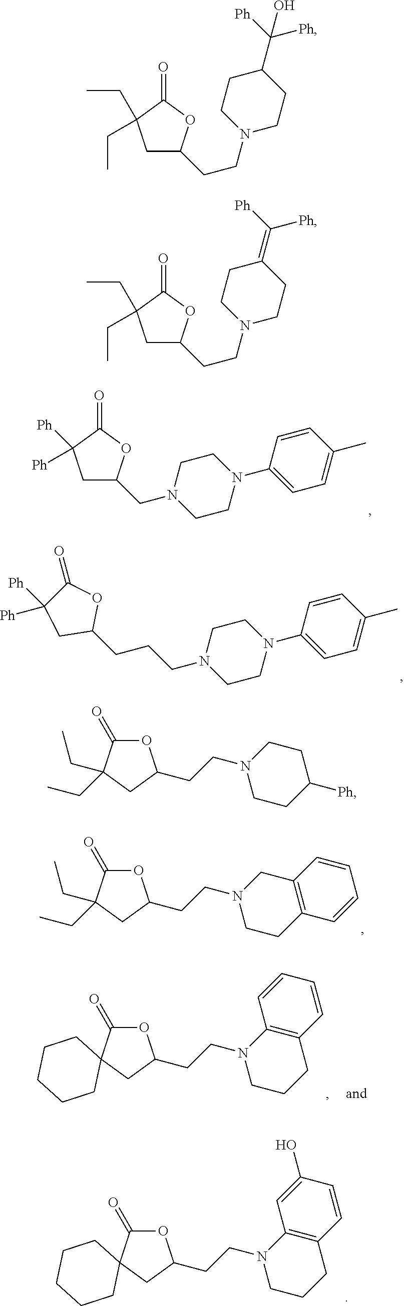

8. The method of claim 1, wherein the at least one compound is selected from the group consisting of:

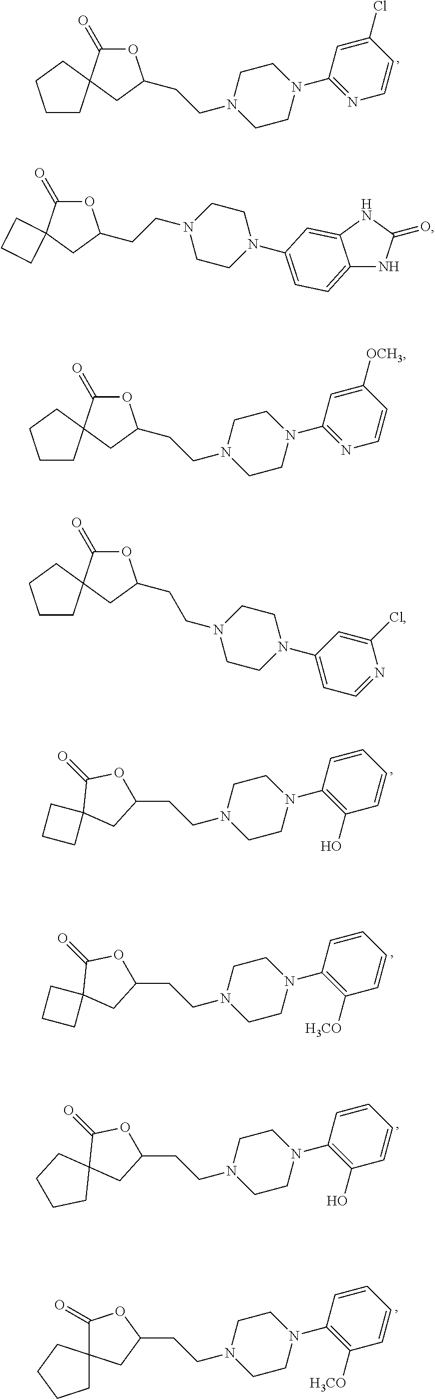

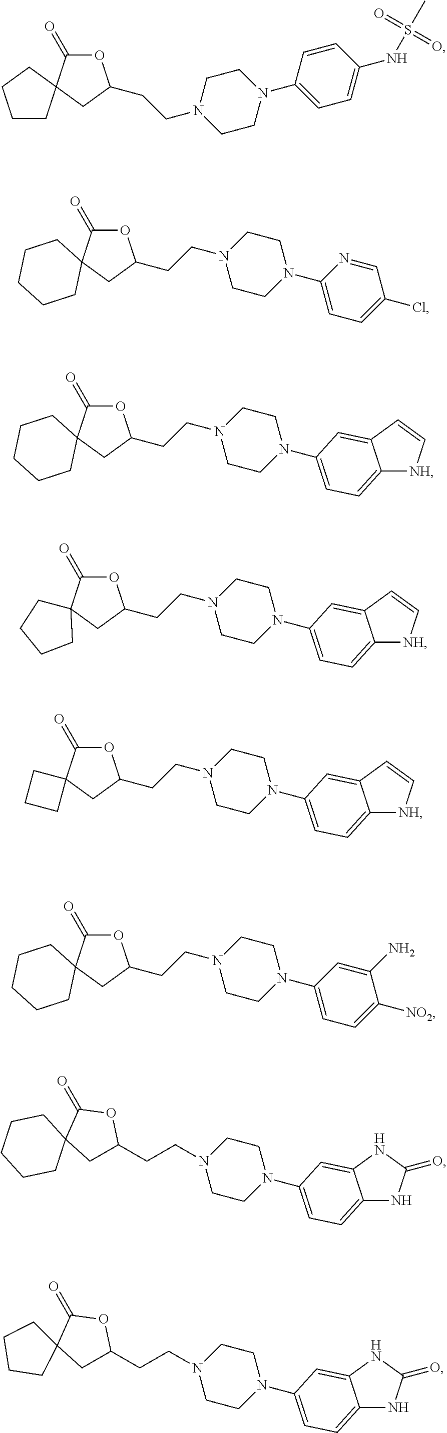

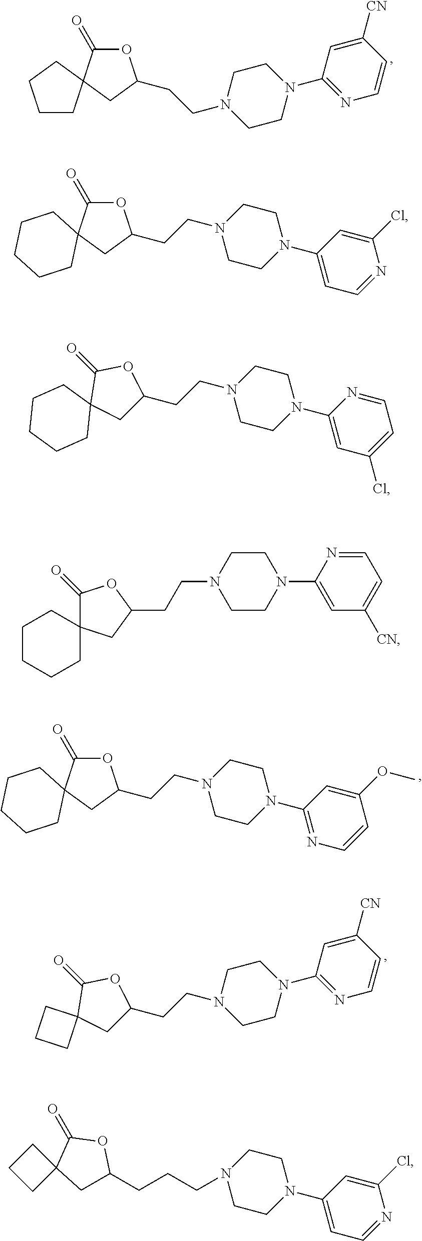

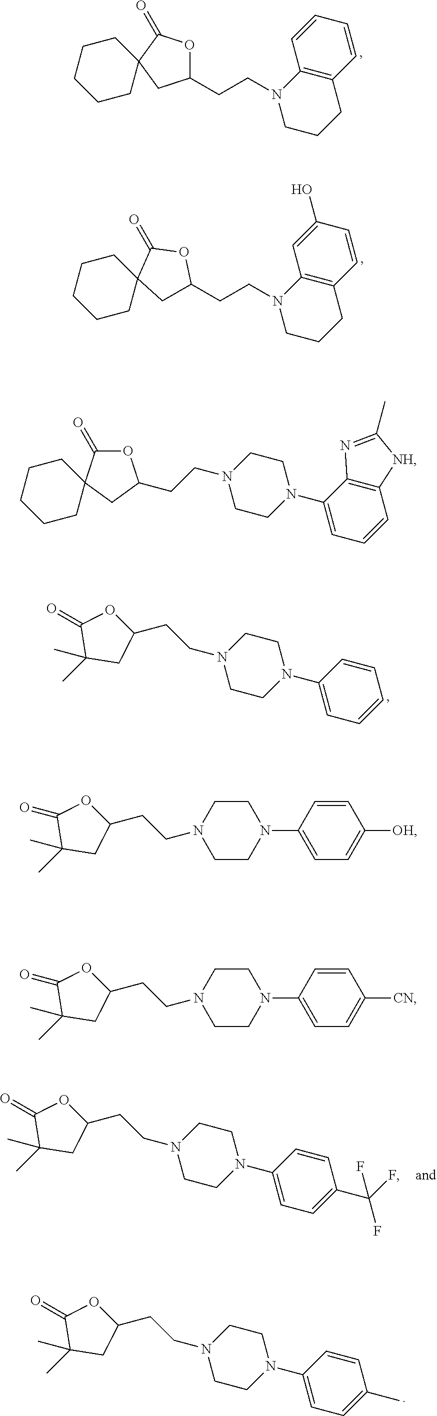

9. The method of claim 1 wherein the at least one compound is administered in a composition further comprising at least one excipient.

10. The method of claim 1, wherein the disease that involves dysregulation of sigma-2 receptor activity is selected from the group consisting of generalized anxiety disorder, social anxiety disorder, panic disorder, agoraphobia, obsessive-compulsive disorder post-traumatic stress disorder, depression, bipolar disorder, anorexia nervosa, bulimia nervosa, a substance use disorder, schizophrenia, Alzheimer's disease, mild cognitive impairment, a memory disorder, and cancer.

11. The method of claim 10, wherein the cancer is selected from the group consisting of pancreatic cancer, lung cancer, breast cancer, melanoma, prostate cancer, and ovarian cancer.
